SEQUENCE LISTING (391 sequences)

SEQ ID NO: 1 - protein, 448 aa, Artificial Sequence
  Description of Artificial Sequence Synthetic polypeptide
EVQLLESGGGLVQPGGSLRLSCAASGFTFSNYWMSWVRQAPGKGLEWVSAISGSGGSTYYADSVKGRFTISRDNSKNTLYLQMNSLRAEDTAVYYCAKDLIGSWGMYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 5 aa, Artificial Sequence
  Description of Artificial Sequence Synthetic peptide
NYWMS

SEQ ID NO: 3 - protein, 17 aa, Artificial Sequence
  Description of Artificial Sequence Synthetic peptide
AISGSGGSTYYADSVKG

SEQ ID NO: 4 - protein, 12 aa, Artificial Sequence
  Description of Artificial Sequence Synthetic peptide
DLIGSWGMYFDY

SEQ ID NO: 5 - protein, 214 aa, Artificial Sequence
  Description of Artificial Sequence Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 11 aa, Artificial Sequence
  Description of Artificial Sequence Synthetic peptide
RASQSVSSYLA

SEQ ID NO: 7 - protein, 7 aa, Artificial Sequence
  Description of Artificial Sequence Synthetic peptide
DASNRAT

SEQ ID NO: 8 - protein, 9 aa, Artificial Sequence
  Description of Artificial Sequence Synthetic peptide
QQRSNWPLT

SEQ ID NO: 9 - protein, 447 aa, Artificial Sequence
  Description of Artificial Sequence Synthetic polypeptide
QVQLVQSGAEVKKPGSSVKVSCKASGGTFSTDAIHWVRQAPGQGLEWMGRIIPISGTANYAQKFQGRVTITADESTSTAYMELSSLRSEDTAVYYCARGRYSYYYYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 5 aa, Artificial Sequence
  Description of Artificial Sequence Synthetic peptide
TDAIH

SEQ ID NO: 11 - protein, 17 aa, Artificial Sequence
  Description of Artificial Sequence Synthetic peptide
RIIPISGTANYAQKFQG

SEQ ID NO: 12 - protein, 11 aa, Artificial Sequence
  Description of Artificial Sequence Synthetic peptide
GRYSYYYYFDY

SEQ ID NO: 13 - protein, 447 aa, Artificial Sequence
  Description of Artificial Sequence Synthetic polypeptide
QVQLVQSGAEVKKPGSSVKVSCKASGGTFSTYAIHWVRQAPGQGLEWMGRIIPIEGRAYYAQKFQGRVTITADESTSTAYMELSSLRSEDTAVYYCARGRYSYRYWFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 5 aa, Artificial Sequence
  Description of Artificial Sequence Synthetic peptide
TYAIH

SEQ ID NO: 15 - protein, 17 aa, Artificial Sequence
  Description of Artificial Sequence Synthetic peptide
RIIPIEGRAYYAQKFQG

SEQ ID NO: 16 - protein, 11 aa, Artificial Sequence
  Description of Artificial Sequence Synthetic peptide
GRYSYRYWFDY

SEQ ID NO: 17 - protein, 447 aa, Artificial Sequence
  Description of Artificial Sequence Synthetic polypeptide
QVQLVQSGAEVKKPGSSVKVSCKASGGTFSDAAIHWVRQAPGQGLEWMGYIIPISGTANYAQKFQGRVTITADESTSTAYMELSSLRSEDTAVYYCARGRARYRWAFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 18 - protein, 5 aa, Artificial Sequence
  Description of Artificial Sequence Synthetic peptide
DAAIH

SEQ ID NO: 19 - protein, 17 aa, Artificial Sequence
  Description of Artificial Sequence Synthetic peptide
YIIPISGTANYAQKFQG

SEQ ID NO: 20 - protein, 11 aa, Artificial Sequence
  Description of Artificial Sequence Synthetic peptide
GRARYRWAFDY

SEQ ID NO: 21 - protein, 454 aa, Artificial Sequence
  Description of Artificial Sequence Synthetic polypeptide
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDGYYSRYRGYFLYYGMDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 5 aa, Artificial Sequence
  Description of Artificial Sequence Synthetic peptide
SYAIS

SEQ ID NO: 23 - protein, 17 aa, Artificial Sequence
  Description of Artificial Sequence Synthetic peptide
GIIPIFGTANYAQKFQG

SEQ ID NO: 24 - protein, 18 aa, Artificial Sequence
  Description of Artificial Sequence Synthetic peptide
DGYYSRYRGYFLYYGMDV

SEQ ID NO: 25 - protein, 444 aa, Artificial Sequence
  Description of Artificial Sequence Synthetic polypeptide
EVQLLESGGGLVQPGGSLRLSCAASGFTFSSYAMSWVRQAPGKGLEWVSAIRYDGGSTYYADSVKGRFTISRDNSKNTLYLQMNSLRAEDTAVYYCAKMFGGQLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 26 - protein, 5 aa, Artificial Sequence
  Description of Artificial Sequence Synthetic peptide
SYAMS

SEQ ID NO: 27 - protein, 17 aa, Artificial Sequence
  Description of Artificial Sequence Synthetic peptide
AIRYDGGSTYYADSVKG

SEQ ID NO: 28 - protein, 8 aa, Artificial Sequence
  Description of Artificial Sequence Synthetic peptide
MFGGQLDY

SEQ ID NO: 29 - protein, 214 aa, Artificial Sequence
  Description of Artificial Sequence Synthetic polypeptide
DIQMTQSPSSLSASVGDRVTITCRASQSIDRHLNWYQQKPGKAPKLLIYKASSLQSGVPSRFSGSGSGTDFTLTISSLQPEDFATYYCQQPHTFPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 11 aa, Artificial Sequence
  Description of Artificial Sequence Synthetic peptide
RASQSIDRHLN

SEQ ID NO: 31 - protein, 7 aa, Artificial Sequence
  Description of Artificial Sequence Synthetic peptide
KASSLQS

SEQ ID NO: 32 - protein, 9 aa, Artificial Sequence
  Description of Artificial Sequence Synthetic peptide
QQPHTFPLT

SEQ ID NO: 33 - protein, 443 aa, Artificial Sequence
  Description of Artificial Sequence Synthetic polypeptide
EVQLLESGGGLVQPGGSLRLSCAASGFTFSNYWMSWVRQAPGKGLEWVSGISYNGGSTYYADSVKGRFTISRDNSKNTLYLQMNSLRAEDTAVYYCAKPYFGFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 34 - protein, 17 aa, Artificial Sequence
  Description of Artificial Sequence Synthetic peptide
GISYNGGSTYYADSVKG

SEQ ID NO: 35 - protein, 7 aa, Artificial Sequence
  Description of Artificial Sequence Synthetic peptide
PYFGFDY

SEQ ID NO: 36 - protein, 214 aa, Artificial Sequence
  Description of Artificial Sequence Synthetic polypeptide
EIVLTQSPATLSLSPGERATLSCRASQSVADFLAWYQQKPGQAPRLLIYDASNRATGIPARFSGSGSGTDFTLTISSLEPEDFAVYYCQQGNRWPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 11 aa, Artificial Sequence
  Description of Artificial Sequence Synthetic peptide
RASQSVADFLA

SEQ ID NO: 38 - protein, 9 aa, Artificial Sequence
  Description of Artificial Sequence Synthetic peptide
QQGNRWPIT

SEQ ID NO: 39 - protein, 443 aa, Artificial Sequence
  Description of Artificial Sequence Synthetic polypeptide
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARTGNKLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 40 - protein, 7 aa, Artificial Sequence
  Description of Artificial Sequence Synthetic peptide
TGNKLDY

SEQ ID NO: 41 - protein, 214 aa, Artificial Sequence
  Description of Artificial Sequence Synthetic polypeptide
EIVLTQSPATLSLSPGERATLSCRASQSVAKFLAWYQQKPGQAPRLLIYFASNRATDIPARFSGSGSGTDFTLTISSLEPEDFAVYYCQQRDHWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 11 aa, Artificial Sequence
  Description of Artificial Sequence Synthetic peptide
RASQSVAKFLA

SEQ ID NO: 43 - protein, 7 aa, Artificial Sequence
  Description of Artificial Sequence Synthetic peptide
FASNRAT

SEQ ID NO: 44 - protein, 9 aa, Artificial Sequence
  Description of Artificial Sequence Synthetic peptide
QQRDHWPLT

SEQ ID NO: 45 - protein, 445 aa, Artificial Sequence
  Description of Artificial Sequence Synthetic polypeptide
QVQLVQSGAEVKKPGSSVKVSCKASGGTFSSYGISWVRQAPGQGLEWMGWISAIFGTANYAQKFQGRVTITADESTSTAYMELSSLRSEDTAVYYCARGVASWYLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 46 - protein, 5 aa, Artificial Sequence
  Description of Artificial Sequence Synthetic peptide
SYGIS

SEQ ID NO: 47 - protein, 17 aa, Artificial Sequence
  Description of Artificial Sequence Synthetic peptide
WISAIFGTANYAQKFQG

SEQ ID NO: 48 - protein, 9 aa, Artificial Sequence
  Description of Artificial Sequence Synthetic peptide
GVASWYLDY

SEQ ID NO: 49 - protein, 214 aa, Artificial Sequence
  Description of Artificial Sequence Synthetic polypeptide
DIQMTQSPSSLSASVGDRVTITCRASQSINKWLNWYQQKPGKAPKLLIYAASSLQSGVPSRFSGSGSGTDFTLTISSLQPEDFATYYCQQDYNFPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 11 aa, Artificial Sequence
  Description of Artificial Sequence Synthetic peptide
RASQSINKWLN

SEQ ID NO: 51 - protein, 7 aa, Artificial Sequence
  Description of Artificial Sequence Synthetic peptide
AASSLQS

SEQ ID NO: 52 - protein, 9 aa, Artificial Sequence
  Description of Artificial Sequence Synthetic peptide
QQDYNFPYT

SEQ ID NO: 53 - protein, 450 aa, Artificial Sequence
  Description of Artificial Sequence Synthetic polypeptide
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARVFASRSGYGIPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 54 - protein, 5 aa, Artificial Sequence
  Description of Artificial Sequence Synthetic peptide
SYWIG

SEQ ID NO: 55 - protein, 17 aa, Artificial Sequence
  Description of Artificial Sequence Synthetic peptide
IIYPGDSDTRYSPSFQG

SEQ ID NO: 56 - protein, 14 aa, Artificial Sequence
  Description of Artificial Sequence Synthetic peptide
VFASRSGYGIPFDY

SEQ ID NO: 57 - protein, 220 aa, Artificial Sequence
  Description of Artificial Sequence Synthetic polypeptide
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 17 aa, Artificial Sequence
  Description of Artificial Sequence Synthetic peptide
KSSQSVLYSSNNKNYLA

SEQ ID NO: 59 - protein, 7 aa, Artificial Sequence
  Description of Artificial Sequence Synthetic peptide
WASTRES

SEQ ID NO: 60 - protein, 9 aa, Artificial Sequence
  Description of Artificial Sequence Synthetic peptide
QQYYSTPLT

SEQ ID NO: 61 - protein, 449 aa, Artificial Sequence
  Description of Artificial Sequence Synthetic polypeptide
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARDVGRWAPGGFLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 62 - protein, 13 aa, Artificial Sequence
  Description of Artificial Sequence Synthetic peptide
DVGRWAPGGFLDY

SEQ ID NO: 63 - protein, 447 aa, Artificial Sequence
  Description of Artificial Sequence Synthetic polypeptide
QVQLVQSGAEVKKPGSSVKVSCKASGGTFDSDAIHWVRQAPGQGLEWMGYIIPISGTANYAQKFQGRVTITADESTSTAYMELSSLRSEDTAVYYCARGRYSYRWTFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 64 - protein, 5 aa, Artificial Sequence
  Description of Artificial Sequence Synthetic peptide
SDAIH

SEQ ID NO: 65 - protein, 11 aa, Artificial Sequence
  Description of Artificial Sequence Synthetic peptide
GRYSYRWTFDY

SEQ ID NO: 66 - protein, 447 aa, Artificial Sequence
  Description of Artificial Sequence Synthetic polypeptide
QVQLVQSGAEVKKPGSSVKVSCKASGGTFKSAYIHWVRQAPGQGLEWMGYISPYYGSAYYAQKFQGRVTITADESTSTAYMELSSLRSEDTAVYYCARGRIAYYYVFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 67 - protein, 5 aa, Artificial Sequence
  Description of Artificial Sequence Synthetic peptide
SAYIH

SEQ ID NO: 68 - protein, 17 aa, Artificial Sequence
  Description of Artificial Sequence Synthetic peptide
YISPYYGSAYYAQKFQG

SEQ ID NO: 69 - protein, 11 aa, Artificial Sequence
  Description of Artificial Sequence Synthetic peptide
GRIAYYYVFDY

SEQ ID NO: 70 - protein, 447 aa, Artificial Sequence
  Description of Artificial Sequence Synthetic polypeptide
QVQLVQSGAEVKKPGSSVKVSCKASGGTFSTDAIHWVRQAPGQGLEWMGYIIPISGNANYAQKFQGRVTITADESTSTAYMELSSLRSEDTAVYYCARGRYAYNWGFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 71 - protein, 17 aa, Artificial Sequence
  Description of Artificial Sequence Synthetic peptide
YIIPISGNANYAQKFQG

SEQ ID NO: 72 - protein, 11 aa, Artificial Sequence
  Description of Artificial Sequence Synthetic peptide
GRYAYNWGFDY

SEQ ID NO: 73 - protein, 446 aa, Artificial Sequence
  Description of Artificial Sequence Synthetic polypeptide
EVQLVQSGAEVKKPGESLKISCKGSGYSFTSYWIGWVRQMPGKGLEWMGIIDPGDSDTRYSPSFQGQVTISADKSISTAYLQWSSLKASDTAMYYCARGNDPWPDFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 74 - protein, 17 aa, Artificial Sequence
  Description of Artificial Sequence Synthetic peptide
IIDPGDSDTRYSPSFQG

SEQ ID NO: 75 - protein, 10 aa, Artificial Sequence
  Description of Artificial Sequence Synthetic peptide
GNDPWPDFDY

SEQ ID NO: 76 - protein, 214 aa, Artificial Sequence
  Description of Artificial Sequence Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - protein, 11 aa, Artificial Sequence
  Description of Artificial Sequence Synthetic peptide
RASQSISSYLN

SEQ ID NO: 78 - protein, 9 aa, Artificial Sequence
  Description of Artificial Sequence Synthetic peptide
QQSYSTPLT

SEQ ID NO: 79 - protein, 445 aa, Artificial Sequence
  Description of Artificial Sequence Synthetic polypeptide
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGQDESGLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 80 - protein, 9 aa, Artificial Sequence
  Description of Artificial Sequence Synthetic peptide
GQDESGLDY

SEQ ID NO: 81 - protein, 214 aa, Artificial Sequence
  Description of Artificial Sequence Synthetic polypeptide
EIVLTQSPATLSLSPGERATLSCRASQSVSDWLAWYQQKPGQAPRLLIYDASNRATGIPARFSGSGSGTDFTLTISSLEPEDFAVYYCQQSGDWPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - protein, 11 aa, Artificial Sequence
  Description of Artificial Sequence Synthetic peptide
RASQSVSDWLA

SEQ ID NO: 83 - protein, 9 aa, Artificial Sequence
  Description of Artificial Sequence Synthetic peptide
QQSGDWPYT

SEQ ID NO: 84 - protein, 444 aa, Artificial Sequence
  Description of Artificial Sequence Synthetic polypeptide
QVQLVQSGAEVKKPGSSVKVSCKASGGTFSSYAISWVRQAPGQGLEWMGGISAIFGNANYAQKFQGRVTITADESTSTAYMELSSLRSEDTAVYYCARLRLGNLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 85 - protein, 17 aa, Artificial Sequence
  Description of Artificial Sequence Synthetic peptide
GISAIFGNANYAQKFQG

SEQ ID NO: 86 - protein, 8 aa, Artificial Sequence
  Description of Artificial Sequence Synthetic peptide
LRLGNLDY

SEQ ID NO: 87 - protein, 214 aa, Artificial Sequence
  Description of Artificial Sequence Synthetic polypeptide
EIVLTQSPATLSLSPGERATLSCRASQSVANFLAWYQQKPGQAPRLLIYFASNRATGIPARFSGSGSGTDFTLTISSLEPEDFAVYYCQQYYGAPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 88 - protein, 11 aa, Artificial Sequence
  Description of Artificial Sequence Synthetic peptide
RASQSVANFLA

SEQ ID NO: 89 - protein, 9 aa, Artificial Sequence
  Description of Artificial Sequence Synthetic peptide
QQYYGAPIT

SEQ ID NO: 90 - protein, 452 aa, Artificial Sequence
  Description of Artificial Sequence Synthetic polypeptide
EVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGK

SEQ ID NO: 91 - protein, 215 aa, Artificial Sequence
  Description of Artificial Sequence Synthetic polypeptide
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 92 - protein, 10 aa, Artificial Sequence
  Description of Artificial Sequence Synthetic peptide
GFTFNTYAMN

SEQ ID NO: 93 - protein, 12 aa, Artificial Sequence
  Description of Artificial Sequence Synthetic peptide
RIRSKYNNYATY

SEQ ID NO: 94 - protein, 14 aa, Artificial Sequence
  Description of Artificial Sequence Synthetic peptide
HGNFGNSYVSWFAY

SEQ ID NO: 95 - protein, 14 aa, Artificial Sequence
  Description of Artificial Sequence Synthetic peptide
RSSTGAVTTSNYAN

SEQ ID NO: 96 - protein, 7 aa, Artificial Sequence
  Description of Artificial Sequence Synthetic peptide
GTNKRAP

SEQ ID NO: 97 - protein, 9 aa, Artificial Sequence
  Description of Artificial Sequence Synthetic peptide
ALWYSNLWV

SEQ ID NO: 98 - protein, 64 aa, Unknown
  Description of Unknown Human or mouse polypeptide
TVSVTKSGRQCQPWNSQYPHTHTFTALRFPELNGGHSYCRNPGNQKEAPWCFTLDENFKSDLCD

SEQ ID NO: 99 - protein, 406 aa, Artificial Sequence
  Description of Artificial Sequence Synthetic polypeptide
QETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACDSKDSKEKNKMEILFGCFCGFILIGLILYISLAIRGGGGSGGGS

SEQ ID NO: 100 - protein, 400 aa, Artificial Sequence
  Description of Artificial Sequence Synthetic polypeptide
EVEVLDPNDPLGPLDGQDGPIPTLKGYFLNFLEPVNNITIVQGQTAILHCKVAGNPPPNVRWLKNDAPVVQEPRRIIIRKTEYGSRLRIQDLDTTDTGYYQCVATNGMKTITATGVLFVRLGPTHSPNHNFQDDYHEDGFCQPYRGIACARFIGNRTIYVDSLQMQGEIENRITAAFTMIGTSTHLSDQCSQFAIPSFCHFVFPLCDARSRTPKPRELCRDECEVLESDLCRQEYTIARSNPLILMRLQLPKCEALPMPESPDAANCMRIGIPAERLGRYHQCYNGSGMDYRGTASTTKSGHQCQPWALQHPHSHHLSSTDFPELGGGHAYCRNPGGQMEGPWCFTQNKNVRMELCDVPSCSPRDSSKMGFGCFCGFILIGLILYISLAIRGGGGSGGGS

SEQ ID NO: 101 - protein, 447 aa, Artificial Sequence; identical to SEQ ID NO: 9
  Description of Artificial Sequence Synthetic polypeptide

SEQ ID NO: 102 - protein, 214 aa, Artificial Sequence
  Description of Artificial Sequence Synthetic polypeptide
EIVLTQSPATLSLSPGERATLSCRASQSVSSYLEWYQQKPGQAPRLLIYDASNRATGIPARFSGSGSGTDFTLTISSLEPEDFAVYYCQQRSNWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - protein, 5 aa, Artificial Sequence
  Description of Artificial Sequence Synthetic peptide
TDAIH

SEQ ID NO: 104 - protein, 17 aa, Artificial Sequence
  Description of Artificial Sequence Synthetic peptide
RIIPISGTANYAQKFQG

SEQ ID NO: 105 - protein, 11 aa, Artificial Sequence
  Description of Artificial Sequence Synthetic peptide
GRYSYYYYFDY

SEQ ID NO: 106 - protein, 11 aa, Artificial Sequence
  Description of Artificial Sequence Synthetic peptide
RASQSVSSYLE

SEQ ID NO: 107 - protein, 7 aa, Artificial Sequence
  Description of Artificial Sequence Synthetic peptide
DASNRAT

SEQ ID NO: 108 - protein, 9 aa, Artificial Sequence
  Description of Artificial Sequence Synthetic peptide
QQRSNWPLT

SEQ ID NO: 109 - protein, 447 aa, Artificial Sequence; identical to SEQ ID NO: 17
  Description of Artificial Sequence Synthetic polypeptide

SEQ ID NO: 110 - protein, 214 aa, Artificial Sequence
  Description of Artificial Sequence Synthetic polypeptide
EIVLTQSPATLSLSPGERATLSCRASQSVRNYLAWYQQKPGQAPRLLIYDASNRATGIPARFSGSGSGTDFTLTISSLEPEDFAVYYCQQRSNWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 111 - protein, 5 aa, Artificial Sequence
  Description of Artificial Sequence Synthetic peptide
DAAIH

SEQ ID NO: 112 - protein, 17 aa, Artificial Sequence
  Description of Artificial Sequence Synthetic peptide
YIIPISGTANYAQKFQG

SEQ ID NO: 113 - protein, 11 aa, Artificial Sequence
  Description of Artificial Sequence Synthetic peptide
GRARYRWAFDY

SEQ ID NO: 114 - protein, 11 aa, Artificial Sequence
  Description of Artificial Sequence Synthetic peptide
RASQSVRNYLA

SEQ ID NO: 115 - protein, 7 aa, Artificial Sequence
  Description of Artificial Sequence Synthetic peptide
DASNRAT

SEQ ID NO: 116 - protein, 9 aa, Artificial Sequence
  Description of Artificial Sequence Synthetic peptide
QQRSNWPLT

SEQ ID NO: 117 - protein, 447 aa, Artificial Sequence; identical to SEQ ID NO: 17
  Description of Artificial Sequence Synthetic polypeptide

SEQ ID NO: 118 - protein, 214 aa, Artificial Sequence
  Description of Artificial Sequence Synthetic polypeptide
EIVLTQSPATLSLSPGERATLSCRASQSVSTYLAWYQQKPGQAPRLLIYDASNRATGIPARFSGSGSGTDFTLTISSLEPEDFAVYYCQQRSNWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 119 - protein, 5 aa, Artificial Sequence
  Description of Artificial Sequence Synthetic peptide
DAAIH

SEQ ID NO: 120 - protein, 17 aa, Artificial Sequence
  Description of Artificial Sequence Synthetic peptide
YIIPISGTANYAQKFQG

SEQ ID NO: 121 - protein, 11 aa, Artificial Sequence
  Description of Artificial Sequence Synthetic peptide
GRARYRWAFDY

SEQ ID NO: 122 - protein, 11 aa, Artificial Sequence
  Description of Artificial Sequence Synthetic peptide
RASQSVSTYLA

SEQ ID NO: 123 - protein, 7 aa, Artificial Sequence
  Description of Artificial Sequence Synthetic peptide
DASNRAT

SEQ ID NO: 124 - protein, 9 aa, Artificial Sequence
  Description of Artificial Sequence Synthetic peptide
QQRSNWPLT

SEQ ID NO: 125 - protein, 447 aa, Artificial Sequence; identical to SEQ ID NO: 17
  Description of Artificial Sequence Synthetic polypeptide

SEQ ID NO: 126 - protein, 214 aa, Artificial Sequence
  Description of Artificial Sequence Synthetic polypeptide
EIVLTQSPATLSLSPGERATLSCRASQSVRNYLAWYQQKPGQAPRLLIEDASNRATGIPARFSGSGSGTDFTLTISSLEPEDFAVYYCQQRSNWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 127 - protein, 5 aa, Artificial Sequence
  Description of Artificial Sequence Synthetic peptide
DAAIH

SEQ ID NO: 128 - protein, 17 aa, Artificial Sequence
  Description of Artificial Sequence Synthetic peptide
YIIPISGTANYAQKFQG

SEQ ID NO: 129 - protein, 11 aa, Artificial Sequence
  Description of Artificial Sequence Synthetic peptide
GRARYRWAFDY

SEQ ID NO: 130 - protein, 11 aa, Artificial Sequence
  Description of Artificial Sequence Synthetic peptide
RASQSVRNYLA

SEQ ID NO: 131 - protein, 7 aa, Artificial Sequence
  Description of Artificial Sequence Synthetic peptide
DASNRAT

SEQ ID NO: 132 - protein, 9 aa, Artificial Sequence
  Description of Artificial Sequence Synthetic peptide
QQRSNWPLT

SEQ ID NO: 133 - protein, 447 aa, Artificial Sequence; identical to SEQ ID NO: 70
  Description of Artificial Sequence Synthetic polypeptide

SEQ ID NO: 134 - protein, 214 aa, Artificial Sequence
  Description of Artificial Sequence Synthetic polypeptide
EIVLTQSPATLSLSPGERATLSCRASQSVSSYLAWYQQKPGQAPRLLIKDASNRATGIPARFSGSGSGTDFTLTISSLEPEDFAVYYCQQRSNWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 135 - protein, 5 aa, Artificial Sequence
  Description of Artificial Sequence Synthetic peptide
TDAIH

SEQ ID NO: 136 - protein, 17 aa, Artificial Sequence
  Description of Artificial Sequence Synthetic peptide
YIIPISGNANYAQKFQG

SEQ ID NO: 137 - protein, 11 aa, Artificial Sequence
  Description of Artificial Sequence Synthetic peptide
GRYAYNWGFDY

SEQ ID NO: 138 - protein, 11 aa, Artificial Sequence
  Description of Artificial Sequence Synthetic peptide
RASQSVSSYLA

SEQ ID NO: 139 - protein, 7 aa, Artificial Sequence
  Description of Artificial Sequence Synthetic peptide
DASNRAT

SEQ ID NO: 140 - protein, 9 aa, Artificial Sequence
  Description of Artificial Sequence Synthetic peptide
QQRSNWPLT

SEQ ID NO: 141 - protein, 447 aa, Artificial Sequence; identical to SEQ ID NO: 70
  Description of Artificial Sequence Synthetic polypeptide

SEQ ID NO: 142 - protein, 214 aa, Artificial Sequence
  Description of Artificial Sequence Synthetic polypeptide
EIVLTQSPATLSLSPGERATLSCRASQSVRNYLAWYQQKPGQAPRLLIKDASNRATGIPARFSGSGSGTDFTLTISSLEPEDFAVYYCQQRSNWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 143 - protein, 5 aa, Artificial Sequence
  Description of Artificial Sequence Synthetic peptide
TDAIH

SEQ ID NO: 144 - protein, 17 aa, Artificial Sequence
  Description of Artificial Sequence Synthetic peptide
YIIPISGNANYAQKFQG

SEQ ID NO: 145 - protein, 11 aa, Artificial Sequence
  Description of Artificial Sequence Synthetic peptide
GRYAYNWGFDY

SEQ ID NO: 146 - protein, 11 aa, Artificial Sequence
  Description of Artificial Sequence Synthetic peptide
RASQSVRNYLA

SEQ ID NO: 147 - protein, 7 aa, Artificial Sequence
  Description of Artificial Sequence Synthetic peptide
DASNRAT

SEQ ID NO: 148 - protein, 9 aa, Artificial Sequence
  Description of Artificial Sequence Synthetic peptide
QQRSNWPLT

SEQ ID NO: 149 - protein, 447 aa, Artificial Sequence; identical to SEQ ID NO: 70
  Description of Artificial Sequence Synthetic polypeptide

SEQ ID NO: 150 - protein, 214 aa, Artificial Sequence
  Description of Artificial Sequence Synthetic polypeptide
EIVLTQSPATLSLSPGERATLSCRASQSVRSYLAWYQQKPGQAPRLLIKDASDRATGIPARFSGSGSGTDFTLTISSLEPEDFAVYYCQQRSNWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 151 - protein, 5 aa, Artificial Sequence
  Description of Artificial Sequence Synthetic peptide
TDAIH

SEQ ID NO: 152 - protein, 17 aa, Artificial Sequence
  Description of Artificial Sequence Synthetic peptide
YIIPISGNANYAQKFQG

SEQ ID NO: 153 - protein, 11 aa, Artificial Sequence
  Description of Artificial Sequence Synthetic peptide
GRYAYNWGFDY

SEQ ID NO: 154 - protein, 11 aa, Artificial Sequence
  Description of Artificial Sequence Synthetic peptide
RASQSVRSYLA

SEQ ID NO: 155 - protein, 7 aa, Artificial Sequence
  Description of Artificial Sequence Synthetic peptide
DASDRAT

SEQ ID NO: 156 - protein, 9 aa, Artificial Sequence
  Description of Artificial Sequence Synthetic peptide
QQRSNWPLT

SEQ ID NO: 157 - protein, 447 aa, Artificial Sequence; identical to SEQ ID NO: 70
  Description of Artificial Sequence Synthetic polypeptide

SEQ ID NO: 158 - protein, 214 aa, Artificial Sequence
  Description of Artificial Sequence Synthetic polypeptide
EIVLTQSPATLSLSPGERATLSCRASQSVSSYLAWYQQKPGQAPRLLIKDASTRATGIPARFSGSGSGTDFTLTISSLEPEDFAVYYCQQRSNWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 159 - protein, 5 aa, Artificial Sequence
  Description of Artificial Sequence Synthetic peptide
TDAIH

SEQ ID NO: 160 - protein, 17 aa, Artificial Sequence
  Description of Artificial Sequence Synthetic peptide
YIIPISGNANYAQKFQG

SEQ ID NO: 161 - protein, 11 aa, Artificial Sequence
  Description of Artificial Sequence Synthetic peptide
GRYAYNWGFDY

SEQ ID NO: 162 - protein, 11 aa, Artificial Sequence
  Description of Artificial Sequence Synthetic peptide
RASQSVSSYLA

SEQ ID NO: 163 - protein, 7 aa, Artificial Sequence
  Description of Artificial Sequence Synthetic peptide
DASTRAT

SEQ ID NO: 164 - protein, 9 aa, Artificial Sequence
  Description of Artificial Sequence Synthetic peptide
QQRSNWPLT

SEQ ID NO: 165 - protein, 447 aa, Artificial Sequence; identical to SEQ ID NO: 70
  Description of Artificial Sequence Synthetic polypeptide

SEQ ID NO: 166 - protein, 214 aa, Artificial Sequence
  Description of Artificial Sequence Synthetic polypeptide
EIVLTQSPATLSLSPGERATLSCRASQSVNTYLAWYQQKPGQAPRLLIKDASTRATGIPARFSGSGSGTDFTLTISSLEPEDFAVYYCQQRSNWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 167 - protein, 5 aa, Artificial Sequence
  Description of Artificial Sequence Synthetic peptide
TDAIH

SEQ ID NO: 168 - protein, 17 aa, Artificial Sequence
  Description of Artificial Sequence Synthetic peptide
YIIPISGNANYAQKFQG

SEQ ID NO: 169 - protein, 11 aa, Artificial Sequence
  Description of Artificial Sequence Synthetic peptide
GRYAYNWGFDY

SEQ ID NO: 170 - protein, 11 aa, Artificial Sequence
  Description of Artificial Sequence Synthetic peptide
RASQSVNTYLA

SEQ ID NO: 171 - protein, 7 aa, Artificial Sequence
  Description of Artificial Sequence Synthetic peptide
DASTRAT

SEQ ID NO: 172 - protein, 9 aa, Artificial Sequence
  Description of Artificial Sequence Synthetic peptide
QQRSNWPLT

SEQ ID NO: 173 - protein, 447 aa, Artificial Sequence; identical to SEQ ID NO: 70
  Description of Artificial Sequence Synthetic polypeptide

SEQ ID NO: 174 - protein, 214 aa, Artificial Sequence
  Description of Artificial Sequence Synthetic polypeptide
EIVLTQSPATLSLSPGERATLSCRASQSVSSYLAWYQQKPGQAPRLLIKDASYRATGIPARFSGSGSGTDFTLTISSLEPEDFAVYYCQQRSNWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 175 - protein, 5 aa, Artificial Sequence
  Description of Artificial Sequence Synthetic peptide
TDAIH

SEQ ID NO: 176 - protein, 17 aa, Artificial Sequence
  Description of Artificial Sequence Synthetic peptide
YIIPISGNANYAQKFQG

SEQ ID NO: 177 - protein, 11 aa, Artificial Sequence
  Description of Artificial Sequence Synthetic peptide
GRYAYNWGFDY

SEQ ID NO: 178 - protein, 11 aa, Artificial Sequence
  Description of Artificial Sequence Synthetic peptide
RASQSVSSYLA

SEQ ID NO: 179 - protein, 7 aa, Artificial Sequence
  Description of Artificial Sequence Synthetic peptide
DASYRAT

SEQ ID NO: 180 - protein, 9 aa, Artificial Sequence
  Description of Artificial Sequence Synthetic peptide
QQRSNWPLT

SEQ ID NO: 181 - protein, 447 aa, Artificial Sequence; identical to SEQ ID NO: 66
  Description of Artificial Sequence Synthetic polypeptide

SEQ ID NO: 182 - protein, 214 aa, Artificial Sequence
  Description of Artificial Sequence Synthetic polypeptide
EIVLTQSPATLSLSPGERATLSCRASQSVSSYLAWYQQKPGQAPRLLIYDASYRATGIPARFSGSGSGTDFTLTISSLEPEDFAVYYCQQRSNWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 183 - protein, 5 aa, Artificial Sequence
  Description of Artificial Sequence Synthetic peptide
SAYIH

SEQ ID NO: 184 - protein, 17 aa, Artificial Sequence
  Description of Artificial Sequence Synthetic peptide
YISPYYGSAYYAQKFQG

SEQ ID NO: 185 - protein, 11 aa, Artificial Sequence
  Description of Artificial Sequence Synthetic peptide
GRIAYYYVFDY

SEQ ID NO: 186 - protein, 11 aa, Artificial Sequence
  Description of Artificial Sequence Synthetic peptide
RASQSVSSYLA

SEQ ID NO: 187 - protein, 7 aa, Artificial Sequence
  Description of Artificial Sequence Synthetic peptide
DASYRAT

SEQ ID NO: 188 - protein, 9 aa, Artificial Sequence
  Description of Artificial Sequence Synthetic peptide
QQRSNWPLT

SEQ ID NO: 189 - protein, 447 aa, Artificial Sequence; identical to SEQ ID NO: 13
  Description of Artificial Sequence Synthetic polypeptide

SEQ ID NO: 190 - protein, 214 aa, Artificial Sequence
  Description of Artificial Sequence Synthetic polypeptide
EIVLTQSPATLSLSPGERATLSCRASQSVSSYLAWYQQKPGQAPRLLIYDASSRATGIPARFSGSGSGTDFTLTISSLEPEDFAVYYCQQRSNWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 191 - protein, 5 aa, Artificial Sequence
  Description of Artificial Sequence Synthetic peptide
TYAIH

SEQ ID NO: 192 - protein, 17 aa, Artificial Sequence
  Description of Artificial Sequence Synthetic peptide
RIIPIEGRAYYAQKFQG

SEQ ID NO: 193 - protein, 11 aa, Artificial Sequence
  Description of Artificial Sequence Synthetic peptide
GRYSYRYWFDY

SEQ ID NO: 194 - protein, 11 aa, Artificial Sequence
  Description of Artificial Sequence Synthetic peptide
RASQSVSSYLA

SEQ ID NO: 195 - protein, 7 aa, Artificial Sequence
  Description of Artificial Sequence Synthetic peptide
DASSRAT

SEQ ID NO: 196 - protein, 9 aa, Artificial Sequence
  Description of Artificial Sequence Synthetic peptide
QQRSNWPLT

SEQ ID NO: 197 - protein, 447 aa, Artificial Sequence; identical to SEQ ID NO: 17
  Description of Artificial Sequence Synthetic polypeptide

SEQ ID NO: 198 - protein, 214 aa, Artificial Sequence
  Description of Artificial Sequence Synthetic polypeptide
EIVLTQSPATLSLSPGERATLSCRASQSVNNYLAWYQQKPGQAPRLLIYDASYRATGIPARFSGSGSGTDFTLTISSLEPEDFAVYYCQQRSNWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 199 - protein, 5 aa, Artificial Sequence
  Description of Artificial Sequence Synthetic peptide
DAAIH

SEQ ID NO: 200 - protein, 17 aa, Artificial Sequence
  Description of Artificial Sequence Synthetic peptide
YIIPISGTANYAQKFQG

SEQ ID NO: 201 - protein, 11 aa, Artificial Sequence
  Description of Artificial Sequence Synthetic peptide
GRARYRWAFDY

SEQ ID NO: 202 - protein, 11 aa, Artificial Sequence
  Description of Artificial Sequence Synthetic peptide
RASQSVNNYLA

SEQ ID NO: 203 - protein, 7 aa, Artificial Sequence
  Description of Artificial Sequence Synthetic peptide
DASYRAT

SEQ ID NO: 204 - protein, 9 aa, Artificial Sequence
  Description of Artificial Sequence Synthetic peptide
QQRSNWPLT

SEQ ID NO: 205 - protein, 447 aa, Artificial Sequence; identical to SEQ ID NO: 13
  Description of Artificial Sequence Synthetic polypeptide

SEQ ID NO: 206 - protein, 214 aa, Artificial Sequence
  Description of Artificial Sequence Synthetic polypeptide
EIVLTQSPATLSLSPGERATLSCRASQSVSTYLAWYQQKPGQAPRLLIHDASYRATGIPARFSGSGSGTDFTLTISSLEPEDFAVYYCQQRAHWPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 207 - protein, 5 aa, Artificial Sequence
  Description of Artificial Sequence Synthetic peptide
TYAIH

SEQ ID NO: 208 - protein, 17 aa, Artificial Sequence
  Description of Artificial Sequence Synthetic peptide
RIIPIEGRAYYAQKFQG

SEQ ID NO: 209 - protein, 11 aa, Artificial Sequence
  Description of Artificial Sequence Synthetic peptide
GRYSYRYWFDY

SEQ ID NO: 210 - protein, 11 aa, Artificial Sequence
  Description of Artificial Sequence Synthetic peptide
RASQSVSTYLA

SEQ ID NO: 211 - protein, 7 aa, Artificial Sequence
  Description of Artificial Sequence Synthetic peptide
DASYRAT

SEQ ID NO: 212 - protein, 9 aa, Artificial Sequence
  Description of Artificial Sequence Synthetic peptide
QQRAHWPIT

SEQ ID NO: 213 - protein, 447 aa, Artificial Sequence; identical to SEQ ID NO: 9
  Description of Artificial Sequence Synthetic polypeptide

SEQ ID NO: 214 - protein, 214 aa, Artificial Sequence
  Description of Artificial Sequence Synthetic polypeptide
EIVLTQSPATLSLSPGERATLSCRASQSVSTYLAWYQQKPGQAPRLLIYDASNRATGIPARFSGSGSGTDFTLTISSLEPEDFAVYYCQQRAHWPNTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 215 - protein, 5 aa, Artificial Sequence
  Description of Artificial Sequence Synthetic peptide
TDAIH

SEQ ID NO: 216 - protein, 17 aa, Artificial Sequence
  Description of Artificial Sequence Synthetic peptide
RIIPISGTANYAQKFQG

SEQ ID NO: 217 - protein, 11 aa, Artificial Sequence
  Description of Artificial Sequence Synthetic peptide
GRYSYYYYFDY

SEQ ID NO: 218 - protein, 11 aa, Artificial Sequence
  Description of Artificial Sequence Synthetic peptide
RASQSVSTYLA

SEQ ID NO: 219 - protein, 7 aa, Artificial Sequence
  Description of Artificial Sequence Synthetic peptide
DASNRAT

SEQ ID NO: 220 - protein, 9 aa, Artificial Sequence
  Description of Artificial Sequence Synthetic peptide
QQRAHWPNT

SEQ ID NO: 221 - protein, 447 aa, Artificial Sequence; identical to SEQ ID NO: 13
  Description of Artificial Sequence Synthetic polypeptide

SEQ ID NO: 222 - protein, 214 aa, Artificial Sequence
  Description of Artificial Sequence Synthetic polypeptide
EIVLTQSPATLSLSPGERATLSCRASQSVSTYLAWYQQKPGQAPRLLIYDASNRATGIPARFSGSGSGTDFTLTISSLEPEDFAVYYCQQRAHSPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 223 - protein, 5 aa, Artificial Sequence
  Description of Artificial Sequence Synthetic peptide
TYAIH

SEQ ID NO: 224 - protein, 17 aa, Artificial Sequence
  Description of Artificial Sequence Synthetic peptide
RIIPIEGRAYYAQKFQG

SEQ ID NO: 225 - protein, 11 aa, Artificial Sequence
  Description of Artificial Sequence Synthetic peptide
GRYSYRYWFDY

SEQ ID NO: 226 - protein, 11 aa, Artificial Sequence
  Description of Artificial Sequence Synthetic peptide
RASQSVSTYLA

SEQ ID NO: 227 - protein, 7 aa, Artificial Sequence
  Description of Artificial Sequence Synthetic peptide
DASNRAT

SEQ ID NO: 228 - protein, 9 aa, Artificial Sequence
  Description of Artificial Sequence Synthetic peptide
QQRAHSPLT

SEQ ID NO: 229 - protein, 447 aa, Artificial Sequence; identical to SEQ ID NO: 13
  Description of Artificial Sequence Synthetic polypeptide

SEQ ID NO: 230 - protein, 214 aa, Artificial Sequence
  Description of Artificial Sequence Synthetic polypeptide
EIVLTQSPATLSLSPGERATLSCRASQSVSNYLAWYQQKPGQAPRLLIHDASNRATGIPARFSGSGSGTDFTLTISSLEPEDFAVYYCQQRAHSPNTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 231 - protein, 5 aa, Artificial Sequence
  Description of Artificial Sequence Synthetic peptide
TYAIH

SEQ ID NO: 232 - protein, 17 aa, Artificial Sequence
  Description of Artificial Sequence Synthetic peptide
RIIPIEGRAYYAQKFQG

SEQ ID NO: 233 - protein, 11 aa, Artificial Sequence
  Description of Artificial Sequence Synthetic peptide
GRYSYRYWFDY

SEQ ID NO: 234 - protein, 11 aa, Artificial Sequence
  Description of Artificial Sequence Synthetic peptide
RASQSVSNYLA

SEQ ID NO: 235 - protein, 7 aa, Artificial Sequence
  Description of Artificial Sequence Synthetic peptide
DASNRAT

SEQ ID NO: 236 - protein, 9 aa, Artificial Sequence
  Description of Artificial Sequence Synthetic peptide
QQRAHSPNT

SEQ ID NO: 237 - protein, 447 aa, Artificial Sequence; identical to SEQ ID NO: 13
  Description of Artificial Sequence Synthetic polypeptide

SEQ ID NO: 238 - protein, 214 aa, Artificial Sequence
  Description of Artificial Sequence Synthetic polypeptide
EIVLTQSPATLSLSPGERATLSCRASQSVSSYLAWYQQKPGQAPRLLIEDASDRATGIPARFSGSGSGTDFTLTISSLEPEDFAVYYCQQRSHWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 239 - protein, 5 aa, Artificial Sequence
  Description of Artificial Sequence Synthetic peptide
TYAIH

SEQ ID NO: 240 - protein, 17 aa, Artificial Sequence
  Description of Artificial Sequence Synthetic peptide
RIIPIEGRAYYAQKFQG

SEQ ID NO: 241 - protein, 11 aa, Artificial Sequence
  Description of Artificial Sequence Synthetic peptide
GRYSYRYWFDY

SEQ ID NO: 242 - protein, 11 aa, Artificial Sequence
  Description of Artificial Sequence Synthetic peptide
RASQSVSSYLA

SEQ ID NO: 243 - protein, 7 aa, Artificial Sequence
  Description of Artificial Sequence Synthetic peptide
DASDRAT

SEQ ID NO: 244 - protein, 9 aa, Artificial Sequence
  Description of Artificial Sequence Synthetic peptide
QQRSHWPLT

SEQ ID NO: 245 - protein, 447 aa, Artificial Sequence; identical to SEQ ID NO: 9
  Description of Artificial Sequence Synthetic polypeptide

SEQ ID NO: 246 - protein, 214 aa, Artificial Sequence
  Description of Artificial Sequence Synthetic polypeptide
EIVLTQSPATLSLSPGERATLSCRASQSVSNYLAWYQQKPGQAPRLLIYDASNRATGIPARFSGSGSGTDFTLTISSLEPEDFAVYYCQQRSHWPNTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 247 - protein, 5 aa, Artificial Sequence
  Description of Artificial Sequence Synthetic peptide
TDAIH

SEQ ID NO: 248 - protein, 17 aa, Artificial Sequence
  Description of Artificial Sequence Synthetic peptide
RIIPISGTANYAQKFQG

SEQ ID NO: 249 - protein, 11 aa, Artificial Sequence
  Description of Artificial Sequence Synthetic peptide
GRYSYYYYFDY

SEQ ID NO: 250 - protein, 11 aa, Artificial Sequence
  Description of Artificial Sequence Synthetic peptide
RASQSVSNYLA

SEQ ID NO: 251 - protein, 7 aa, Artificial Sequence
  Description of Artificial Sequence Synthetic peptide
DASNRAT

SEQ ID NO: 252 - protein, 9 aa, Artificial Sequence
  Description of Artificial Sequence Synthetic peptide
QQRSHWPNT

SEQ ID NO: 253 - protein, 447 aa, Artificial Sequence; identical to SEQ ID NO: 9
  Description of Artificial Sequence Synthetic polypeptide

SEQ ID NO: 254 - protein, 214 aa, Artificial Sequence
  Description of Artificial Sequence Synthetic polypeptide
EIVLTQSPATLSLSPGERATLSCRASQSVNNYLAWYQQKPGQAPRLLIYDASNRATGIPARFSGSGSGTDFTLTISSLEPEDFAVYYCQQRSHWPNTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 255 - protein, 5 aa, Artificial Sequence
  Description of Artificial Sequence Synthetic peptide
TDAIH

SEQ ID NO: 256 - protein, 17 aa, Artificial Sequence
  Description of Artificial Sequence Synthetic peptide
RIIPISGTANYAQKFQG

SEQ ID NO: 257 - protein, 11 aa, Artificial Sequence
  Description of Artificial Sequence Synthetic peptide
GRYSYYYYFDY

SEQ ID NO: 258 - protein, 11 aa, Artificial Sequence
  Description of Artificial Sequence Synthetic peptide
RASQSVNNYLA

SEQ ID NO: 259 - protein, 7 aa, Artificial Sequence
  Description of Artificial Sequence Synthetic peptide
DASNRAT

SEQ ID NO: 260 - protein, 9 aa, Artificial Sequence
  Description of Artificial Sequence Synthetic peptide
QQRSHWPNT

SEQ ID NO: 261 - protein, 447 aa, Artificial Sequence; identical to SEQ ID NO: 13
  Description of Artificial Sequence Synthetic polypeptide

SEQ ID NO: 262 - protein, 214 aa, Artificial Sequence
  Description of Artificial Sequence Synthetic polypeptide
EIVLTQSPATLSLSPGERATLSCRASQSVSSYLAWYQQKPGQAPRLLIYDASYRATGIPARFSGSGSGTDFTLTISSLEPEDFAVYYCQQRSHSPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 263 - protein, 5 aa, Artificial Sequence
  Description of Artificial Sequence Synthetic peptide
TYAIH

SEQ ID NO: 264 - protein, 17 aa, Artificial Sequence
  Description of Artificial Sequence Synthetic peptide
RIIPIEGRAYYAQKFQG

SEQ ID NO: 265 - protein, 11 aa, Artificial Sequence
  Description of Artificial Sequence Synthetic peptide
GRYSYRYWFDY

SEQ ID NO: 266 - protein, 11 aa, Artificial Sequence
  Description of Artificial Sequence Synthetic peptide
RASQSVSSYLA

SEQ ID NO: 267 - protein, 7 aa, Artificial Sequence
  Description of Artificial Sequence Synthetic peptide
DASYRAT

SEQ ID NO: 268 - protein, 9 aa, Artificial Sequence
  Description of Artificial Sequence Synthetic peptide
QQRSHSPLT

SEQ ID NO: 269 - protein, 447 aa, Artificial Sequence; identical to SEQ ID NO: 63
  Description of Artificial Sequence Synthetic polypeptide

SEQ ID NO: 270 - protein, 214 aa, Artificial Sequence
  Description of Artificial Sequence Synthetic polypeptide
EIVLTQSPATLSLSPGERATLSCRASQSVRNYLAWYQQKPGQAPRLLIEDASNRATGIPARFSGSGSGTDFTLTISSLEPEDFAVYYCQQRSHSPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 271 - protein, 5 aa, Artificial Sequence
  Description of Artificial Sequence Synthetic peptide
SDAIH

SEQ ID NO: 272 - protein, 17 aa, Artificial Sequence
  Description of Artificial Sequence Synthetic peptide
YIIPISGTANYAQKFQG

SEQ ID NO: 273 - protein, 11 aa, Artificial Sequence
  Description of Artificial Sequence Synthetic peptide
GRYSYRWTFDY

SEQ ID NO: 274 - protein, 11 aa, Artificial Sequence
  Description of Artificial Sequence Synthetic peptide
RASQSVRNYLA

SEQ ID NO: 275 - protein, 7 aa, Artificial Sequence
  Description of Artificial Sequence Synthetic peptide
DASNRAT

SEQ ID NO: 276 - protein, 9 aa, Artificial Sequence
  Description of Artificial Sequence Synthetic peptide
QQRSHSPIT

SEQ ID NO: 277 - protein, 447 aa, Artificial Sequence; identical to SEQ ID NO: 66
  Description of Artificial Sequence Synthetic polypeptide

SEQ ID NO: 278 - protein, 214 aa, Artificial Sequence
  Description of Artificial Sequence Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 279 - protein, 5 aa, Artificial Sequence
  Description of Artificial Sequence Synthetic peptide
SAYIH

SEQ ID NO: 280 - protein, 17 aa, Artificial Sequence
  Description of Artificial Sequence Synthetic peptide
YISPYYGSAYYAQKFQG

SEQ ID NO: 281 - protein, 11 aa, Artificial Sequence
  Description of Artificial Sequence Synthetic peptide
GRIAYYYVFDY

SEQ ID NO: 282 - protein, 11 aa, Artificial Sequence
  Description of Artificial Sequence Synthetic peptide
RASQSVSSYLA

SEQ ID NO: 283 - protein, 7 aa, Artificial Sequence
  Description of Artificial Sequence Synthetic peptide
DASNRAT

SEQ ID NO: 284 - protein, 9 aa, Artificial Sequence
  Description of Artificial Sequence Synthetic peptide
QQRSNWPIT

SEQ ID NO: 285 - protein, 447 aa, Artificial Sequence; identical to SEQ ID NO: 63
  Description of Artificial Sequence Synthetic polypeptide

SEQ ID NO: 286 - protein, 214 aa, Artificial Sequence
  Description of Artificial Sequence Synthetic polypeptide
EIVLTQSPATLSLSPGERATLSCRASQSVSNYLAWYQQKPGQAPRLLIYDASNRATGIPARFSGSGSGTDFTLTISSLEPEDFAVYYCQQRSNWPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 287 - protein, 5 aa, Artificial Sequence
  Description of Artificial Sequence Synthetic peptide
SDAIH

SEQ ID NO: 288 - protein, 17 aa, Artificial Sequence
  Description of Artificial Sequence Synthetic peptide
YIIPISGTANYAQKFQG

SEQ ID NO: 289 - protein, 11 aa, Artificial Sequence
  Description of Artificial Sequence Synthetic peptide
GRYSYRWTFDY

SEQ ID NO: 290 - protein, 11 aa, Artificial Sequence
  Description of Artificial Sequence Synthetic peptide
RASQSVSNYLA

SEQ ID NO: 291 - protein, 7 aa, Artificial Sequence
  Description of Artificial Sequence Synthetic peptide
DASNRAT

SEQ ID NO: 292 - protein, 9 aa, Artificial Sequence
  Description of Artificial Sequence Synthetic peptide
QQRSNWPIT

SEQ ID NO: 293 - protein, 447 aa, Artificial Sequence; identical to SEQ ID NO: 9
  Description of Artificial Sequence Synthetic polypeptide

SEQ ID NO: 294 - protein, 214 aa, Artificial Sequence
  Description of Artificial Sequence Synthetic polypeptide
EIVLTQSPATLSLSPGERATLSCRASQSVRSYLAWYQQKPGQAPRLLIYDASNRATGIPARFSGSGSGTDFTLTISSLEPEDFAVYYCQQRSNWPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 295 - protein, 5 aa, Artificial Sequence
  Description of Artificial Sequence Synthetic peptide
TDAIH

SEQ ID NO: 296 - protein, 17 aa, Artificial Sequence
  Description of Artificial Sequence Synthetic peptide
RIIPISGTANYAQKFQG

SEQ ID NO: 297 - protein, 11 aa, Artificial Sequence
  Description of Artificial Sequence Synthetic peptide
GRYSYYYYFDY

SEQ ID NO: 298 - protein, 11 aa, Artificial Sequence
  Description of Artificial Sequence Synthetic peptide
RASQSVRSYLA

SEQ ID NO: 299 - protein, 7 aa, Artificial Sequence
  Description of Artificial Sequence Synthetic peptide
DASNRAT

SEQ ID NO: 300 - protein, 9 aa, Artificial Sequence
  Description of Artificial Sequence Synthetic peptide
QQRSNWPIT

SEQ ID NO: 301 - protein, 447 aa, Artificial Sequence; identical to SEQ ID NO: 66
  Description of Artificial Sequence Synthetic polypeptide

SEQ ID NO: 302 - protein, 214 aa, Artificial Sequence
  Description of Artificial Sequence Synthetic polypeptide
EIVLTQSPATLSLSPGERATLSCRASQSVRNYLAWYQQKPGQAPRLLIYDASNRATGIPARFSGSGSGTDFTLTISSLEPEDFAVYYCQQRSNWPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 303 - protein, 5 aa, Artificial Sequence
  Description of Artificial Sequence Synthetic peptide
SAYIH

SEQ ID NO: 304 - protein, 17 aa, Artificial Sequence
  Description of Artificial Sequence Synthetic peptide
YISPYYGSAYYAQKFQG

SEQ ID NO: 305 - protein, 11 aa, Artificial Sequence
  Description of Artificial Sequence Synthetic peptide
GRIAYYYVFDY

SEQ ID NO: 306 - protein, 11 aa, Artificial Sequence
  Description of Artificial Sequence Synthetic peptide
RASQSVRNYLA

SEQ ID NO: 307 - protein, 7 aa, Artificial Sequence
  Description of Artificial Sequence Synthetic peptide
DASNRAT

SEQ ID NO: 308 - protein, 9 aa, Artificial Sequence
  Description of Artificial Sequence Synthetic peptide
QQRSNWPIT

SEQ ID NO: 309 - protein, 447 aa, Artificial Sequence; identical to SEQ ID NO: 70
  Description of Artificial Sequence Synthetic polypeptide

SEQ ID NO: 310 - protein, 214 aa, Artificial Sequence
  Description of Artificial Sequence Synthetic polypeptide
EIVLTQSPATLSLSPGERATLSCRASQSVSTYLAWYQQKPGQAPRLLIKDASNRATGIPARFSGSGSGTDFTLTISSLEPEDFAVYYCQQRSNWPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 311 - protein, 5 aa, Artificial Sequence
  Description of Artificial Sequence Synthetic peptide
TDAIH

SEQ ID NO: 312 - protein, 17 aa, Artificial Sequence
  Description of Artificial Sequence Synthetic peptide
YIIPISGNANYAQKFQG

SEQ ID NO: 313 - protein, 11 aa, Artificial Sequence
  Description of Artificial Sequence Synthetic peptide
GRYAYNWGFDY

SEQ ID NO: 314 - protein, 11 aa, Artificial Sequence
  Description of Artificial Sequence Synthetic peptide
RASQSVSTYLA

SEQ ID NO: 315 - protein, 7 aa, Artificial Sequence
  Description of Artificial Sequence Synthetic peptide
DASNRAT

SEQ ID NO: 316 - protein, 9 aa, Artificial Sequence
  Description of Artificial Sequence Synthetic peptide
QQRSNWPIT

SEQ ID NO: 317 - protein, 447 aa, Artificial Sequence; identical to SEQ ID NO: 66
  Description of Artificial Sequence Synthetic polypeptide

SEQ ID NO: 318 - protein, 214 aa, Artificial Sequence
  Description of Artificial Sequence Synthetic polypeptide
EIVLTQSPATLSLSPGERATLSCRASQSVSSYLAWYQQKPGQAPRLLIKDASDRATGIPARFSGSGSGTDFTLTISSLEPEDFAVYYCQQRSNWPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 319 - protein, 5 aa, Artificial Sequence
  Description of Artificial Sequence Synthetic peptide
SAYIH

SEQ ID NO: 320 - protein, 17 aa, Artificial Sequence
  Description of Artificial Sequence Synthetic peptide
YISPYYGSAYYAQKFQG

SEQ ID NO: 321 - protein, 11 aa, Artificial Sequence
  Description of Artificial Sequence Synthetic peptide
GRIAYYYVFDY

SEQ ID NO: 322 - protein, 11 aa, Artificial Sequence
  Description of Artificial Sequence Synthetic peptide
RASQSVSSYLA

SEQ ID NO: 323 - protein, 7 aa, Artificial Sequence
  Description of Artificial Sequence Synthetic peptide
DASDRAT

SEQ ID NO: 324 - protein, 9 aa, Artificial Sequence
  Description of Artificial Sequence Synthetic peptide
QQRSNWPIT

SEQ ID NO: 325 - protein, 447 aa, Artificial Sequence; identical to SEQ ID NO: 13
  Description of Artificial Sequence Synthetic polypeptide

SEQ ID NO: 326 - protein, 214 aa, Artificial Sequence
  Description of Artificial Sequence Synthetic polypeptide
EIVLTQSPATLSLSPGERATLSCRASQSVSNYLAWYQQKPGQAPRLLIYDASNRATGIPARFSGSGSGTDFTLTISSLEPEDFAVYYCQQRSNWPNTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 327 - protein, 5 aa, Artificial Sequence
  Description of Artificial Sequence Synthetic peptide
TYAIH

SEQ ID NO: 328 - protein, 17 aa, Artificial Sequence
  Description of Artificial Sequence Synthetic peptide
RIIPIEGRAYYAQKFQG

SEQ ID NO: 329 - protein, 11 aa, Artificial Sequence
  Description of Artificial Sequence Synthetic peptide
GRYSYRYWFDY

SEQ ID NO: 330 - protein, 11 aa, Artificial Sequence
  Description of Artificial Sequence Synthetic peptide
RASQSVSNYLA

SEQ ID NO: 331 - protein, 7 aa, Artificial Sequence
  Description of Artificial Sequence Synthetic peptide
DASNRAT

SEQ ID NO: 332 - protein, 9 aa, Artificial Sequence
  Description of Artificial Sequence Synthetic peptide
QQRSNWPNT

SEQ ID NO: 333 - protein, 447 aa, Artificial Sequence; identical to SEQ ID NO: 13
  Description of Artificial Sequence Synthetic polypeptide

SEQ ID NO: 334 - protein, 214 aa, Artificial Sequence
  Description of Artificial Sequence Synthetic polypeptide
EIVLTQSPATLSLSPGERATLSCRASQSVSNYLAWYQQKPGQAPRLLIHDASNRATGIPARFSGSGSGTDFTLTISSLEPEDFAVYYCQQRSNWPNTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 335 - protein, 5 aa, Artificial Sequence
  Description of Artificial Sequence Synthetic peptide
TYAIH

SEQ ID NO: 336 - protein, 17 aa, Artificial Sequence
  Description of Artificial Sequence Synthetic peptide
RIIPIEGRAYYAQKFQG

SEQ ID NO: 337 - protein, 11 aa, Artificial Sequence
  Description of Artificial Sequence Synthetic peptide
GRYSYRYWFDY

SEQ ID NO: 338 - protein, 11 aa, Artificial Sequence
  Description of Artificial Sequence Synthetic peptide
RASQSVSNYLA

SEQ ID NO: 339 - protein, 7 aa, Artificial Sequence
  Description of Artificial Sequence Synthetic peptide
DASNRAT

SEQ ID NO: 340 - protein, 9 aa, Artificial Sequence
  Description of Artificial Sequence Synthetic peptide
QQRSNWPNT

SEQ ID NO: 341 - protein, 447 aa, Artificial Sequence; identical to SEQ ID NO: 9
  Description of Artificial Sequence Synthetic polypeptide

SEQ ID NO: 342 - protein, 214 aa, Artificial Sequence
  Description of Artificial Sequence Synthetic polypeptide
EIVLTQSPATLSLSPGERATLSCRASQSVSTYLAWYQQKPGQAPRLLIYDASNRATGIPARFSGSGSGTDFTLTISSLEPEDFAVYYCQQRSNWPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 343 - protein, 5 aa, Artificial Sequence
  Description of Artificial Sequence Synthetic peptide
TDAIH

SEQ ID NO: 344 - protein, 17 aa, Artificial Sequence
  Description of Artificial Sequence Synthetic peptide
RIIPISGTANYAQKFQG

SEQ ID NO: 345 - protein, 11 aa, Artificial Sequence
  Description of Artificial Sequence Synthetic peptide
GRYSYYYYFDY

SEQ ID NO: 346 - protein, 11 aa, Artificial Sequence
  Description of Artificial Sequence Synthetic peptide
RASQSVSTYLA

SEQ ID NO: 347 - protein, 7 aa, Artificial Sequence
  Description of Artificial Sequence Synthetic peptide
DASNRAT

SEQ ID NO: 348 - protein, 9 aa, Artificial Sequence
  Description of Artificial Sequence Synthetic peptide
QQRSNWPIT

SEQ ID NO: 349 - protein, 447 aa, Artificial Sequence; identical to SEQ ID NO: 63
  Description of Artificial Sequence Synthetic polypeptide

SEQ ID NO: 350 - protein, 214 aa, Artificial Sequence
  Description of Artificial Sequence Synthetic polypeptide
EIVLTQSPATLSLSPGERATLSCRASQSVSTYLAWYQQKPGQAPRLLIYDASNRATGIPARFSGSGSGTDFTLTISSLEPEDFAVYYCQQRSNSPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 351 - protein, 5 aa, Artificial Sequence
  Description of Artificial Sequence Synthetic peptide
SDAIH

SEQ ID NO: 352 - protein, 17 aa, Artificial Sequence
  Description of Artificial Sequence Synthetic peptide
YIIPISGTANYAQKFQG

SEQ ID NO: 353 - protein, 11 aa, Artificial Sequence
  Description of Artificial Sequence Synthetic peptide
GRYSYRWTFDY

SEQ ID NO: 354 - protein, 11 aa, Artificial Sequence
  Description of Artificial Sequence Synthetic peptide
RASQSVSTYLA

SEQ ID NO: 355 - protein, 7 aa, Artificial Sequence
  Description of Artificial Sequence Synthetic peptide
DASNRAT

SEQ ID NO: 356 - protein, 9 aa, Artificial Sequence
  Description of Artificial Sequence Synthetic peptide
QQRSNSPIT

SEQ ID NO: 357 - protein, 447 aa, Artificial Sequence; identical to SEQ ID NO: 63
  Description of Artificial Sequence Synthetic polypeptide

SEQ ID NO: 358 - protein, 214 aa, Artificial Sequence
  Description of Artificial Sequence Synthetic polypeptide
EIVLTQSPATLSLSPGERATLSCRASQSVSTYLAWYQQKPGQAPRLLIEDASYRATGIPARFSGSGSGTDFTLTISSLEPEDFAVYYCQQRSNSPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 359 - protein, 5 aa, Artificial Sequence
  Description of Artificial Sequence Synthetic peptide
SDAIH

SEQ ID NO: 360 - protein, 17 aa, Artificial Sequence
  Description of Artificial Sequence Synthetic peptide
YIIPISGTANYAQKFQG

SEQ ID NO: 361 - protein, 11 aa, Artificial Sequence
  Description of Artificial Sequence Synthetic peptide
GRYSYRWTFDY

SEQ ID NO: 362 - protein, 11 aa, Artificial Sequence
  Description of Artificial Sequence Synthetic peptide
RASQSVSTYLA

SEQ ID NO: 363 - protein, 7 aa, Artificial Sequence
  Description of Artificial Sequence Synthetic peptide
DASYRAT

SEQ ID NO: 364 - protein, 9 aa, Artificial Sequence
  Description of Artificial Sequence Synthetic peptide
QQRSNSPIT

SEQ ID NO: 365 - protein, 447 aa, Artificial Sequence; identical to SEQ ID NO: 17
  Description of Artificial Sequence Synthetic polypeptide

SEQ ID NO: 366 - protein, 214 aa, Artificial Sequence
  Description of Artificial Sequence Synthetic polypeptide
EIVLTQSPATLSLSPGERATLSCRASQSVSNYLAWYQQKPGQAPRLLIEDASNRATGIPARFSGSGSGTDFTLTISSLEPEDFAVYYCQQRSNYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 367 - protein, 5 aa, Artificial Sequence
  Description of Artificial Sequence Synthetic peptide
DAAIH

SEQ ID NO: 368 - protein, 17 aa, Artificial Sequence
  Description of Artificial Sequence Synthetic peptide
YIIPISGTANYAQKFQG

SEQ ID NO: 369 - protein, 11 aa, Artificial Sequence
  Description of Artificial Sequence Synthetic peptide
GRARYRWAFDY

SEQ ID NO: 370 - protein, 11 aa, Artificial Sequence
  Description of Artificial Sequence Synthetic peptide
RASQSVSNYLA

SEQ ID NO: 371 - protein, 7 aa, Artificial Sequence
  Description of Artificial Sequence Synthetic peptide
DASNRAT

SEQ ID NO: 372 - protein, 9 aa, Artificial Sequence
  Description of Artificial Sequence Synthetic peptide
QQRSNYPLT

SEQ ID NO: 373 - protein, 447 aa, Artificial Sequence; identical to SEQ ID NO: 63
  Description of Artificial Sequence Synthetic polypeptide

SEQ ID NO: 374 - protein, 214 aa, Artificial Sequence
  Description of Artificial Sequence Synthetic polypeptide
EIVLTQSPATLSLSPGERATLSCRASQSVRNYLAWYQQKPGQAPRLLIYDASSRATGIPARFSGSGSGTDFTLTISSLEPEDFAVYYCQQRSNYPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 375 - protein, 5 aa, Artificial Sequence
  Description of Artificial Sequence Synthetic peptide
SDAIH

SEQ ID NO: 376 - protein, 17 aa, Artificial Sequence
  Description of Artificial Sequence Synthetic peptide
YIIPISGTANYAQKFQG

SEQ ID NO: 377 - protein, 11 aa, Artificial Sequence
  Description of Artificial Sequence Synthetic peptide
GRYSYRWTFDY

SEQ ID NO: 378 - protein, 11 aa, Artificial Sequence
  Description of Artificial Sequence Synthetic peptide
RASQSVRNYLA

SEQ ID NO: 379 - protein, 7 aa, Artificial Sequence
  Description of Artificial Sequence Synthetic peptide
DASSRAT

SEQ ID NO: 380 - protein, 9 aa, Artificial Sequence
  Description of Artificial Sequence Synthetic peptide
QQRSNYPIT

SEQ ID NO: 381 - protein, 447 aa, Artificial Sequence; identical to SEQ ID NO: 63
  Description of Artificial Sequence Synthetic polypeptide

SEQ ID NO: 382 - protein, 214 aa, Artificial Sequence; identical to SEQ ID NO: 302
  Description of Artificial Sequence Synthetic polypeptide

SEQ ID NO: 383 - protein, 5 aa, Artificial Sequence
  Description of Artificial Sequence Synthetic peptide
SDAIH

SEQ ID NO: 384 - protein, 17 aa, Artificial Sequence
  Description of Artificial Sequence Synthetic peptide
YIIPISGTANYAQKFQG

SEQ ID NO: 385 - protein, 11 aa, Artificial Sequence
  Description of Artificial Sequence Synthetic peptide
GRYSYRWTFDY

SEQ ID NO: 386 - protein, 11 aa, Artificial Sequence
  Description of Artificial Sequence Synthetic peptide
RASQSVRNYLA

SEQ ID NO: 387 - protein, 7 aa, Artificial Sequence
  Description of Artificial Sequence Synthetic peptide
DASNRAT

SEQ ID NO: 388 - protein, 9 aa, Artificial Sequence
  Description of Artificial Sequence Synthetic peptide
QQRSNWPIT

SEQ ID NO: 389 - protein, 377 aa, Unknown
  Description of Unknown Human or mouse polypeptide
QETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACDSKDSKEKNKMEILY

SEQ ID NO: 390 - protein, 223 aa, Artificial Sequence
  Description of Artificial Sequence Synthetic polypeptide
QVQLVQSGAEVKKPGSSVKVSCKASGGTFSTYAIHWVRQAPGQGLEWMGRIIPIEGRAYYAQKFQGRVTITADESTSTAYMELSSLRSEDTAVYYCARGRYSYRYWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 391 - protein, 214 aa, Artificial Sequence; identical to SEQ ID NO: 5
  Description of Artificial Sequence Synthetic polypeptide

(19) United States
(12) Patent Application Publication
(10) Pub. No.: US 2017/0210799 A1
(43) Pub. Date: Jul. 27, 2017

(54) ANTI-ROR1, ANTIBODIES, ROR1 X CD3 BISPECIFIC ANTIBODIES, AND METHODS OF USING THE SAME

(71) Applicant: JANSSEN BIOTECH, INC., Horsham, PA (US)

(72) Inventors: Glenn Mark ANDERSON, Eagleville, PA (US); Eric T. BALDWIN, Upper Holland, PA (US); Benjamin HARMAN, Oaklyn, NJ (US); Rosa M.F. CARDOSO, North Wales, PA (US); Jennifer F. NEMETH-SEAY, Fort Washington, PA (US); Ricardo ATTAR, Lawrenceville, NJ (US); Francois GAUDET, Princeton, NJ (US); Yingzhe LI, Dresher, PA (US); Jinquan LUO, Malvern, PA (US); Ronan MCDAID, Eagleville, PA (US); Steven C. POMERANTZ, Huntingdon Valley, PA (US); Susan H. TAM, Garnet Valley, PA (US); Alexey TEPLYAKOV, Phoenixville, PA (US); Sheng-Jiun WU, Broomall, PA (US)

(21) Appl. No.: 15/409,630

(22) Filed: Jan. 19, 2017

Related U.S. Application Data
(60) Provisional application No. 62/286,121, filed on Jan. 22, 2016.

(51) Int. Cl. C07K 16/28

(52) U.S. Cl. CPC C07K 16/28; C07K 16/2809; C07K 2317/565; C07K 2317/31; C07K 2317/34; C07K 2317/92

(57) ABSTRACT

Provided herein are isolated antibodies that immunospecifically bind to ROR1, bispecific antibodies comprising an antigen-binding site that immunospecifically binds to ROR1 and an antigen-binding site that immunospecifically binds to CD3, and methods of using the same.

### TECHNICAL FIELD

Provided herein are isolated antibodies that immunospecifically bind to ROR1, bispecific antibodies comprising an antigen-binding site that immunospecifically binds to ROR1 and an antigen-binding site that immunospecifically binds to CD3, and methods of using the same.

### BACKGROUND

Receptor Tyrosine Kinase-Like Orphan Receptor 1 (ROR1) is a 106-kDa member of the receptor tyrosine kinase family. Structurally, the extracellular domain of the ROR1 receptor is composed of three distinct domains: a membrane-distal Immunoglobulin-Like Domain; a membrane-proximal Kringle Domain; and an intervening Frizzled Domain. Studies on ROR1-deficient mice suggest that this receptor plays a role in lung development during embryogenesis, but its expression in the adult is severely restricted, with expression limited to low-level expression in the lung and pancreas, as well as adipocytic and B cell lineages. While ROR1 expression is tightly regulated in normal adult tissues, high levels have been noted in both hematological and solid tumors. ROR1 is normally expressed during early development, but becomes activated by tumor specific mechanisms and may contribute to disease progression in the adult.

The ligands of ROR1 are believed to be wnt5a and NKX1-2. Wnt5a has been shown to bind to the Frizzled Domain in the extracellular part of ROR1 and, in transfected cells, has been shown to modulate NF-κB activation and proliferation of normal and lung tumor cell lines. Binding of NKX1-2 to ROR1 has been shown to play a role in the survival of lung cancer cell lines through both kinase-dependent and kinase-independent mechanisms. ROR1 has been shown to interact with EGFR through the Kringle domain, and this interaction modulates signaling pathways that control apoptosis in lung cancer cell lines. While ROR1 expression does correlate with a worse prognosis in ovarian cancer, no link between ROR1 expression and clinical stage or reduced survival has been shown for lung cancer. Furthermore, although ROR1 siRNA knockdown of lung tumor cell lines leads to reduced viability in vitro, there is no evidence that targeting of ROR1 on primary lung cancer cells results in increased cell death.

### SUMMARY

Disclosed herein are isolated antibodies, or antigen-binding fragments thereof, that immunospecifically bind to ROR1, the antibodies or antigen-binding fragments thereof comprising:
a. a heavy chain CDR1 comprising the amino acid sequence of SEQ ID NO:2, a heavy chain CDR2 comprising the amino acid sequence of SEQ ID NO:3, a heavy chain CDR3 comprising the amino acid sequence of SEQ ID NO:4, a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8;
b. a heavy chain CDR1 comprising the amino acid sequence of SEQ ID NO:10, a heavy chain CDR2 comprising the amino acid sequence of SEQ ID NO:11, a heavy chain CDR3 comprising the amino acid sequence of SEQ ID NO:12, a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8;
c. a heavy chain CDR1 comprising the amino acid sequence of SEQ ID NO:14, a heavy chain CDR2 comprising the amino acid sequence of SEQ ID NO:15, a heavy chain CDR3 comprising the amino acid sequence of SEQ ID NO:16, a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8;
d. a heavy chain CDR1 comprising the amino acid sequence of SEQ ID NO:18, a heavy chain CDR2 comprising the amino acid sequence of SEQ ID NO:19, a heavy chain CDR3 comprising the amino acid sequence of SEQ ID NO:20, a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8;
e. a heavy chain CDR1 comprising the amino acid sequence of SEQ ID NO:22, a heavy chain CDR2 comprising the amino acid sequence of SEQ ID NO:23, a heavy chain CDR3 comprising the amino acid sequence of SEQ ID NO:24, a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8;
f. a heavy chain CDR1 comprising the amino acid sequence of SEQ ID NO:26, a heavy chain CDR2 comprising the amino acid sequence of SEQ ID NO:27, a heavy chain CDR3 comprising the amino acid sequence of SEQ ID NO:28, a light chain CDR1 comprising the amino acid sequence of SEQ ID NO:30, a light chain CDR2 comprising the amino acid sequence of SEQ ID NO:31, and a light chain CDR3 comprising the amino acid sequence of SEQ ID NO:32;
g. a heavy chain CDR1 comprising the amino acid sequence of SEQ ID NO:2, a heavy chain CDR2 comprising the amino acid sequence of SEQ ID NO:34, a heavy chain CDR3 comprising the amino acid sequence of SEQ ID NO:35, a light chain CDR1 comprising the amino acid sequence of SEQ ID NO:37, a light chain CDR2 comprising the amino acid sequence of SEQ ID NO:7, and a light chain CDR3 comprising the amino acid sequence of SEQ ID NO:38;
h. a heavy chain CDR1 comprising the amino acid sequence of SEQ ID NO:22, a heavy chain CDR2 comprising the amino acid sequence of SEQ ID NO:23, a heavy chain CDR3 comprising the amino acid sequence of SEQ ID NO:40, a light chain CDR1 comprising the amino acid sequence of SEQ ID NO:42, a light chain CDR2 comprising the amino acid sequence of SEQ ID NO:43, and a light chain CDR3 comprising the amino acid sequence of SEQ ID NO:44;
i. a heavy chain CDR1 comprising the amino acid sequence of SEQ ID NO:46, a heavy chain CDR2 comprising the amino acid sequence of SEQ ID NO:47, a heavy chain CDR3 comprising the amino acid sequence of SEQ ID NO:48, a light chain CDR1 comprising the amino acid sequence of SEQ ID NO:50, a light chain CDR2 comprising the amino acid sequence of SEQ ID NO:51, and a light chain CDR3 comprising the amino acid sequence of SEQ ID NO:52;
j. a heavy chain CDR1 comprising the amino acid sequence of SEQ ID NO:54, a heavy chain CDR2 comprising the amino acid sequence of SEQ ID NO:55, a heavy chain CDR3 comprising the amino acid sequence of SEQ ID NO:56, a light chain CDR1 comprising the amino acid sequence of SEQ ID NO:58, a light chain CDR2 comprising the amino acid sequence of SEQ ID NO:59, and a light chain CDR3 comprising the amino acid sequence of SEQ ID NO:60;
k. a heavy chain CDR1 comprising the amino acid sequence of SEQ ID NO:54, a heavy chain CDR2 comprising the amino acid sequence of SEQ ID NO:55, a heavy chain CDR3 comprising the amino acid sequence of SEQ ID NO:62, a light chain CDR1 comprising the amino acid sequence of SEQ ID NO:58, a light chain CDR2 comprising the amino acid sequence of SEQ ID NO:59, and a light chain CDR3 comprising the amino acid sequence of SEQ ID NO:60;
l. a heavy chain CDR1 comprising the amino acid sequence of SEQ ID NO:64, a heavy chain CDR2 comprising the amino acid sequence of SEQ ID NO:19, a heavy chain CDR3 comprising the amino acid sequence of SEQ ID NO:65, a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8;
m. a heavy chain CDR1 comprising the amino acid sequence of SEQ ID NO:67, a heavy chain CDR2 comprising the amino acid sequence of SEQ ID NO:68, a heavy chain CDR3 comprising the amino acid sequence of SEQ ID NO:69, a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8;
n. a heavy chain CDR1 comprising the amino acid sequence of SEQ ID NO:10, a heavy chain CDR2 comprising the amino acid sequence of SEQ ID NO:71, a heavy chain CDR3 comprising the amino acid sequence of SEQ ID NO:72, a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8;
o. a heavy chain CDR1 comprising the amino acid sequence of SEQ ID NO:54, a heavy chain CDR2 comprising the amino acid sequence of SEQ ID NO:74, a heavy chain CDR3 comprising the amino acid sequence of SEQ ID NO:75, a light chain CDR1 comprising the amino acid sequence of SEQ ID NO:77, a light chain CDR2 comprising the amino acid sequence of SEQ ID NO:51, and a light chain CDR3 comprising the amino acid sequence of SEQ ID NO:78;
p. a heavy chain CDR1 comprising the amino acid sequence of SEQ ID NO:22, a heavy chain CDR2 comprising the amino acid sequence of SEQ ID NO:23, a heavy chain CDR3 comprising the amino acid sequence of SEQ ID NO:80, a light chain CDR1 comprising the amino acid sequence of SEQ ID NO:82, a light chain CDR2 comprising the amino acid sequence of SEQ ID NO:7, and a light chain CDR3 comprising the amino acid sequence of SEQ ID NO:83; or
q. a heavy chain CDR1 comprising the amino acid sequence of SEQ ID NO:22, a heavy chain CDR2 comprising the amino acid sequence of SEQ ID NO:85, a heavy chain CDR3 comprising the amino acid sequence of SEQ ID NO:86, a light chain CDR1 comprising the amino acid sequence of SEQ ID NO:88, a light chain CDR2 comprising the amino acid sequence of SEQ ID NO:43, and a light chain CDR3 comprising the amino acid sequence of SEQ ID NO:89;
   wherein the CDRs are defined according to Kabat.

Further provided are isolated antibodies, or antigen-binding fragments thereof, that immunospecifically bind to ROR1, the antibodies or antigen-binding fragments thereof comprising:
a. a heavy chain comprising an amino acid sequence that is at least 90% identical to the amino acid sequence of SEQ ID NO:1 and a light chain comprising an amino acid sequence that is at least 90% identical to the amino acid sequence of SEQ ID NO:5;
b. a heavy chain comprising an amino acid sequence that is at least 90% identical to the amino acid sequence of SEQ ID NO:9 and a light chain comprising an amino acid sequence that is at least 90% identical to the amino acid sequence of SEQ ID NO:5;
c. a heavy chain comprising an amino acid sequence that is at least 90% identical to the amino acid sequence of SEQ ID NO:13 and a light chain comprising an amino acid sequence that is at least 90% identical to the amino acid sequence of SEQ ID NO:5;
d. a heavy chain comprising an amino acid sequence that is at least 90% identical to the amino acid sequence of SEQ ID NO:17 and a light chain comprising an amino acid sequence that is at least 90% identical to the amino acid sequence of SEQ ID NO:5;
e. a heavy chain comprising an amino acid sequence that is at least 90% identical to the amino acid sequence of SEQ ID NO:21 and a light chain comprising an amino acid sequence that is at least 90% identical to the amino acid sequence of SEQ ID NO:5;
f. a heavy chain comprising an amino acid sequence that is at least 90% identical to the amino acid sequence of SEQ ID NO:25 and a light chain comprising an amino acid sequence that is at least 90% identical to the amino acid sequence of SEQ ID NO:29;
g. a heavy chain comprising an amino acid sequence that is at least 90% identical to the amino acid sequence of SEQ ID NO:33 and a light chain comprising an amino acid sequence that is at least 90% identical to the amino acid sequence of SEQ ID NO:36;
h. a heavy chain comprising an amino acid sequence that is at least 90% identical to the amino acid sequence of SEQ ID NO:39 and a light chain comprising an amino acid sequence that is at least 90% identical to the amino acid sequence of SEQ ID NO:41;
i. a heavy chain comprising an amino acid sequence that is at least 90% identical to the amino acid sequence of SEQ ID NO:45 and a light chain comprising an amino acid sequence that is at least 90% identical to the amino acid sequence of SEQ ID NO:49;
j. a heavy chain comprising an amino acid sequence that is at least 90% identical to the amino acid sequence of SEQ ID NO:53 and a light chain comprising an amino acid sequence that is at least 90% identical to the amino acid sequence of SEQ ID NO:57;
k. a heavy chain comprising an amino acid sequence that is at least 90% identical to the amino acid sequence of SEQ ID NO:61 and a light chain comprising an amino acid sequence that is at least 90% identical to the amino acid sequence of SEQ ID NO:57;
l. a heavy chain comprising an amino acid sequence that is at least 90% identical to the amino acid sequence of SEQ ID NO:63 and a light chain comprising an amino acid sequence that is at least 90% identical to the amino acid sequence of SEQ ID NO:5;
m. a heavy chain comprising an amino acid sequence that is at least 90% identical to the amino acid sequence of SEQ ID NO:66 and a light chain comprising an amino acid sequence that is at least 90% identical to the amino acid sequence of SEQ ID NO:5;
n. a heavy chain comprising an amino acid sequence that is at least 90% identical to the amino acid sequence of SEQ ID NO:70 and a light chain comprising an amino acid sequence that is at least 90% identical to the amino acid sequence of SEQ ID NO:5;
o. a heavy chain comprising an amino acid sequence that is at least 90% identical to the amino acid sequence of SEQ ID NO:73 and a light chain comprising an amino acid sequence that is at least 90% identical to the amino acid sequence of SEQ ID NO:76;
p. a heavy chain comprising an amino acid sequence that is at least 90% identical to the amino acid sequence of SEQ ID NO:79 and a light chain comprising an amino acid sequence that is at least 90% identical to the amino acid sequence of SEQ ID NO:81; or
q. a heavy chain comprising an amino acid sequence that is at least 90% identical to the amino acid sequence of SEQ ID NO:84 and a light chain comprising an amino acid sequence that is at least 90% identical to the amino acid sequence of SEQ ID NO:87.

Isolated antibodies, or antigen-binding fragments thereof, that bind to an epitope on ROR1 comprising T324, V325, S326, V327, T328, S330, G331, R332, Q333, P336, N338, S339, Y341, H359, S360, Y361, L377, D378, and D387 are disclosed.

Further provided are nucleic acid molecules encoding the disclosed isolated antibodies or antigen-binding fragments thereof, vectors comprising the nucleic acid molecules, and cells expressing the isolated antibodies, or antigen-binding fragments thereof.

Disclosed are isolated antibodies, or antigen-binding fragments thereof, that compete for binding to ROR1 with a reference antibody or antigen-binding fragment thereof, the reference antibody or antigen-binding fragment thereof comprising:
a. a heavy chain comprising the amino acid sequence of SEQ ID NO:1 and a light chain comprising the amino acid sequence of SEQ ID NO:5;
b. a heavy chain comprising the amino acid sequence of SEQ ID NO:9 and a light chain comprising the amino acid sequence of SEQ ID NO:5;
c. a heavy chain comprising the amino acid sequence of SEQ ID NO:13 and a light chain comprising the amino acid sequence of SEQ ID NO:5;
d. a heavy chain comprising the amino acid sequence of SEQ ID NO:17 and a light chain comprising the amino acid sequence of SEQ ID NO:5;
e. a heavy chain comprising the amino acid sequence of SEQ ID NO:21 and a light chain comprising the amino acid sequence of SEQ ID NO:5;
f. a heavy chain comprising the amino acid sequence of SEQ ID NO:25 and a light chain comprising the amino acid sequence of SEQ ID NO:29;
g. a heavy chain comprising the amino acid sequence of SEQ ID NO:33 and a light chain comprising the amino acid sequence of SEQ ID NO:36;
h. a heavy chain comprising the amino acid sequence of SEQ ID NO:39 and a light chain comprising the amino acid sequence of SEQ ID NO:41;
i. a heavy chain comprising the amino acid sequence of SEQ ID NO:45 and a light chain comprising the amino acid sequence of SEQ ID NO:49;
j. a heavy chain comprising the amino acid sequence of SEQ ID NO:53 and a light chain comprising the amino acid sequence of SEQ ID NO:57;
k. a heavy chain comprising the amino acid sequence of SEQ ID NO:61 and a light chain comprising the amino acid sequence of SEQ ID NO:57;
l. a heavy chain comprising the amino acid sequence of SEQ ID NO:63 and a light chain comprising the amino acid sequence of SEQ ID NO:5;
m. a heavy chain comprising the amino acid sequence of SEQ ID NO:66 and a light chain comprising the amino acid sequence of SEQ ID NO:5;
n. a heavy chain comprising the amino acid sequence of SEQ ID NO:70 and a light chain comprising the amino acid sequence of SEQ ID NO:5;
o. a heavy chain comprising the amino acid sequence of SEQ ID NO:73 and a light chain comprising the amino acid sequence of SEQ ID NO:76;
p. a heavy chain comprising the amino acid sequence of SEQ ID NO:79 and a light chain comprising the amino acid sequence of SEQ ID NO:81; or
q. a heavy chain comprising the amino acid sequence of SEQ ID NO:84 and a light chain comprising the amino acid sequence of SEQ ID NO:87.

Isolated antibodies, or antigen-binding fragments thereof, that compete for binding to ROR1 with a reference antibody or antigen-binding fragment thereof, wherein the reference antibody or antigen-binding fragment binds to an epitope on ROR1 comprising T324, V325, S326, V327, T328, S330, G331, R332, Q333, P336, N338, S339, Y341, H359, S360, Y361, L377, D378, and D387 are provided herein.

Disclosed are isolated ROR1×CD3 bispecific antibodies and bispecific antigen-binding fragments thereof. In some embodiments, the isolated ROR1×CD3 bispecific antibodies or bispecific antigen-binding fragments thereof comprise: a) a first antigen-binding site that immunospecifically binds ROR1, the first antigen-binding site comprising a heavy chain CDR1, CDR2, and CDR3 and a light chain CDR1, CDR2, and CDR3; and b) a second antigen-binding site that immunospecifically binds CD3, the second antigen-binding site comprising a heavy chain CDR1, CDR2, and CDR3 and a light chain CDR1, CDR2, and CDR3. Suitable first antigen-binding sites that immunospecifically bind ROR1 include those comprising:
a. a heavy chain CDR1 comprising the amino acid sequence of SEQ ID NO:2, a heavy chain CDR2 comprising the amino acid sequence of SEQ ID NO:3, a heavy chain CDR3 comprising the amino acid sequence of SEQ ID NO:4, a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8;
b. a heavy chain CDR1 comprising the amino acid sequence of SEQ ID NO:10, a heavy chain CDR2 comprising the amino acid sequence of SEQ ID NO:11, a heavy chain CDR3 comprising the amino acid sequence of SEQ ID NO:12, a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8;
c. a heavy chain CDR1 comprising the amino acid sequence of SEQ ID NO:14, a heavy chain CDR2 comprising the amino acid sequence of SEQ ID NO:15, a heavy chain CDR3 comprising the amino acid sequence of SEQ ID NO:16, a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8;
d. a heavy chain CDR1 comprising the amino acid sequence of SEQ ID NO:18, a heavy chain CDR2 comprising the amino acid sequence of SEQ ID NO:19, a heavy chain CDR3 comprising the amino acid sequence of SEQ ID NO:20, a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8;
e. a heavy chain CDR1 comprising the amino acid sequence of SEQ ID NO:22, a heavy chain CDR2 comprising the amino acid sequence of SEQ ID NO:23, a heavy chain CDR3 comprising the amino acid sequence of SEQ ID NO:24, a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8;
f. a heavy chain CDR1 comprising the amino acid sequence of SEQ ID NO:26, a heavy chain CDR2 comprising the amino acid sequence of SEQ ID NO:27, a heavy chain CDR3 comprising the amino acid sequence of SEQ ID NO:28, a light chain CDR1 comprising the amino acid sequence of SEQ ID NO:30, a light chain CDR2 comprising the amino acid sequence of SEQ ID NO:31, and a light chain CDR3 comprising the amino acid sequence of SEQ ID NO:32;
g. a heavy chain CDR1 comprising the amino acid sequence of SEQ ID NO:2, a heavy chain CDR2 comprising the amino acid sequence of SEQ ID NO:34, a heavy chain CDR3 comprising the amino acid sequence of SEQ ID NO:35, a light chain CDR1 comprising the amino acid sequence of SEQ ID NO:37, a light chain CDR2 comprising the amino acid sequence of SEQ ID NO:7, and a light chain CDR3 comprising the amino acid sequence of SEQ ID NO:38;
h. a heavy chain CDR1 comprising the amino acid sequence of SEQ ID NO:22, a heavy chain CDR2 comprising the amino acid sequence of SEQ ID NO:23, a heavy chain CDR3 comprising the amino acid sequence of SEQ ID NO:40, a light chain CDR1 comprising the amino acid sequence of SEQ ID NO:42, a light chain CDR2 comprising the amino acid sequence of SEQ ID NO:43, and a light chain CDR3 comprising the amino acid sequence of SEQ ID NO:44;
i. a heavy chain CDR1 comprising the amino acid sequence of SEQ ID NO:46, a heavy chain CDR2 comprising the amino acid sequence of SEQ ID NO:47, a heavy chain CDR3 comprising the amino acid sequence of SEQ ID NO:48, a light chain CDR1 comprising the amino acid sequence of SEQ ID NO:50, a light chain CDR2 comprising the amino acid sequence of SEQ ID NO:51, and a light chain CDR3 comprising the amino acid sequence of SEQ ID NO:52;
j. a heavy chain CDR1 comprising the amino acid sequence of SEQ ID NO:54, a heavy chain CDR2 comprising the amino acid sequence of SEQ ID NO:55, a heavy chain CDR3 comprising the amino acid sequence of SEQ ID NO:56, a light chain CDR1 comprising the amino acid sequence of SEQ ID NO:58, a light chain CDR2 comprising the amino acid sequence of SEQ ID NO:59, and a light chain CDR3 comprising the amino acid sequence of SEQ ID NO:60;
k. a heavy chain CDR1 comprising the amino acid sequence of SEQ ID NO:54, a heavy chain CDR2 comprising the amino acid sequence of SEQ ID NO:55, a heavy chain CDR3 comprising the amino acid sequence of SEQ ID NO:62, a light chain CDR1 comprising the amino acid sequence of SEQ ID NO:58, a light chain CDR2 comprising the amino acid sequence of SEQ ID NO:59, and a light chain CDR3 comprising the amino acid sequence of SEQ ID NO:60;
l. a heavy chain CDR1 comprising the amino acid sequence of SEQ ID NO:64, a heavy chain CDR2 comprising the amino acid sequence of SEQ ID NO:19, a heavy chain CDR3 comprising the amino acid sequence of SEQ ID NO:65, a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8;
m. a heavy chain CDR1 comprising the amino acid sequence of SEQ ID NO:67, a heavy chain CDR2 comprising the amino acid sequence of SEQ ID NO:68, a heavy chain CDR3 comprising the amino acid sequence of SEQ ID NO:69, a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8;
n. a heavy chain CDR1 comprising the amino acid sequence of SEQ ID NO:10, a heavy chain CDR2 comprising the amino acid sequence of SEQ ID NO:71, a heavy chain CDR3 comprising the amino acid sequence of SEQ ID NO:72, a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8;
o. a heavy chain CDR1 comprising the amino acid sequence of SEQ ID NO:54, a heavy chain CDR2 comprising the amino acid sequence of SEQ ID NO:74, a heavy chain CDR3 comprising the amino acid sequence of SEQ ID NO:75, a light chain CDR1 comprising the amino acid sequence of SEQ ID NO:77, a light chain CDR2 comprising the amino acid sequence of SEQ ID NO:51, and a light chain CDR3 comprising the amino acid sequence of SEQ ID NO:78;
p. a heavy chain CDR1 comprising the amino acid sequence of SEQ ID NO:22, a heavy chain CDR2 comprising the amino acid sequence of SEQ ID NO:23, a heavy chain CDR3 comprising the amino acid sequence of SEQ ID NO:80, a light chain CDR1 comprising the amino acid sequence of SEQ ID NO:82, a light chain CDR2 comprising the amino acid sequence of SEQ ID NO:7, and a light chain CDR3 comprising the amino acid sequence of SEQ ID NO:83; or
q. a heavy chain CDR1 comprising the amino acid sequence of SEQ ID NO:22, a heavy chain CDR2 comprising the amino acid sequence of SEQ ID NO:85, a heavy chain CDR3 comprising the amino acid sequence of SEQ ID NO:86, a light chain CDR1 comprising the amino acid sequence of SEQ ID NO:88, a light chain CDR2 comprising the amino acid sequence of SEQ ID NO:43, and a light chain CDR3 comprising the amino acid sequence of SEQ ID NO:89;
   wherein the CDRs are defined according to Kabat.

In the ROR1×CD3 bispecific antibodies, or bispecific antigen-binding fragments thereof, the second antigen-binding site that immunospecifically bind CD3 can have a heavy chain CDR1 comprising the amino acid sequence of SEQ ID NO:92, a heavy chain CDR2 comprising the amino acid sequence of SEQ ID NO:93, a heavy chain CDR3 comprising the amino acid sequence of SEQ ID NO:94, a light chain CDR1 comprising the amino acid sequence of SEQ ID NO:95, a light chain CDR2 comprising the amino acid sequence of SEQ ID NO:96, and a light chain CDR3 comprising the amino acid sequence of SEQ ID NO:97, wherein the CDRs are defined according to Kabat.

In some embodiments, the isolated ROR1×CD3 bispecific antibodies, or bispecific antigen-binding fragments thereof, can comprise: a first heavy chain (HC1); a second heavy chain (HC2); a first light chain (LC1); and a second light chain (LC2), wherein the HC1 and the LC1 form a first antigen-binding site that immunospecifically binds ROR1, and the HC2 and the LC2 form a second antigen-binding site that immunospecifically binds CD3. In some aspects, the ROR1×CD3 bispecific antibodies or bispecific antigen-binding fragments thereof, comprise a HC1 and LC1 wherein:
a. the HC1 has a heavy chain CDR1 comprising the amino acid sequence of SEQ ID NO:2, a heavy chain CDR2 comprising the amino acid sequence of SEQ ID NO:3, a heavy chain CDR3 comprising the amino acid sequence of SEQ ID NO:4, and the LC1 has a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8;
b. the HC1 has a heavy chain CDR1 comprising the amino acid sequence of SEQ ID NO:10, a heavy chain CDR2 comprising the amino acid sequence of SEQ ID NO:11, a heavy chain CDR3 comprising the amino acid sequence of SEQ ID NO:12, and the LC1 has a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8;
c. the HC1 has a heavy chain CDR1 comprising the amino acid sequence of SEQ ID NO:14, a heavy chain CDR2 comprising the amino acid sequence of SEQ ID NO:15, a heavy chain CDR3 comprising the amino acid sequence of SEQ ID NO:16, and the LC1 has a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8;
d. the HC1 has a heavy chain CDR1 comprising the amino acid sequence of SEQ ID NO:18, a heavy chain CDR2 comprising the amino acid sequence of SEQ ID NO:19, a heavy chain CDR3 comprising the amino acid sequence of SEQ ID NO:20, and the LC1 has a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8;
e. the HC1 has a heavy chain CDR1 comprising the amino acid sequence of SEQ ID NO:22, a heavy chain CDR2 comprising the amino acid sequence of SEQ ID NO:23, a heavy chain CDR3 comprising the amino acid sequence of SEQ ID NO:24, and the LC1 has a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8;
f. the HC1 has a heavy chain CDR1 comprising the amino acid sequence of SEQ ID NO:26, a heavy chain CDR2 comprising the amino acid sequence of SEQ ID NO:27, a heavy chain CDR3 comprising the amino acid sequence of SEQ ID NO:28, and the LC1 has a light chain CDR1 comprising the amino acid sequence of SEQ ID NO:30, a light chain CDR2 comprising the amino acid sequence of SEQ ID NO:31, and a light chain CDR3 comprising the amino acid sequence of SEQ ID NO:32;
g. the HC1 has a heavy chain CDR1 comprising the amino acid sequence of SEQ ID NO:2, a heavy chain CDR2 comprising the amino acid sequence of SEQ ID NO:34, a heavy chain CDR3 comprising the amino acid sequence of SEQ ID NO:35, and the LC1 has a light chain CDR1 comprising the amino acid sequence of SEQ ID NO:37, a light chain CDR2 comprising the amino acid sequence of SEQ ID NO:7, and a light chain CDR3 comprising the amino acid sequence of SEQ ID NO:38;
h. the HC1 has a heavy chain CDR1 comprising the amino acid sequence of SEQ ID NO:22, a heavy chain CDR2 comprising the amino acid sequence of SEQ ID NO:23, a heavy chain CDR3 comprising the amino acid sequence of SEQ ID NO:40, and the LC1 has a light chain CDR1 comprising the amino acid sequence of SEQ ID NO:42, a light chain CDR2 comprising the amino acid sequence of SEQ ID NO:43, and a light chain CDR3 comprising the amino acid sequence of SEQ ID NO:44;
i. the HC1 has a heavy chain CDR1 comprising the amino acid sequence of SEQ ID NO:46, a heavy chain CDR2 comprising the amino acid sequence of SEQ ID NO:47, a heavy chain CDR3 comprising the amino acid sequence of SEQ ID NO:48, and the LC1 has a light chain CDR1 comprising the amino acid sequence of SEQ ID NO:50, a light chain CDR2 comprising the amino acid sequence of SEQ ID NO:51, and a light chain CDR3 comprising the amino acid sequence of SEQ ID NO:52;
j. the HC1 has a heavy chain CDR1 comprising the amino acid sequence of SEQ ID NO:54, a heavy chain CDR2 comprising the amino acid sequence of SEQ ID NO:55, a heavy chain CDR3 comprising the amino acid sequence of SEQ ID NO:56, and the LC1 has a light chain CDR1 comprising the amino acid sequence of SEQ ID NO:58, a light chain CDR2 comprising the amino acid sequence of SEQ ID NO:59, and a light chain CDR3 comprising the amino acid sequence of SEQ ID NO:60;
k. the HC1 has a heavy chain CDR1 comprising the amino acid sequence of SEQ ID NO:54, a heavy chain CDR2 comprising the amino acid sequence of SEQ ID NO:55, a heavy chain CDR3 comprising the amino acid sequence of SEQ ID NO:62, and the LC1 has a light chain CDR1 comprising the amino acid sequence of SEQ ID NO:58, a light chain CDR2 comprising the amino acid sequence of SEQ ID NO:59, and a light chain CDR3 comprising the amino acid sequence of SEQ ID NO:60;
l. the HC1 has a heavy chain CDR1 comprising the amino acid sequence of SEQ ID NO:64, a heavy chain CDR2 comprising the amino acid sequence of SEQ ID NO:19, a heavy chain CDR3 comprising the amino acid sequence of SEQ ID NO:65, and the LC1 has a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8;
m. the HC1 has a heavy chain CDR1 comprising the amino acid sequence of SEQ ID NO:67, a heavy chain CDR2 comprising the amino acid sequence of SEQ ID NO:68, a heavy chain CDR3 comprising the amino acid sequence of SEQ ID NO:69, and the LC1 has a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8;
n. the HC1 has a heavy chain CDR1 comprising the amino acid sequence of SEQ ID NO:10, a heavy chain CDR2 comprising the amino acid sequence of SEQ ID NO:71, a heavy chain CDR3 comprising the amino acid sequence of SEQ ID NO:72, and the LC1 has a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8;
o. the HC1 has a heavy chain CDR1 comprising the amino acid sequence of SEQ ID NO:54, a heavy chain CDR2 comprising the amino acid sequence of SEQ ID NO:74, a heavy chain CDR3 comprising the amino acid sequence of SEQ ID NO:75, and the LC1 has a light chain CDR1 comprising the amino acid sequence of SEQ ID NO:77, a light chain CDR2 comprising the amino acid sequence of SEQ ID NO:51, and a light chain CDR3 comprising the amino acid sequence of SEQ ID NO:78;
p. the HC1 has a heavy chain CDR1 comprising the amino acid sequence of SEQ ID NO:22, a heavy chain CDR2 comprising the amino acid sequence of SEQ ID NO:23, a heavy chain CDR3 comprising the amino acid sequence of SEQ ID NO:80, and the LC1 has a light chain CDR1 comprising the amino acid sequence of SEQ ID NO:82, a light chain CDR2 comprising the amino acid sequence of SEQ ID NO:7, and a light chain CDR3 comprising the amino acid sequence of SEQ ID NO:83; or
q. the HC1 has a heavy chain CDR1 comprising the amino acid sequence of SEQ ID NO:22, a heavy chain CDR2 comprising the amino acid sequence of SEQ ID NO:85, a heavy chain CDR3 comprising the amino acid sequence of SEQ ID NO:86, and the LC1 has a light chain CDR1 comprising the amino acid sequence of SEQ ID NO:88, a light chain CDR2 comprising the amino acid sequence of SEQ ID NO:43, and a light chain CDR3 comprising the amino acid sequence of SEQ ID NO:89;
   wherein the CDRs are defined according to Kabat.

In some aspects, the ROR1×CD3 bispecific antibodies or bispecific antigen-binding fragments thereof comprise an HC1 and LC1 wherein
a. the HC1 comprises an amino acid sequence that is at least 90% identical to the amino acid sequence of SEQ ID NO:1 and the LC1 comprises an amino acid sequence that is at least 90% identical to the amino acid sequence of SEQ ID NO:5;
b. the HC1 comprises an amino acid sequence that is at least 90% identical to the amino acid sequence of SEQ ID NO:9 and the LC1 comprises an amino acid sequence that is at least 90% identical to the amino acid sequence of SEQ ID NO:5;
c. the HC1 comprises an amino acid sequence that is at least 90% identical to the amino acid sequence of SEQ ID NO:13 and the LC1 comprises an amino acid sequence that is at least 90% identical to the amino acid sequence of SEQ ID NO:5;
d. the HC1 comprises an amino acid sequence that is at least 90% identical to the amino acid sequence of SEQ ID NO:17 and the LC1 comprises an amino acid sequence that is at least 90% identical to the amino acid sequence of SEQ ID NO:5;
e. the HC1 comprises an amino acid sequence that is at least 90% identical to the amino acid sequence of SEQ ID NO:21 and the LC1 comprises an amino acid sequence that is at least 90% identical to the amino acid sequence of SEQ ID NO:5;
f. the HC1 comprises an amino acid sequence that is at least 90% identical to the amino acid sequence of SEQ ID NO:25 and the LC1 comprises an amino acid sequence that is at least 90% identical to the amino acid sequence of SEQ ID NO:29;
g. the HC1 comprises an amino acid sequence that is at least 90% identical to the amino acid sequence of SEQ ID NO:33 and the LC1 comprises an amino acid sequence that is at least 90% identical to the amino acid sequence of SEQ ID NO:36;
h. the HC1 comprises an amino acid sequence that is at least 90% identical to the amino acid sequence of SEQ ID NO:39 and the LC1 comprises an amino acid sequence that is at least 90% identical to the amino acid sequence of SEQ ID NO:41;
i. the HC1 comprises an amino acid sequence that is at least 90% identical to the amino acid sequence of SEQ ID NO:45 and the LC1 comprises an amino acid sequence that is at least 90% identical to the amino acid sequence of SEQ ID NO:49;
j. the HC1 comprises an amino acid sequence that is at least 90% identical to the amino acid sequence of SEQ ID NO:53 and the LC1 comprises an amino acid sequence that is at least 90% identical to the amino acid sequence of SEQ ID NO:57;
k. the HC1 comprises an amino acid sequence that is at least 90% identical to the amino acid sequence of SEQ ID NO:61 and the LC1 comprises an amino acid sequence that is at least 90% identical to the amino acid sequence of SEQ ID NO:57;
l. the HC1 comprises an amino acid sequence that is at least 90% identical to the amino acid sequence of SEQ ID NO:63 and the LC1 comprises an amino acid sequence that is at least 90% identical to the amino acid sequence of SEQ ID NO:5;
m. the HC1 comprises an amino acid sequence that is at least 90% identical to the amino acid sequence of SEQ ID NO:66 and the LC1 comprises an amino acid sequence that is at least 90% identical to the amino acid sequence of SEQ ID NO:5;
n. the HC1 comprises an amino acid sequence that is at least 90% identical to the amino acid sequence of SEQ ID NO:70 and the LC1 comprises an amino acid sequence that is at least 90% identical to the amino acid sequence of SEQ ID NO:5;
o. the HC1 comprises an amino acid sequence that is at least 90% identical to the amino acid sequence of SEQ ID NO:73 and the LC1 comprises an amino acid sequence that is at least 90% identical to the amino acid sequence of SEQ ID NO:76;
p. the HC1 comprises an amino acid sequence that is at least 90% identical to the amino acid sequence of SEQ ID NO:79 and the LC1 comprises an amino acid sequence that is at least 90% identical to the amino acid sequence of SEQ ID NO:81; or
q. the HC1 comprises an amino acid sequence that is at least 90% identical to the amino acid sequence of SEQ ID NO:84 and the LC1 comprises an amino acid sequence that is at least 90% identical to the amino acid sequence of SEQ ID NO:87.

In some embodiments, the HC2 has a heavy chain CDR1 comprising the amino acid sequence of SEQ ID NO:92, a heavy chain CDR2 comprising the amino acid sequence of SEQ ID NO:93, a heavy chain CDR3 comprising the amino acid sequence of SEQ ID NO:94, and the LC2 has a light chain CDR1 comprising the amino acid sequence of SEQ ID NO:95, a light chain CDR2 comprising the amino acid sequence of SEQ ID NO:96, and a light chain CDR3 comprising the amino acid sequence of SEQ ID NO:97, wherein the CDRs are defined according to Kabat. In some embodiments, the HC2 comprises an amino acid sequence that is at least 90% identical to SEQ ID NO:90 and the LC2 comprises an amino acid sequence that is at least 90% identical to SEQ ID NO:91.

Also disclosed are nucleic acid molecules encoding the disclosed ROR1×CD3 bispecific antibodies and bispecific antigen-binding fragments thereof, vectors comprising the nucleic acid molecules, and cells expressing the ROR1×CD3 bispecific antibodies and bispecific antigen-binding fragments thereof.

Provided are methods of treating a subject having cancer, the methods comprising administering to the subject a therapeutically effective amount of any of the disclosed the ROR1×CD3 bispecific antibodies or bispecific antigen-binding fragments thereof.

Further disclosed is the use of an effective amount of any of the disclosed ROR1×CD3 bispecific antibodies or bispecific antigen-binding fragments thereof in the treatment of cancer, and the use of any of the disclosed ROR1×CD3 bispecific antibodies or bispecific antigen-binding fragments thereof in the manufacture of a composition for the treatment of cancer.

### BRIEF DESCRIPTION OF THE DRAWINGS

The summary, as well as the following detailed description, is further understood when read in conjunction with the appended drawings. For the purpose of illustrating the disclosed isolated antibodies, isolated bispecific antibodies, and methods, there are shown in the drawings exemplary embodiments of the isolated antibodies, isolated bispecific antibodies, and methods; however, the isolated antibodies, isolated bispecific antibodies, and methods are not limited to the specific embodiments disclosed. In the drawings:

FIG. 1 illustrates the expression of cell-surface ROR1 in various lung tumor cell lines: small cell lung cancer (NCI-H1417); NSCLC adenocarcinoma (HCC-827); NSCLC squamous cell carcinoma (SK-MES-1); and bronchioalveolar carcinoma (NCI-H358). Flow cytometry expression analysis was performed using 0.5 μl 2A2 anti-ROR1 clone (Biolegend cat#357806). MFI values were normalized using binding of an mIgG1 isotype control antibody (Biolegend cat#400120) at the same concentration to calculate the fold-change over background. Single replicates were run for each sample.

FIG. 2A, FIG. 2B, and FIG. 2C illustrate the binning of the 24 anti-ROR1 antibodies that were advanced to expression and purification as IgG4 PAA molecules. (A) Ig Domain binders. (B) Frizzled Domain binders. (C) Kringle Domain binders.

FIG. 3 illustrates cross competition data for the Kringle domain binders for human ROR1. In this experiment 1 μM competing mAb displaced the signal from the labeled RR1B69 molecule.

FIG. 4A, FIG. 4B, and FIG. 4C illustrate the binding affinity of the anti-ROR1 antibodies evaluated by flow cytometry to HCC827 cells. (A) Ig Domain binders. (B) Frizzled Domain binders. (C) Kringle Domain binders.

FIG. 5A, FIG. 5B, and FIG. 5C illustrate in vitro target cell killing data for the specified ROR1×CD3 bispecific antibodies. The data suggests that the Kringle domain binders were most effective in target cell killing. (A) Ig Domain binders. (B) Frizzled Domain binders. (C) Kringle Domain binders.

FIG. 6A, FIG. 6B, and FIG. 6C illustrate the DiscoveRx® T cell mediated cytotoxicity assay carried out with the specified ROR1×CD3 bispecific antibodies. In this experiment, modified SK-MES-1 cells were used. The data suggests that the Kringle domain binders were most effective in target cell killing. (A) Ig Domain binders. (B) Frizzled Domain binders. (C) Kringle Domain binders.

FIG. 7A and FIG. 7B illustrate the binding profiles of ROR1×CD3 bispecific antibody, RCDB5, and its parental anti-ROR1 antibody, RR1B67, as obtained by Biacore (top left and bottom left panels, respectively) using recombinant ROR1 and via MSD-CAT (top right and bottom right panels, respectively) using HEK293 cells engineered to overexpress ROR1. The data shown here is an example for one of 3 or more experiments.

FIG. 8 illustrates the admixture in vivo study in athymic nude mice. The data suggested a dose dependent effect in a solid tumor model.

FIG. 9A and FIG. 9B illustrate the anti-ROR1 parental RR1B67 and ROR1×CD3 bispecific antibody RCDB5 binding to FcγRIIa. (A) n=1; (B) n=2.

FIG. 10A and FIG. 10B illustrate anti-ROR1 parental RR1B67 and ROR1×CD3 bispecific antibody RCDB5 binding to FcγRIIIa. (A) n=1; (B) n=2.

FIG. 11A, FIG. 11B, FIG. 11C, and FIG. 11D illustrate the RR1B67 epitope location and interactions with human ROR1 based on the crystal structure for the RR1B67 Fab/ROR1 kringle (A) Overall structure of RR1B677 Fab bound to the membrane-proximal kringle domain of human ROR1. A schematic representation of the extracellular domains (ECD) of ROR1 is shown for reference. (B) Overview of the RR1B67 epitope location on the ROR1 kringle domain. The three disulfide bonds and single glycosylation site on the human ROR1 kringle domain are also shown. (C) Interaction map showing direct contacts between ROR1 and RR1B67. Van der Waals interactions are shown as dashed lines and hydrogen bonds as solid lines with arrows pointing to the backbone atoms. (D) Close view of the interface between ROR1 and the RR1B67 light and heavy chains. Hydrogen bonds are shown as dashed lines

FIG. 12 illustrates the epitope and paratope residues of RR1B67. The CDR regions (Kabat definition) and Ig-like, frizzled and kringle domains are underlined. The epitope and paratope residues are shaded. Only the sequences for the extracellular region of human ROR1 and the RR1B67 Fab are shown.

FIGS. 13A, 13B, and 13C illustrate the redirected T cell killing of lung tumor cells activated by ROR1×CD3 bispecific antibodies RCDB5 and a prior art antibody, published in WO2014167022A1, Example 3C, Bispecific (Fab)₂×(Fab) antibody bivalent for ROR1 and monovalent for CD3, with Fc, for brevity termed “Engmab” antibody. (A) Percent of live lung tumor cells out of total number of plated cells after treatments with either RCDB5 or Engmab antibodies. (B) Total number of surviving tumor cells per well at the highest antibody concentration used (6.67 nM). (C) Activation of T (effector) cells, as indicated by the percent of CD25+ T cells, at different antibody concentrations.

FIGS. 14A and 14B illustrate ROR1 expression in heme cancer cell lines. (A) ROR1 expression presented as mean fluorescence intensity (MFI) in MCL/CLL—black bar, B lymphoma—grey bar and MM—dashed bar by flow cytometry. Corresponding Fluorescence Minus One (FMO) staining—white bar, served as negative control. (B) Representative ROR1 expression in MAVER-1 cell line (open histogram) and FMO—(filled histogram).

FIG. 15 illustrates ROR1 receptor density (number of ROR1 molecules per cell) in MCL cell lines by flow cytometry.

FIGS. 16A, 16B, 16C, 16D, and 16E illustrate ROR1 expression on primary cryopreserved CLL and MCL samples by flow cytometry. (A) % of tumor cells in lymphoid gate. Tumor cells were defined as % CD19⁺CD5⁺ live cells. (B) % of ROR1⁺ cells of CD19⁺CD5⁺ tumor cells. (C) ROR1 MFI measured on ROR1⁺ population. (D) Representative ROR1 expression on CLL, donor ID 110029386. (E) Representative ROR1 expression on MCL, donor ID 120137190 tumor cells. Open histogram—ROR1, filled histogram—FMO control.

FIG. 17A-F illustrate ROR1×CD3-mediated killing of MCL lines in whole blood cytotoxicity assay in vitro. (A), (B), and (C) show % cytotoxicity (100%−% Live CFSE+ cells) in MAVER-1, JeKo-1, and Z-138 cells, respectively. (D), (E), and (F) show T cell activation (% CD25 expression on CD4+ and CD8+ lymphocytes) in MAVER-1, JeKo-1, and Z-138 cells, respectively. D1—donor 10347; D2—donor 10207.

FIG. 18A-D illustrate ROR1×CD3-mediated killing of MCL lines in cytotoxicity assay in vitro using PBMC as effector cells. (A) % Cytotoxicity (100%−% Live CFSE+ cells) in MAVER-1 cells. (B) % Cytotoxicity (100%−% Live CFSE+ cells) in Z-138 cells. (C) T cell activation (% CD25 expression on CD4+ and CD8+ lymphocytes) in MAVER-1 cells. (D) T cell activation (% CD25 expression on CD4+ and CD8+ lymphocytes) in Z-138 cells. D1—donor M7444; D2—donor M7267.

### DETAILED DESCRIPTION OF ILLUSTRATIVE EMBODIMENTS

The disclosed isolated antibodies, isolated bispecific antibodies, and methods may be understood more readily by reference to the following detailed description taken in connection with the accompanying figures, which form a part of this disclosure. It is to be understood that the disclosed isolated antibodies, isolated bispecific antibodies, and methods are not limited to those described and/or shown herein, and that the terminology used herein is for the purpose of describing particular embodiments by way of example only and is not intended to be limiting of the claimed isolated antibodies, isolated bispecific antibodies, and methods.

Unless specifically stated otherwise, any description as to a possible mechanism or mode of action or reason for improvement is meant to be illustrative only, and the disclosed isolated antibodies, isolated bispecific antibodies, and methods are not to be constrained by the correctness or incorrectness of any such suggested mechanism or mode of action or reason for improvement.

Throughout this text, the descriptions refer to antibodies and methods of using said antibodies. Where the disclosure describes or claims a feature or embodiment associated with an antibody, such a feature or embodiment is equally applicable to the methods of using the antibody. Likewise, where the disclosure describes or claims a feature or embodiment associated with a method of using an antibody, such a feature or embodiment is equally applicable to the antibody.

When a range of values is expressed, another embodiment includes from the one particular value and/or to the other particular value. Further, reference to values stated in ranges include each and every value within that range. All ranges are inclusive and combinable. When values are expressed as approximations, by use of the antecedent “about,” it will be understood that the particular value forms another embodiment. Reference to a particular numerical value includes at least that particular value, unless the context clearly dictates otherwise.

It is to be appreciated that certain features of the disclosed isolated antibodies, isolated bispecific antibodies, and methods which are, for clarity, described herein in the context of separate embodiments, may also be provided in combination in a single embodiment. Conversely, various features of the disclosed isolated antibodies, isolated bispecific antibodies, and methods that are, for brevity, described in the context of a single embodiment, may also be provided separately or in any subcombination.

As used herein, the singular forms “a,” “an,” and “the” include the plural.

Various terms relating to aspects of the description are used throughout the specification and claims. Such terms are to be given their ordinary meaning in the art unless otherwise indicated. Other specifically defined terms are to be construed in a manner consistent with the definitions provided herein.

The term “about” when used in reference to numerical ranges, cutoffs, or specific values is used to indicate that the recited values may vary by up to as much as 10% from the listed value. Thus, the term “about” is used to encompass variations of ±10% or less, variations of ±5% or less, variations of ±1% or less, variations of ±0.5% or less, or variations of ±0.1% or less from the specified value.

“Antibody” refers to all isotypes of immunoglobulins (IgG, IgA, IgE, IgM, IgD, and IgY) including various monomeric, polymeric and chimeric forms, unless otherwise specified. Specifically encompassed by the term “antibody” are polyclonal antibodies, monoclonal antibodies (mAbs), and antibody-like polypeptides, such as chimeric antibodies and humanized antibodies.

“Antigen-binding fragments” or “bispecific antigen-binding fragments” are any proteinaceous structure that may exhibit binding affinity for a particular antigen. Antigen-binding fragments include those provided by any known technique, such as enzymatic cleavage, peptide synthesis, and recombinant techniques. Some antigen-binding fragments are composed of portions of intact antibodies that retain antigen-binding specificity of the parent antibody molecule. For example, antigen-binding fragments may comprise at least one variable region (either a heavy chain or light chain variable region) or one or more CDRs of an antibody known to bind a particular antigen. Examples of suitable antigen-binding fragments include, without limitation, diabodies and single-chain molecules as well as Fab, F(ab′)2, Fc, Fabc, and Fv molecules, single chain (Sc) antibodies, individual antibody light chains, individual antibody heavy chains, chimeric fusions between antibody chains or CDRs and other proteins, protein scaffolds, heavy chain monomers or dimers, light chain monomers or dimers, dimers consisting of one heavy and one light chain, a monovalent fragment consisting of the VL, VH, CL and CH1 domains, or a monovalent antibody as described in WO2007059782, bivalent fragments comprising two Fab fragments linked by a disulfide bridge at the hinge region, a Fd fragment consisting essentially of the V.sub.H and C.sub.H1 domains; a Fv fragment consisting essentially of the VL and VH domains of a single arm of an antibody, a dAb fragment (Ward et al., Nature 341, 544-546 (1989)), which consists essentially of a VH domain and also called domain antibodies (Holt et al; Trends Biotechnol. 2003 November; 21(11):484-90); camelid or nanobodies (Revets et al; Expert Opin Biol Ther. 2005 January; 5(1):111-24); an isolated complementarity determining region (CDR), and the like. All antibody isotypes may be used to produce antigen-binding fragments. Additionally, antigen-binding fragments may include non-antibody proteinaceous frameworks that may successfully incorporate polypeptide segments in an orientation that confers affinity for a given antigen of interest, such as protein scaffolds. Antigen-binding fragments may be recombinantly produced or produced by enzymatic or chemical cleavage of intact antibodies. The phrase “an antibody or antigen-binding fragment thereof” may be used to denote that a given antigen-binding fragment incorporates one or more amino acid segments of the antibody referred to in the phrase.

When used herein in the context of two or more antibodies or antigen-binding fragments, the term “competes with” or “cross-competes with” indicates that the two or more antibodies or antigen-binding fragments compete for binding to ROR1, e.g. compete for ROR1 binding in the assay described in the disclosed Examples.

The term “CD3” refers to the human CD3 protein multi-subunit complex. The CD3 protein multi-subunit complex is composed to 6 distinctive polypeptide chains. These include a CD3γ chain (SwissProt P09693), a CD3δ chain (SwissProt P04234), two CD3ε chains (SwissProt P07766), and one CD3ζ chain homodimer (SwissProt 20963), and which is associated with the T cell receptor α and β chain. The term “CD3” includes any CD3 variant, isoform and species homolog which is naturally expressed by cells (including T cells) or can be expressed on cells transfected with genes or cDNA encoding those polypeptides, unless noted.

“Effective amount” or “therapeutically effective amount” refers to an amount effective, at dosages and for periods of time necessary, to achieve a desired therapeutic result. A therapeutically effective amount of a ROR1×CD3 bispecific antibody or bispecific antigen-binding fragment thereof may vary according to factors such as the disease state, age, sex, and weight of the individual, and the ability of the antibody to elicit a desired response in the individual. A therapeutically effective amount is also one in which any toxic or detrimental effects of the antibody or antibody portion are outweighed by the therapeutically beneficial effects.

The term “epitope” means a protein determinant capable of specific binding to an antibody. Epitopes usually consist of surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents. The epitope may comprise amino acid residues directly involved in the binding and other amino acid residues, which are not directly involved in the binding, such as amino acid residues which are effectively blocked or covered by the specifically antigen binding peptide (in other words, the amino acid residue is within the footprint of the specifically antigen binding peptide).

“Immunospecifically” when used in the context of antibodies, or antibody fragments, represents binding via domains encoded by immunoglobulin genes or fragments of immunoglobulin genes to one or more epitopes of a protein of interest, without preferentially binding other molecules in a sample containing a mixed population of molecules. Typically, an antibody binds to a cognate antigen with a K_{d} of less than about 1×10⁻⁸ M, as measured by a surface plasmon resonance assay or a cell binding assay. Phrases such as “anti-[antigen] antibody” (e.g., anti-ROR1 antibody) are meant to convey that the recited antibody specifically binds the recited antigen.

“Isolated” means a biological component (such as an antibody) has been substantially separated, produced apart from, or purified away from other biological components of the organism in which the component naturally occurs, i.e., other chromosomal and extrachromosomal DNA and RNA, and proteins. Antibodies that have been “isolated” thus include antibodies purified by standard purification methods. “Isolated antibodies” can be part of a composition and still be isolated if such composition is not part of the native environment of the antibody. The term also embraces antibodies prepared by recombinant expression in a host cell as well as chemically synthesized antibodies. An “isolated antibody or antigen-binding fragment thereof,” as used herein, is intended to refer to an antibody or antigen-binding fragment thereof which is substantially free of other antibodies or antigen-binding fragments having different antigenic specificities (for instance, an isolated antibody that specifically binds to ROR1 is substantially free of antibodies that specifically bind antigens other than ROR1). An isolated antibody that specifically binds to an epitope, isoform or variant of ROR1 may, however, have cross-reactivity to other related antigens, for instance from other species (such as ROR1 species homologs).

The term “k_{d}” (sec⁻¹), as used herein, refers to the dissociation rate constant of a particular antibody-antigen interaction. Said value is also referred to as the k_{off} value.

The term “kₐ” (M⁻¹ sec⁻¹), as used herein, refers to the association rate constant of a particular antibody-antigen interaction.

The term “K_{D}” (M), as used herein, refers to the dissociation equilibrium constant of a particular antibody-antigen interaction.

The term “K_{A}” (M⁻¹), as used herein, refers to the association equilibrium constant of a particular antibody-antigen interaction and is obtained by dividing the kₐ by the k_{d}.

“Subject” refers to human and non-human animals, including all vertebrates, e.g., mammals and non-mammals, such as non-human primates, mice, rabbits, sheep, dogs, cats, horses, cows, chickens, amphibians, and reptiles. In many embodiments of the described methods, the subject is a human.

“ROR1” (Receptor Tyrosine Kinase-Like Orphan Receptor 1) refers to the 106-kDa member of the receptor tyrosine kinase family having a UniProt Accession Number Q01973 (human) and Q9Z139 (mouse).

“Treating” or “treatment” refer to any success or indicia of success in the attenuation or amelioration of an injury, pathology, or condition, including any objective or subjective parameter such as abatement, remission, diminishing of symptoms or making the condition more tolerable to the patient, slowing in the rate of degeneration or decline, making the final point of degeneration less debilitating, improving a subject's physical or mental well-being, or prolonging the length of survival. The treatment may be assessed by objective or subjective parameters, including the results of a physical examination, neurological examination, or psychiatric evaluations.

The following abbreviations are used herein: Receptor Tyrosine Kinase-Like Orphan Receptor 1 (ROR1); small cell lung cancer (SCLC), non-small cell lung cancer (NSCLC); circulating tumor cell (CTC); extracellular domain (ECD).

### ROR1 Specific Antibodies and Antigen-Binding Fragments

Disclosed herein are isolated antibodies, or antigen-binding fragments thereof, that immunospecifically bind to ROR1. The disclosed isolated antibodies, or antigen-binding fragments thereof, include those provided in Table 20.

The isolated antibody or antigen-binding fragment thereof can comprise a heavy chain comprising an amino acid sequence that is at least 90%, 95%, or 99% identical to the amino acid sequence of SEQ ID NO:1 and a light chain comprising an amino acid sequence that is at least 90%, 95%, or 99% identical to the amino acid sequence of SEQ ID NO:5. In some embodiments, the heavy chain can comprise, consist of, or consist essentially of the amino acid sequence of SEQ ID NO:1 and the light chain can comprise, consist of, or consist essentially of the amino acid sequence of SEQ ID NO:5.

The isolated antibody or antigen-binding fragment thereof can comprise:
a. a heavy chain CDR1 comprising, consisting of, or consisting essentially of the amino acid sequence of SEQ ID NO:2,
b. a heavy chain CDR2 comprising, consisting of, or consisting essentially of the amino acid sequence of SEQ ID NO:3,
c. a heavy chain CDR3 comprising, consisting of, or consisting essentially of the amino acid sequence of SEQ ID NO:4,
d. a light chain CDR1 comprising, consisting of, or consisting essentially of the amino acid sequence of SEQ ID NO:6,
e. a light chain CDR2 comprising, consisting of, or consisting essentially of the amino acid sequence of SEQ ID NO:7, and
f. a light chain CDR3 comprising, consisting of, or consisting essentially of the amino acid sequence of SEQ ID NO:8,

wherein the CDRs are defined according to Kabat.

The isolated antibody or antigen-binding fragment thereof can further comprise a heavy chain and a light chain that, apart from the CDRs, comprise:
a. a heavy chain amino acid sequence that is at least 90%, 95%, or 99% identical to the amino acid sequence of SEQ ID NO:1 and a light chain amino acid sequence that is at least 90%, 95%, or 99% identical to the amino acid sequence of SEQ ID NO:5; or
b. a heavy chain that comprises, consists of, or consists essentially of the amino acid sequence of SEQ ID NO:1 and a light chain that comprises, consists of, or consists essentially of the amino acid sequence of SEQ ID NO:5.

In some embodiments, the isolated antibody, or antigen-binding fragment thereof, that immunospecifically binds to ROR1, is RR1B65 or an antigen-binding fragment thereof.

The isolated antibody or antigen-binding fragment thereof can comprise a heavy chain comprising an amino acid sequence that is at least 90%, 95%, or 99% identical to the amino acid sequence of SEQ ID NO:9 and a light chain comprising an amino acid sequence that is at least 90%, 95%, or 99% identical to the amino acid sequence of SEQ ID NO:5. In some aspects, the heavy chain can comprise, consist of, or consist essentially of the amino acid sequence of SEQ ID NO:9 and the light chain can comprise, consist of, or consist essentially of the amino acid sequence of SEQ ID NO:5.

The isolated antibody or antigen-binding fragment thereof can comprise:
a. a heavy chain CDR1 comprising, consisting of, or consisting essentially of the amino acid sequence of SEQ ID NO:10,
b. a heavy chain CDR2 comprising, consisting of, or consisting essentially of the amino acid sequence of SEQ ID NO:11,
c. a heavy chain CDR3 comprising, consisting of, or consisting essentially of the amino acid sequence of SEQ ID NO:12,
d. a light chain CDR1 comprising, consisting of, or consisting essentially of the amino acid sequence of SEQ ID NO:6,
e. a light chain CDR2 comprising, consisting of, or consisting essentially of the amino acid sequence of SEQ ID NO:7, and
f. a light chain CDR3 comprising, consisting of, or consisting essentially of the amino acid sequence of SEQ ID NO:8,

wherein the CDRs are defined according to Kabat.

The isolated antibody or antigen-binding fragment thereof can further comprise a heavy chain and a light chain that, apart from the CDRs, comprise:
a. a heavy chain amino acid sequence that is at least 90%, 95%, or 99% identical to the amino acid sequence of SEQ ID NO:9 and a light chain amino acid sequence that is at least 90%, 95%, or 99% identical to the amino acid sequence of SEQ ID NO:5; or
b. a heavy chain that comprises, consists of, or consists essentially of the amino acid sequence of SEQ ID NO:9 and a light chain that comprises, consists of, or consists essentially of the amino acid sequence of SEQ ID NO:5.

In some embodiments, the isolated antibody, or antigen-binding fragment thereof, that immunospecifically binds to ROR1, can be RR1B66 or an antigen-binding fragment thereof.

The isolated antibody or antigen-binding fragment thereof can comprise a heavy chain comprising an amino acid sequence that is at least 90%, 95%, or 99% identical to the amino acid sequence of SEQ ID NO:13 and a light chain comprising an amino acid sequence that is at least 90%, 95%, or 99% identical to the amino acid sequence of SEQ ID NO:5. In some aspects, the heavy chain can comprise, consist of, or consist essentially of the amino acid sequence of SEQ ID NO:13 and the light chain can comprise, consist of, or consist essentially of the amino acid sequence of SEQ ID NO:5.

The isolated antibody or antigen-binding fragment thereof can comprise:
a. a heavy chain CDR1 comprising, consisting of, or consisting essentially of the amino acid sequence of SEQ ID NO:14,
b. a heavy chain CDR2 comprising, consisting of, or consisting essentially of the amino acid sequence of SEQ ID NO:15,
c. a heavy chain CDR3 comprising, consisting of, or consisting essentially of the amino acid sequence of SEQ ID NO:16,
d. a light chain CDR1 comprising, consisting of, or consisting essentially of the amino acid sequence of SEQ ID NO:6,
e. a light chain CDR2 comprising, consisting of, or consisting essentially of the amino acid sequence of SEQ ID NO:7, and
f. a light chain CDR3 comprising, consisting of, or consisting essentially of the amino acid sequence of SEQ ID NO:8,

wherein the CDRs are defined according to Kabat.

In some embodiments, the isolated antibody or antigen-binding fragment thereof can further comprise a heavy chain and light chain that, apart from the CDRs, comprise:
a. a heavy chain amino acid sequence that is at least 90%, 95%, or 99% identical to the amino acid sequence of SEQ ID NO:13 and a light chain amino acid sequence that is at least 90%, 95%, or 99% identical to the amino acid sequence of SEQ ID NO:5; or
b. a heavy chain that comprises, consists of, or consists essentially of the amino acid sequence of SEQ ID NO:13 and a light chain that comprises, consists of, or consists essentially of the amino acid sequence of SEQ ID NO:5.

In some embodiments, the isolated antibody, or antigen-binding fragment thereof, that immunospecifically binds to ROR1, can be RR1B67 or an antigen-binding fragment thereof.

The isolated antibody or antigen-binding fragment thereof can comprise a heavy chain comprising an amino acid sequence that is at least 90%, 95%, or 99% identical to the amino acid sequence of SEQ ID NO:17 and a light chain comprising an amino acid sequence that is at least 90%, 95%, or 99% identical to the amino acid sequence of SEQ ID NO:5. In some aspects, the heavy chain can comprise, consist of, or consist essentially of the amino acid sequence of SEQ ID NO:17 and the light chain can comprise, consist of, or consist essentially of the amino acid sequence of SEQ ID NO:5.

The isolated antibody or antigen-binding fragment thereof can comprise:
a. a heavy chain CDR1 comprising, consisting of, or consisting essentially of the amino acid sequence of SEQ ID NO:18,
b. a heavy chain CDR2 comprising, consisting of, or consisting essentially of the amino acid sequence of SEQ ID NO:19,
c. a heavy chain CDR3 comprising, consisting of, or consisting essentially of the amino acid sequence of SEQ ID NO:20,
d. a light chain CDR1 comprising, consisting of, or consisting essentially of the amino acid sequence of SEQ ID NO:6,
e. a light chain CDR2 comprising, consisting of, or consisting essentially of the amino acid sequence of SEQ ID NO:7, and
f. a light chain CDR3 comprising, consisting of, or consisting essentially of the amino acid sequence of SEQ ID NO:8,

wherein the CDRs are defined according to Kabat.

In some embodiments, the isolated antibody or antigen-binding fragment thereof can further comprise a heavy chain and light chain that, apart from the CDRs, comprise:
a. a heavy chain amino acid sequence that is at least 90%, 95%, or 99% identical to the amino acid sequence of SEQ ID NO:17 and a light chain amino acid sequence that is at least 90%, 95%, or 99% identical to the amino acid sequence of SEQ ID NO:5; or
b. a heavy chain that comprises, consists of, or consist essentially of the amino acid sequence of SEQ ID NO:17 and a light chain that comprises, consists of, or consists essentially of the amino acid sequence of SEQ ID NO:5.

In some embodiments, the isolated antibody, or antigen-binding fragment thereof, that immunospecifically binds to ROR1 is RR1B69 or an antigen-binding fragment thereof.

The isolated antibody or antigen-binding fragment thereof can comprise a heavy chain comprising an amino acid sequence that is at least 90%, 95%, or 99% identical to the amino acid sequence of SEQ ID NO:21 and the light chain comprising an amino acid sequence that is at least 90%, 95%, or 99% identical to the amino acid sequence of SEQ ID NO:5. In some aspects, the heavy chain can comprise, consist of, or consist essentially of the amino acid sequence of SEQ ID NO:21 and the light chain can comprise, consist of, or consist essentially of the amino acid sequence of SEQ ID NO:5.

The isolated antibody or antigen-binding fragment thereof can comprise:
a. a heavy chain CDR1 comprising, consisting of, or consisting essentially of the amino acid sequence of SEQ ID NO:22,
b. a heavy chain CDR2 comprising, consisting of, or consisting essentially of the amino acid sequence of SEQ ID NO:23,
c. a heavy chain CDR3 comprising, consisting of, or consisting essentially of the amino acid sequence of SEQ ID NO:24,
d. a light chain CDR1 comprising, consisting of, or consisting essentially of the amino acid sequence of SEQ ID NO:6,
e. a light chain CDR2 comprising, consisting of, or consisting essentially of the amino acid sequence of SEQ ID NO:7, and
f. a light chain CDR3 comprising, consisting of, or consisting essentially of the amino acid sequence of SEQ ID NO:8,

wherein the CDRs are defined according to Kabat.

In some embodiments, the isolated antibody or antigen-binding fragment thereof can further comprise a heavy chain and light chain that, apart from the CDRs, comprise:
a. a heavy chain amino acid sequence that is at least 90%, 95%, or 99% identical to the amino acid sequence of SEQ ID NO:21 and a light chain amino acid sequence that is at least 90%, 95%, or 99% identical to the amino acid sequence of SEQ ID NO:5; or
b. a heavy chain that comprises, consists of, or consists essentially of the amino acid sequence of SEQ ID NO:21 and a light chain that comprises, consists of, or consists essentially of the amino acid sequence of SEQ ID NO:5.

In some embodiments, the isolated antibody, or antigen-binding fragment thereof, that immunospecifically binds to ROR1 is RR1B70 or an antigen-binding fragment thereof.

The isolated antibody or antigen-binding fragment thereof can comprise a heavy chain comprising an amino acid sequence that is at least 90%, 95%, or 99% identical to the amino acid sequence of SEQ ID NO:25 and a light chain comprising an amino acid sequence that is at least 90%, 95%, or 99% identical to the amino acid sequence of SEQ ID NO:29. In some aspects, the heavy chain can comprise, consist of, or consist essentially of the amino acid sequence of SEQ ID NO:25 and the light chain can comprise, consist of, or consist essentially of the amino acid sequence of SEQ ID NO:29.

The isolated antibody or antigen-binding fragment thereof can comprise:
a. a heavy chain CDR1 comprising, consisting of, or consisting essentially of the amino acid sequence of SEQ ID NO:26,
b. a heavy chain CDR2 comprising, consisting of, or consisting essentially of the amino acid sequence of SEQ ID NO:27,
c. a heavy chain CDR3 comprising, consisting of, or consisting essentially of the amino acid sequence of SEQ ID NO:28,
d. a light chain CDR1 comprising, consisting of, or consisting essentially of the amino acid sequence of SEQ ID NO:30,
e. a light chain CDR2 comprising, consisting of, or consisting essentially of the amino acid sequence of SEQ ID NO:31, and
f. a light chain CDR3 comprising, consisting of, or consisting essentially of the amino acid sequence of SEQ ID NO:32,

wherein the CDRs are defined according to Kabat.

In some embodiments, the isolated antibody or antigen-binding fragment thereof can further comprise a heavy chain and light chain that, apart from the CDRs, comprise:
a. a heavy chain amino acid sequence that is at least 90%, 95%, or 99% identical to the amino acid sequence of SEQ ID NO:25 and a light chain amino acid sequence that is at least 90%, 95%, or 99% identical to the amino acid sequence of SEQ ID NO:29; or
b. a heavy chain that comprises, consists of, or consists essentially of the amino acid sequence of SEQ ID NO:25 and a light chain that comprises, consists of, or consists essentially of the amino acid sequence of SEQ ID NO:29.

In some embodiments, the isolated antibody, or antigen-binding fragment thereof, that immunospecifically binds to ROR1 is RR1B71 or an antigen-binding fragment thereof.

The isolated antibody or antigen-binding fragment thereof can comprise a heavy chain comprising an amino acid sequence that is at least 90%, 95%, or 99% identical to the amino acid sequence of SEQ ID NO:33 and a light chain comprising an amino acid sequence that is at least 90%, 95%, or 99% identical to the amino acid sequence of SEQ ID NO:36. In some aspects, the heavy chain can comprise, consist of, or consist essentially of the amino acid sequence of SEQ ID NO:33 and the light chain can comprise, consist of, or consist essentially of the amino acid sequence of SEQ ID NO:36.

The isolated antibody or antigen-binding fragment thereof can comprise:
a. a heavy chain CDR1 comprising, consisting of, or consisting essentially of the amino acid sequence of SEQ ID NO:2,
b. a heavy chain CDR2 comprising, consisting of, or consisting essentially of the amino acid sequence of SEQ ID NO:34,
c. a heavy chain CDR3 comprising, consisting of, or consisting essentially of the amino acid sequence of SEQ ID NO:35,
d. a light chain CDR1 comprising, consisting of, or consisting essentially of the amino acid sequence of SEQ ID NO:37,
e. a light chain CDR2 comprising, consisting of, or consisting essentially of the amino acid sequence of SEQ ID NO:7, and
f. a light chain CDR3 comprising, consisting of, or consisting essentially of the amino acid sequence of SEQ ID NO:38,

wherein the CDRs are defined according to Kabat.

In some embodiments, the isolated antibody or antigen-binding fragment thereof can further comprise a heavy chain and light chain that, apart from the CDRs, comprise:
a. a heavy chain amino acid sequence that is at least 90%, 95%, or 99% identical to the amino acid sequence of SEQ ID NO:33 and a light chain amino acid sequence that is at least 90%, 95%, or 99% identical to the amino acid sequence of SEQ ID NO:36; or
b. a heavy chain that comprises, consists of, or consists essentially of the amino acid sequence of SEQ ID NO:33 and a light chain that comprises, consists of, or consists essentially of the amino acid sequence of SEQ ID NO:36.

In some embodiments, the isolated antibody, or antigen-binding fragment thereof, that immunospecifically binds to ROR1 is RR1B72 or an antigen-binding fragment thereof.

The isolated antibody or antigen-binding fragment thereof can comprise a heavy chain comprising an amino acid sequence that is at least 90%, 95%, or 99% identical to the amino acid sequence of SEQ ID NO:39 and a light chain comprising an amino acid sequence that is at least 90%, 95%, or 99% identical to the amino acid sequence of SEQ ID NO:41. In some aspects, the heavy chain can comprise, consist of, or consist essentially of the amino acid sequence of SEQ ID NO:39 and the light chain can comprise, consist of, or consist essentially of the amino acid sequence of SEQ ID NO:41.

The isolated antibody or antigen-binding fragment thereof can comprise:
a. a heavy chain CDR1 comprising, consisting of, or consisting essentially of the amino acid sequence of SEQ ID NO:22,
b. a heavy chain CDR2 comprising, consisting of, or consisting essentially of the amino acid sequence of SEQ ID NO:23,
c. a heavy chain CDR3 comprising, consisting of, or consisting essentially of the amino acid sequence of SEQ ID NO:40,
d. a light chain CDR1 comprising, consisting of, or consisting essentially of the amino acid sequence of SEQ ID NO:42,
e. a light chain CDR2 comprising, consisting of, or consisting essentially of the amino acid sequence of SEQ ID NO:43, and
f. a light chain CDR3 comprising, consisting of, or consisting essentially of the amino acid sequence of SEQ ID NO:44,

wherein the CDRs are defined according to Kabat.

In some embodiments, the isolated antibody or antigen-binding fragment thereof can further comprise a heavy chain and light chain that, apart from the CDRs, comprise:
a. a heavy chain amino acid sequence that is at least 90%, 95%, or 99% identical to the amino acid sequence of SEQ ID NO:39 and a light chain amino acid sequence that is at least 90%, 95%, or 99% identical to the amino acid sequence of SEQ ID NO:41; or
b. a heavy chain that comprises, consists of, or consists essentially of the amino acid sequence of SEQ ID NO:39 and a light chain that comprises, consists of, or consists essentially of the amino acid sequence of SEQ ID NO:41.

In some embodiments, the isolated antibody, or antigen-binding fragment thereof, that immunospecifically binds to ROR1 is RR1B74 or an antigen-binding fragment thereof.

The isolated antibody or antigen-binding fragment thereof can comprise a heavy chain comprising an amino acid sequence that is at least 90%, 95%, or 99% identical to the amino acid sequence of SEQ ID NO:45 and a light chain comprising an amino acid sequence that is at least 90%, 95%, or 99% identical to the amino acid sequence of SEQ ID NO:49. In some aspects, the heavy chain can comprise, consist of, or consist essentially of the amino acid sequence of SEQ ID NO:45 and the light chain can comprise, consist of, or consist essentially of the amino acid sequence of SEQ ID NO:49.

The isolated antibody or antigen-binding fragment thereof can comprise:
a. a heavy chain CDR1 comprising, consisting of, or consisting essentially of the amino acid sequence of SEQ ID NO:46,
b. a heavy chain CDR2 comprising, consisting of, or consisting essentially of the amino acid sequence of SEQ ID NO:47,
c. a heavy chain CDR3 comprising, consisting of, or consisting essentially of the amino acid sequence of SEQ ID NO:48,
d. a light chain CDR1 comprising, consisting of, or consisting essentially of the amino acid sequence of SEQ ID NO:50,
e. a light chain CDR2 comprising, consisting of, or consisting essentially of the amino acid sequence of SEQ ID NO:51, and
f. a light chain CDR3 comprising, consisting of, or consisting essentially of the amino acid sequence of SEQ ID NO:52,

wherein the CDRs are defined according to Kabat.

In some embodiments, the isolated antibody or antigen-binding fragment thereof can further comprise a heavy chain and light chain that, apart from the CDRs, comprise:
a. a heavy chain amino acid sequence that is at least 90%, 95%, or 99% identical to the amino acid sequence of SEQ ID NO:45 and a light chain amino acid sequence that is at least 90%, 95%, or 99% identical to the amino acid sequence of SEQ ID NO:49; or
b. a heavy chain that comprises, consists of, or consists essentially of the amino acid sequence of SEQ ID NO:45 and a light chain that comprises, consists of, or consists essentially of the amino acid sequence of SEQ ID NO:49.

In some embodiments, the isolated antibody, or antigen-binding fragment thereof, that immunospecifically binds to ROR1 is RR1B76 or an antigen-binding fragment thereof.

The isolated antibody and antigen-binding fragment thereof can comprise a heavy chain comprising an amino acid sequence that is at least 90%, 95%, or 99% identical to the amino acid sequence of SEQ ID NO:53 and a light chain comprising an amino acid sequence that is at least 90%, 95%, or 99% identical to the amino acid sequence of SEQ ID NO:57. In some aspects, the heavy chain can comprise, consist of, or consist essentially of the amino acid sequence of SEQ ID NO:53 and the light chain can comprise, consist of, or consist essentially of the amino acid sequence of SEQ ID NO:57.

The isolated antibody or antigen-binding fragment thereof can comprise:
a. a heavy chain CDR1 comprising, consisting of, or consisting essentially of the amino acid sequence of SEQ ID NO:54,
b. a heavy chain CDR2 comprising, consisting of, or consisting essentially of the amino acid sequence of SEQ ID NO:55,
c. a heavy chain CDR3 comprising, consisting of, or consisting essentially of the amino acid sequence of SEQ ID NO:56,
d. a light chain CDR1 comprising, consisting of, or consisting essentially of the amino acid sequence of SEQ ID NO:58,
e. a light chain CDR2 comprising, consisting of, or consisting essentially of the amino acid sequence of SEQ ID NO:59, and
f. a light chain CDR3 comprising, consisting of, or consisting essentially of the amino acid sequence of SEQ ID NO:60,

wherein the CDRs are defined according to Kabat.

In some embodiments, the isolated antibody or antigen-binding fragment can further comprise a heavy chain and light chain that, apart from the CDRs, comprise:
a. a heavy chain amino acid sequence that is at least 90%, 95%, or 99% identical to the amino acid sequence of SEQ ID NO:53 and a light chain amino acid sequence that is at least 90%, 95%, or 99% identical to the amino acid sequence of SEQ ID NO:57; or
b. a heavy chain that comprises, consists of, or consists essentially of the amino acid sequence of SEQ ID NO:53 and a light chain that comprises, consists of, or consists essentially of the amino acid sequence of SEQ ID NO:57.

In some embodiments, the isolated antibody, or antigen-binding fragment thereof, that immunospecifically binds to ROR1 is RR1B77 or an antigen-binding fragment thereof.

The isolated antibody or antigen-binding fragment thereof can comprise a heavy chain comprising an amino acid sequence that is at least 90%, 95%, or 99% identical to the amino acid sequence of SEQ ID NO:61 and a light chain comprising an amino acid sequence that is at least 90%, 95%, or 99% identical to the amino acid sequence of SEQ ID NO:57. In some aspects, the heavy chain can comprise, consist of, or consist essentially of the amino acid sequence of SEQ ID NO:61 and the light chain can comprise, consist of, or consist essentially of the amino acid sequence of SEQ ID NO:57.

The isolated antibody or antigen-binding fragment thereof can comprise:
a. a heavy chain CDR1 comprising, consisting of, or consisting essentially of the amino acid sequence of SEQ ID NO:54,
b. a heavy chain CDR2 comprising, consisting of, or consisting essentially of the amino acid sequence of SEQ ID NO:55,
c. a heavy chain CDR3 comprising, consisting of, or consisting essentially of the amino acid sequence of SEQ ID NO:62,
d. a light chain CDR1 comprising, consisting of, or consisting essentially of the amino acid sequence of SEQ ID NO:58,
e. a light chain CDR2 comprising, consisting of, or consisting essentially of the amino acid sequence of SEQ ID NO:59, and
f. a light chain CDR3 comprising, consisting of, or consisting essentially of the amino acid sequence of SEQ ID NO:60,

wherein the CDRs are defined according to Kabat.

In some embodiments, the isolated antibody or antigen-binding fragment thereof can further have a heavy chain and light chain that, apart from the CDRs, comprise:
a. a heavy chain amino acid sequence that is at least 90%, 95%, or 99% identical to the amino acid sequence of SEQ ID NO:61 and a light chain amino acid sequence that is at least 90%, 95%, or 99% identical to the amino acid sequence of SEQ ID NO:57; or
b. a heavy chain that comprises, consists of, or consists essentially of the amino acid sequence of SEQ ID NO:61 and a light chain that comprises, consists of, or consists essentially of the amino acid sequence of SEQ ID NO:57.

In some embodiments, the isolated antibody, or antigen-binding fragment thereof, that immunospecifically binds to ROR1 is RR1B78 or an antigen-binding fragment thereof.

The isolated antibody or antigen-binding fragment thereof can comprise a heavy chain comprising an amino acid sequence that is at least 90%, 95%, or 99% identical to the amino acid sequence of SEQ ID NO:63 and a light chain comprising an amino acid sequence that is at least 90%, 95%, or 99% identical to the amino acid sequence of SEQ ID NO:5. In some aspects, the heavy chain can comprise, consist of, or consist essentially of the amino acid sequence of SEQ ID NO:63 and the light chain can comprise, consist of, or consist essentially of the amino acid sequence of SEQ ID NO:5.

The isolated antibody or antigen-binding fragment thereof can comprise:
a. a heavy chain CDR1 comprising, consisting of, or consisting essentially of the amino acid sequence of SEQ ID NO:64,
b. a heavy chain CDR2 comprising, consisting of, or consisting essentially of the amino acid sequence of SEQ ID NO:19,
c. a heavy chain CDR3 comprising, consisting of, or consisting essentially of the amino acid sequence of SEQ ID NO:65,
d. a light chain CDR1 comprising, consisting of, or consisting essentially of the amino acid sequence of SEQ ID NO:6,
e. a light chain CDR2 comprising, consisting of, or consisting essentially of the amino acid sequence of SEQ ID NO:7, and
f. a light chain CDR3 comprising, consisting of, or consisting essentially of the amino acid sequence of SEQ ID NO:8,

wherein the CDRs are defined according to Kabat.

In some embodiments, the isolated antibody or antigen-binding fragment thereof can further comprise a heavy chain and light chain that, apart from the CDRs, comprise:
a. a heavy chain amino acid sequence that is at least 90%, 95%, or 99% identical to the amino acid sequence of SEQ ID NO:63 and a light chain amino acid sequence that is at least 90%, 95%, or 99% identical to the amino acid sequence of SEQ ID NO:5; or
b. a heavy chain that comprises, consists of, or consists essentially of the amino acid sequence of SEQ ID NO:63 and a light chain that comprises, consists of, or consists essentially of the amino acid sequence of SEQ ID NO:5.

In some embodiments, the isolated antibody, or antigen-binding fragment thereof, that immunospecifically binds to ROR1 is RR1B82 or an antigen-binding fragment thereof.

The isolated antibody or antigen-binding fragment thereof can comprise a heavy chain comprising an amino acid sequence that is at least 90%, 95%, or 99% identical to the amino acid sequence of SEQ ID NO:66 and a light chain comprising an amino acid sequence that is at least 90%, 95%, or 99% identical to the amino acid sequence of SEQ ID NO:5. In some aspects, the heavy chain can comprise, consist of, or consist essentially of the amino acid sequence of SEQ ID NO:66 and the light chain can comprise, consist of, or consist essentially of the amino acid sequence of SEQ ID NO:5.

The isolated antibody or antigen-binding fragment thereof can comprise:
a. a heavy chain CDR1 comprising, consisting of, or consisting essentially of the amino acid sequence of SEQ ID NO:67,
b. a heavy chain CDR2 comprising, consisting of, or consisting essentially of the amino acid sequence of SEQ ID NO:68,
c. a heavy chain CDR3 comprising, consisting of, or consisting essentially of the amino acid sequence of SEQ ID NO:69,
d. a light chain CDR1 comprising, consisting of, or consisting essentially of the amino acid sequence of SEQ ID NO:6,
e. a light chain CDR2 comprising, consisting of, or consisting essentially of the amino acid sequence of SEQ ID NO:7, and
f. a light chain CDR3 comprising, consisting of, or consisting essentially of the amino acid sequence of SEQ ID NO:8,

wherein the CDRs are defined according to Kabat.

In some embodiments, the isolated antibody or antigen-binding fragment thereof can further comprise a heavy chain and light chain that, apart from the CDRs, comprise:
a. a heavy chain amino acid sequence that is at least 90%, 95%, or 99% identical to the amino acid sequence of SEQ ID NO:66 and a light chain amino acid sequence that is at least 90%, 95%, or 99% identical to the amino acid sequence of SEQ ID NO:5; or
b. a heavy chain that comprises, consists of, or consists essentially of the amino acid sequence of SEQ ID NO:66 and a light chain that comprises, consists of, or consists essentially of the amino acid sequence of SEQ ID NO:5.

In some embodiments, the isolated antibody, or antigen-binding fragment thereof, that immunospecifically binds to ROR1 is RR1B83 or an antigen-binding fragment thereof.

The isolated antibody or antigen-binding fragment thereof can comprise a heavy chain comprising an amino acid sequence that is at least 90%, 95%, or 99% identical to the amino acid sequence of SEQ ID NO:70 and a light chain comprising an amino acid sequence that is at least 90%, 95%, or 99% identical to the amino acid sequence of SEQ ID NO:5. In some aspects, the heavy chain can comprise, consist of, or consist essentially of the amino acid sequence of SEQ ID NO:70 and the light chain can comprise, consist of, or consist essentially of the amino acid sequence of SEQ ID NO:5.

The isolated antibody or antigen-binding fragment thereof can comprise:
a. a heavy chain CDR1 comprising, consisting of, or consisting essentially of the amino acid sequence of SEQ ID NO:10,
b. a heavy chain CDR2 comprising, consisting of, or consisting essentially of the amino acid sequence of SEQ ID NO:71,
c. a heavy chain CDR3 comprising, consisting of, or consisting essentially of the amino acid sequence of SEQ ID NO:72,
d. a light chain CDR1 comprising, consisting of, or consisting essentially of the amino acid sequence of SEQ ID NO:6,
e. a light chain CDR2 comprising, consisting of, or consisting essentially of the amino acid sequence of SEQ ID NO:7, and
f. a light chain CDR3 comprising, consisting of, or consisting essentially of the amino acid sequence of SEQ ID NO:8,

wherein the CDRs are defined according to Kabat.

In some embodiments, the isolated antibody or antigen-binding fragment thereof can further comprise a heavy chain and light chain that, apart from the CDRs, comprise:
a. a heavy chain amino acid sequence that is at least 90%, 95%, or 99% identical to the amino acid sequence of SEQ ID NO:70 and a light chain amino acid sequence that is at least 90%, 95%, or 99% identical to the amino acid sequence of SEQ ID NO:5; or
b. a heavy chain that comprises, consists of, or consists essentially of the amino acid sequence of SEQ ID NO:70 and a light chain that comprises, consists of, or consists essentially of the amino acid sequence of SEQ ID NO:5.

In some embodiments, the isolated antibody, or antigen-binding fragment thereof, that immunospecifically binds to ROR1 is RR1B84 or an antigen-binding fragment thereof.

The isolated antibody or antigen-binding fragment thereof can comprise a heavy chain comprising an amino acid sequence that is at least 90%, 95%, or 99% identical to the amino acid sequence of SEQ ID NO:73 and a light chain comprising an amino acid sequence that is at least 90%, 95%, or 99% identical to the amino acid sequence of SEQ ID NO:76. In some aspects, the heavy chain can comprise, consist of, or consist essentially of the amino acid sequence of SEQ ID NO:73 and the light chain can comprise, consist of, or consist essentially of the amino acid sequence of SEQ ID NO:76.

The isolated antibody or antigen-binding fragment thereof can comprise:
a. a heavy chain CDR1 comprising, consisting of, or consisting essentially of the amino acid sequence of SEQ ID NO:54,
b. a heavy chain CDR2 comprising, consisting of, or consisting essentially of the amino acid sequence of SEQ ID NO:74,
c. a heavy chain CDR3 comprising, consisting of, or consisting essentially of the amino acid sequence of SEQ ID NO:75,
d. a light chain CDR1 comprising, consisting of, or consisting essentially of the amino acid sequence of SEQ ID NO:77,
e. a light chain CDR2 comprising, consisting of, or consisting essentially of the amino acid sequence of SEQ ID NO:51, and
f. a light chain CDR3 comprising, consisting of, or consisting essentially of the amino acid sequence of SEQ ID NO:78,

wherein the CDRs are defined according to Kabat.

In some embodiments, the isolated antibody or antigen-binding fragment thereof can further comprise a heavy chain and light chain that, apart from the CDRs, comprise:
a. a heavy chain amino acid sequence that is at least 90%, 95%, or 99% identical to the amino acid sequence of SEQ ID NO:73 and a light chain amino acid sequence that is at least 90%, 95%, or 99% identical to the amino acid sequence of SEQ ID NO:76; or
b. a heavy chain that comprises, consists of, or consists essentially of the amino acid sequence of SEQ ID NO:73 and a light chain that comprises, consists of, or consists essentially of the amino acid sequence of SEQ ID NO:76.

In some embodiments, the isolated antibody, or antigen-binding fragment thereof, that immunospecifically binds to ROR1 is RR1B85 or an antigen-binding fragment thereof.

The isolated antibody or antigen-binding fragment thereof can comprise a heavy chain comprising an amino acid sequence that is at least 90%, 95%, or 99% identical to the amino acid sequence of SEQ ID NO:79 and a light chain comprising an amino acid sequence that is at least 90%, 95%, or 99% identical to the amino acid sequence of SEQ ID NO:81. In some aspects, the heavy chain can comprise, consist of, or consist essentially of the amino acid sequence of SEQ ID NO:79 and the light chain can comprise, consist of, or consist essentially of the amino acid sequence of SEQ ID NO:81.

The isolated antibody or antigen-binding fragment thereof can comprise:
a. a heavy chain CDR1 comprising, consisting of, or consisting essentially of the amino acid sequence of SEQ ID NO:22,
b. a heavy chain CDR2 comprising, consisting of, or consisting essentially of the amino acid sequence of SEQ ID NO:23,
c. a heavy chain CDR3 comprising, consisting of, or consisting essentially of the amino acid sequence of SEQ ID NO:80,
d. a light chain CDR1 comprising, consisting of, or consisting essentially of the amino acid sequence of SEQ ID NO:82,
e. a light chain CDR2 comprising, consisting of, or consisting essentially of the amino acid sequence of SEQ ID NO:7, and
f. a light chain CDR3 comprising, consisting of, or consisting essentially of the amino acid sequence of SEQ ID NO:83,

wherein the CDRs are defined according to Kabat.

In some embodiments, the isolated antibody or antigen-binding fragment thereof can further comprise a heavy chain and light chain that, apart from the CDRs, comprise:
a. a heavy chain amino acid sequence that is at least 90%, 95%, or 99% identical to the amino acid sequence of SEQ ID NO:79 and a light chain amino acid sequence that is at least 90%, 95%, or 99% identical to the amino acid sequence of SEQ ID NO:81; or
b. a heavy chain that comprises, consists of, or consists essentially of the amino acid sequence of SEQ ID NO:79 and a light chain that comprises, consists of, or consists essentially of the amino acid sequence of SEQ ID NO:81.

In some embodiments, the isolated antibody, or antigen-binding fragment thereof, that immunospecifically binds to ROR1 is RR1B86 or an antigen-binding fragment thereof.

The isolated antibody or antigen-binding fragment thereof can comprise a heavy chain comprising an amino acid sequence that is at least 90%, 95%, or 99% identical to the amino acid sequence of SEQ ID NO:84 and a light chain comprising an amino acid sequence that is at least 90%, 95%, or 99% identical to the amino acid sequence of SEQ ID NO:87. In some aspects, the heavy chain can comprise, consist of, or consist essentially of the amino acid sequence of SEQ ID NO:84 and the light chain can comprise, consist of, or consist essentially of the amino acid sequence of SEQ ID NO:87.

The isolated antibody or antigen-binding fragment thereof can comprise:
a. a heavy chain CDR1 comprising, consisting of, or consisting essentially of the amino acid sequence of SEQ ID NO:22,
b. a heavy chain CDR2 comprising, consisting of, or consisting essentially of the amino acid sequence of SEQ ID NO:85,
c. a heavy chain CDR3 comprising, consisting of, or consisting essentially of the amino acid sequence of SEQ ID NO:86,
d. a light chain CDR1 comprising, consisting of, or consisting essentially of the amino acid sequence of SEQ ID NO:88,
e. a light chain CDR2 comprising, consisting of, or consisting essentially of the amino acid sequence of SEQ ID NO:43, and
f. a light chain CDR3 comprising, consisting of, or consisting essentially of the amino acid sequence of SEQ ID NO:89,

wherein the CDRs are defined according to Kabat.

In some embodiments, the isolated antibody or antigen-binding fragment thereof can further comprise a heavy chain and light chain that, apart from the CDRs, comprise:
a. a heavy chain amino acid sequence that is at least 90%, 95%, or 99% identical to the amino acid sequence of SEQ ID NO:84 and a light chain amino acid sequence that is at least 90%, 95%, or 99% identical to the amino acid sequence of SEQ ID NO:87; or
b. a heavy chain that comprises, consists of, or consists essentially of the amino acid sequence of SEQ ID NO:84 and a light chain that comprises, consists of, or consists essentially of the amino acid sequence of SEQ ID NO:87.

In some embodiments, the isolated antibody, or antigen-binding fragment thereof, that immunospecifically binds to ROR1 is RR1B88 or an antigen-binding fragment thereof.

The isolated antibody, or antigen-binding fragment thereof bind to an epitope within the extracellular domain (ECD) of ROR1. In some embodiments, the isolated antibody or antigen-binding fragment thereof can bind to the Ig-like Domain. In some embodiments, the isolated antibody or antigen-binding fragment thereof can bind to the Frizzled Domain. In some embodiments, the isolated antibody or antigen-binding fragment thereof can bind to the Kringle Domain.

The isolated antibody, or antigen-binding fragment thereof, can bind to a region of the ROR1 extracellular domain (ECD) spanning residues 324-387 (TVSVTKSGRQCQPWNSQYPHTHTFTALRFPELNGGHSYCRNPGNQKEAPWCFTLDEN FKSDLCD—SEQ ID NO:98). In some aspects, the epitope of the isolated antibody or antigen-binding fragment thereof comprises SEQ ID NO:98. In some aspects, the epitope of the isolated antibody or antigen-binding fragment thereof consists essentially of SEQ ID NO:98. In some aspects, the epitope of the isolated antibody or antigen-binding fragment thereof consists of SEQ ID NO:98. In some aspects, the epitope of the isolated antibody or antigen-binding fragment thereof is 90%, 95%, or 99% identical to the amino acid sequence of SEQ ID NO:98.

In some embodiments, the isolated antibody or antigen-binding fragment thereof can bind to an epitope on ROR1 comprising T324, V325, S326, V327, T328, S330, G331, R332, Q333, P336, N338, S339, Y341, H359, S360, Y361, L377, D378, and D387. In some embodiments, the isolated antibody or antigen-binding fragment thereof can bind to an epitope on ROR1 consisting essentially of T324, V325, S326, V327, T328, S330, G331, R332, Q333, P336, N338, S339, Y341, H359, S360, Y361, L377, D378, and D387. In some embodiments, the isolated antibody or antigen-binding fragment thereof can bind to an epitope on ROR1 consisting of T324, V325, S326, V327, T328, S330, G331, R332, Q333, P336, N338, S339, Y341, H359, S360, Y361, L377, D378, and D387.

Also provided are isolated antibodies or antigen-binding fragments thereof that compete for binding to ROR1 with a reference antibody or antigen-binding fragment thereof. Suitable reference antibodies include any of the isolated anti-ROR1 antibodies or antigen-binding fragments thereof disclosed above. In some embodiments, the reference antibody or antigen-binding fragment thereof can comprise a heavy chain comprising, consisting essentially of, or consisting of the amino acid sequence of SEQ ID NO:1 and a light chain comprising, consisting essentially of, or consisting of the amino acid sequence of SEQ ID NO:5. Thus, the isolated antibody or antigen-binding fragment thereof can compete for binding to ROR1 with a reference antibody or antigen-binding fragment thereof, the reference antibody or antigen-binding fragment thereof comprising a heavy chain comprising, consisting essentially of, or consisting of the amino acid sequence of SEQ ID NO:1 and a light chain comprising, consisting essentially of, or consisting of the amino acid sequence of SEQ ID NO:5.

In some embodiments, the reference antibody or antigen-binding fragment thereof can comprise a heavy chain comprising, consisting essentially of, or consisting of the amino acid sequence of SEQ ID NO:9 and a light chain comprising, consisting essentially of, or consisting of the amino acid sequence of SEQ ID NO:5. Thus, the isolated antibody or antigen-binding fragment thereof can compete for binding to ROR1 with a reference antibody or antigen-binding fragment thereof, the reference antibody or antigen-binding fragment thereof comprising a heavy chain comprising, consisting essentially of, or consisting of the amino acid sequence of SEQ ID NO:9 and a light chain comprising, consisting essentially of, or consisting of the amino acid sequence of SEQ ID NO:5.

In some embodiments, the reference antibody or antigen-binding fragment thereof can comprise a heavy chain comprising, consisting essentially of, or consisting of the amino acid sequence of SEQ ID NO:13 and a light chain comprising, consisting essentially of, or consisting of the amino acid sequence of SEQ ID NO:5. Thus, the isolated antibody or antigen-binding fragment thereof can compete for binding to ROR1 with a reference antibody or antigen-binding fragment thereof, the reference antibody or antigen-binding fragment thereof comprising a heavy chain comprising, consisting essentially of, or consisting of the amino acid sequence of SEQ ID NO:13 and a light chain comprising, consisting essentially of, or consisting of the amino acid sequence of SEQ ID NO:5.

In some embodiments, the reference antibody or antigen-binding fragment thereof can comprise a heavy chain comprising, consisting essentially of, or consisting of the amino acid sequence of SEQ ID NO:17 and a light chain comprising, consisting essentially of, or consisting of the amino acid sequence of SEQ ID NO:5. Thus, the isolated antibody or antigen-binding fragment thereof can compete for binding to ROR1 with a reference antibody or antigen-binding fragment thereof, the reference antibody or antigen-binding fragment thereof comprising a heavy chain comprising, consisting essentially of, or consisting of the amino acid sequence of SEQ ID NO:17 and a light chain comprising, consisting essentially of, or consisting of the amino acid sequence of SEQ ID NO:5.

In some embodiments, the reference antibody or antigen-binding fragment thereof can comprise a heavy chain comprising, consisting essentially of, or consisting of the amino acid sequence of SEQ ID NO:21 and a light chain comprising, consisting essentially of, or consisting of the amino acid sequence of SEQ ID NO:5. Thus, the isolated antibody or antigen-binding fragment thereof can compete for binding to ROR1 with a reference antibody or antigen-binding fragment thereof, the reference antibody or antigen-binding fragment thereof comprising a heavy chain comprising, consisting essentially of, or consisting of the amino acid sequence of SEQ ID NO:21 and a light chain comprising, consisting essentially of, or consisting of the amino acid sequence of SEQ ID NO:5.

In some embodiments, the reference antibody or antigen-binding fragment thereof can comprise a heavy chain comprising, consisting essentially of, or consisting of the amino acid sequence of SEQ ID NO:25 and a light chain comprising, consisting essentially of, or consisting of the amino acid sequence of SEQ ID NO:29. Thus, the isolated antibody or antigen-binding fragment thereof can compete for binding to ROR1 with a reference antibody or antigen-binding fragment thereof, the reference antibody or antigen-binding fragment thereof comprising a heavy chain comprising, consisting essentially of, or consisting of the amino acid sequence of SEQ ID NO:25 and a light chain comprising, consisting essentially of, or consisting of the amino acid sequence of SEQ ID NO:29.

In some embodiments, the reference antibody or antigen-binding fragment thereof can comprise a heavy chain comprising, consisting essentially of, or consisting of the amino acid sequence of SEQ ID NO:33 and a light chain comprising, consisting essentially of, or consisting of the amino acid sequence of SEQ ID NO:36. Thus, the isolated antibody or antigen-binding fragment thereof can compete for binding to ROR1 with a reference antibody or antigen-binding fragment thereof, the reference antibody or antigen-binding fragment thereof comprising a heavy chain comprising, consisting essentially of, or consisting of the amino acid sequence of SEQ ID NO:33 and a light chain comprising, consisting essentially of, or consisting of the amino acid sequence of SEQ ID NO:36.

In some embodiments, the reference antibody or antigen-binding fragment thereof can comprise a heavy chain comprising, consisting essentially of, or consisting of the amino acid sequence of SEQ ID NO:39 and a light chain comprising, consisting essentially of, or consisting of the amino acid sequence of SEQ ID NO:41. Thus, the isolated antibody or antigen-binding fragment thereof can compete for binding to ROR1 with a reference antibody or antigen-binding fragment thereof, the reference antibody or antigen-binding fragment thereof comprising a heavy chain comprising, consisting essentially of, or consisting of the amino acid sequence of SEQ ID NO:39 and a light chain comprising, consisting essentially of, or consisting of the amino acid sequence of SEQ ID NO:41.

In some embodiments, the reference antibody or antigen-binding fragment thereof can comprise a heavy chain comprising, consisting essentially of, or consisting of the amino acid sequence of SEQ ID NO:45 and a light chain comprising, consisting essentially of, or consisting of the amino acid sequence of SEQ ID NO:49. Thus, the isolated antibody or antigen-binding fragment thereof can compete for binding to ROR1 with a reference antibody or antigen-binding fragment thereof, the reference antibody or antigen-binding fragment thereof comprising a heavy chain comprising, consisting essentially of, or consisting of the amino acid sequence of SEQ ID NO:45 and a light chain comprising, consisting essentially of, or consisting of the amino acid sequence of SEQ ID NO:49.

In some embodiments, the reference antibody or antigen-binding fragment thereof can comprise a heavy chain comprising, consisting essentially of, or consisting of the amino acid sequence of SEQ ID NO:53 and a light chain comprising, consisting essentially of, or consisting of the amino acid sequence of SEQ ID NO:57. Thus, the isolated antibody or antigen-binding fragment thereof can compete for binding to ROR1 with a reference antibody or antigen-binding fragment thereof, the reference antibody or antigen-binding fragment thereof comprising a heavy chain comprising, consisting essentially of, or consisting of the amino acid sequence of SEQ ID NO:53 and a light chain comprising, consisting essentially of, or consisting of the amino acid sequence of SEQ ID NO:57.

In some embodiments, the reference antibody or antigen-binding fragment thereof can comprise a heavy chain comprising, consisting essentially of, or consisting of the amino acid sequence of SEQ ID NO:61 and a light chain comprising, consisting essentially of, or consisting of the amino acid sequence of SEQ ID NO:57. Thus, the isolated antibody or antigen-binding fragment thereof can compete for binding to ROR1 with a reference antibody or antigen-binding fragment thereof, the reference antibody or antigen-binding fragment thereof comprising a heavy chain comprising, consisting essentially of, or consisting of the amino acid sequence of SEQ ID NO:61 and a light chain comprising, consisting essentially of, or consisting of the amino acid sequence of SEQ ID NO:57.

In some embodiments, the reference antibody or antigen-binding fragment thereof can comprise a heavy chain comprising, consisting essentially of, or consisting of the amino acid sequence of SEQ ID NO:63 and a light chain comprising, consisting essentially of, or consisting of the amino acid sequence of SEQ ID NO:5. Thus, the isolated antibody or antigen-binding fragment thereof can compete for binding to ROR1 with a reference antibody or antigen-binding fragment thereof, the reference antibody or antigen-binding fragment thereof comprising a heavy chain comprising, consisting essentially of, or consisting of the amino acid sequence of SEQ ID NO:63 and a light chain comprising, consisting essentially of, or consisting of the amino acid sequence of SEQ ID NO:5.

In some embodiments, the reference antibody or antigen-binding fragment thereof can comprise a heavy chain comprising, consisting essentially of, or consisting of the amino acid sequence of SEQ ID NO:66 and a light chain comprising, consisting essentially of, or consisting of the amino acid sequence of SEQ ID NO:5. Thus, the isolated antibody or antigen-binding fragment thereof can compete for binding to ROR1 with a reference antibody or antigen-binding fragment thereof, the reference antibody or antigen-binding fragment thereof comprising a heavy chain comprising, consisting essentially of, or consisting of the amino acid sequence of SEQ ID NO:66 and a light chain comprising, consisting essentially of, or consisting of the amino acid sequence of SEQ ID NO:5.

In some embodiments, the reference antibody or antigen-binding fragment thereof can comprise a heavy chain comprising, consisting essentially of, or consisting of the amino acid sequence of SEQ ID NO:70 and a light chain comprising, consisting essentially of, or consisting of the amino acid sequence of SEQ ID NO:5. Thus, the isolated antibody or antigen-binding fragment thereof can compete for binding to ROR1 with a reference antibody or antigen-binding fragment thereof, the reference antibody or antigen-binding fragment thereof comprising a heavy chain comprising, consisting essentially of, or consisting of the amino acid sequence of SEQ ID NO:70 and a light chain comprising, consisting essentially of, or consisting of the amino acid sequence of SEQ ID NO:5.

In some embodiments, the reference antibody or antigen-binding fragment thereof can comprise a heavy chain comprising, consisting essentially of, or consisting of the amino acid sequence of SEQ ID NO:73 and a light chain comprising, consisting essentially of, or consisting of the amino acid sequence of SEQ ID NO:76. Thus, the isolated antibody or antigen-binding fragment thereof can compete for binding to ROR1 with a reference antibody or antigen-binding fragment thereof, the reference antibody or antigen-binding fragment thereof comprising a heavy chain comprising, consisting essentially of, or consisting of the amino acid sequence of SEQ ID NO:73 and a light chain comprising, consisting essentially of, or consisting of the amino acid sequence of SEQ ID NO:76.

In some embodiments, the reference antibody or antigen-binding fragment thereof can comprise a heavy chain comprising, consisting essentially of, or consisting of the amino acid sequence of SEQ ID NO:79 and a light chain comprising, consisting essentially of, or consisting of the amino acid sequence of SEQ ID NO:81. Thus, the isolated antibody or antigen-binding fragment thereof can compete for binding to ROR1 with a reference antibody or antigen-binding fragment thereof, the reference antibody or antigen-binding fragment thereof comprising a heavy chain comprising, consisting essentially of, or consisting of the amino acid sequence of SEQ ID NO:79 and a light chain comprising, consisting essentially of, or consisting of the amino acid sequence of SEQ ID NO:81.

In some embodiments, the reference antibody or antigen-binding fragment thereof can comprise a heavy chain comprising, consisting essentially of, or consisting of the amino acid sequence of SEQ ID NO:84 and a light chain comprising, consisting essentially of, or consisting of the amino acid sequence of SEQ ID NO:87. Thus, the isolated antibody or antigen-binding fragment thereof can compete for binding to ROR1 with a reference antibody or antigen-binding fragment thereof, the reference antibody or antigen-binding fragment thereof comprising a heavy chain comprising, consisting essentially of, or consisting of the amino acid sequence of SEQ ID NO:84 and a light chain comprising, consisting essentially of, or consisting of the amino acid sequence of SEQ ID NO:87.

Also provided herein are isolated antibodies, or antigen-binding fragments thereof, that compete for binding to ROR1 with a reference antibody or antigen-binding fragment thereof, wherein the reference antibody or antigen-binding fragment thereof binds an epitope within residues 324-387 (TVSVTKSGRQCQPWNSQYPHTHTFTALRFPELNGGHSYCRNPGNQKEAPWCFTLDEN FKSDLCD—SEQ ID NO:98) of the ROR1 extracellular domain (ECD). In some aspects, the isolated antibodies, or antigen-binding fragments thereof, compete for binding to ROR1 with a reference antibody or antigen-binding fragment thereof, wherein the reference antibody or antigen-binding fragment thereof binds an epitope comprising SEQ ID NO:98. In some aspects, the isolated antibodies, or antigen-binding fragments thereof, compete for binding to ROR1 with a reference antibody or antigen-binding fragment thereof, wherein the reference antibody or antigen-binding fragment thereof binds an epitope consisting essentially of SEQ ID NO:98. In some aspects, the isolated antibodies, or antigen-binding fragments thereof, compete for binding to ROR1 with a reference antibody or antigen-binding fragment thereof, wherein the reference antibody or antigen-binding fragment thereof binds an epitope consisting of SEQ ID NO:98. In some aspects, the isolated antibodies, or antigen-binding fragments thereof, compete for binding to ROR1 with a reference antibody or antigen-binding fragment thereof, wherein the reference antibody or antigen-binding fragment thereof binds an epitope that is 90%, 95%, or 99% identical to the amino acid sequence of SEQ ID NO:98.

The isolated antibody, or antigen-binding fragment thereof, can compete for binding to ROR1 with a reference antibody or antigen-binding fragment thereof, wherein the reference antibody or antigen-binding fragment binds to an epitope on ROR1 comprising T324, V325, S326, V327, T328, S330, G331, R332, Q333, P336, N338, S339, Y341, H359, S360, Y361, L377, D378, and D387. In some embodiments, the isolated antibody, or antigen-binding fragment thereof, can compete for binding to ROR1 with a reference antibody or antigen-binding fragment thereof, wherein the reference antibody or antigen-binding fragment binds to an epitope on ROR1 consisting essentially of T324, V325, S326, V327, T328, S330, G331, R332, Q333, P336, N338, S339, Y341, H359, S360, Y361, L377, D378, and D387. In some embodiments, the isolated antibody, or antigen-binding fragment thereof, can compete for binding to ROR1 with a reference antibody or antigen-binding fragment thereof, wherein the reference antibody or antigen-binding fragment binds to an epitope on ROR1 consisting of T324, V325, S326, V327, T328, S330, G331, R332, Q333, P336, N338, S339, Y341, H359, S360, Y361, L377, D378, and D387. In some aspects, the reference antibody or antigen-binding fragment thereof comprises a heavy chain comprising, consisting essentially of, or consisting of the amino acid sequence of SEQ ID NO:13 and a light chain comprising, consisting essentially of, or consisting of the amino acid sequence of SEQ ID NO:5.

The disclosed anti-ROR1 antibodies or antigen-binding fragments thereof include all isotypes, IgA, IgD, IgE, IgG and IgM, and synthetic multimers of the four-chain immunoglobulin (Ig) structure. The disclosed antibodies or antigen-binding fragments also include the IgY isotype generally found in hen or turkey serum and hen or turkey egg yolk.

The disclosed antibodies or antigen-binding fragments can also be derived from any of the Ig subclasss. For example, the disclosed antibodies, or antigen-binding fragments thereof, can be derived from IgG1, IgG2, IgG3, and IgG4 isotypes. These subtypes share more than 95% homology in the amino acid sequences of the Fc regions but show major differences in the amino acid composition and structure of the hinge region. The Fc region mediates effector functions, such as antibody-dependent cellular cytotoxicity (ADCC) and complement-dependent cytotoxicity (CDC). In ADCC, the Fc region of an antibody binds to Fc receptors (FcgRs) on the surface of immune effector cells such as natural killers and macrophages, leading to the phagocytosis or lysis of the targeted cells. In CDC, the antibodies kill the targeted cells by triggering the complement cascade at the cell surface. The disclosed antibodies include antibodies with the described features of the variable domains in combination with any of the IgG isotypes, including modified versions in which the Fc sequence has been modified to effect different effector functions.

For many applications of therapeutic antibodies, Fc-mediated effector functions are not part of the mechanism of action. These Fc-mediated effector functions can be detrimental and potentially pose a safety risk by causing off-mechanism toxicity. Modifying effector functions can be achieved by engineering the Fc regions to reduce their binding to FcgRs or the complement factors. The binding of IgG to the activating (FcgRI, FcgRIIa, FcgRIIIa and FcgRIIIb) and inhibitory (FcgRIIb) FcgRs or the first component of complement (C1q) depends on residues located in the hinge region and the CH2 domain. Mutations can be introduced in IgG1, IgG2 and IgG4 to reduce or silence Fc functionalities. Silencing mutations can include, but are not limited to IgG1 AA (F234A, L235A), IgG4 PAA (S228P, F234A, L235A), IgG2 AA (V234A, G237A), IgG1 FEA (L234F, L235E, D265A), or IgG1 FES (L234F/L235E/P331S). In some embodiments, the disclosed antibody or antigen-binding fragment thereof can contain the IgG1 AA (F234A, L235A) mutation. In some embodiments, the disclosed antibody or antigen-binding fragment thereof can contain the IgG4 PAA (S228P, F234A, L235A) mutation. In some embodiments, the disclosed antibody or antigen-binding fragment thereof can contain the IgG2 AA (V234A, G237A) mutation. In some embodiments, the disclosed antibody or antigen-binding fragment thereof can contain the IgG1 FEA (L234F, L235E, D265A) mutation. In some embodiments, the disclosed antibody or antigen-binding fragment thereof can contain the IgG1 FES (L234F/L235E/P331S) mutation. In some embodiments, the disclosed antibody or antigen-binding fragment thereof can contain the IgG1 L234A, L235A, and/or F405L mutations. In some embodiments, the disclosed antibody or antigen-binding fragment thereof can contain the S228P, L234A, L235A, F405L, and/or R409K mutations. In some embodiments, the disclosed antibody or antigen-binding fragment thereof can contain the IgG-AA Fc-L234A, L235A, and F405L.

The disclosed antibodies or antigen-binding fragments thereof can comprise an Fc region with one or more of the following properties: (a) reduced effector function when compared to the parent Fc; (b) reduced affinity to Fcg RI, Fcg RIIa, Fcg RIIb, Fcg RIIIb and/or Fcg RIIIa; (c) reduced affinity to FcgRI; (d) reduced affinity to FcgRIIa; (e) reduced affinity to FcgRIIb; (f) reduced affinity to Fcg RIIIb; or (g) reduced affinity to FcgRIIIa.

The anti-ROR1 antibodies and antigen-binding fragments thereof may be derived from any species by recombinant means. For example, the antibodies or antigen-binding fragments may be mouse, rat, goat, horse, swine, bovine, chicken, rabbit, camelid, donkey, human, or chimeric versions thereof. For use in administration to humans, non-human derived antibodies or antigen-binding fragments may be genetically or structurally altered to be less antigenic upon administration to the human patient.

In some embodiments, the antibodies or antigen-binding fragments can be chimeric. As used herein, the term “chimeric” refers to an antibody, or antigen-binding fragment thereof, having at least some portion of at least one variable domain derived from the antibody amino acid sequence of a non-human mammal, a rodent, or a reptile, while the remaining portions of the antibody, or antigen-binding fragment thereof, are derived from a human.

In some embodiments, the antibodies can be humanized antibodies. Humanized antibodies may be chimeric immunoglobulins, immunoglobulin chains or fragments thereof (such as Fv, Fab, Fab′, F(ab′)2 or other antigen-binding subsequences of antibodies) that contain minimal sequence derived from non-human immunoglobulin. For the most part, humanized antibodies are human immunoglobulins (recipient antibody) in which residues from a complementary-determining region (CDR) of the recipient antibody are replaced by residues from a CDR of a non-human species (donor antibody) such as mouse, rat or rabbit having the desired specificity, affinity, and capacity. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin sequence. The humanized antibody may include at least a portion of an immunoglobulin constant region (Fc), typically that of a human immunoglobulin.

The anti-ROR1 antibodies or antigen-binding fragments thereof described herein can have binding affinities for ROR1 that include a dissociation constant (K_{D}) of less than about 5×10⁻⁷ M, preferably less than about 5×10⁻⁸ M. In some embodiments, the anti-ROR1 antibodies or antigen-binding fragments thereof described herein can have binding affinities for ROR1 that include a dissociation constant (K_{D}) of less than about 5×10⁻⁷ M, preferably less than about 5×10⁻⁸ M. The affinity of the described anti-ROR1 antibodies or antigen-binding fragments thereof may be determined by a variety of methods known in the art, such as surface plasmon resonance or ELISA-based methods. Assays for measuring affinity by SPR include assays performed using a BIAcore T200 machine, where the assay is performed at room temperature (e.g. at or near 25° C.), wherein the antibody capable of binding to ROR1 is captured on the Biacore sensor chip by an anti-Fc antibody (e.g. goat anti-human IgG Fc specific antibody Jackson ImmunoResearch laboratories Prod #109-005-098) to a level around 300 RUs, followed by the collection of association and dissociation data at a flow rate of 50 μl/min.

In addition to the described anti-ROR1 antibodies and antigen-binding fragments thereof, also provided are polynucleotide sequences encoding the disclosed antibodies and antigen-binding fragments thereof.

Vectors comprising the polynucleotides are also provided. The vectors can be expression vectors. Recombinant expression vectors containing a sequence encoding the disclosed antibodies or antigen-binding fragments thereof are thus contemplated as within the scope of this disclosure. The expression vector may contain one or more additional sequences such as, but not limited, to regulatory sequences (e.g., promoter, enhancer), selection markers, and polyadenylation signals. Vectors for transforming a wide variety of host cells are well known and include, but are not limited to, plasmids, phagemids, cosmids, baculoviruses, bacmids, bacterial artificial chromosomes (BACs), yeast artificial chromosomes (YACs), as well as other bacterial, yeast and viral vectors.

Also described are cells expressing, and capable of expressing, the disclosed vectors. These cells may be mammalian cells (such as 293F cells, CHO cells), insect cells (such as Sf7 cells), yeast cells, plant cells, or bacteria cells (such as E. coli). The disclosed antibodies may also be produced by hybridoma cells.

### ROR1×CD3 Bispecific Antibodies and Bispecific Antigen Binding Fragments

Disclosed herein are isolated bispecific antibodies, or bispecific antigen-binding fragments thereof, that bind to ROR1 and CD3 (ROR1×CD3 bispecific antibodies). The ROR1×CD3 bispecific antibodies have at least a first antigen-binding site that immunospecifically binds ROR1 (ROR1 arm) and a second antigen-binding site that immunospecifically binds CD3 (CD3 arm).

The isolated ROR1×CD3 bispecific antibodies, or bispecific antigen-binding fragments thereof, can comprise:
a) a first antigen-binding site that immunospecifically binds ROR1, the first antigen-binding site comprising a heavy chain CDR1, CDR2, and CDR3 and a light chain CDR1, CDR2, and CDR3; and
b) a second antigen-binding site that immunospecifically binds CD3, the second antigen-binding site comprising a heavy chain CDR1, CDR2, and CDR3 and a light chain CDR1, CDR2, and CDR3.

Suitable first antigen-binding sites that immunospecifically bind ROR1 include any of the above disclosed anti-ROR1 antibodies. In some embodiments, the first antigen-binding site that immunospecifically binds ROR1 has:
a. a heavy chain CDR1 comprising, consisting essentially of, or consisting of the amino acid sequence of SEQ ID NO:2, a heavy chain CDR2 comprising, consisting essentially of, or consisting of the amino acid sequence of SEQ ID NO:3, a heavy chain CDR3 comprising, consisting essentially of, or consisting of the amino acid sequence of SEQ ID NO:4, a light chain CDR1 comprising, consisting essentially of, or consisting of the amino acid sequence of SEQ ID NO:6, a light chain CDR2 comprising, consisting essentially of, or consisting of the amino acid sequence of SEQ ID NO:7, and a light chain CDR3 comprising, consisting essentially of, or consisting of the amino acid sequence of SEQ ID NO:8, wherein the CDRs are defined according to Kabat;
b. a heavy chain CDR1 comprising, consisting essentially of, or consisting of the amino acid sequence of SEQ ID NO:10, a heavy chain CDR2 comprising, consisting essentially of, or consisting of the amino acid sequence of SEQ ID NO:11, a heavy chain CDR3 comprising, consisting essentially of, or consisting of the amino acid sequence of SEQ ID NO:12, a light chain CDR1 comprising, consisting essentially of, or consisting of the amino acid sequence of SEQ ID NO:6, a light chain CDR2 comprising, consisting essentially of, or consisting of the amino acid sequence of SEQ ID NO:7, and a light chain CDR3 comprising, consisting essentially of, or consisting of the amino acid sequence of SEQ ID NO:8, wherein the CDRs are defined according to Kabat;
c. a heavy chain CDR1 comprising, consisting essentially of, or consisting of the amino acid sequence of SEQ ID NO:14, a heavy chain CDR2 comprising, consisting essentially of, or consisting of the amino acid sequence of SEQ ID NO:15, a heavy chain CDR3 comprising, consisting essentially of, or consisting of the amino acid sequence of SEQ ID NO:16, a light chain CDR1 comprising, consisting essentially of, or consisting of the amino acid sequence of SEQ ID NO:6, a light chain CDR2 comprising, consisting essentially of, or consisting of the amino acid sequence of SEQ ID NO:7, and a light chain CDR3 comprising, consisting essentially of, or consisting of the amino acid sequence of SEQ ID NO:8, wherein the CDRs are defined according to Kabat;
d. a heavy chain CDR1 comprising, consisting essentially of, or consisting of the amino acid sequence of SEQ ID NO:18, a heavy chain CDR2 comprising, consisting essentially of, or consisting of the amino acid sequence of SEQ ID NO:19, a heavy chain CDR3 comprising, consisting essentially of, or consisting of the amino acid sequence of SEQ ID NO:20, a light chain CDR1 comprising, consisting essentially of, or consisting of the amino acid sequence of SEQ ID NO:6, a light chain CDR2 comprising, consisting essentially of, or consisting of the amino acid sequence of SEQ ID NO:7, and a light chain CDR3 comprising, consisting essentially of, or consisting of the amino acid sequence of SEQ ID NO:8, wherein the CDRs are defined according to Kabat;
e. a heavy chain CDR1 comprising, consisting essentially of, or consisting of the amino acid sequence of SEQ ID NO:22, a heavy chain CDR2 comprising, consisting essentially of, or consisting of the amino acid sequence of SEQ ID NO:23, a heavy chain CDR3 comprising, consisting essentially of, or consisting of the amino acid sequence of SEQ ID NO:24, a light chain CDR1 comprising, consisting essentially of, or consisting of the amino acid sequence of SEQ ID NO:6, a light chain CDR2 comprising, consisting essentially of, or consisting of the amino acid sequence of SEQ ID NO:7, and a light chain CDR3 comprising, consisting essentially of, or consisting of the amino acid sequence of SEQ ID NO:8, wherein the CDRs are defined according to Kabat;
f. a heavy chain CDR1 comprising, consisting essentially of, or consisting of the amino acid sequence of SEQ ID NO:26, a heavy chain CDR2 comprising, consisting essentially of, or consisting of the amino acid sequence of SEQ ID NO:27, a heavy chain CDR3 comprising, consisting essentially of, or consisting of the amino acid sequence of SEQ ID NO:28, a light chain CDR1 comprising, consisting essentially of, or consisting of the amino acid sequence of SEQ ID NO:30, a light chain CDR2 comprising, consisting essentially of, or consisting of the amino acid sequence of SEQ ID NO:31, and a light chain CDR3 comprising, consisting essentially of, or consisting of the amino acid sequence of SEQ ID NO:32, wherein the CDRs are defined according to Kabat;
g. a heavy chain CDR1 comprising, consisting essentially of, or consisting of the amino acid sequence of SEQ ID NO:2, a heavy chain CDR2 comprising, consisting essentially of, or consisting of the amino acid sequence of SEQ ID NO:34, a heavy chain CDR3 comprising, consisting essentially of, or consisting of the amino acid sequence of SEQ ID NO:35, a light chain CDR1 comprising, consisting essentially of, or consisting of the amino acid sequence of SEQ ID NO:37, a light chain CDR2 comprising, consisting essentially of, or consisting of the amino acid sequence of SEQ ID NO:7, and a light chain CDR3 comprising, consisting essentially of, or consisting of the amino acid sequence of SEQ ID NO:38, wherein the CDRs are defined according to Kabat;
h. a heavy chain CDR1 comprising, consisting essentially of, or consisting of the amino acid sequence of SEQ ID NO:22, a heavy chain CDR2 comprising, consisting essentially of, or consisting of the amino acid sequence of SEQ ID NO:23, a heavy chain CDR3 comprising, consisting essentially of, or consisting of the amino acid sequence of SEQ ID NO:40, a light chain CDR1 comprising, consisting essentially of, or consisting of the amino acid sequence of SEQ ID NO:42, a light chain CDR2 comprising, consisting essentially of, or consisting of the amino acid sequence of SEQ ID NO:43, and a light chain CDR3 comprising, consisting essentially of, or consisting of the amino acid sequence of SEQ ID NO:44, wherein the CDRs are defined according to Kabat;
i. a heavy chain CDR1 comprising, consisting essentially of, or consisting of the amino acid sequence of SEQ ID NO:46, a heavy chain CDR2 comprising, consisting essentially of, or consisting of the amino acid sequence of SEQ ID NO:47, a heavy chain CDR3 comprising, consisting essentially of, or consisting of the amino acid sequence of SEQ ID NO:48, a light chain CDR1 comprising, consisting essentially of, or consisting of the amino acid sequence of SEQ ID NO:50, a light chain CDR2 comprising, consisting essentially of, or consisting of the amino acid sequence of SEQ ID NO:51, and a light chain CDR3 comprising, consisting essentially of, or consisting of the amino acid sequence of SEQ ID NO:52, wherein the CDRs are defined according to Kabat;
j. a heavy chain CDR1 comprising, consisting essentially of, or consisting of the amino acid sequence of SEQ ID NO:54, a heavy chain CDR2 comprising, consisting essentially of, or consisting of the amino acid sequence of SEQ ID NO:55, a heavy chain CDR3 comprising, consisting essentially of, or consisting of the amino acid sequence of SEQ ID NO:56, a light chain CDR1 comprising, consisting essentially of, or consisting of the amino acid sequence of SEQ ID NO:58, a light chain CDR2 comprising, consisting essentially of, or consisting of the amino acid sequence of SEQ ID NO:59, and a light chain CDR3 comprising, consisting essentially of, or consisting of the amino acid sequence of SEQ ID NO:60, wherein the CDRs are defined according to Kabat;
k. a heavy chain CDR1 comprising, consisting essentially of, or consisting of the amino acid sequence of SEQ ID NO:54, a heavy chain CDR2 comprising, consisting essentially of, or consisting of the amino acid sequence of SEQ ID NO:55, a heavy chain CDR3 comprising, consisting essentially of, or consisting of the amino acid sequence of SEQ ID NO:62, a light chain CDR1 comprising, consisting essentially of, or consisting of the amino acid sequence of SEQ ID NO:58, a light chain CDR2 comprising, consisting essentially of, or consisting of the amino acid sequence of SEQ ID NO:59, and a light chain CDR3 comprising, consisting essentially of, or consisting of the amino acid sequence of SEQ ID NO:60, wherein the CDRs are defined according to Kabat;
l. a heavy chain CDR1 comprising, consisting essentially of, or consisting of the amino acid sequence of SEQ ID NO:64, a heavy chain CDR2 comprising, consisting essentially of, or consisting of the amino acid sequence of SEQ ID NO:19, a heavy chain CDR3 comprising, consisting essentially of, or consisting of the amino acid sequence of SEQ ID NO:65, a light chain CDR1 comprising, consisting essentially of, or consisting of the amino acid sequence of SEQ ID NO:6, a light chain CDR2 comprising, consisting essentially of, or consisting of the amino acid sequence of SEQ ID NO:7, and a light chain CDR3 comprising, consisting essentially of, or consisting of the amino acid sequence of SEQ ID NO:8, wherein the CDRs are defined according to Kabat;
m. a heavy chain CDR1 comprising, consisting essentially of, or consisting of the amino acid sequence of SEQ ID NO:67, a heavy chain CDR2 comprising, consisting essentially of, or consisting of the amino acid sequence of SEQ ID NO:68, a heavy chain CDR3 comprising, consisting essentially of, or consisting of the amino acid sequence of SEQ ID NO:69, a light chain CDR1 comprising, consisting essentially of, or consisting of the amino acid sequence of SEQ ID NO:6, a light chain CDR2 comprising, consisting essentially of, or consisting of the amino acid sequence of SEQ ID NO:7, and a light chain CDR3 comprising, consisting essentially of, or consisting of the amino acid sequence of SEQ ID NO:8, wherein the CDRs are defined according to Kabat;
n. a heavy chain CDR1 comprising, consisting essentially of, or consisting of the amino acid sequence of SEQ ID NO:10, a heavy chain CDR2 comprising, consisting essentially of, or consisting of the amino acid sequence of SEQ ID NO:71, a heavy chain CDR3 comprising, consisting essentially of, or consisting of the amino acid sequence of SEQ ID NO:72, a light chain CDR1 comprising, consisting essentially of, or consisting of the amino acid sequence of SEQ ID NO:6, a light chain CDR2 comprising, consisting essentially of, or consisting of the amino acid sequence of SEQ ID NO:7, and a light chain CDR3 comprising, consisting essentially of, or consisting of the amino acid sequence of SEQ ID NO:8, wherein the CDRs are defined according to Kabat;
o. a heavy chain CDR1 comprising, consisting essentially of, or consisting of the amino acid sequence of SEQ ID NO:54, a heavy chain CDR2 comprising, consisting essentially of, or consisting of the amino acid sequence of SEQ ID NO:74, a heavy chain CDR3 comprising, consisting essentially of, or consisting of the amino acid sequence of SEQ ID NO:75, a light chain CDR1 comprising, consisting essentially of, or consisting of the amino acid sequence of SEQ ID NO:77, a light chain CDR2 comprising, consisting essentially of, or consisting of the amino acid sequence of SEQ ID NO:51, and a light chain CDR3 comprising, consisting essentially of, or consisting of the amino acid sequence of SEQ ID NO:78, wherein the CDRs are defined according to Kabat;
p. a heavy chain CDR1 comprising, consisting essentially of, or consisting of the amino acid sequence of SEQ ID NO:22, a heavy chain CDR2 comprising, consisting essentially of, or consisting of the amino acid sequence of SEQ ID NO:23, a heavy chain CDR3 comprising, consisting essentially of, or consisting of the amino acid sequence of SEQ ID NO:80, a light chain CDR1 comprising, consisting essentially of, or consisting of the amino acid sequence of SEQ ID NO:82, a light chain CDR2 comprising, consisting essentially of, or consisting of the amino acid sequence of SEQ ID NO:7, and a light chain CDR3 comprising, consisting essentially of, or consisting of the amino acid sequence of SEQ ID NO:83, wherein the CDRs are defined according to Kabat; or
q. a heavy chain CDR1 comprising, consisting essentially of, or consisting of the amino acid sequence of SEQ ID NO:22, a heavy chain CDR2 comprising, consisting essentially of, or consisting of the amino acid sequence of SEQ ID NO:85, a heavy chain CDR3 comprising, consisting essentially of, or consisting of the amino acid sequence of SEQ ID NO:86, a light chain CDR1 comprising, consisting essentially of, or consisting of the amino acid sequence of SEQ ID NO:88, a light chain CDR2 comprising, consisting essentially of, or consisting of the amino acid sequence of SEQ ID NO:43, and a light chain CDR3 comprising, consisting essentially of, or consisting of the amino acid sequence of SEQ ID NO:89, wherein the CDRs are defined according to Kabat.

The second antigen-binding site that immunospecifically binds CD3 can have a heavy chain CDR1 comprising, consisting essentially of, or consisting of the amino acid sequence of SEQ ID NO:92, a heavy chain CDR2 comprising, consisting essentially of, or consisting of the amino acid sequence of SEQ ID NO:93, a heavy chain CDR3 comprising, consisting essentially of, or consisting of the amino acid sequence of SEQ ID NO:94, a light chain CDR1 comprising, consisting essentially of, or consisting of the amino acid sequence of SEQ ID NO:95, a light chain CDR2 comprising, consisting essentially of, or consisting of the amino acid sequence of SEQ ID NO:96, and a light chain CDR3 comprising, consisting essentially of, or consisting of the amino acid sequence of SEQ ID NO:97, wherein the CDRs are defined according to Kabat. The second antigen-binding site that immunospecifically binds CD3 can be derived from CD3B219. The second antigen-binding site that immunospecifically binds CD3 can be derived from the CD3 antibodies disclosed in U.S. Pat. No. 8,236,308. The second antigen-binding site that immunospecifically binds CD3 can be derived from the CD3 antibodies disclosed in U.S. Patent App. Pub. No. 2010/0260668. The second antigen-binding site that immunospecifically binds CD3 can be derived from the CD3 antibodies disclosed in U.S. Patent App. Pub. No. 2013/0018174. The second antigen-binding site that immunospecifically binds CD3 can be derived from the CD3 antibodies disclosed in EP2647707. The second antigen-binding site that immunospecifically binds CD3 can be derived from the CD3 antibodies disclosed in U.S. Patent App. Pub. No. 2012/0321626. The second antigen-binding site that immunospecifically binds CD3 can be derived from the CD3 antibodies disclosed in Int'l Pub. No. WO2012/162067. The second antigen-binding site that immunospecifically binds CD3 can be derived from the CD3 antibodies disclosed in U.S. Patent App. Pub. No. 2013/0060011. The second antigen-binding site that immunospecifically binds CD3 can be derived from the CD3 antibodies disclosed in U.S. Patent App. Pub. No. 2013/0058936. The second antigen-binding site that immunospecifically binds CD3 can be derived from the CD3 antibodies disclosed in U.S. Patent App. Pub. No. 2013/0078249. The second antigen-binding site that immunospecifically binds CD3 can be derived from the CD3 antibodies disclosed in U.S. Patent App. Pub. No. 2013/0058937. The second antigen-binding site that immunospecifically binds CD3 can be derived from the CD3 antibodies disclosed in Int'l Pub. No. WO2013/065708.

In some embodiments, the second antigen-binding site that immunospecifically binds CD3 can comprise mutations in the Fc region including, but not limited to, IgG1 AA (F234A, L235A), IgG4 PAA (S228P, F234A, L235A), IgG2 AA (V234A, G237A), IgG1 FEA (L234F, L235E, D265A), or IgG1 FES (L234F/L235E/P331S). In some embodiments, the second antigen-binding site that immunospecifically binds CD3 can contain the IgG1 AA (F234A, L235A) mutation. In some embodiments, the second antigen-binding site that immunospecifically binds CD3 can contain the IgG4 PAA (S228P, F234A, L235A) mutation. In some embodiments, the second antigen-binding site that immunospecifically binds CD3 can contain the IgG2 AA (V234A, G237A) mutation. In some embodiments, the second antigen-binding site that immunospecifically binds CD3 can contain the IgG1 FEA (L234F, L235E, D265A) mutation. In some embodiments, the second antigen-binding site that immunospecifically binds CD3 can contain the IgG1 FES (L234F/L235E/P331S) mutation. In some embodiments, the second antigen-binding site that immunospecifically binds CD3 can contain the IgG1 L234A, L235A, and/or F405L mutations. In some embodiments, the second antigen-binding site that immunospecifically binds CD3 can contain the S228P, L234A, L235A, F405L, and/or R409K mutations. In some embodiments, the second antigen-binding site that immunospecifically binds CD3 can contain the IgG-AA Fc-L234A, L235A, and F405L.

In some embodiments, the isolated ROR1×CD3 bispecific antibody, or bispecific antigen-binding fragment thereof, can comprise:
a. a first antigen-binding site that immunospecifically binds ROR1, the first antigen-binding site having a heavy chain CDR1 comprising, consisting essentially of, or consisting of the amino acid sequence of SEQ ID NO:2, a heavy chain CDR2 comprising, consisting essentially of, or consisting of the amino acid sequence of SEQ ID NO:3, a heavy chain CDR3 comprising, consisting essentially of, or consisting of the amino acid sequence of SEQ ID NO:4, a light chain CDR1 comprising, consisting essentially of, or consisting of the amino acid sequence of SEQ ID NO:6, a light chain CDR2 comprising, consisting essentially of, or consisting of the amino acid sequence of SEQ ID NO:7, and a light chain CDR3 comprising, consisting essentially of, or consisting of the amino acid sequence of SEQ ID NO:8; and
b. a second antigen-binding site that immunospecifically binds CD3, the second antigen-binding site having a heavy chain CDR1 comprising, consisting essentially of, or consisting of the amino acid sequence of SEQ ID NO:92, a heavy chain CDR2 comprising, consisting essentially of, or consisting of the amino acid sequence of SEQ ID NO:93, a heavy chain CDR3 comprising, consisting essentially of, or consisting of the amino acid sequence of SEQ ID NO:94, a light chain CDR1 comprising, consisting essentially of, or consisting of the amino acid sequence of SEQ ID NO:95, a light chain CDR2 comprising, consisting essentially of, or consisting of the amino acid sequence of SEQ ID NO:96, and a light chain CDR3 comprising, consisting essentially of, or consisting of the amino acid sequence of SEQ ID NO:97,

wherein the CDRs are defined according to Kabat.

In some embodiments, the isolated ROR1×CD3 bispecific antibody, or bispecific antigen-binding fragment thereof, can comprise:
a. a first antigen-binding site that immunospecifically binds ROR1, the first antigen-binding site having a heavy chain CDR1 comprising, consisting essentially of, or consisting of the amino acid sequence of SEQ ID NO:10, a heavy chain CDR2 comprising, consisting essentially of, or consisting of the amino acid sequence of SEQ ID NO:11, a heavy chain CDR3 comprising, consisting essentially of, or consisting of the amino acid sequence of SEQ ID NO:12, a light chain CDR1 comprising, consisting essentially of, or consisting of the amino acid sequence of SEQ ID NO:6, a light chain CDR2 comprising, consisting essentially of, or consisting of the amino acid sequence of SEQ ID NO:7, and a light chain CDR3 comprising, consisting essentially of, or consisting of the amino acid sequence of SEQ ID NO:8; and
b. a second antigen-binding site that immunospecifically binds CD3, the second antigen-binding site having a heavy chain CDR1 comprising, consisting essentially of, or consisting of the amino acid sequence of SEQ ID NO:92, a heavy chain CDR2 comprising, consisting essentially of, or consisting of the amino acid sequence of SEQ ID NO:93, a heavy chain CDR3 comprising, consisting essentially of, or consisting of the amino acid sequence of SEQ ID NO:94, a light chain CDR1 comprising, consisting essentially of, or consisting of the amino acid sequence of SEQ ID NO:95, a light chain CDR2 comprising, consisting essentially of, or consisting of the amino acid sequence of SEQ ID NO:96, and a light chain CDR3 comprising, consisting essentially of, or consisting of the amino acid sequence of SEQ ID NO:97,

wherein the CDRs are defined according to Kabat.

In some embodiments, the isolated ROR1×CD3 bispecific antibody, or bispecific antigen-binding fragment thereof, can comprise:
a. a first antigen-binding site that immunospecifically binds ROR1, the first antigen-binding site having a heavy chain CDR1 comprising, consisting essentially of, or consisting of the amino acid sequence of SEQ ID NO:14, a heavy chain CDR2 comprising, consisting essentially of, or consisting of the amino acid sequence of SEQ ID NO:15, a heavy chain CDR3 comprising, consisting essentially of, or consisting of the amino acid sequence of SEQ ID NO:16, a light chain CDR1 comprising, consisting essentially of, or consisting of the amino acid sequence of SEQ ID NO:6, a light chain CDR2 comprising, consisting essentially of, or consisting of the amino acid sequence of SEQ ID NO:7, and a light chain CDR3 comprising, consisting essentially of, or consisting of the amino acid sequence of SEQ ID NO:8; and
b. a second antigen-binding site that immunospecifically binds CD3, the second antigen-binding site having a heavy chain CDR1 comprising, consisting essentially of, or consisting of the amino acid sequence of SEQ ID NO:92, a heavy chain CDR2 comprising, consisting essentially of, or consisting of the amino acid sequence of SEQ ID NO:93, a heavy chain CDR3 comprising, consisting essentially of, or consisting of the amino acid sequence of SEQ ID NO:94, a light chain CDR1 comprising, consisting essentially of, or consisting of the amino acid sequence of SEQ ID NO:95, a light chain CDR2 comprising, consisting essentially of, or consisting of the amino acid sequence of SEQ ID NO:96, and a light chain CDR3 comprising, consisting essentially of, or consisting of the amino acid sequence of SEQ ID NO:97,

wherein the CDRs are defined according to Kabat.

In some embodiments, the isolated ROR1×CD3 bispecific antibody, or bispecific antigen-binding fragment thereof, can comprise:
a. a first antigen-binding site that immunospecifically binds ROR1, the first antigen-binding site having a heavy chain CDR1 comprising, consisting essentially of, or consisting of the amino acid sequence of SEQ ID NO:18, a heavy chain CDR2 comprising, consisting essentially of, or consisting of the amino acid sequence of SEQ ID NO:19, a heavy chain CDR3 comprising, consisting essentially of, or consisting of the amino acid sequence of SEQ ID NO:20, a light chain CDR1 comprising, consisting essentially of, or consisting of the amino acid sequence of SEQ ID NO:6, a light chain CDR2 comprising, consisting essentially of, or consisting of the amino acid sequence of SEQ ID NO:7, and a light chain CDR3 comprising, consisting essentially of, or consisting of the amino acid sequence of SEQ ID NO:8; and
b. a second antigen-binding site that immunospecifically binds CD3, the second antigen-binding site having a heavy chain CDR1 comprising, consisting essentially of, or consisting of the amino acid sequence of SEQ ID NO:92, a heavy chain CDR2 comprising, consisting essentially of, or consisting of the amino acid sequence of SEQ ID NO:93, a heavy chain CDR3 comprising, consisting essentially of, or consisting of the amino acid sequence of SEQ ID NO:94, a light chain CDR1 comprising, consisting essentially of, or consisting of the amino acid sequence of SEQ ID NO:95, a light chain CDR2 comprising, consisting essentially of, or consisting of the amino acid sequence of SEQ ID NO:96, and a light chain CDR3 comprising, consisting essentially of, or consisting of the amino acid sequence of SEQ ID NO:97,

wherein the CDRs are defined according to Kabat.

In some embodiments, the isolated ROR1×CD3 bispecific antibody, or bispecific antigen-binding fragment thereof, can comprise:
a. a first antigen-binding site that immunospecifically binds ROR1, the first antigen-binding site having a heavy chain CDR1 comprising, consisting essentially of, or consisting of the amino acid sequence of SEQ ID NO:22, a heavy chain CDR2 comprising, consisting essentially of, or consisting of the amino acid sequence of SEQ ID NO:23, a heavy chain CDR3 comprising, consisting essentially of, or consisting of the amino acid sequence of SEQ ID NO:24, a light chain CDR1 comprising, consisting essentially of, or consisting of the amino acid sequence of SEQ ID NO:6, a light chain CDR2 comprising, consisting essentially of, or consisting of the amino acid sequence of SEQ ID NO:7, and a light chain CDR3 comprising, consisting essentially of, or consisting of the amino acid sequence of SEQ ID NO:8; and
b. a second antigen-binding site that immunospecifically binds CD3, the second antigen-binding site having a heavy chain CDR1 comprising, consisting essentially of, or consisting of the amino acid sequence of SEQ ID NO:92, a heavy chain CDR2 comprising, consisting essentially of, or consisting of the amino acid sequence of SEQ ID NO:93, a heavy chain CDR3 comprising, consisting essentially of, or consisting of the amino acid sequence of SEQ ID NO:94, a light chain CDR1 comprising, consisting essentially of, or consisting of the amino acid sequence of SEQ ID NO:95, a light chain CDR2 comprising, consisting essentially of, or consisting of the amino acid sequence of SEQ ID NO:96, and a light chain CDR3 comprising, consisting essentially of, or consisting of the amino acid sequence of SEQ ID NO:97,

wherein the CDRs are defined according to Kabat.

In some embodiments, the isolated ROR1×CD3 bispecific antibody, or bispecific antigen-binding fragment thereof, can comprise:
a. a first antigen-binding site that immunospecifically binds ROR1, the first antigen-binding site having a heavy chain CDR1 comprising, consisting essentially of, or consisting of the amino acid sequence of SEQ ID NO:26, a heavy chain CDR2 comprising, consisting essentially of, or consisting of the amino acid sequence of SEQ ID NO:27, a heavy chain CDR3 comprising, consisting essentially of, or consisting of the amino acid sequence of SEQ ID NO:28, a light chain CDR1 comprising, consisting essentially of, or consisting of the amino acid sequence of SEQ ID NO:30, a light chain CDR2 comprising, consisting essentially of, or consisting of the amino acid sequence of SEQ ID NO:31, and a light chain CDR3 comprising, consisting essentially of, or consisting of the amino acid sequence of SEQ ID NO:32; and
b. a second antigen-binding site that immunospecifically binds CD3, the second antigen-binding site having a heavy chain CDR1 comprising, consisting essentially of, or consisting of the amino acid sequence of SEQ ID NO:92, a heavy chain CDR2 comprising, consisting essentially of, or consisting of the amino acid sequence of SEQ ID NO:93, a heavy chain CDR3 comprising, consisting essentially of, or consisting of the amino acid sequence of SEQ ID NO:94, a light chain CDR1 comprising, consisting essentially of, or consisting of the amino acid sequence of SEQ ID NO:95, a light chain CDR2 comprising, consisting essentially of, or consisting of the amino acid sequence of SEQ ID NO:96, and a light chain CDR3 comprising, consisting essentially of, or consisting of the amino acid sequence of SEQ ID NO:97,

wherein the CDRs are defined according to Kabat.

In some embodiments, the isolated ROR1×CD3 bispecific antibody, or bispecific antigen-binding fragment thereof, can comprise:
a. a first antigen-binding site that immunospecifically binds ROR1, the first antigen-binding site having a heavy chain CDR1 comprising, consisting essentially of, or consisting of the amino acid sequence of SEQ ID NO:2, a heavy chain CDR2 comprising, consisting essentially of, or consisting of the amino acid sequence of SEQ ID NO:34, a heavy chain CDR3 comprising, consisting essentially of, or consisting of the amino acid sequence of SEQ ID NO:35, a light chain CDR1 comprising, consisting essentially of, or consisting of the amino acid sequence of SEQ ID NO:37, a light chain CDR2 comprising, consisting essentially of, or consisting of the amino acid sequence of SEQ ID NO:7, and a light chain CDR3 comprising, consisting essentially of, or consisting of the amino acid sequence of SEQ ID NO:38; and
b. a second antigen-binding site that immunospecifically binds CD3, the second antigen-binding site having a heavy chain CDR1 comprising, consisting essentially of, or consisting of the amino acid sequence of SEQ ID NO:92, a heavy chain CDR2 comprising, consisting essentially of, or consisting of the amino acid sequence of SEQ ID NO:93, a heavy chain CDR3 comprising, consisting essentially of, or consisting of the amino acid sequence of SEQ ID NO:94, a light chain CDR1 comprising, consisting essentially of, or consisting of the amino acid sequence of SEQ ID NO:95, a light chain CDR2 comprising, consisting essentially of, or consisting of the amino acid sequence of SEQ ID NO:96, and a light chain CDR3 comprising, consisting essentially of, or consisting of the amino acid sequence of SEQ ID NO:97,

wherein the CDRs are defined according to Kabat.

In some embodiments, the isolated ROR1×CD3 bispecific antibody, or bispecific antigen-binding fragment thereof, can comprise:
a. a first antigen-binding site that immunospecifically binds ROR1, the first antigen-binding site having a heavy chain CDR1 comprising, consisting essentially of, or consisting of the amino acid sequence of SEQ ID NO:22, a heavy chain CDR2 comprising, consisting essentially of, or consisting of the amino acid sequence of SEQ ID NO:23, a heavy chain CDR3 comprising, consisting essentially of, or consisting of the amino acid sequence of SEQ ID NO:40, a light chain CDR1 comprising, consisting essentially of, or consisting of the amino acid sequence of SEQ ID NO:42, a light chain CDR2 comprising, consisting essentially of, or consisting of the amino acid sequence of SEQ ID NO:43, and a light chain CDR3 comprising, consisting essentially of, or consisting of the amino acid sequence of SEQ ID NO:44; and
b. a second antigen-binding site that immunospecifically binds CD3, the second antigen-binding site having a heavy chain CDR1 comprising, consisting essentially of, or consisting of the amino acid sequence of SEQ ID NO:92, a heavy chain CDR2 comprising, consisting essentially of, or consisting of the amino acid sequence of SEQ ID NO:93, a heavy chain CDR3 comprising, consisting essentially of, or consisting of the amino acid sequence of SEQ ID NO:94, a light chain CDR1 comprising, consisting essentially of, or consisting of the amino acid sequence of SEQ ID NO:95, a light chain CDR2 comprising, consisting essentially of, or consisting of the amino acid sequence of SEQ ID NO:96, and a light chain CDR3 comprising, consisting essentially of, or consisting of the amino acid sequence of SEQ ID NO:97,

wherein the CDRs are defined according to Kabat.

In some embodiments, the isolated ROR1×CD3 bispecific antibody, or bispecific antigen-binding fragment thereof, can comprise:
a. a first antigen-binding site that immunospecifically binds ROR1, the first antigen-binding site having a heavy chain CDR1 comprising, consisting essentially of, or consisting of the amino acid sequence of SEQ ID NO:46, a heavy chain CDR2 comprising, consisting essentially of, or consisting of the amino acid sequence of SEQ ID NO:47, a heavy chain CDR3 comprising, consisting essentially of, or consisting of the amino acid sequence of SEQ ID NO:48, a light chain CDR1 comprising, consisting essentially of, or consisting of the amino acid sequence of SEQ ID NO:50, a light chain CDR2 comprising, consisting essentially of, or consisting of the amino acid sequence of SEQ ID NO:51, and a light chain CDR3 comprising, consisting essentially of, or consisting of the amino acid sequence of SEQ ID NO:52; and
b. a second antigen-binding site that immunospecifically binds CD3, the second antigen-binding site having a heavy chain CDR1 comprising, consisting essentially of, or consisting of the amino acid sequence of SEQ ID NO:92, a heavy chain CDR2 comprising, consisting essentially of, or consisting of the amino acid sequence of SEQ ID NO:93, a heavy chain CDR3 comprising, consisting essentially of, or consisting of the amino acid sequence of SEQ ID NO:94, a light chain CDR1 comprising, consisting essentially of, or consisting of the amino acid sequence of SEQ ID NO:95, a light chain CDR2 comprising, consisting essentially of, or consisting of the amino acid sequence of SEQ ID NO:96, and a light chain CDR3 comprising, consisting essentially of, or consisting of the amino acid sequence of SEQ ID NO:97,

wherein the CDRs are defined according to Kabat.

In some embodiments, the isolated ROR1×CD3 bispecific antibody, or bispecific antigen-binding fragment thereof, can comprise:
a. a first antigen-binding site that immunospecifically binds ROR1, the first antigen-binding site having a heavy chain CDR1 comprising, consisting essentially of, or consisting of the amino acid sequence of SEQ ID NO:54, a heavy chain CDR2 comprising, consisting essentially of, or consisting of the amino acid sequence of SEQ ID NO:55, a heavy chain CDR3 comprising, consisting essentially of, or consisting of the amino acid sequence of SEQ ID NO:56, a light chain CDR1 comprising, consisting essentially of, or consisting of the amino acid sequence of SEQ ID NO:58, a light chain CDR2 comprising, consisting essentially of, or consisting of the amino acid sequence of SEQ ID NO:59, and a light chain CDR3 comprising, consisting essentially of, or consisting of the amino acid sequence of SEQ ID NO:60; and
b. a second antigen-binding site that immunospecifically binds CD3, the second antigen-binding site having a heavy chain CDR1 comprising, consisting essentially of, or consisting of the amino acid sequence of SEQ ID NO:92, a heavy chain CDR2 comprising, consisting essentially of, or consisting of the amino acid sequence of SEQ ID NO:93, a heavy chain CDR3 comprising, consisting essentially of, or consisting of the amino acid sequence of SEQ ID NO:94, a light chain CDR1 comprising, consisting essentially of, or consisting of the amino acid sequence of SEQ ID NO:95, a light chain CDR2 comprising, consisting essentially of, or consisting of the amino acid sequence of SEQ ID NO:96, and a light chain CDR3 comprising, consisting essentially of, or consisting of the amino acid sequence of SEQ ID NO:97,

wherein the CDRs are defined according to Kabat.

In some embodiments, the isolated ROR1×CD3 bispecific antibody, or bispecific antigen-binding fragment thereof, can comprise:
a. a first antigen-binding site that immunospecifically binds ROR1, the first antigen-binding site having a heavy chain CDR1 comprising, consisting essentially of, or consisting of the amino acid sequence of SEQ ID NO:54, a heavy chain CDR2 comprising, consisting essentially of, or consisting of the amino acid sequence of SEQ ID NO:55, a heavy chain CDR3 comprising, consisting essentially of, or consisting of the amino acid sequence of SEQ ID NO:62, a light chain CDR1 comprising, consisting essentially of, or consisting of the amino acid sequence of SEQ ID NO:58, a light chain CDR2 comprising, consisting essentially of, or consisting of the amino acid sequence of SEQ ID NO:59, and a light chain CDR3 comprising, consisting essentially of, or consisting of the amino acid sequence of SEQ ID NO:60; and
b. a second antigen-binding site that immunospecifically binds CD3, the second antigen-binding site having a heavy chain CDR1 comprising, consisting essentially of, or consisting of the amino acid sequence of SEQ ID NO:92, a heavy chain CDR2 comprising, consisting essentially of, or consisting of the amino acid sequence of SEQ ID NO:93, a heavy chain CDR3 comprising, consisting essentially of, or consisting of the amino acid sequence of SEQ ID NO:94, a light chain CDR1 comprising, consisting essentially of, or consisting of the amino acid sequence of SEQ ID NO:95, a light chain CDR2 comprising, consisting essentially of, or consisting of the amino acid sequence of SEQ ID NO:96, and a light chain CDR3 comprising, consisting essentially of, or consisting of the amino acid sequence of SEQ ID NO:97,

wherein the CDRs are defined according to Kabat.

In some embodiments, the isolated ROR1×CD3 bispecific antibody, or bispecific antigen-binding fragment thereof, can comprise:
a. a first antigen-binding site that immunospecifically binds ROR1, the first antigen-binding site having a heavy chain CDR1 comprising, consisting essentially of, or consisting of the amino acid sequence of SEQ ID NO:64, a heavy chain CDR2 comprising, consisting essentially of, or consisting of the amino acid sequence of SEQ ID NO:19, a heavy chain CDR3 comprising, consisting essentially of, or consisting of the amino acid sequence of SEQ ID NO:65, a light chain CDR1 comprising, consisting essentially of, or consisting of the amino acid sequence of SEQ ID NO:6, a light chain CDR2 comprising, consisting essentially of, or consisting of the amino acid sequence of SEQ ID NO:7, and a light chain CDR3 comprising, consisting essentially of, or consisting of the amino acid sequence of SEQ ID NO:8;
b. a second antigen-binding site that immunospecifically binds CD3, the second antigen-binding site having a heavy chain CDR1 comprising, consisting essentially of, or consisting of the amino acid sequence of SEQ ID NO:92, a heavy chain CDR2 comprising, consisting essentially of, or consisting of the amino acid sequence of SEQ ID NO:93, a heavy chain CDR3 comprising, consisting essentially of, or consisting of the amino acid sequence of SEQ ID NO:94, a light chain CDR1 comprising, consisting essentially of, or consisting of the amino acid sequence of SEQ ID NO:95, a light chain CDR2 comprising, consisting essentially of, or consisting of the amino acid sequence of SEQ ID NO:96, and a light chain CDR3 comprising, consisting essentially of, or consisting of the amino acid sequence of SEQ ID NO:97,

wherein the CDRs are defined according to Kabat.

In some embodiments, the isolated ROR1×CD3 bispecific antibody, or bispecific antigen-binding fragment thereof, can comprise:
a. a first antigen-binding site that immunospecifically binds ROR1, the first antigen-binding site having a heavy chain CDR1 comprising, consisting essentially of, or consisting of the amino acid sequence of SEQ ID NO:67, a heavy chain CDR2 comprising, consisting essentially of, or consisting of the amino acid sequence of SEQ ID NO:68, a heavy chain CDR3 comprising, consisting essentially of, or consisting of the amino acid sequence of SEQ ID NO:69, a light chain CDR1 comprising, consisting essentially of, or consisting of the amino acid sequence of SEQ ID NO:6, a light chain CDR2 comprising, consisting essentially of, or consisting of the amino acid sequence of SEQ ID NO:7, and a light chain CDR3 comprising, consisting essentially of, or consisting of the amino acid sequence of SEQ ID NO:8; and
b. a second antigen-binding site that immunospecifically binds CD3, the second antigen-binding site having a heavy chain CDR1 comprising, consisting essentially of, or consisting of the amino acid sequence of SEQ ID NO:92, a heavy chain CDR2 comprising, consisting essentially of, or consisting of the amino acid sequence of SEQ ID NO:93, a heavy chain CDR3 comprising, consisting essentially of, or consisting of the amino acid sequence of SEQ ID NO:94, a light chain CDR1 comprising, consisting essentially of, or consisting of the amino acid sequence of SEQ ID NO:95, a light chain CDR2 comprising, consisting essentially of, or consisting of the amino acid sequence of SEQ ID NO:96, and a light chain CDR3 comprising, consisting essentially of, or consisting of the amino acid sequence of SEQ ID NO:97, wherein the CDRs are defined according to Kabat.

In some embodiments, the isolated ROR1×CD3 bispecific antibody, or bispecific antigen-binding fragment thereof, can comprise:
a. a first antigen-binding site that immunospecifically binds ROR1, the first antigen-binding site having a heavy chain CDR1 comprising, consisting essentially of, or consisting of the amino acid sequence of SEQ ID NO:10, a heavy chain CDR2 comprising, consisting essentially of, or consisting of the amino acid sequence of SEQ ID NO:71, a heavy chain CDR3 comprising, consisting essentially of, or consisting of the amino acid sequence of SEQ ID NO:72, a light chain CDR1 comprising, consisting essentially of, or consisting of the amino acid sequence of SEQ ID NO:6, a light chain CDR2 comprising, consisting essentially of, or consisting of the amino acid sequence of SEQ ID NO:7, and a light chain CDR3 comprising, consisting essentially of, or consisting of the amino acid sequence of SEQ ID NO:8; and
b. a second antigen-binding site that immunospecifically binds CD3, the second antigen-binding site having a heavy chain CDR1 comprising, consisting essentially of, or consisting of the amino acid sequence of SEQ ID NO:92, a heavy chain CDR2 comprising, consisting essentially of, or consisting of the amino acid sequence of SEQ ID NO:93, a heavy chain CDR3 comprising, consisting essentially of, or consisting of the amino acid sequence of SEQ ID NO:94, a light chain CDR1 comprising, consisting essentially of, or consisting of the amino acid sequence of SEQ ID NO:95, a light chain CDR2 comprising, consisting essentially of, or consisting of the amino acid sequence of SEQ ID NO:96, and a light chain CDR3 comprising, consisting essentially of, or consisting of the amino acid sequence of SEQ ID NO:97,

wherein the CDRs are defined according to Kabat.

In some embodiments, the isolated ROR1×CD3 bispecific antibody, or bispecific antigen-binding fragment thereof, can comprise:
a. a first antigen-binding site that immunospecifically binds ROR1, the first antigen-binding site having a heavy chain CDR1 comprising, consisting essentially of, or consisting of the amino acid sequence of SEQ ID NO:54, a heavy chain CDR2 comprising, consisting essentially of, or consisting of the amino acid sequence of SEQ ID NO:74, a heavy chain CDR3 comprising, consisting essentially of, or consisting of the amino acid sequence of SEQ ID NO:75, a light chain CDR1 comprising, consisting essentially of, or consisting of the amino acid sequence of SEQ ID NO:77, a light chain CDR2 comprising, consisting essentially of, or consisting of the amino acid sequence of SEQ ID NO:51, and a light chain CDR3 comprising, consisting essentially of, or consisting of the amino acid sequence of SEQ ID NO:78; and
b. a second antigen-binding site that immunospecifically binds CD3, the second antigen-binding site having a heavy chain CDR1 comprising, consisting essentially of, or consisting of the amino acid sequence of SEQ ID NO:92, a heavy chain CDR2 comprising, consisting essentially of, or consisting of the amino acid sequence of SEQ ID NO:93, a heavy chain CDR3 comprising, consisting essentially of, or consisting of the amino acid sequence of SEQ ID NO:94, a light chain CDR1 comprising, consisting essentially of, or consisting of the amino acid sequence of SEQ ID NO:95, a light chain CDR2 comprising, consisting essentially of, or consisting of the amino acid sequence of SEQ ID NO:96, and a light chain CDR3 comprising, consisting essentially of, or consisting of the amino acid sequence of SEQ ID NO:97,

wherein the CDRs are defined according to Kabat.

In some embodiments, the isolated ROR1×CD3 bispecific antibody, or bispecific antigen-binding fragment thereof, can comprise:
a. a first antigen-binding site that immunospecifically binds ROR1, the first antigen-binding site having a heavy chain CDR1 comprising, consisting essentially of, or consisting of the amino acid sequence of SEQ ID NO:22, a heavy chain CDR2 comprising, consisting essentially of, or consisting of the amino acid sequence of SEQ ID NO:23, a heavy chain CDR3 comprising, consisting essentially of, or consisting of the amino acid sequence of SEQ ID NO:80, a light chain CDR1 comprising, consisting essentially of, or consisting of the amino acid sequence of SEQ ID NO:82, a light chain CDR2 comprising, consisting essentially of, or consisting of the amino acid sequence of SEQ ID NO:7, and a light chain CDR3 comprising, consisting essentially of, or consisting of the amino acid sequence of SEQ ID NO:83; and
b. a second antigen-binding site that immunospecifically binds CD3, the second antigen-binding site having a heavy chain CDR1 comprising, consisting essentially of, or consisting of the amino acid sequence of SEQ ID NO:92, a heavy chain CDR2 comprising, consisting essentially of, or consisting of the amino acid sequence of SEQ ID NO:93, a heavy chain CDR3 comprising, consisting essentially of, or consisting of the amino acid sequence of SEQ ID NO:94, a light chain CDR1 comprising, consisting essentially of, or consisting of the amino acid sequence of SEQ ID NO:95, a light chain CDR2 comprising, consisting essentially of, or consisting of the amino acid sequence of SEQ ID NO:96, and a light chain CDR3 comprising, consisting essentially of, or consisting of the amino acid sequence of SEQ ID NO:97, wherein the CDRs are defined according to Kabat.

In some embodiments, the isolated ROR1×CD3 bispecific antibody, or bispecific antigen-binding fragment thereof, can comprise:
a. a first antigen-binding site that immunospecifically binds ROR1, the first antigen-binding site having a heavy chain CDR1 comprising, consisting essentially of, or consisting of the amino acid sequence of SEQ ID NO:22, a heavy chain CDR2 comprising, consisting essentially of, or consisting of the amino acid sequence of SEQ ID NO:85, a heavy chain CDR3 comprising, consisting essentially of, or consisting of the amino acid sequence of SEQ ID NO:86, a light chain CDR1 comprising, consisting essentially of, or consisting of the amino acid sequence of SEQ ID NO:88, a light chain CDR2 comprising, consisting essentially of, or consisting of the amino acid sequence of SEQ ID NO:43, and a light chain CDR3 comprising, consisting essentially of, or consisting of the amino acid sequence of SEQ ID NO:89; and
b. a second antigen-binding site that immunospecifically binds CD3, the second antigen-binding site having a heavy chain CDR1 comprising, consisting essentially of, or consisting of the amino acid sequence of SEQ ID NO:92, a heavy chain CDR2 comprising, consisting essentially of, or consisting of the amino acid sequence of SEQ ID NO:93, a heavy chain CDR3 comprising, consisting essentially of, or consisting of the amino acid sequence of SEQ ID NO:94, a light chain CDR1 comprising, consisting essentially of, or consisting of the amino acid sequence of SEQ ID NO:95, a light chain CDR2 comprising, consisting essentially of, or consisting of the amino acid sequence of SEQ ID NO:96, and a light chain CDR3 comprising, consisting essentially of, or consisting of the amino acid sequence of SEQ ID NO:97,

wherein the CDRs are defined according to Kabat.

Also provided are isolated ROR1×CD3 bispecific antibodies, or bispecific antigen-binding fragments thereof, comprising:

a. a first heavy chain (HC1);

b. a second heavy chain (HC2);

c. a first light chain (LC1); and

d. a second light chain (LC2),

wherein the HC1 and the LC1 form a first antigen-binding site that immunospecifically binds ROR1, and the HC2 and the LC2 form a second antigen-binding site that immunospecifically binds CD3.

In some embodiments, the first antigen-binding site that immunospecifically binds ROR1 is formed from a HC1 and a LC1, wherein:
a. the HC1 has a heavy chain CDR1 comprising, consisting essentially of, or consisting of the amino acid sequence of SEQ ID NO:2, a heavy chain CDR2 comprising, consisting essentially of, or consisting of the amino acid sequence of SEQ ID NO:3, and a heavy chain CDR3 comprising, consisting essentially of, or consisting of the amino acid sequence of SEQ ID NO:4, and the LC1 has a light chain CDR1 comprising, consisting essentially of, or consisting of the amino acid sequence of SEQ ID NO:6, a light chain CDR2 comprising, consisting essentially of, or consisting of the amino acid sequence of SEQ ID NO:7, and a light chain CDR3 comprising, consisting essentially of, or consisting of the amino acid sequence of SEQ ID NO:8, wherein the CDRs are defined according to Kabat;
b. the HC1 has a heavy chain CDR1 comprising, consisting essentially of, or consisting of the amino acid sequence of SEQ ID NO:10, a heavy chain CDR2 comprising, consisting essentially of, or consisting of the amino acid sequence of SEQ ID NO:11, and a heavy chain CDR3 comprising, consisting essentially of, or consisting of the amino acid sequence of SEQ ID NO:12, and the LC1 has a light chain CDR1 comprising, consisting essentially of, or consisting of the amino acid sequence of SEQ ID NO:6, a light chain CDR2 comprising, consisting essentially of, or consisting of the amino acid sequence of SEQ ID NO:7, and a light chain CDR3 comprising, consisting essentially of, or consisting of the amino acid sequence of SEQ ID NO:8, wherein the CDRs are defined according to Kabat;
c. the HC1 has a heavy chain CDR1 comprising, consisting essentially of, or consisting of the amino acid sequence of SEQ ID NO:14, a heavy chain CDR2 comprising, consisting essentially of, or consisting of the amino acid sequence of SEQ ID NO:15, and a heavy chain CDR3 comprising, consisting essentially of, or consisting of the amino acid sequence of SEQ ID NO:16, and the LC1 has a light chain CDR1 comprising, consisting essentially of, or consisting of the amino acid sequence of SEQ ID NO:6, a light chain CDR2 comprising, consisting essentially of, or consisting of the amino acid sequence of SEQ ID NO:7, and a light chain CDR3 comprising, consisting essentially of, or consisting of the amino acid sequence of SEQ ID NO:8, wherein the CDRs are defined according to Kabat;
d. the HC1 has a heavy chain CDR1 comprising, consisting essentially of, or consisting of the amino acid sequence of SEQ ID NO:18, a heavy chain CDR2 comprising, consisting essentially of, or consisting of the amino acid sequence of SEQ ID NO:19, and a heavy chain CDR3 comprising, consisting essentially of, or consisting of the amino acid sequence of SEQ ID NO:20, and the LC1 has a light chain CDR1 comprising, consisting essentially of, or consisting of the amino acid sequence of SEQ ID NO:6, a light chain CDR2 comprising, consisting essentially of, or consisting of the amino acid sequence of SEQ ID NO:7, and a light chain CDR3 comprising, consisting essentially of, or consisting of the amino acid sequence of SEQ ID NO:8, wherein the CDRs are defined according to Kabat;
e. the HC1 has a heavy chain CDR1 comprising, consisting essentially of, or consisting of the amino acid sequence of SEQ ID NO:22, a heavy chain CDR2 comprising, consisting essentially of, or consisting of the amino acid sequence of SEQ ID NO:23, and a heavy chain CDR3 comprising, consisting essentially of, or consisting of the amino acid sequence of SEQ ID NO:24, and the LC1 has a light chain CDR1 comprising, consisting essentially of, or consisting of the amino acid sequence of SEQ ID NO:6, a light chain CDR2 comprising, consisting essentially of, or consisting of the amino acid sequence of SEQ ID NO:7, and a light chain CDR3 comprising, consisting essentially of, or consisting of the amino acid sequence of SEQ ID NO:8, wherein the CDRs are defined according to Kabat;
f. the HC1 has a heavy chain CDR1 comprising, consisting essentially of, or consisting of the amino acid sequence of SEQ ID NO:26, a heavy chain CDR2 comprising, consisting essentially of, or consisting of the amino acid sequence of SEQ ID NO:27, and a heavy chain CDR3 comprising, consisting essentially of, or consisting of the amino acid sequence of SEQ ID NO:28, and the LC1 has a light chain CDR1 comprising, consisting essentially of, or consisting of the amino acid sequence of SEQ ID NO:30, a light chain CDR2 comprising, consisting essentially of, or consisting of the amino acid sequence of SEQ ID NO:31, and a light chain CDR3 comprising, consisting essentially of, or consisting of the amino acid sequence of SEQ ID NO:32, wherein the CDRs are defined according to Kabat;
g. the HC1 has a heavy chain CDR1 comprising, consisting essentially of, or consisting of the amino acid sequence of SEQ ID NO:2, a heavy chain CDR2 comprising, consisting essentially of, or consisting of the amino acid sequence of SEQ ID NO:34, and a heavy chain CDR3 comprising, consisting essentially of, or consisting of the amino acid sequence of SEQ ID NO:35, and the LC1 has a light chain CDR1 comprising, consisting essentially of, or consisting of the amino acid sequence of SEQ ID NO:37, a light chain CDR2 comprising, consisting essentially of, or consisting of the amino acid sequence of SEQ ID NO:7, and a light chain CDR3 comprising, consisting essentially of, or consisting of the amino acid sequence of SEQ ID NO:38, wherein the CDRs are defined according to Kabat;
h. the HC1 has a heavy chain CDR1 comprising, consisting essentially of, or consisting of the amino acid sequence of SEQ ID NO:22, a heavy chain CDR2 comprising, consisting essentially of, or consisting of the amino acid sequence of SEQ ID NO:23, and a heavy chain CDR3 comprising, consisting essentially of, or consisting of the amino acid sequence of SEQ ID NO:40, and the LC1 has a light chain CDR1 comprising, consisting essentially of, or consisting of the amino acid sequence of SEQ ID NO:42, a light chain CDR2 comprising, consisting essentially of, or consisting of the amino acid sequence of SEQ ID NO:43, and a light chain CDR3 comprising, consisting essentially of, or consisting of the amino acid sequence of SEQ ID NO:44, wherein the CDRs are defined according to Kabat;
i. the HC1 has a heavy chain CDR1 comprising, consisting essentially of, or consisting of the amino acid sequence of SEQ ID NO:46, a heavy chain CDR2 comprising, consisting essentially of, or consisting of the amino acid sequence of SEQ ID NO:47, and a heavy chain CDR3 comprising, consisting essentially of, or consisting of the amino acid sequence of SEQ ID NO:48, and the LC1 has a light chain CDR1 comprising, consisting essentially of, or consisting of the amino acid sequence of SEQ ID NO:50, a light chain CDR2 comprising, consisting essentially of, or consisting of the amino acid sequence of SEQ ID NO:51, and a light chain CDR3 comprising, consisting essentially of, or consisting of the amino acid sequence of SEQ ID NO:52, wherein the CDRs are defined according to Kabat;
j. the HC1 has a heavy chain CDR1 comprising, consisting essentially of, or consisting of the amino acid sequence of SEQ ID NO:54, a heavy chain CDR2 comprising, consisting essentially of, or consisting of the amino acid sequence of SEQ ID NO:55, and a heavy chain CDR3 comprising, consisting essentially of, or consisting of the amino acid sequence of SEQ ID NO:56, and the LC1 has a light chain CDR1 comprising, consisting essentially of, or consisting of the amino acid sequence of SEQ ID NO:58, a light chain CDR2 comprising, consisting essentially of, or consisting of the amino acid sequence of SEQ ID NO:59, and a light chain CDR3 comprising, consisting essentially of, or consisting of the amino acid sequence of SEQ ID NO:60, wherein the CDRs are defined according to Kabat;
k. the HC1 has a heavy chain CDR1 comprising, consisting essentially of, or consisting of the amino acid sequence of SEQ ID NO:54, a heavy chain CDR2 comprising, consisting essentially of, or consisting of the amino acid sequence of SEQ ID NO:55, and a heavy chain CDR3 comprising, consisting essentially of, or consisting of the amino acid sequence of SEQ ID NO:62, and the LC1 has a light chain CDR1 comprising, consisting essentially of, or consisting of the amino acid sequence of SEQ ID NO:58, a light chain CDR2 comprising, consisting essentially of, or consisting of the amino acid sequence of SEQ ID NO:59, and a light chain CDR3 comprising, consisting essentially of, or consisting of the amino acid sequence of SEQ ID NO:60, wherein the CDRs are defined according to Kabat;
l. the HC1 has a heavy chain CDR1 comprising, consisting essentially of, or consisting of the amino acid sequence of SEQ ID NO:64, a heavy chain CDR2 comprising, consisting essentially of, or consisting of the amino acid sequence of SEQ ID NO:19, and a heavy chain CDR3 comprising, consisting essentially of, or consisting of the amino acid sequence of SEQ ID NO:65, and the LC1 has a light chain CDR1 comprising, consisting essentially of, or consisting of the amino acid sequence of SEQ ID NO:6, a light chain CDR2 comprising, consisting essentially of, or consisting of the amino acid sequence of SEQ ID NO:7, and a light chain CDR3 comprising, consisting essentially of, or consisting of the amino acid sequence of SEQ ID NO:8, wherein the CDRs are defined according to Kabat;
m. the HC1 has a heavy chain CDR1 comprising, consisting essentially of, or consisting of the amino acid sequence of SEQ ID NO:67, a heavy chain CDR2 comprising, consisting essentially of, or consisting of the amino acid sequence of SEQ ID NO:68, and a heavy chain CDR3 comprising, consisting essentially of, or consisting of the amino acid sequence of SEQ ID NO:69, and the LC1 has a light chain CDR1 comprising, consisting essentially of, or consisting of the amino acid sequence of SEQ ID NO:6, a light chain CDR2 comprising, consisting essentially of, or consisting of the amino acid sequence of SEQ ID NO:7, and a light chain CDR3 comprising, consisting essentially of, or consisting of the amino acid sequence of SEQ ID NO:8, wherein the CDRs are defined according to Kabat;
n. the HC1 has a heavy chain CDR1 comprising, consisting essentially of, or consisting of the amino acid sequence of SEQ ID NO:10, a heavy chain CDR2 comprising, consisting essentially of, or consisting of the amino acid sequence of SEQ ID NO:71, and a heavy chain CDR3 comprising, consisting essentially of, or consisting of the amino acid sequence of SEQ ID NO:72, and the LC1 has a light chain CDR1 comprising, consisting essentially of, or consisting of the amino acid sequence of SEQ ID NO:6, a light chain CDR2 comprising, consisting essentially of, or consisting of the amino acid sequence of SEQ ID NO:7, and a light chain CDR3 comprising, consisting essentially of, or consisting of the amino acid sequence of SEQ ID NO:8, wherein the CDRs are defined according to Kabat;
o. the HC1 has a heavy chain CDR1 comprising, consisting essentially of, or consisting of the amino acid sequence of SEQ ID NO:54, a heavy chain CDR2 comprising, consisting essentially of, or consisting of the amino acid sequence of SEQ ID NO:74, and a heavy chain CDR3 comprising, consisting essentially of, or consisting of the amino acid sequence of SEQ ID NO:75, and the LC1 has a light chain CDR1 comprising, consisting essentially of, or consisting of the amino acid sequence of SEQ ID NO:77, a light chain CDR2 comprising, consisting essentially of, or consisting of the amino acid sequence of SEQ ID NO:51, and a light chain CDR3 comprising, consisting essentially of, or consisting of the amino acid sequence of SEQ ID NO:78, wherein the CDRs are defined according to Kabat;
p. the HC1 has a heavy chain CDR1 comprising, consisting essentially of, or consisting of the amino acid sequence of SEQ ID NO:22, a heavy chain CDR2 comprising, consisting essentially of, or consisting of the amino acid sequence of SEQ ID NO:23, and a heavy chain CDR3 comprising, consisting essentially of, or consisting of the amino acid sequence of SEQ ID NO:80, and the LC1 has a light chain CDR1 comprising, consisting essentially of, or consisting of the amino acid sequence of SEQ ID NO:82, a light chain CDR2 comprising, consisting essentially of, or consisting of the amino acid sequence of SEQ ID NO:7, and a light chain CDR3 comprising, consisting essentially of, or consisting of the amino acid sequence of SEQ ID NO:83, wherein the CDRs are defined according to Kabat; or
q. the HC1 has a heavy chain CDR1 comprising, consisting essentially of, or consisting of the amino acid sequence of SEQ ID NO:22, a heavy chain CDR2 comprising, consisting essentially of, or consisting of the amino acid sequence of SEQ ID NO:85, and a heavy chain CDR3 comprising, consisting essentially of, or consisting of the amino acid sequence of SEQ ID NO:86, and the LC1 has a light chain CDR1 comprising, consisting essentially of, or consisting of the amino acid sequence of SEQ ID NO:88, a light chain CDR2 comprising, consisting essentially of, or consisting of the amino acid sequence of SEQ ID NO:43, and a light chain CDR3 comprising, consisting essentially of, or consisting of the amino acid sequence of SEQ ID NO:89, wherein the CDRs are defined according to Kabat.

In some embodiments, the first antigen-binding site that immunospecifically binds ROR1 is formed from:

a. a HC1 and a LC1, wherein
i. the HC1 is at least 90%, 95%, or 99% identical to the amino acid sequence of SEQ ID NO:1 and the LC1 is at least 90%, 95%, or 99% identical to the amino acid sequence of SEQ ID NO:5; or
ii. the HC1 comprises, consists essentially of, or consists of the amino acid sequence of SEQ ID NO:1 and the LC1 comprises, consists essentially of, or consists of the amino acid sequence of SEQ ID NO:5;

b. a HC1 and a LC1, wherein
i. the HC1 is at least 90%, 95%, or 99% identical to the amino acid sequence of SEQ ID NO:9 and the LC1 is at least 90%, 95%, or 99% identical to the amino acid sequence of SEQ ID NO:5; or
ii. the HC1 comprises, consists essentially of, or consists of the amino acid sequence of SEQ ID NO:9 and the LC1 comprises, consists essentially of, or consists of the amino acid sequence of SEQ ID NO:5

c. a HC1 and a LC1, wherein
i. the HC1 is at least 90%, 95%, or 99% identical to the amino acid sequence of SEQ ID NO:13 and the LC1 is at least 90%, 95%, or 99% identical to the amino acid sequence of SEQ ID NO:5; or
ii. the HC1 comprises, consists essentially of, or consists of the amino acid sequence of SEQ ID NO:13 and the LC1 comprises, consists essentially of, or consists of the amino acid sequence of SEQ ID NO:5

d. a HC1 and a LC1, wherein
i. the HC1 is at least 90%, 95%, or 99% identical to the amino acid sequence of SEQ ID NO:17 and the LC1 is at least 90%, 95%, or 99% identical to the amino acid sequence of SEQ ID NO:5; or
ii. the HC1 comprises, consists essentially of, or consists of the amino acid sequence of SEQ ID NO:17 and the LC1 comprises, consists essentially of, or consists of the amino acid sequence of SEQ ID NO:5

e. a HC1 and a LC1, wherein
i. the HC1 is at least 90%, 95%, or 99% identical to the amino acid sequence of SEQ ID NO:21 and the LC1 is at least 90%, 95%, or 99% identical to the amino acid sequence of SEQ ID NO:5; or
ii. the HC1 comprises, consists essentially of, or consists of the amino acid sequence of SEQ ID NO:21 and the LC1 comprises, consists essentially of, or consists of the amino acid sequence of SEQ ID NO:5

f. a HC1 and a LC1, wherein
i. the HC1 is at least 90%, 95%, or 99% identical to the amino acid sequence of SEQ ID NO:25 and the LC1 is at least 90%, 95%, or 99% identical to the amino acid sequence of SEQ ID NO:29; or
ii. the HC1 comprises, consists essentially of, or consists of the amino acid sequence of SEQ ID NO:25 and the LC1 comprises, consists essentially of, or consists of the amino acid sequence of SEQ ID NO:29

g. a HC1 and a LC1, wherein
i. the HC1 is at least 90%, 95%, or 99% identical to the amino acid sequence of SEQ ID NO:33 and the LC1 is at least 90%, 95%, or 99% identical to the amino acid sequence of SEQ ID NO:36; or
ii. the HC1 comprises, consists essentially of, or consists of the amino acid sequence of SEQ ID NO:33 and the LC1 comprises, consists essentially of, or consists of the amino acid sequence of SEQ ID NO:36

h. a HC1 and a LC1, wherein
i. the HC1 is at least 90%, 95%, or 99% identical to the amino acid sequence of SEQ ID NO:39 and the LC1 is at least 90%, 95%, or 99% identical to the amino acid sequence of SEQ ID NO:41; or
ii. the HC1 comprises, consists essentially of, or consists of the amino acid sequence of SEQ ID NO:39 and the LC1 comprises, consists essentially of, or consists of the amino acid sequence of SEQ ID NO:41

i. a HC1 and a LC1, wherein
i. the HC1 is at least 90%, 95%, or 99% identical to the amino acid sequence of SEQ ID NO:45 and the LC1 is at least 90%, 95%, or 99% identical to the amino acid sequence of SEQ ID NO:49; or
ii. the HC1 comprises, consists essentially of, or consists of the amino acid sequence of SEQ ID NO:45 and the LC1 comprises, consists essentially of, or consists of the amino acid sequence of SEQ ID NO:49

j. a HC1 and a LC1, wherein
i. the HC1 is at least 90%, 95%, or 99% identical to the amino acid sequence of SEQ ID NO:53 and the LC1 is at least 90%, 95%, or 99% identical to the amino acid sequence of SEQ ID NO:57; or
ii. the HC1 comprises, consists essentially of, or consists of the amino acid sequence of SEQ ID NO:53 and the LC1 comprises, consists essentially of, or consists of the amino acid sequence of SEQ ID NO:57

k. a HC1 and a LC1, wherein
i. the HC1 is at least 90%, 95%, or 99% identical to the amino acid sequence of SEQ ID NO:61 and the LC1 is at least 90%, 95%, or 99% identical to the amino acid sequence of SEQ ID NO:57; or
ii. the HC1 comprises, consists essentially of, or consists of the amino acid sequence of SEQ ID NO:61 and the LC1 comprises, consists essentially of, or consists of the amino acid sequence of SEQ ID NO:57

l. a HC1 and a LC1, wherein
i. the HC1 is at least 90%, 95%, or 99% identical to the amino acid sequence of SEQ ID NO:63 and the LC1 is at least 90%, 95%, or 99% identical to the amino acid sequence of SEQ ID NO:5; or
ii. the HC1 comprises, consists essentially of, or consists of the amino acid sequence of SEQ ID NO:63 and the LC1 comprises, consists essentially of, or consists of the amino acid sequence of SEQ ID NO:5

m. a HC1 and a LC1, wherein
i. the HC1 is at least 90%, 95%, or 99% identical to the amino acid sequence of SEQ ID NO:66 and the LC1 is at least 90%, 95%, or 99% identical to the amino acid sequence of SEQ ID NO:5; or
ii. the HC1 comprises, consists essentially of, or consists of the amino acid sequence of SEQ ID NO:66 and the LC1 comprises, consists essentially of, or consists of the amino acid sequence of SEQ ID NO:5

n. a HC1 and a LC1, wherein
i. the HC1 is at least 90%, 95%, or 99% identical to the amino acid sequence of SEQ ID NO:70 and the LC1 is at least 90%, 95%, or 99% identical to the amino acid sequence of SEQ ID NO:5; or
ii. the HC1 comprises, consists essentially of, or consists of the amino acid sequence of SEQ ID NO:70 and the LC1 comprises, consists essentially of, or consists of the amino acid sequence of SEQ ID NO:5

o. a HC1 and a LC1, wherein
i. the HC1 is at least 90%, 95%, or 99% identical to the amino acid sequence of SEQ ID NO:73 and the LC1 is at least 90%, 95%, or 99% identical to the amino acid sequence of SEQ ID NO:76; or
ii. the HC1 comprises, consists essentially of, or consists of the amino acid sequence of SEQ ID NO:73 and the LC1 comprises, consists essentially of, or consists of the amino acid sequence of SEQ ID NO:76

p. a HC1 and a LC1, wherein
i. the HC1 is at least 90%, 95%, or 99% identical to the amino acid sequence of SEQ ID NO:79 and the LC1 is at least 90%, 95%, or 99% identical to the amino acid sequence of SEQ ID NO:81; or
ii. the HC1 comprises, consists essentially of, or consists of the amino acid sequence of SEQ ID NO:79 and the LC1 comprises, consists essentially of, or consists of the amino acid sequence of SEQ ID NO:81

q. a HC1 and a LC1, wherein
i. the HC1 is at least 90%, 95%, or 99% identical to the amino acid sequence of SEQ ID NO:84 and the LC1 is at least 90%, 95%, or 99% identical to the amino acid sequence of SEQ ID NO:87; or
ii. the HC1 comprises, consists essentially of, or consists of the amino acid sequence of SEQ ID NO:84 and the LC1 comprises, consists essentially of, or consists of the amino acid sequence of SEQ ID NO:87.

In some embodiments, the first antigen-binding site that immunospecifically binds to ROR1 can comprise:

a. the heavy chain CDRs and light chain CDRs of any one of a-q above and

b. a heavy chain and light chain that, apart from the CDRs, comprises:
i. the HC1 and LC1 of any one of a.i.-q.i. above or
ii. the HC1 and LC1 of any one of a.ii.-q.ii. above.

For example, and without intent to be limiting, the first antigen-binding site that immunospecifically binds to ROR1 can comprise:
a HC1 having a heavy chain CDR1 comprising, consisting essentially of, or consisting of the amino acid sequence of SEQ ID NO:14, a heavy chain CDR2 comprising, consisting essentially of, or consisting of the amino acid sequence of SEQ ID NO:15, and and a heavy chain CDR3 comprising, consisting essentially of, or consisting of the amino acid sequence of SEQ ID NO:16, and a LC1 having a light chain CDR1 comprising, consisting essentially of, or consisting of the amino acid sequence of SEQ ID NO:6, a light chain CDR2 comprising, consisting essentially of, or consisting of the amino acid sequence of SEQ ID NO:7, and a light chain CDR3 comprising, consisting essentially of, or consisting of the amino acid sequence of SEQ ID NO:8, wherein the CDRs are defined according to Kabat; and
a HC1 that, apart from the CDRs, is at least 90%, 95%, or 99% identical to the amino acid sequence of SEQ ID NO:13 and a LC1 that, apart from the CDRs, is at least 90%, 95%, or 99% identical to the amino acid sequence of SEQ ID NO:5.

The second antigen-binding site that immunospecifically binds CD3 can be formed from a HC2 and a LC2, wherein the HC2 has a heavy chain CDR1 comprising, consisting essentially of, or consisting of the amino acid sequence of SEQ ID NO:92, a heavy chain CDR2 comprising, consisting essentially of, or consisting of the amino acid sequence of SEQ ID NO:93, and a heavy chain CDR3 comprising, consisting essentially of, or consisting of the amino acid sequence of SEQ ID NO:94, and the LC2 has a light chain CDR1 comprising, consisting essentially of, or consisting of the amino acid sequence of SEQ ID NO:95, a light chain CDR2 comprising, consisting essentially of, or consisting of the amino acid sequence of SEQ ID NO:96, and a light chain CDR3 comprising, consisting essentially of, or consisting of the amino acid sequence of SEQ ID NO:97, wherein the CDRs are defined according to Kabat.

In some embodiments, the second antigen-binding site that immunospecifically binds CD3 can be formed from a HC2 and a LC2, wherein the HC2 is at least 90%, 95%, or 99% identical to SEQ ID NO:90 and the LC2 is at least 90%, 95%, or 99% identical to SEQ ID NO:91. In some embodiments, the second antigen-binding site that immunospecifically binds CD3 is formed from a HC2 and a LC2, wherein the HC2 comprises, consists essentially of, or consists the amino acid sequence of SEQ ID NO:90 and the LC2 comprises, consists essentially of, or consists the amino acid sequence of SEQ ID NO:91.

The ROR1×CD3 bispecific antibody, or bispecific antigen-binding fragment thereof, can comprise:
a. a first antigen-binding site that immunospecifically binds ROR1, the first antigen-binding site formed from a HC1 and a LC1, wherein
   i. the HC1 has a heavy chain CDR1 comprising, consisting essentially of, or consisting of the amino acid sequence of SEQ ID NO:2, a heavy chain CDR2 comprising, consisting essentially of, or consisting of the amino acid sequence of SEQ ID NO:3, and a heavy chain CDR3 comprising, consisting essentially of, or consisting of the amino acid sequence of SEQ ID NO:4, and the LC1 has a light chain CDR1 comprising, consisting essentially of, or consisting of the amino acid sequence of SEQ ID NO:6, a light chain CDR2 comprising, consisting essentially of, or consisting of the amino acid sequence of SEQ ID NO:7, and a light chain CDR3 comprising, consisting essentially of, or consisting of the amino acid sequence of SEQ ID NO:8;
   ii. the HC1 comprises an amino acid sequence that is at least 90%, 95%, or 99% identical to the amino acid sequence of SEQ ID NO:1 and the LC1 comprises an amino acid sequence that is at least 90%, 95%, or 99% identical to the amino acid sequence of SEQ ID NO:5; or
   iii. the HC1 comprises, consists essentially of, or consists of the amino acid sequence of SEQ ID NO:1 and the LC1 comprises, consists essentially of, or consists of the amino acid sequence of SEQ ID NO:5; and
b. a second antigen-binding site that immunospecifically binds CD3, the second antigen-binding site formed from a HC2 and a LC2, wherein
   i. the HC2 has a heavy chain CDR1 comprising, consisting essentially of, or consisting of the amino acid sequence of SEQ ID NO:92, a heavy chain CDR2 comprising, consisting essentially of, or consisting of the amino acid sequence of SEQ ID NO:93, and a heavy chain CDR3 comprising, consisting essentially of, or consisting of the amino acid sequence of SEQ ID NO:94, and the LC2 has a light chain CDR1 comprising, consisting essentially of, or consisting of the amino acid sequence of SEQ ID NO:95, a light chain CDR2 comprising, consisting essentially of, or consisting of the amino acid sequence of SEQ ID NO:96, and a light chain CDR3 comprising, consisting essentially of, or consisting of the amino acid sequence of SEQ ID NO:97;
   ii. the HC2 comprises an amino acid sequence that is at least 90%, 95%, or 99% identical to the amino acid sequence of SEQ ID NO:90 and the LC2 comprises an amino acid sequence that is at least 90%, 95%, or 99% identical to the amino acid sequence of SEQ ID NO:91; or
   iii. the HC2 comprises, consists essentially of, or consists of the amino acid sequence of SEQ ID NO:90 and the LC2 comprises, consists essentially of, or consists of the amino acid sequence of SEQ ID NO:91,
      wherein the CDRs are defined according to Kabat.

The ROR1×CD3 bispecific antibody, or bispecific antigen-binding fragment thereof, can comprise:
a. a first antigen-binding site that immunospecifically binds ROR1, the first antigen-binding site formed from a HC1 and a LC1, wherein
   i. the HC1 has a heavy chain CDR1 comprising, consisting essentially of, or consisting of the amino acid sequence of SEQ ID NO:10, a heavy chain CDR2 comprising, consisting essentially of, or consisting of the amino acid sequence of SEQ ID NO:11, and a heavy chain CDR3 comprising, consisting essentially of, or consisting of the amino acid sequence of SEQ ID NO:12, and the LC1 has a light chain CDR1 comprising, consisting essentially of, or consisting of the amino acid sequence of SEQ ID NO:6, a light chain CDR2 comprising, consisting essentially of, or consisting of the amino acid sequence of SEQ ID NO:7, and a light chain CDR3 comprising, consisting essentially of, or consisting of the amino acid sequence of SEQ ID NO:8;
   ii. the HC1 comprises an amino acid sequence that is at least 90%, 95%, or 99% identical to the amino acid sequence of SEQ ID NO:9 and the LC1 comprises an amino acid sequence that is at least 90%, 95%, or 99% identical to the amino acid sequence of SEQ ID NO:5; or
   iii. the HC1 comprises, consists essentially of, or consists of the amino acid sequence of SEQ ID NO:9 and the LC1 comprises, consists essentially of, or consists of the amino acid sequence of SEQ ID NO:5; and
b. a second antigen-binding site that immunospecifically binds CD3, the second antigen-binding site formed from a HC2 and a LC2, wherein
   i. the HC2 has a heavy chain CDR1 comprising, consisting essentially of, or consisting of the amino acid sequence of SEQ ID NO:92, a heavy chain CDR2 comprising, consisting essentially of, or consisting of the amino acid sequence of SEQ ID NO:93, and a heavy chain CDR3 comprising, consisting essentially of, or consisting of the amino acid sequence of SEQ ID NO:94, and the LC2 has a light chain CDR1 comprising, consisting essentially of, or consisting of the amino acid sequence of SEQ ID NO:95, a light chain CDR2 comprising, consisting essentially of, or consisting of the amino acid sequence of SEQ ID NO:96, and a light chain CDR3 comprising, consisting essentially of, or consisting of the amino acid sequence of SEQ ID NO:97;
   ii. the HC2 comprises an amino acid sequence that is at least 90%, 95%, or 99% identical to the amino acid sequence of SEQ ID NO:90 and the LC2 comprises an amino acid sequence that is at least 90%, 95%, or 99% identical to the amino acid sequence of SEQ ID NO:91; or
   iii. the HC2 comprises, consists essentially of, or consists of the amino acid sequence of SEQ ID NO:90 and the LC2 comprises, consists essentially of, or consists of the amino acid sequence of SEQ ID NO:91,
      wherein the CDRs are defined according to Kabat.

The ROR1×CD3 bispecific antibody, or bispecific antigen-binding fragment thereof, can comprise:
a. a first antigen-binding site that immunospecifically binds ROR1, the first antigen-binding site formed from a HC1 and a LC1, wherein
   i. the HC1 has a heavy chain CDR1 comprising, consisting essentially of, or consisting of the amino acid sequence of SEQ ID NO:14, a heavy chain CDR2 comprising, consisting essentially of, or consisting of the amino acid sequence of SEQ ID NO:15, and a heavy chain CDR3 comprising, consisting essentially of, or consisting of the amino acid sequence of SEQ ID NO:16, and the LC1 has a light chain CDR1 comprising, consisting essentially of, or consisting of the amino acid sequence of SEQ ID NO:6, a light chain CDR2 comprising, consisting essentially of, or consisting of the amino acid sequence of SEQ ID NO:7, and a light chain CDR3 comprising, consisting essentially of, or consisting of the amino acid sequence of SEQ ID NO:8;
   ii. the HC1 comprises an amino acid sequence that is at least 90%, 95%, or 99% identical to the amino acid sequence of SEQ ID NO:13 and the LC1 comprises an amino acid sequence that is at least 90%, 95%, or 99% identical to the amino acid sequence of SEQ ID NO:5; or
   iii. the HC1 comprises, consists essentially of, or consists of the amino acid sequence of SEQ ID NO:13 and the LC1 comprises, consists essentially of, or consists of the amino acid sequence of SEQ ID NO:5; and
b. a second antigen-binding site that immunospecifically binds CD3, the second antigen-binding site formed from a HC2 and a LC2, wherein
   i. the HC2 has a heavy chain CDR1 comprising, consisting essentially of, or consisting of the amino acid sequence of SEQ ID NO:92, a heavy chain CDR2 comprising, consisting essentially of, or consisting of the amino acid sequence of SEQ ID NO:93, and a heavy chain CDR3 comprising, consisting essentially of, or consisting of the amino acid sequence of SEQ ID NO:94, and the LC2 has a light chain CDR1 comprising, consisting essentially of, or consisting of the amino acid sequence of SEQ ID NO:95, a light chain CDR2 comprising, consisting essentially of, or consisting of the amino acid sequence of SEQ ID NO:96, and a light chain CDR3 comprising, consisting essentially of, or consisting of the amino acid sequence of SEQ ID NO:97;
   ii. the HC2 comprises an amino acid sequence that is at least 90%, 95%, or 99% identical to the amino acid sequence of SEQ ID NO:90 and the LC2 comprises an amino acid sequence that is at least 90%, 95%, or 99% identical to the amino acid sequence of SEQ ID NO:91; or
   iii. the HC2 comprises, consists essentially of, or consists of the amino acid sequence of SEQ ID NO:90 and the LC2 comprises, consists essentially of, or consists of the amino acid sequence of SEQ ID NO:91,
      wherein the CDRs are defined according to Kabat.

The ROR1×CD3 bispecific antibody, or bispecific antigen-binding fragment thereof, can comprise:
a. a first antigen-binding site that immunospecifically binds ROR1, the first antigen-binding site formed from a HC1 and a LC1, wherein
   i. the HC1 has a heavy chain CDR1 comprising, consisting essentially of, or consisting of the amino acid sequence of SEQ ID NO:18, a heavy chain CDR2 comprising, consisting essentially of, or consisting of the amino acid sequence of SEQ ID NO:19, and a heavy chain CDR3 comprising, consisting essentially of, or consisting of the amino acid sequence of SEQ ID NO:20, and the LC1 has a light chain CDR1 comprising, consisting essentially of, or consisting of the amino acid sequence of SEQ ID NO:6, a light chain CDR2 comprising, consisting essentially of, or consisting of the amino acid sequence of SEQ ID NO:7, and a light chain CDR3 comprising, consisting essentially of, or consisting of the amino acid sequence of SEQ ID NO:8;
   ii. the HC1 comprises an amino acid sequence that is at least 90%, 95%, or 99% identical to the amino acid sequence of SEQ ID NO:17 and the LC1 comprises an amino acid sequence that is at least 90%, 95%, or 99% identical to the amino acid sequence of SEQ ID NO:5; or
   iii. the HC1 comprises, consists essentially of, or consists of the amino acid sequence of SEQ ID NO:17 and the LC1 comprises, consists essentially of, or consists of the amino acid sequence of SEQ ID NO:5; and
b. a second antigen-binding site that immunospecifically binds CD3, the second antigen-binding site formed from a HC2 and a LC2, wherein
   i. the HC2 has a heavy chain CDR1 comprising, consisting essentially of, or consisting of the amino acid sequence of SEQ ID NO:92, a heavy chain CDR2 comprising, consisting essentially of, or consisting of the amino acid sequence of SEQ ID NO:93, and a heavy chain CDR3 comprising, consisting essentially of, or consisting of the amino acid sequence of SEQ ID NO:94, and the LC2 has a light chain CDR1 comprising, consisting essentially of, or consisting of the amino acid sequence of SEQ ID NO:95, a light chain CDR2 comprising, consisting essentially of, or consisting of the amino acid sequence of SEQ ID NO:96, and a light chain CDR3 comprising, consisting essentially of, or consisting of the amino acid sequence of SEQ ID NO:97;
   ii. the HC2 comprises an amino acid sequence that is at least 90%, 95%, or 99% identical to the amino acid sequence of SEQ ID NO:90 and the LC2 comprises an amino acid sequence that is at least 90%, 95%, or 99% identical to the amino acid sequence of SEQ ID NO:91; or
   iii. the HC2 comprises, consists essentially of, or consists of the amino acid sequence of SEQ ID NO:90 and the LC2 comprises, consists essentially of, or consists of the amino acid sequence of SEQ ID NO:91,
      wherein the CDRs are defined according to Kabat.

The ROR1×CD3 bispecific antibody, or bispecific antigen-binding fragment thereof, can comprise:
a. a first antigen-binding site that immunospecifically binds ROR1, the first antigen-binding site formed from a HC1 and a LC1, wherein
   i. the HC1 has a heavy chain CDR1 comprising, consisting essentially of, or consisting of the amino acid sequence of SEQ ID NO:22, a heavy chain CDR2 comprising, consisting essentially of, or consisting of the amino acid sequence of SEQ ID NO:23, and a heavy chain CDR3 comprising, consisting essentially of, or consisting of the amino acid sequence of SEQ ID NO:24, and the LC1 has a light chain CDR1 comprising, consisting essentially of, or consisting of the amino acid sequence of SEQ ID NO:6, a light chain CDR2 comprising, consisting essentially of, or consisting of the amino acid sequence of SEQ ID NO:7, and a light chain CDR3 comprising, consisting essentially of, or consisting of the amino acid sequence of SEQ ID NO:8;
   ii. the HC1 comprises an amino acid sequence that is at least 90%, 95%, or 99% identical to the amino acid sequence of SEQ ID NO:21 and the LC1 comprises an amino acid sequence that is at least 90%, 95%, or 99% identical to the amino acid sequence of SEQ ID NO:5; or
   iii. the HC1 comprises, consists essentially of, or consists of the amino acid sequence of SEQ ID NO:21 and the LC1 comprises, consists essentially of, or consists of the amino acid sequence of SEQ ID NO:5; and
b. a second antigen-binding site that immunospecifically binds CD3, the second antigen-binding site formed from a HC2 and a LC2, wherein
   i. the HC2 has a heavy chain CDR1 comprising, consisting essentially of, or consisting of the amino acid sequence of SEQ ID NO:92, a heavy chain CDR2 comprising, consisting essentially of, or consisting of the amino acid sequence of SEQ ID NO:93, and a heavy chain CDR3 comprising, consisting essentially of, or consisting of the amino acid sequence of SEQ ID NO:94, and the LC2 has a light chain CDR1 comprising, consisting essentially of, or consisting of the amino acid sequence of SEQ ID NO:95, a light chain CDR2 comprising, consisting essentially of, or consisting of the amino acid sequence of SEQ ID NO:96, and a light chain CDR3 comprising, consisting essentially of, or consisting of the amino acid sequence of SEQ ID NO:97;
   ii. the HC2 comprises an amino acid sequence that is at least 90%, 95%, or 99% identical to the amino acid sequence of SEQ ID NO:90 and the LC2 comprises an amino acid sequence that is at least 90%, 95%, or 99% identical to the amino acid sequence of SEQ ID NO:91; or
   iii. the HC2 comprises, consists essentially of, or consists of the amino acid sequence of SEQ ID NO:90 and the LC2 comprises, consists essentially of, or consists of the amino acid sequence of SEQ ID NO:91,
      wherein the CDRs are defined according to Kabat.

The ROR1×CD3 bispecific antibody, or bispecific antigen-binding fragment thereof, can comprise:
a. a first antigen-binding site that immunospecifically binds ROR1, the first antigen-binding site formed from a HC1 and a LC1, wherein
   i. the HC1 has a heavy chain CDR1 comprising, consisting essentially of, or consisting of the amino acid sequence of SEQ ID NO:26, a heavy chain CDR2 comprising, consisting essentially of, or consisting of the amino acid sequence of SEQ ID NO:27, and a heavy chain CDR3 comprising, consisting essentially of, or consisting of the amino acid sequence of SEQ ID NO:28, and the LC1 has a light chain CDR1 comprising, consisting essentially of, or consisting of the amino acid sequence of SEQ ID NO:30, a light chain CDR2 comprising, consisting essentially of, or consisting of the amino acid sequence of SEQ ID NO:31, and a light chain CDR3 comprising, consisting essentially of, or consisting of the amino acid sequence of SEQ ID NO:32;
   ii. the HC1 comprises an amino acid sequence that is at least 90%, 95%, or 99% identical to the amino acid sequence of SEQ ID NO:25 and the LC1 comprises an amino acid sequence that is at least 90%, 95%, or 99% identical to the amino acid sequence of SEQ ID NO:29; or
   iii. the HC1 comprises, consists essentially of, or consists of the amino acid sequence of SEQ ID NO:25 and the LC1 comprises, consists essentially of, or consists of the amino acid sequence of SEQ ID NO:29; and
b. a second antigen-binding site that immunospecifically binds CD3, the second antigen-binding site formed from a HC2 and a LC2, wherein
   i. the HC2 has a heavy chain CDR1 comprising, consisting essentially of, or consisting of the amino acid sequence of SEQ ID NO:92, a heavy chain CDR2 comprising, consisting essentially of, or consisting of the amino acid sequence of SEQ ID NO:93, and a heavy chain CDR3 comprising, consisting essentially of, or consisting of the amino acid sequence of SEQ ID NO:94, and the LC2 has a light chain CDR1 comprising, consisting essentially of, or consisting of the amino acid sequence of SEQ ID NO:95, a light chain CDR2 comprising, consisting essentially of, or consisting of the amino acid sequence of SEQ ID NO:96, and a light chain CDR3 comprising, consisting essentially of, or consisting of the amino acid sequence of SEQ ID NO:97;
   ii. the HC2 comprises an amino acid sequence that is at least 90%, 95%, or 99% identical to the amino acid sequence of SEQ ID NO:90 and the LC2 comprises an amino acid sequence that is at least 90%, 95%, or 99% identical to the amino acid sequence of SEQ ID NO:91; or
   iii. the HC2 comprises, consists essentially of, or consists of the amino acid sequence of SEQ ID NO:90 and the LC2 comprises, consists essentially of, or consists of the amino acid sequence of SEQ ID NO:91,
      wherein the CDRs are defined according to Kabat.

The ROR1×CD3 bispecific antibody, or bispecific antigen-binding fragment thereof, can comprise:
a. a first antigen-binding site that immunospecifically binds ROR1, the first antigen-binding site formed from a HC1 and a LC1, wherein
   i. the HC1 has a heavy chain CDR1 comprising, consisting essentially of, or consisting of the amino acid sequence of SEQ ID NO:2, a heavy chain CDR2 comprising, consisting essentially of, or consisting of the amino acid sequence of SEQ ID NO:34, and a heavy chain CDR3 comprising, consisting essentially of, or consisting of the amino acid sequence of SEQ ID NO:35, and the LC1 has a light chain CDR1 comprising, consisting essentially of, or consisting of the amino acid sequence of SEQ ID NO:37, a light chain CDR2 comprising, consisting essentially of, or consisting of the amino acid sequence of SEQ ID NO:7, and a light chain CDR3 comprising, consisting essentially of, or consisting of the amino acid sequence of SEQ ID NO:38;
   ii. the HC1 comprises an amino acid sequence that is at least 90%, 95%, or 99% identical to the amino acid sequence of SEQ ID NO:33 and the LC1 comprises an amino acid sequence that is at least 90%, 95%, or 99% identical to the amino acid sequence of SEQ ID NO:36; or
   iii. the HC1 comprises, consists essentially of, or consists of the amino acid sequence of SEQ ID NO:33 and the LC1 comprises, consists essentially of, or consists of the amino acid sequence of SEQ ID NO:36; and
b. a second antigen-binding site that immunospecifically binds CD3, the second antigen-binding site formed from a HC2 and a LC2, wherein
   i. the HC2 has a heavy chain CDR1 comprising, consisting essentially of, or consisting of the amino acid sequence of SEQ ID NO:92, a heavy chain CDR2 comprising, consisting essentially of, or consisting of the amino acid sequence of SEQ ID NO:93, and a heavy chain CDR3 comprising, consisting essentially of, or consisting of the amino acid sequence of SEQ ID NO:94, and the LC2 has a light chain CDR1 comprising, consisting essentially of, or consisting of the amino acid sequence of SEQ ID NO:95, a light chain CDR2 comprising, consisting essentially of, or consisting of the amino acid sequence of SEQ ID NO:96, and a light chain CDR3 comprising, consisting essentially of, or consisting of the amino acid sequence of SEQ ID NO:97;
   ii. the HC2 comprises an amino acid sequence that is at least 90%, 95%, or 99% identical to the amino acid sequence of SEQ ID NO:90 and the LC2 comprises an amino acid sequence that is at least 90%, 95%, or 99% identical to the amino acid sequence of SEQ ID NO:91; or
   iii. the HC2 comprises, consists essentially of, or consists of the amino acid sequence of SEQ ID NO:90 and the LC2 comprises, consists essentially of, or consists of the amino acid sequence of SEQ ID NO:91,
      wherein the CDRs are defined according to Kabat.

The ROR1×CD3 bispecific antibody, or bispecific antigen-binding fragment thereof, can comprise:
a. a first antigen-binding site that immunospecifically binds ROR1, the first antigen-binding site formed from a HC1 and a LC1, wherein
   i. the HC1 has a heavy chain CDR1 comprising, consisting essentially of, or consisting of the amino acid sequence of SEQ ID NO:22, a heavy chain CDR2 comprising, consisting essentially of, or consisting of the amino acid sequence of SEQ ID NO:23, and a heavy chain CDR3 comprising, consisting essentially of, or consisting of the amino acid sequence of SEQ ID NO:40, and the LC1 has a light chain CDR1 comprising, consisting essentially of, or consisting of the amino acid sequence of SEQ ID NO:42, a light chain CDR2 comprising, consisting essentially of, or consisting of the amino acid sequence of SEQ ID NO:43, and a light chain CDR3 comprising, consisting essentially of, or consisting of the amino acid sequence of SEQ ID NO:44;
   ii. the HC1 comprises an amino acid sequence that is at least 90%, 95%, or 99% identical to the amino acid sequence of SEQ ID NO:39 and the LC1 comprises an amino acid sequence that is at least 90%, 95%, or 99% identical to the amino acid sequence of SEQ ID NO:41; or
   iii. the HC1 comprises, consists essentially of, or consists of the amino acid sequence of SEQ ID NO:39 and the LC1 comprises, consists essentially of, or consists of the amino acid sequence of SEQ ID NO:41; and
b. a second antigen-binding site that immunospecifically binds CD3, the second antigen-binding site formed from a HC2 and a LC2, wherein
   i. the HC2 has a heavy chain CDR1 comprising, consisting essentially of, or consisting of the amino acid sequence of SEQ ID NO:92, a heavy chain CDR2 comprising, consisting essentially of, or consisting of the amino acid sequence of SEQ ID NO:93, and a heavy chain CDR3 comprising, consisting essentially of, or consisting of the amino acid sequence of SEQ ID NO:94, and the LC2 has a light chain CDR1 comprising, consisting essentially of, or consisting of the amino acid sequence of SEQ ID NO:95, a light chain CDR2 comprising, consisting essentially of, or consisting of the amino acid sequence of SEQ ID NO:96, and a light chain CDR3 comprising, consisting essentially of, or consisting of the amino acid sequence of SEQ ID NO:97;
   ii. the HC2 comprises an amino acid sequence that is at least 90%, 95%, or 99% identical to the amino acid sequence of SEQ ID NO:90 and the LC2 comprises an amino acid sequence that is at least 90%, 95%, or 99% identical to the amino acid sequence of SEQ ID NO:91; or
   iii. the HC2 comprises, consists essentially of, or consists of the amino acid sequence of SEQ ID NO:90 and the LC2 comprises, consists essentially of, or consists of the amino acid sequence of SEQ ID NO:91,
      wherein the CDRs are defined according to Kabat.

The ROR1×CD3 bispecific antibody, or bispecific antigen-binding fragment thereof, can comprise:
a. a first antigen-binding site that immunospecifically binds ROR1, the first antigen-binding site formed from a HC1 and a LC1, wherein
   i. the HC1 has a heavy chain CDR1 comprising, consisting essentially of, or consisting of the amino acid sequence of SEQ ID NO:46, a heavy chain CDR2 comprising, consisting essentially of, or consisting of the amino acid sequence of SEQ ID NO:47, and a heavy chain CDR3 comprising, consisting essentially of, or consisting of the amino acid sequence of SEQ ID NO:48, and the LC1 has a light chain CDR1 comprising, consisting essentially of, or consisting of the amino acid sequence of SEQ ID NO:50, a light chain CDR2 comprising, consisting essentially of, or consisting of the amino acid sequence of SEQ ID NO:51, and a light chain CDR3 comprising, consisting essentially of, or consisting of the amino acid sequence of SEQ ID NO:52;
   ii. the HC1 comprises an amino acid sequence that is at least 90%, 95%, or 99% identical to the amino acid sequence of SEQ ID NO:45 and the LC1 comprises an amino acid sequence that is at least 90%, 95%, or 99% identical to the amino acid sequence of SEQ ID NO:49; or
   iii. the HC1 comprises, consists essentially of, or consists of the amino acid sequence of SEQ ID NO:45 and the LC1 comprises, consists essentially of, or consists of the amino acid sequence of SEQ ID NO:49; and
b. a second antigen-binding site that immunospecifically binds CD3, the second antigen-binding site formed from a HC2 and a LC2, wherein
   i. the HC2 has a heavy chain CDR1 comprising, consisting essentially of, or consisting of the amino acid sequence of SEQ ID NO:92, a heavy chain CDR2 comprising, consisting essentially of, or consisting of the amino acid sequence of SEQ ID NO:93, and a heavy chain CDR3 comprising, consisting essentially of, or consisting of the amino acid sequence of SEQ ID NO:94, and the LC2 has a light chain CDR1 comprising, consisting essentially of, or consisting of the amino acid sequence of SEQ ID NO:95, a light chain CDR2 comprising, consisting essentially of, or consisting of the amino acid sequence of SEQ ID NO:96, and a light chain CDR3 comprising, consisting essentially of, or consisting of the amino acid sequence of SEQ ID NO:97;
   ii. the HC2 comprises an amino acid sequence that is at least 90%, 95%, or 99% identical to the amino acid sequence of SEQ ID NO:90 and the LC2 comprises an amino acid sequence that is at least 90%, 95%, or 99% identical to the amino acid sequence of SEQ ID NO:91; or
   iii. the HC2 comprises, consists essentially of, or consists of the amino acid sequence of SEQ ID NO:90 and the LC2 comprises, consists essentially of, or consists of the amino acid sequence of SEQ ID NO:91,
      wherein the CDRs are defined according to Kabat.

The ROR1×CD3 bispecific antibody, or bispecific antigen-binding fragment thereof, can comprise:
a. a first antigen-binding site that immunospecifically binds ROR1, the first antigen-binding site formed from a HC1 and a LC1, wherein
   i. the HC1 has a heavy chain CDR1 comprising, consisting essentially of, or consisting of the amino acid sequence of SEQ ID NO:54, a heavy chain CDR2 comprising, consisting essentially of, or consisting of the amino acid sequence of SEQ ID NO:55, and a heavy chain CDR3 comprising, consisting essentially of, or consisting of the amino acid sequence of SEQ ID NO:56, and the LC1 has a light chain CDR1 comprising, consisting essentially of, or consisting of the amino acid sequence of SEQ ID NO:58, a light chain CDR2 comprising, consisting essentially of, or consisting of the amino acid sequence of SEQ ID NO:59, and a light chain CDR3 comprising, consisting essentially of, or consisting of the amino acid sequence of SEQ ID NO:60;
   ii. the HC1 comprises an amino acid sequence that is at least 90%, 95%, or 99% identical to the amino acid sequence of SEQ ID NO:53 and the LC1 comprises an amino acid sequence that is at least 90%, 95%, or 99% identical to the amino acid sequence of SEQ ID NO:57; or
   iii. the HC1 comprises, consists essentially of, or consists of the amino acid sequence of SEQ ID NO:53 and the LC1 comprises, consists essentially of, or consists of the amino acid sequence of SEQ ID NO:57; and
b. a second antigen-binding site that immunospecifically binds CD3, the second antigen-binding site formed from a HC2 and a LC2, wherein
   i. the HC2 has a heavy chain CDR1 comprising, consisting essentially of, or consisting of the amino acid sequence of SEQ ID NO:92, a heavy chain CDR2 comprising, consisting essentially of, or consisting of the amino acid sequence of SEQ ID NO:93, and a heavy chain CDR3 comprising, consisting essentially of, or consisting of the amino acid sequence of SEQ ID NO:94, and the LC2 has a light chain CDR1 comprising, consisting essentially of, or consisting of the amino acid sequence of SEQ ID NO:95, a light chain CDR2 comprising, consisting essentially of, or consisting of the amino acid sequence of SEQ ID NO:96, and a light chain CDR3 comprising, consisting essentially of, or consisting of the amino acid sequence of SEQ ID NO:97;
   ii. the HC2 comprises an amino acid sequence that is at least 90%, 95%, or 99% identical to the amino acid sequence of SEQ ID NO:90 and the LC2 comprises an amino acid sequence that is at least 90%, 95%, or 99% identical to the amino acid sequence of SEQ ID NO:91; or
   iii. the HC2 comprises, consists essentially of, or consists of the amino acid sequence of SEQ ID NO:90 and the LC2 comprises, consists essentially of, or consists of the amino acid sequence of SEQ ID NO:91,
      wherein the CDRs are defined according to Kabat.

The ROR1×CD3 bispecific antibody, or bispecific antigen-binding fragment thereof, can comprise:
a. a first antigen-binding site that immunospecifically binds ROR1, the first antigen-binding site formed from a HC1 and a LC1, wherein
   i. the HC1 has a heavy chain CDR1 comprising, consisting essentially of, or consisting of the amino acid sequence of SEQ ID NO:54, a heavy chain CDR2 comprising, consisting essentially of, or consisting of the amino acid sequence of SEQ ID NO:55, and a heavy chain CDR3 comprising, consisting essentially of, or consisting of the amino acid sequence of SEQ ID NO:62, and the LC1 has a light chain CDR1 comprising, consisting essentially of, or consisting of the amino acid sequence of SEQ ID NO:58, a light chain CDR2 comprising, consisting essentially of, or consisting of the amino acid sequence of SEQ ID NO:59, and a light chain CDR3 comprising, consisting essentially of, or consisting of the amino acid sequence of SEQ ID NO:60;
   ii. the HC1 comprises an amino acid sequence that is at least 90%, 95%, or 99% identical to the amino acid sequence of SEQ ID NO:61 and the LC1 comprises an amino acid sequence that is at least 90%, 95%, or 99% identical to the amino acid sequence of SEQ ID NO:57; or
   iii. the HC1 comprises, consists essentially of, or consists of the amino acid sequence of SEQ ID NO:61 and the LC1 comprises, consists essentially of, or consists of the amino acid sequence of SEQ ID NO:57; and
b. a second antigen-binding site that immunospecifically binds CD3, the second antigen-binding site formed from a HC2 and a LC2, wherein
   i. the HC2 has a heavy chain CDR1 comprising, consisting essentially of, or consisting of the amino acid sequence of SEQ ID NO:92, a heavy chain CDR2 comprising, consisting essentially of, or consisting of the amino acid sequence of SEQ ID NO:93, and a heavy chain CDR3 comprising, consisting essentially of, or consisting of the amino acid sequence of SEQ ID NO:94, and the LC2 has a light chain CDR1 comprising, consisting essentially of, or consisting of the amino acid sequence of SEQ ID NO:95, a light chain CDR2 comprising, consisting essentially of, or consisting of the amino acid sequence of SEQ ID NO:96, and a light chain CDR3 comprising, consisting essentially of, or consisting of the amino acid sequence of SEQ ID NO:97;
   ii. the HC2 comprises an amino acid sequence that is at least 90%, 95%, or 99% identical to the amino acid sequence of SEQ ID NO:90 and the LC2 comprises an amino acid sequence that is at least 90%, 95%, or 99% identical to the amino acid sequence of SEQ ID NO:91; or
   iii. the HC2 comprises, consists essentially of, or consists of the amino acid sequence of SEQ ID NO:90 and the LC2 comprises, consists essentially of, or consists of the amino acid sequence of SEQ ID NO:91,
      wherein the CDRs are defined according to Kabat.

The ROR1×CD3 bispecific antibody, or bispecific antigen-binding fragment thereof, can comprise:
a. a first antigen-binding site that immunospecifically binds ROR1, the first antigen-binding site formed from a HC1 and a LC1, wherein
   i. the HC1 has a heavy chain CDR1 comprising, consisting essentially of, or consisting of the amino acid sequence of SEQ ID NO:64, a heavy chain CDR2 comprising, consisting essentially of, or consisting of the amino acid sequence of SEQ ID NO:19, and a heavy chain CDR3 comprising, consisting essentially of, or consisting of the amino acid sequence of SEQ ID NO:65, and the LC1 has a light chain CDR1 comprising, consisting essentially of, or consisting of the amino acid sequence of SEQ ID NO:6, a light chain CDR2 comprising, consisting essentially of, or consisting of the amino acid sequence of SEQ ID NO:7, and a light chain CDR3 comprising, consisting essentially of, or consisting of the amino acid sequence of SEQ ID NO:8;
   ii. the HC1 comprises an amino acid sequence that is at least 90%, 95%, or 99% identical to the amino acid sequence of SEQ ID NO:63 and the LC1 comprises an amino acid sequence that is at least 90%, 95%, or 99% identical to the amino acid sequence of SEQ ID NO:5; or
   iii. the HC1 comprises, consists essentially of, or consists of the amino acid sequence of SEQ ID NO:63 and the LC1 comprises, consists essentially of, or consists of the amino acid sequence of SEQ ID NO:5; and
b. a second antigen-binding site that immunospecifically binds CD3, the second antigen-binding site formed from a HC2 and a LC2, wherein
   i. the HC2 has a heavy chain CDR1 comprising, consisting essentially of, or consisting of the amino acid sequence of SEQ ID NO:92, a heavy chain CDR2 comprising, consisting essentially of, or consisting of the amino acid sequence of SEQ ID NO:93, and a heavy chain CDR3 comprising, consisting essentially of, or consisting of the amino acid sequence of SEQ ID NO:94, and the LC2 has a light chain CDR1 comprising, consisting essentially of, or consisting of the amino acid sequence of SEQ ID NO:95, a light chain CDR2 comprising, consisting essentially of, or consisting of the amino acid sequence of SEQ ID NO:96, and a light chain CDR3 comprising, consisting essentially of, or consisting of the amino acid sequence of SEQ ID NO:97;
   ii. the HC2 comprises an amino acid sequence that is at least 90%, 95%, or 99% identical to the amino acid sequence of SEQ ID NO:90 and the LC2 comprises an amino acid sequence that is at least 90%, 95%, or 99% identical to the amino acid sequence of SEQ ID NO:91; or
   iii. the HC2 comprises, consists essentially of, or consists of the amino acid sequence of SEQ ID NO:90 and the LC2 comprises, consists essentially of, or consists of the amino acid sequence of SEQ ID NO:91,
      wherein the CDRs are defined according to Kabat.

The ROR1×CD3 bispecific antibody, or bispecific antigen-binding fragment thereof, can comprise:
a. a first antigen-binding site that immunospecifically binds ROR1, the first antigen-binding site formed from a HC1 and a LC1, wherein
   i. the HC1 has a heavy chain CDR1 comprising, consisting essentially of, or consisting of the amino acid sequence of SEQ ID NO:67, a heavy chain CDR2 comprising, consisting essentially of, or consisting of the amino acid sequence of SEQ ID NO:68, and a heavy chain CDR3 comprising, consisting essentially of, or consisting of the amino acid sequence of SEQ ID NO:69, and the LC1 has a light chain CDR1 comprising, consisting essentially of, or consisting of the amino acid sequence of SEQ ID NO:6, a light chain CDR2 comprising, consisting essentially of, or consisting of the amino acid sequence of SEQ ID NO:7, and a light chain CDR3 comprising, consisting essentially of, or consisting of the amino acid sequence of SEQ ID NO:8;
   ii. the HC1 comprises an amino acid sequence that is at least 90%, 95%, or 99% identical to the amino acid sequence of SEQ ID NO:66 and the LC1 comprises an amino acid sequence that is at least 90%, 95%, or 99% identical to the amino acid sequence of SEQ ID NO:5; or
   iii. the HC1 comprises, consists essentially of, or consists of the amino acid sequence of SEQ ID NO:66 and the LC1 comprises, consists essentially of, or consists of the amino acid sequence of SEQ ID NO:5; and
b. a second antigen-binding site that immunospecifically binds CD3, the second antigen-binding site formed from a HC2 and a LC2, wherein
   i. the HC2 has a heavy chain CDR1 comprising, consisting essentially of, or consisting of the amino acid sequence of SEQ ID NO:92, a heavy chain CDR2 comprising, consisting essentially of, or consisting of the amino acid sequence of SEQ ID NO:93, and a heavy chain CDR3 comprising, consisting essentially of, or consisting of the amino acid sequence of SEQ ID NO:94, and the LC2 has a light chain CDR1 comprising, consisting essentially of, or consisting of the amino acid sequence of SEQ ID NO:95, a light chain CDR2 comprising, consisting essentially of, or consisting of the amino acid sequence of SEQ ID NO:96, and a light chain CDR3 comprising, consisting essentially of, or consisting of the amino acid sequence of SEQ ID NO:97;
   ii. the HC2 comprises an amino acid sequence that is at least 90%, 95%, or 99% identical to the amino acid sequence of SEQ ID NO:90 and the LC2 comprises an amino acid sequence that is at least 90%, 95%, or 99% identical to the amino acid sequence of SEQ ID NO:91; or
   iii. the HC2 comprises, consists essentially of, or consists of the amino acid sequence of SEQ ID NO:90 and the LC2 comprises, consists essentially of, or consists of the amino acid sequence of SEQ ID NO:91,
      wherein the CDRs are defined according to Kabat.

The ROR1×CD3 bispecific antibody, or bispecific antigen-binding fragment thereof, can comprise:
a. a first antigen-binding site that immunospecifically binds ROR1, the first antigen-binding site formed from a HC1 and a LC1, wherein
   i. the HC1 has a heavy chain CDR1 comprising, consisting essentially of, or consisting of the amino acid sequence of SEQ ID NO:10, a heavy chain CDR2 comprising, consisting essentially of, or consisting of the amino acid sequence of SEQ ID NO:71, and a heavy chain CDR3 comprising, consisting essentially of, or consisting of the amino acid sequence of SEQ ID NO:72, and the LC1 has a light chain CDR1 comprising, consisting essentially of, or consisting of the amino acid sequence of SEQ ID NO:6, a light chain CDR2 comprising, consisting essentially of, or consisting of the amino acid sequence of SEQ ID NO:7, and a light chain CDR3 comprising, consisting essentially of, or consisting of the amino acid sequence of SEQ ID NO:8;
   ii. the HC1 comprises an amino acid sequence that is at least 90%, 95%, or 99% identical to the amino acid sequence of SEQ ID NO:70 and the LC1 comprises an amino acid sequence that is at least 90%, 95%, or 99% identical to the amino acid sequence of SEQ ID NO:5; or
   iii. the HC1 comprises, consists essentially of, or consists of the amino acid sequence of SEQ ID NO:70 and the LC1 comprises, consists essentially of, or consists of the amino acid sequence of SEQ ID NO:5; and
b. a second antigen-binding site that immunospecifically binds CD3, the second antigen-binding site formed from a HC2 and a LC2, wherein
   i. the HC2 has a heavy chain CDR1 comprising, consisting essentially of, or consisting of the amino acid sequence of SEQ ID NO:92, a heavy chain CDR2 comprising, consisting essentially of, or consisting of the amino acid sequence of SEQ ID NO:93, and a heavy chain CDR3 comprising, consisting essentially of, or consisting of the amino acid sequence of SEQ ID NO:94, and the LC2 has a light chain CDR1 comprising, consisting essentially of, or consisting of the amino acid sequence of SEQ ID NO:95, a light chain CDR2 comprising, consisting essentially of, or consisting of the amino acid sequence of SEQ ID NO:96, and a light chain CDR3 comprising, consisting essentially of, or consisting of the amino acid sequence of SEQ ID NO:97;
   ii. the HC2 comprises an amino acid sequence that is at least 90%, 95%, or 99% identical to the amino acid sequence of SEQ ID NO:90 and the LC2 comprises an amino acid sequence that is at least 90%, 95%, or 99% identical to the amino acid sequence of SEQ ID NO:91; or
   iii. the HC2 comprises, consists essentially of, or consists of the amino acid sequence of SEQ ID NO:90 and the LC2 comprises, consists essentially of, or consists of the amino acid sequence of SEQ ID NO:91,
      wherein the CDRs are defined according to Kabat.

The ROR1×CD3 bispecific antibody, or bispecific antigen-binding fragment thereof, can comprise:
a. a first antigen-binding site that immunospecifically binds ROR1, the first antigen-binding site formed from a HC1 and a LC1, wherein
   i. the HC1 has a heavy chain CDR1 comprising, consisting essentially of, or consisting of the amino acid sequence of SEQ ID NO:54, a heavy chain CDR2 comprising, consisting essentially of, or consisting of the amino acid sequence of SEQ ID NO:74, and a heavy chain CDR3 comprising, consisting essentially of, or consisting of the amino acid sequence of SEQ ID NO:75, and the LC1 has a light chain CDR1 comprising, consisting essentially of, or consisting of the amino acid sequence of SEQ ID NO:77, a light chain CDR2 comprising, consisting essentially of, or consisting of the amino acid sequence of SEQ ID NO:51, and a light chain CDR3 comprising, consisting essentially of, or consisting of the amino acid sequence of SEQ ID NO:78;
   ii. the HC1 comprises an amino acid sequence that is at least 90%, 95%, or 99% identical to the amino acid sequence of SEQ ID NO:73 and the LC1 comprises an amino acid sequence that is at least 90%, 95%, or 99% identical to the amino acid sequence of SEQ ID NO:76; or
   iii. the HC1 comprises, consists essentially of, or consists of the amino acid sequence of SEQ ID NO:73 and the LC1 comprises, consists essentially of, or consists of the amino acid sequence of SEQ ID NO:76; and
b. a second antigen-binding site that immunospecifically binds CD3, the second antigen-binding site formed from a HC2 and a LC2, wherein
   i. the HC2 has a heavy chain CDR1 comprising, consisting essentially of, or consisting of the amino acid sequence of SEQ ID NO:92, a heavy chain CDR2 comprising, consisting essentially of, or consisting of the amino acid sequence of SEQ ID NO:93, and a heavy chain CDR3 comprising, consisting essentially of, or consisting of the amino acid sequence of SEQ ID NO:94, and the LC2 has a light chain CDR1 comprising, consisting essentially of, or consisting of the amino acid sequence of SEQ ID NO:95, a light chain CDR2 comprising, consisting essentially of, or consisting of the amino acid sequence of SEQ ID NO:96, and a light chain CDR3 comprising, consisting essentially of, or consisting of the amino acid sequence of SEQ ID NO:97;
   ii. the HC2 comprises an amino acid sequence that is at least 90%, 95%, or 99% identical to the amino acid sequence of SEQ ID NO:90 and the LC2 comprises an amino acid sequence that is at least 90%, 95%, or 99% identical to the amino acid sequence of SEQ ID NO:91; or
   iii. the HC2 comprises, consists essentially of, or consists of the amino acid sequence of SEQ ID NO:90 and the LC2 comprises, consists essentially of, or consists of the amino acid sequence of SEQ ID NO:91,
      wherein the CDRs are defined according to Kabat.

The ROR1×CD3 bispecific antibody, or bispecific antigen-binding fragment thereof, can comprise:
a. a first antigen-binding site that immunospecifically binds ROR1, the first antigen-binding site formed from a HC1 and a LC1, wherein
   i. the HC1 has a heavy chain CDR1 comprising, consisting essentially of, or consisting of the amino acid sequence of SEQ ID NO:22, a heavy chain CDR2 comprising, consisting essentially of, or consisting of the amino acid sequence of SEQ ID NO:23, and a heavy chain CDR3 comprising, consisting essentially of, or consisting of the amino acid sequence of SEQ ID NO:80, and the LC1 has a light chain CDR1 comprising, consisting essentially of, or consisting of the amino acid sequence of SEQ ID NO:82, a light chain CDR2 comprising, consisting essentially of, or consisting of the amino acid sequence of SEQ ID NO:7, and a light chain CDR3 comprising, consisting essentially of, or consisting of the amino acid sequence of SEQ ID NO:83;
   ii. the HC1 comprises an amino acid sequence that is at least 90%, 95%, or 99% identical to the amino acid sequence of SEQ ID NO:79 and the LC1 comprises an amino acid sequence that is at least 90%, 95%, or 99% identical to the amino acid sequence of SEQ ID NO:81; or
   iii. the HC1 comprises, consists essentially of, or consists of the amino acid sequence of SEQ ID NO:79 and the LC1 comprises, consists essentially of, or consists of the amino acid sequence of SEQ ID NO:81; and
b. a second antigen-binding site that immunospecifically binds CD3, the second antigen-binding site formed from a HC2 and a LC2, wherein
   i. the HC2 has a heavy chain CDR1 comprising, consisting essentially of, or consisting of the amino acid sequence of SEQ ID NO:92, a heavy chain CDR2 comprising, consisting essentially of, or consisting of the amino acid sequence of SEQ ID NO:93, and a heavy chain CDR3 comprising, consisting essentially of, or consisting of the amino acid sequence of SEQ ID NO:94, and the LC2 has a light chain CDR1 comprising, consisting essentially of, or consisting of the amino acid sequence of SEQ ID NO:95, a light chain CDR2 comprising, consisting essentially of, or consisting of the amino acid sequence of SEQ ID NO:96, and a light chain CDR3 comprising, consisting essentially of, or consisting of the amino acid sequence of SEQ ID NO:97;
   ii. the HC2 comprises an amino acid sequence that is at least 90%, 95%, or 99% identical to the amino acid sequence of SEQ ID NO:90 and the LC2 comprises an amino acid sequence that is at least 90%, 95%, or 99% identical to the amino acid sequence of SEQ ID NO:91; or
   iii. the HC2 comprises, consists essentially of, or consists of the amino acid sequence of SEQ ID NO:90 and the LC2 comprises, consists essentially of, or consists of the amino acid sequence of SEQ ID NO:91,
      wherein the CDRs are defined according to Kabat.

The ROR1×CD3 bispecific antibody, or bispecific antigen-binding fragment thereof, can comprise:
a. a first antigen-binding site that immunospecifically binds ROR1, the first antigen-binding site formed from a HC1 and a LC1, wherein
   i. the HC1 has a heavy chain CDR1 comprising, consisting essentially of, or consisting of the amino acid sequence of SEQ ID NO:22, a heavy chain CDR2 comprising, consisting essentially of, or consisting of the amino acid sequence of SEQ ID NO:85, and a heavy chain CDR3 comprising, consisting essentially of, or consisting of the amino acid sequence of SEQ ID NO:86, and the LC1 has a light chain CDR1 comprising, consisting essentially of, or consisting of the amino acid sequence of SEQ ID NO:88, a light chain CDR2 comprising, consisting essentially of, or consisting of the amino acid sequence of SEQ ID NO:43, and a light chain CDR3 comprising, consisting essentially of, or consisting of the amino acid sequence of SEQ ID NO:89;
   ii. the HC1 comprises an amino acid sequence that is at least 90%, 95%, or 99% identical to the amino acid sequence of SEQ ID NO:84 and the LC1 comprises an amino acid sequence that is at least 90%, 95%, or 99% identical to the amino acid sequence of SEQ ID NO:87; or
   iii. the HC1 comprises, consists essentially of, or consists of the amino acid sequence of SEQ ID NO:84 and the LC1 comprises, consists essentially of, or consists of the amino acid sequence of SEQ ID NO:87; and
b. a second antigen-binding site that immunospecifically binds CD3, the second antigen-binding site formed from a HC2 and a LC2, wherein
   i. the HC2 has a heavy chain CDR1 comprising, consisting essentially of, or consisting of the amino acid sequence of SEQ ID NO:92, a heavy chain CDR2 comprising, consisting essentially of, or consisting of the amino acid sequence of SEQ ID NO:93, and a heavy chain CDR3 comprising, consisting essentially of, or consisting of the amino acid sequence of SEQ ID NO:94, and the LC2 has a light chain CDR1 comprising, consisting essentially of, or consisting of the amino acid sequence of SEQ ID NO:95, a light chain CDR2 comprising, consisting essentially of, or consisting of the amino acid sequence of SEQ ID NO:96, and a light chain CDR3 comprising, consisting essentially of, or consisting of the amino acid sequence of SEQ ID NO:97;
   ii. the HC2 comprises an amino acid sequence that is at least 90%, 95%, or 99% identical to the amino acid sequence of SEQ ID NO:90 and the LC2 comprises an amino acid sequence that is at least 90%, 95%, or 99% identical to the amino acid sequence of SEQ ID NO:91; or
   iii. the HC2 comprises, consists essentially of, or consists of the amino acid sequence of SEQ ID NO:90 and the LC2 comprises, consists essentially of, or consists of the amino acid sequence of SEQ ID NO:91,
      wherein the CDRs are defined according to Kabat.

The disclosed ROR1×CD3 bispecific antibodies or bispecific antigen-binding fragments thereof can bind to ROR1 with a K_{D} of less than about 100 nM, less than about 75 nM, less than about 50 nM, less than about 30 nM, less than about 25 nM, less than about 20 nM, less than about 15 nM, less than about 10 nM, or less than about 7.5 nM as measured by Biacore. The disclosed ROR1×CD3 bispecific antibodies or bispecific antigen-binding fragments thereof can bind ROR1 with a K_{D} of about 5 nM to about 100 nM as measured by Biacore. The disclosed ROR1×CD3 bispecific antibodies or bispecific antigen-binding fragments thereof can bind ROR1 with a K_{D} of about 5 nM to about 75 nM as measured by Biacore. The disclosed ROR1×CD3 bispecific antibodies or bispecific antigen-binding fragments thereof can bind ROR1 with a K_{D} of about 5 nM to about 50 nM as measured by Biacore. The disclosed ROR1×CD3 bispecific antibodies or bispecific antigen-binding fragments thereof can bind ROR1 with a K_{D} of about 5 nM to about 30 nM as measured by Biacore. The disclosed ROR1×CD3 bispecific antibodies or bispecific antigen-binding fragments thereof can bind ROR1 with a K_{D} of about 5 nM to about 25 nM as measured by Biacore. The disclosed ROR1×CD3 bispecific antibodies or bispecific antigen-binding fragments thereof can bind ROR1 with a K_{D} of about 5 nM to about 20 nM as measured by Biacore. The disclosed ROR1×CD3 bispecific antibodies or bispecific antigen-binding fragments thereof can bind ROR1 with a K_{D} of about 5 nM to about 15 nM as measured by Biacore. The disclosed ROR1×CD3 bispecific antibodies or bispecific antigen-binding fragments thereof can bind ROR1 with a K_{D} of about 5 nM to about 10 nM as measured by Biacore. The disclosed ROR1×CD3 bispecific antibodies or bispecific antigen-binding fragments thereof can bind ROR1 with a K_{D} of about 10 nM to about 100 nM as measured by Biacore. The disclosed ROR1×CD3 bispecific antibodies or bispecific antigen-binding fragments thereof can bind ROR1 with a K_{D} of about 15 nM to about 100 nM as measured by Biacore. The disclosed ROR1×CD3 bispecific antibodies or bispecific antigen-binding fragments thereof can bind ROR1 with a K_{D} of about 20 nM to about 100 nM as measured by Biacore. The disclosed ROR1×CD3 bispecific antibodies or bispecific antigen-binding fragments thereof can bind ROR1 with a K_{D} of about 25 nM to about 100 nM as measured by Biacore. The disclosed ROR1×CD3 bispecific antibodies or bispecific antigen-binding fragments thereof can bind ROR1 with a K_{D} of about 30 nM to about 100 nM as measured by Biacore. The disclosed ROR1×CD3 bispecific antibodies or bispecific antigen-binding fragments thereof can bind ROR1 with a K_{D} of about 50 nM to about 100 nM as measured by Biacore. The disclosed ROR1×CD3 bispecific antibodies or bispecific antigen-binding fragments thereof can bind ROR1 with a K_{D} of about 75 nM to about 100 nM as measured by Biacore.

In some embodiments, the first antigen-binding site that immunospecifically binds ROR1 can be derived from an IgG having one or more of the following mutations: IgG1 AA (F234A, L235A); IgG4 PAA (S228P, F234A, L235A); IgG2 AA (V234A, G237A); IgG1 FEA (L234F, L235E, D265A); or IgG1 FES (L234F/L235E/P331S). In some embodiments, the first antigen-binding site that immunospecifically binds ROR1 can contain the IgG1 AA (F234A, L235A) mutation. In some embodiments, the first antigen-binding site that immunospecifically binds ROR1 can contain the IgG4 PAA (S228P, F234A, L235A) mutation. In some embodiments, the first antigen-binding site that immunospecifically binds ROR1 can contain the IgG2 AA (V234A, G237A) mutation. In some embodiments, the first antigen-binding site that immunospecifically binds ROR1 can contain the IgG1 FEA (L234F, L235E, D265A) mutation. In some embodiments, the first antigen-binding site that immunospecifically binds ROR1 can contain the IgG1 FES (L234F/L235E/P331S) mutation. In some embodiments, the first antigen-binding site that immunospecifically binds ROR1 can contain the IgG1 L234A, L235A, and/or F405L mutations. In some embodiments the first antigen-binding site that immunospecifically binds ROR1 can contain the S228P, L234A, L235A, F405L, and/or R409K mutations. In some embodiments, the first antigen-binding site that immunospecifically binds ROR1 can contain the IgG-AA Fc-L234A, L235A, and F405L.

In some embodiments, the second antigen-binding site that immunospecifically binds CD3 can be derived from an IgG having one or more of the following mutations: IgG1 AA (F234A, L235A); IgG4 PAA (S228P, F234A, L235A); IgG2 AA (V234A, G237A); IgG1 FEA (L234F, L235E, D265A); or IgG1 FES (L234F/L235E/P331S). In some embodiments, the second antigen-binding site that immunospecifically binds CD3 can contain the IgG1 AA (F234A, L235A) mutation. In some embodiments, the second antigen-binding site that immunospecifically binds CD3 can contain the IgG4 PAA (S228P, F234A, L235A) mutation. In some embodiments, the second antigen-binding site that immunospecifically binds CD3 can contain the IgG2 AA (V234A, G237A) mutation. In some embodiments, the second antigen-binding site that immunospecifically binds CD3 can contain the IgG1 FEA (L234F, L235E, D265A) mutation. In some embodiments, the second antigen-binding site that immunospecifically binds CD3 can contain the IgG1 FES (L234F/L235E/P331S) mutation. In some embodiments, the second antigen-binding site that immunospecifically binds CD3 can contain the IgG1 L234A, L235A, and/or F405L mutations. In some embodiments the second antigen-binding site that immunospecifically binds CD3 can contain the S228P, L234A, L235A, F405L, and/or R409K mutations. In some embodiments, the second antigen-binding site that immunospecifically binds CD3 can contain the IgG-AA Fc-L234A, L235A, and F405L.

In some embodiments, the the second antigen-binding site that immunospecifically binds CD3 can bind CD3E on primary human T cells and/or primary cynomolgus T cells. In some embodiments, the second antigen-binding site that immunospecifically binds CD3 activates primary human CD4+ T cells and/or primary cynomolgus CD4+ T cells.

In some embodiments, the disclosed ROR1×CD3 bispecific antibodies are capable of binding to CD3 on human or cynomolgous monkey T-cells with a dissociation constant of less than 500, or less than 100 or less that 20 nM as determined by competition binding with a labeled anti-CD3 antibody with known affinity.

The ROR1×CD3 bispecific antibodies, or bispecific antigen-binding fragments thereof, can be single chain bispecific antibodies or bispecific antigen-binding fragments thereof. The ROR1×CD3 bispecific antibodies, or bispecific antigen-binding fragments thereof, can be BITEs (Micromet). The ROR1×CD3 bispecific antibodies, or bispecific antigen-binding fragments thereof, can be DARTs (MacroGenics). The ROR1×CD3 bispecific antibodies, or bispecific antigen-binding fragments thereof, can be Fcab and Mab2 (F-star). The ROR1×CD3 bispecific antibodies, or bispecific antigen-binding fragments thereof, can be Fc-engineered IgG1s (Xencor). The ROR1×CD3 bispecific antibodies, or bispecific antigen-binding fragments thereof, can be DuoBodies (Genmab). The ROR1×CD3 bispecific antibodies, or bispecific antigen-binding fragments thereof, can be TetBiAbs (Merck).

Methods of preparing bispecific antibodies invention include those described in WO2008/119353, WO2011/131746, van der Neut-Kolfschoten et al. (Science. 2007 Sep. 14; 317(5844):1554-7), PCT/US2015/051314, WO2005/061547, US2014/0170148, and US2016/0009824.

In addition to the disclosed ROR1×CD3 bispecific antibodies and bispecific antigen-binding fragments thereof, provided are polynucleotide sequences encoding the described ROR1×CD3 bispecific antibodies or bispecific antigen-binding fragments thereof. In some embodiments, the polynucleotide encoding the HC1, the HC2, the LC1 or the LC2 of the ROR1×CD3 bispecific antibody or bispecific antigen-binding fragment is provided. Vectors comprising the described polynucleotides are also provided, as are cells expressing the ROR1×CD3 bispecific antibodies or bispecific antigen-binding fragments thereof. These cells may be mammalian cells (such as 293F cells, CHO cells), insect cells (such as Sf7 cells), yeast cells, plant cells, or bacteria cells (such as E. coli). The disclosed ROR1×CD3 bispecific antibodies and bispecific antigen-binding fragments thereof may also be produced by hybridoma cells. In some embodiments, methods for generating the ROR1×CD3 bispecific antibodies or bispecific antigen-binding fragments by culturing cells is provided.

Further provided herein are pharmaceutical compositions comprising the ROR1×CD3 bispecific antibodies or bispecific antigen-binding fragments and a pharmaceutically acceptable carrier.

### Method of Treating Cancer

Disclosed herein are methods of treating a subject having cancer, the method comprising administering to the subject a therapeutically effective amount of any of the above disclosed ROR1×CD3 bispecific antibodies, or bispecific antigen-binding fragments thereof.

The use of an effective amount of any of the disclosed ROR1×CD3 bispecific antibody or bispecific antigen-binding fragment thereof in the treatment of cancer is also provided.

The disclosed ROR1×CD3 bispecific antibodies and bispecific antigen-binding fragments thereof can be used to inhibit the growth and/or proliferation of cancer cells or other diseased cells that express ROR1. Provided are methods for inhibiting growth or proliferation of cancer cells comprising administering a therapeutically effective amount of any of the disclosed the ROR1×CD3 bispecific antibodies or bispecific antigen-binding fragments to inhibit the growth or proliferation of cancer cells.

The disclosed ROR1×CD3 bispecific antibodies and bispecific antigen-binding fragments thereof can further be used to enhance the killing of ROR1-expressing diseased cells, such as cancer cells, by targeting CD3 expressing T cells to the ROR1-expressing cell. Provided herein are methods of redirecting a T cell to a ROR1-expressing cancer cell comprising administering a therapeutically effective amount of any of the disclosed the ROR1×CD3 bispecific antibodies or bispecific antigen-binding fragments to redirect a T cell to a cancer.

In some embodiments, the cancer is a ROR1-expressing cancer, such as lung cancer, hematological cancer, breast cancer, prostate cancer, pancreatic cancer, colon cancer, ovarian cancer, renal cancer, uterine cancer, or melanoma. The ROR1-expressing cancer can be a lung cancer, such as non-small cell lung cancer (NSCLC) or small cell lung cancer (SCLC). The ROR1-expressing cancer can be a hematological cancer, such as acute myeloid leukemia (AML), myelodysplastic syndrome (MDS, low or high risk), acute lymphocytic leukemia (ALL, including all subtypes), diffuse large B-cell lymphoma (DLBCL), chronic myeloid leukemia (CML), or blastic plasmacytoid dendritic cell neoplasm (DPDCN). The ROR1-expressing cancer can be breast cancer. The ROR1-expressing cancer can be prostate cancer. The ROR1-expressing cancer can be pancreatic cancer. The ROR1-expressing cancer can be colon cancer. The ROR1-expressing cancer can be ovarian cancer. The ROR1-expressing cancer can be renal cancer. The ROR1-expressing cancer can be uterine cancer. The ROR1-expressing cancer can be melanoma.

In some embodiments, the ROR1×CD3 bispecific antibody or bispecific antigen-binding fragment thereof can be administered to the subject as a pharmaceutical composition. Thus, also disclosed is the use of any of the disclosed ROR1×CD3 bispecific antibodies or bispecific antigen-binding fragments thereof in the manufacture of a composition for the treatment of cancer.

The pharmaceutical compositions provided herein can comprise: a) an effective amount of a ROR1×CD3 bispecific antibody or bispecific antigen-binding fragment thereof, and b) a pharmaceutically acceptable carrier, which may be inert or physiologically active. As used herein, the term “pharmaceutically acceptable carriers” includes any and all solvents, dispersion media, coatings, antibacterial and antifungal agents, and the like that are physiologically compatible. Examples of suitable carriers, diluents and/or excipients include one or more of water, saline, phosphate buffered saline, dextrose, glycerol, ethanol, and the like, as well as any combination thereof. In many cases, it will be preferable to include isotonic agents, such as sugars, polyalcohols, or sodium chloride in the composition. In particular, relevant examples of suitable carriers include: (1) Dulbecco's phosphate buffered saline, pH.about.7.4, containing or not containing about 1 mg/mL to 25 mg/mL human serum albumin, (2) 0.9% saline (0.9% w/v sodium chloride (NaCl)), and (3) 5% (w/v) dextrose; and may also contain an antioxidant such as tryptamine and a stabilizing agent such as Tween 20®.

The ROR1×CD3 bispecific antibody, bispecific antigen-binding fragment, or composition comprising the same may also contain a further therapeutic agent, as necessary for the particular disorder being treated. The ROR1×CD3 bispecific antibody or bispecific antigen-binding fragment thereof and the further therapeutic agent preferably have complementary activities that do not adversely affect each other. In a preferred embodiment, the further therapeutic agent can be cytarabine, an anthracycline, histamine dihydrochloride, or interleukin 2. In a preferred embodiment, the further therapeutic agent is a chemotherapeutic agent.

The ROR1×CD3 bispecific antibody, bispecific antigen-binding fragment, or composition comprising the same may be in a variety of forms including, for example, liquid, semi-solid, and solid dosage forms. The preferred form depends on the intended mode of administration and therapeutic application. The ROR1×CD3 bispecific antibody, bispecific antigen-binding fragment, or composition comprising the same can be in the form of injectable or infusible solutions. The ROR1×CD3 bispecific antibody, bispecific antigen-binding fragment, or composition comprising the same can be administered parenteraly (e.g. intravenous, intramuscular, intraperinoneal, subcutaneous). The ROR1×CD3 bispecific antibody, bispecific antigen-binding fragment, or composition comprising the same can be administered intravenously as a bolus or by continuous infusion over a period of time. The ROR1×CD3 bispecific antibody, bispecific antigen-binding fragment, or composition comprising the same can be injected by intramuscular, subcutaneous, intra-articular, intrasynovial, intratumoral, peritumoral, intralesional, or perilesional routes, to exert local as well as systemic therapeutic effects. The ROR1×CD3 bispecific antibody, bispecific antigen-binding fragment, or composition comprising the same can be administered orally.

Sterile preparations for parenteral administration can be prepared by incorporating the antibody, or antigen-binding fragment thereof, in the required amount in the appropriate solvent, followed by sterilization by microfiltration. As solvent or vehicle, there may be used water, saline, phosphate buffered saline, dextrose, glycerol, ethanol, and the like, as well as combination thereof. In many cases, isotonic agents, such as sugars, polyalcohols, or sodium chloride can be included in the composition. These compositions may also contain adjuvants, in particular wetting, isotonizing, emulsifying, dispersing and stabilizing agents. Sterile compositions for parenteral administration may also be prepared in the form of sterile solid compositions which may be dissolved at the time of use in sterile water or any other injectable sterile medium.

Solids for oral administration, including tablets, pills, powders (gelatine capsules, sachets) or granules may be used. In these compositions, the bispecific antibody or antigen-binding fragment thereof can be mixed with one or more inert diluents, such as starch, cellulose, sucrose, lactose or silica, under an argon stream. These compositions may also comprise substances other than diluents, for example one or more lubricants such as magnesium stearate or talc, a coloring, a coating (sugar-coated tablet) or a glaze.

For liquid compositions for oral administration, there may be used pharmaceutically acceptable solutions, suspensions, emulsions, syrups and elixirs containing inert diluents such as water, ethanol, glycerol, vegetable oils or paraffin oil. These compositions may comprise substances other than diluents, for example wetting, sweetening, thickening, flavoring or stabilizing products.

The dose of the ROR1×CD3 bispecific antibody, bispecific antigen-binding fragment, or composition comprising the same depends on the desired effect, the duration of the treatment, and the route of administration used. In general, the doctor will determine the appropriate dosage depending on the age, weight and any other factors specific to the subject to be treated.

Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation. Parenteral compositions may be formulated in dosage unit form for ease of administration and uniformity of dosage.

The ROR1×CD3 bispecific antibody or bispecific antigen-binding fragment thereof may also be administered in combination therapy, i.e., combined with other therapeutic agents relevant for the disease or condition to be treated. In some embodiments is provided a method for treating or preventing cancer, the method comprises administering to a subject a therapeutically effective amount of any of the disclosed ROR1×CD3 bispecific antibodies or bispecific antigen-binding fragments thereof, and a chemotherapeutic agent. In some embodiments, the other therapeutic agent is cytarabine, an anthracycline, histamine dihydrochloride, or interleukin 2. In some embodiments is provided a method for treating or preventing cancer, the method comprising administering to a subject a therapeutically effective amount of any of the disclosed ROR1×CD3 bispecific antibodies or bispecific antigen-binding fragments thereof and radiotherapy. Radiotherapy may comprise radiation or associated administration of radiopharmaceuticals. The source of radiation may be either external or internal to the patient being treated (radiation treatment may, for example, be in the form of external beam radiation therapy (EBRT) or brachytherapy (BT)). Radioactive elements include, e.g., radium, cesium-137, iridium-192, americium-241, gold-198, cobalt-57, copper-67, technetium-99, iodide-123, iodide-131, and indium-111. Combined administration of the disclosed bispecific ROR1×CD3 bispecific antibodies or bispecific antigen-binding fragments thereof and the other therapeutic agent may be simultaneous, separate or sequential, in any order. For simultaneous administration, the agents may be administered as one composition or as separate compositions, as appropriate.

### Examples

The following examples are provided to further describe some of the embodiments disclosed herein. The examples are intended to illustrate, not to limit, the disclosed embodiments.

### General Methods

### Transient Transfection and Expression of ROR in HEK Expi

ROR1 extracellular domain c-Mer proto-oncogene tyrosine kinase transmembrane domain (ROR1 ECD MERTK tm) was transiently expressed in cells for anti-ROR1 antibody reactivity confirmation (HEK293F), for characterization of commercial anti-ROR1 antibodies (HEK293F and CHO-S), to test phage and hybridoma panels against ROR2 cross-screen (CHO-S), to check cross-reactivity of ROR1 mAb hits against ROR2-ECD MERTK (CHO-S), to test for binding of anti-CD3 and anti-ROR1 antibodies to ROR1 transiently transfected cells (HEK293F), and to check cross-reactivity of ROR1 mAb hits against ROR2-ECD MERTK (CHO-S). ROR1 ECD MERTK tm (referred to herein as RR1W1; SEQ ID NO:99) has the following amino acid sequence:

QETELSVSAELVPTSSWNISSELNKDSYLTLDEPMNNITTSLGQTAELHC
KVSGNPPPTIRWFKNDAPVVQEPRRLSFRSTIYGSRLRIRNLDTTDTGYF
QCVATNGKEVVSSTGVLFVKFGPPPTASPGYSDEYEEDGFCQPYRGIACA
RFIGNRTVYMESLHMQGEIENQITAAFTMIGTSSHLSDKCSQFAIPSLCH
YAFPYCDETSSVPKPRDLCRDECEILENVLCQTEYIFARSNPMILMRLKL
PNCEDLPQPESPEAANCIRIGIPMADPINKNHKCYNSTGVDYRGTVSVTK
SGRQCQPWNSQYPHTHTFTALRFPELNGGHSYCRNPGNQKEAPWCFTLDE
NFKSDLCDIPACDSKDSKEKNKMEILFGCFCGFILIGLILYISLAIRGGG
GSGGGS

ROR2-ECD MERTK (referred to herein as RR1W2; SEQ ID NO:100) has the following amino acid sequence:

EVEVLDPNDPLGPLDGQDGPIPTLKGYFLNFLEPVNNITIVQGQTAILHC
KVAGNPPPNVRWLKNDAPVVQEPRRIIIRKTEYGSRLRIQDLDTTDTGYY
QCVATNGMKTITATGVLFVRLGPTHSPNHNFQDDYHEDGFCQPYRGIACA
RFIGNRTIYVDSLQMQGEIENRITAAFTMIGTSTHLSDQCSQFAIPSFCH
FVFPLCDARSRTPKPRELCRDECEVLESDLCRQEYTIARSNPLILMRLQL
PKCEALPMPESPDAANCMRIGIPAERLGRYHQCYNGSGMDYRGTASTTKS
GHQCQPWALQHPHSHHLSSTDFPELGGGHAYCRNPGGQMEGPWCFTQNKN
VRMELCDVPSCSPRDSSKMGFGCFCGFILIGLILYISLAIRGGGGSGGGS

HEK 293F cells were placed in Freestyle™ 293 media (Gibco #12338) at a density of 6e⁵ cells/ml to a volume of 30 mls in a 125 ml vented cap shake flask with shaking at 130 RPM, 24 hours prior to transfection. On the day of transfection, the cells were counted by Cedex and determined to have a density between 8e⁵ cells/ml and 1.2e⁶ cells/ml and a viability over 98%. Transfection was carried out using Freestyle max reagent (Invitrogen #16447). For a single 30 ml transfection, in one tube 37.5 μl of freestyle max reagent was diluted in 1 ml of OptiMEM media (Gibco #31985). In a separate tube, 37.5 μg of DNA (18.75 μg target and 18.75 μg pAdVAntage) was mixed into 1 ml OptiMEM. The two tubes were then mixed together, incubated in the biosafety cabinet for 3 minutes and then the mixture added directly to the flask of HEK293F cells.

For CHO-S transfections, CHO-S cells were placed in Freestyle CHO media (Gibco #12651) at a density of 6e⁵ cells/ml to a volume of 30 mls in a 125 ml vented cap shake flask with shaking at 130 RPM, 24 hours prior to transfection. On the day of transfection, the cells were counted by Cedex and determined to have a density between 8e⁵ cells/ml and 1.2e⁶ cells/ml and a viability over 98%. Transfection was carried out using Freestyle max reagent (Invitrogen #16447). For a single 30 ml transfection, in one tube 37.5 μl of freestyle max reagent was diluted in 1 ml of OptiMEM media (Gibco #31985). In a separate tube, 37.5 μg of DNA (18.75 μg target and 18.75 μg pAdVAntage) was mixed into 1 ml OptiMEM. The two tubes were then mixed together, incubated in the biosafety cabinet for 3 minutes and then the mixture added directly to the flask of CHO-S cells.

### Expression of Antibodies in HEK Expi Cells

Transient Expression was performed with the Expi293F transfection process (Expi293 Expression System Kit (Life Technologies Corporation Cat # A14635)). Expi293F cells (Life Technologies Corporation Cat #A14527) were grown at 37° C.; 7% CO₂; 130 RPM in Expi293 Expression Medium (Life Technologies Corporation Cat # A14351-01). Two days prior to transfection, cells were split at 7e⁵ cells/ml. At the time of transfection, cells were counted and verified to be at a concentration of at least 30e⁵ cells/ml and above 95% viable. For each 30-mL transfection, 30 μg of plasmid DNA was mixed with in Opti-MEM I Reduced Serum Medium (Life Technologies Corporation Cat #31985-070) to a total volume of 1.5 mL. (15 μg of pAdvantage DNA and 15 μg of expression vector DNA (for antibodies this is 1:3 ratio of HC:LC expression constructs). 81 μL of ExpiFectamine 293 Reagent (Life Technologies Corporation Cat # A14525) was then diluted in Opti-MEM I medium to a total volume of 1.5 mL. The diluted DNA and ExpiFectamine solutions were then mixed gently and incubated for 5 minutes at room temperature. The diluted DNA was added to the diluted ExpiFectamine 293 Reagent, mixed gently, and incubated for 20 minutes at room temperature. After the incubation, the mixture was then added to 25.5 ml of cells in a 125 ml shake flask. Immediately following transfection, 150 μL of ExpiFectamine 293 Transfection Enhancer 1 and 1.5 mL of ExpiFectamine 293 Transfection Enhancer 2 were added to each flask (Life Technologies Corporation Cat # A14525). Five days post transfection, cells supernatant was harvested by centrifugation and clarified through a 0.2 micron filter.

### Purification of Antibodies

The antibody in the clarified culture supernatant was captured by MabSelect SuRe™ Protein A resin and eluted with 100 mM sodium acetate (pH 3.5). The fractions containing the antibody were pooled and promptly neutralized with 2.5 M Tris HC1 (pH 7.2), then buffer exchanged into 1×D-PBS or other desired buffers if specified. The protein concentration was determined by measurement of OD280 on a NanoDrop spectrophotometer and calculated using its absorbance coefficient. The purity and homogeneity of the antibody was assessed by SDS-PAGE and SE-HPLC. Usually an SEC polishing step using Superdex 200 was performed if the monomer fell below 95% per SE-HPLC.

### MSD Cell Binding

Meso scale discovery (MSD) Streptavidin-Standard (SA-STD) plate was blocked with 50 μL per well of assay buffer for 5 minutes. The plate was inverted to remove assay buffer and tapped on paper towels. 1 μg/mL of 50 μL of biotinylated ROR1 in assay buffer were added to each well of the plate and incubated overnight at 4° C. 150 μL of assay buffer was added to each well of the coated plates without removing the coating reagent and incubated for ˜one hour. The plate was washed three times with wash buffer. The plate was tapped lightly on paper towels to remove residual wash buffer. 50 μL of human anti-ROR1 DuoBody® Ab in assay buffer were added to each well of the plate and incubated ˜one hour at ambient temperature. The plate was washed three times with wash buffer. 50 μL of 1.4 μg/mL of ruthenium-labeled anti-human IgG (H+L) antibody in assay buffer were added to each well of the plates. The plate was incubated for ˜one hour with gentle vortexing at ambient temp. The plate was washed three times with wash buffer. 150 μL of read buffer were added to each well of the plate. The plate was immediately read on the MSD sector imager 6000ä Reader for luminescence levels.

### Flow Cytometry Analysis of Cytotoxicity

Evaluation of cytotoxicity was performed using a flow cytometry assay. To do this, GFP labeled NCI-H358 target cells were plated in 96-well flat-bottomed plates at 2×10⁴ cells/well. The next day, 1×10⁵ primary pan T cells were added to each well in combination with the appropriate bispecific molecule in culture media. Cocultures were incubated at 37° C. for 72 hours prior to analysis. Cells were harvested using cell dissociation buffer (Life Technologies, USA) and labeled with fixable live/dead dye (Life Technologies, USA) and anti-CD25. These markers were used to evaluate death of target cells by gating on GFP⁺ cells and assessment of T cell activation by gating on GFP⁻ T cells, respectively. Samples were collected on an IntelliCyt iQue High Throughput Flow Cytometry HTFC system and analyzed using ForeCyt software. EC₅₀ values were calculated in GraphPad Prism V6. Acceptance criteria for non-linear regression curve fitting was a confidence interval (CI) range of less than 1.4.

### SPR Binding Affinity Studies

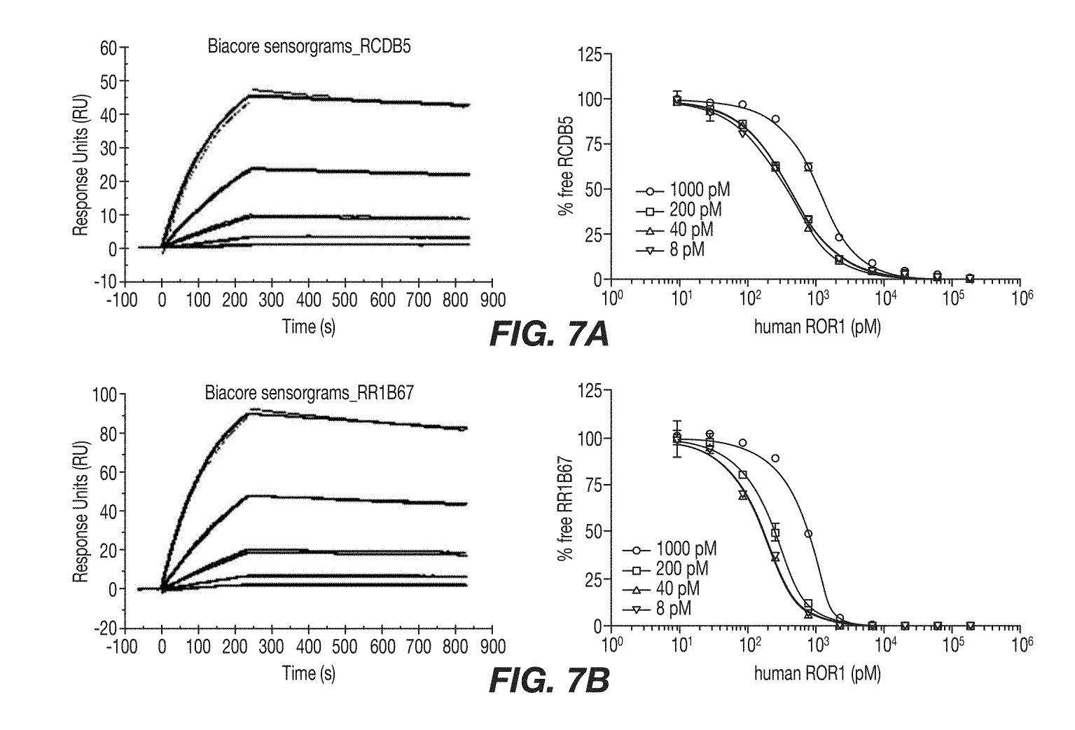

SPR experiments were performed with a four-channel Biacore T200 optical biosensor system at 25° C. For experiments with soluble ROR1, the four flowcells of a CMS sensor chip were immobilized with high levels (>6000 response units (RU)) of goat anti-human Fc antibody (Jackson ImmunoResearch, cat #109-005-098). Following this step, the control and test antibodies were captured on flow-cells 2, 3 and 4 until desired capture levels to generate sufficient antigen binding response were obtained (˜350 RU). Flow cell 1 did not have any captured antibody and was used as a reference surface. Recombinant human ROR1 was prepared in filtered and degassed PBSTE buffer (Bio-Rad #176-2730) starting from 400 nM to 5 nM at 3-fold dilutions (FIG. 7A, left; FIG. 7B, left). These solutions were injected over all 4 flow-cells at a flow rate of 50 μL/min and the association was monitored for 4 minutes followed by dissociation for 10 minutes. After each interaction, the sensor-chip surface was regenerated using glycine pH 1.5 to yield a stable baseline for following cycles.

Binding kinetics analyses of the anti-ROR1 antibody interactions with ROR1 were performed by global kinetics fitting of the sensorgrams using 1:1 Langmuir Model.

### Analysis of ROR1 Expression on Various Cells Lines

### ROR1 Receptor Expression in Lung, Colon, Prostate and Heme Cancer Cell Lines

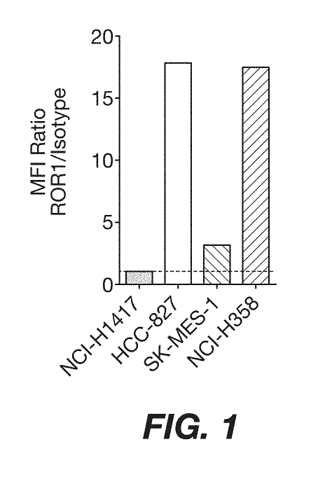

To evaluate the link between ROR1 expression and lung cancer, 70 lung cancer cell lines, including NSCLC derived from adenocarcinoma and squamous cell subtypes, and SCLC-derived lines, were evaluated for ROR1 expression. Binding of a commercially available phycoerythrin (PE) conjugated monoclonal anti-ROR1-antibody (2A2; Biolegend catalog #357803/357804) that does not crossreact with ROR2 was evaluated using a Fluorescence-Activated Cell Sorting (FACS)-based approach. Positivity was scored as showing a mean fluorescent intensity (MFI) signal that was 2-fold greater than a PE-conjugated mIgG1 isotype control. A significant percentage of the cell lines tested (54%) demonstrated expression of ROR1, including both NSCLC and SCLC-derived lines (data not shown). Within the positive lines, there was a range of expression from cell lines that expressed high levels of ROR1 (such as NCI-1155, LU1901R2 and NCI-H446), intermediate levels (such as NCI-H1975 and NCI-H358), and low to no expression (such as SKMES-1, NCI-H520 and NCI-H1417), using this detection method. Several lines with predominantly intermediate to low expression were selected for use in in vitro and in vivo assays disclosed herein. FIG. 1 illustrates the ROR1 expression from select lung cancer cell lines.

Colon, prostate, and cell lines were also evaluated for ROR1 expression. While the colon and prostate panels were limited, several lines with high ROR1 expression were identified that could be used in binding and functional assays, including HT-29.

TABLE 1
Summary of ROR1 expression data on in vitro tumor cell lines

| Tumor Cell Origin | Results | Expression > 2-fold over Isotype |
|---|---|---|
| Lung | 40/74 | 54% |
| Colon | 9/19 | 47% |
| Prostate | 4/6 | 67% |

Expression of ROR1 on lung, colon, and prostate-derived tumor cell lines by flow cytometry. A monoclonal anti-ROR1 antibody was used for detection of ROR1 on unfixed samples. For this analysis, MFI values were normalized using binding of an mIgG1 isotype control antibody (Biolegend cat# 400120) at the same concentration to calculate the fold-change over background.

### ROR1 Expression in Heme Cancer Cell Lines

Confluent Mantle Cell Lymphoma (MCL), B Cell Lymphoma, and Multiple Myeloma (MM) cells were stained for ROR1 expression using an in-house developed anti-ROR1 monoclonal antibody (RR1B121) that was conjugated to Alexa Fluor 647 according to manufacture's instructions (A647, ThermoFisher). Cells were washed twice in phosphate-buffered saline (PBS) and stained with Live/Dead (Aqua; ThermoFisher) for 10 minutes at room temperature. Live/Dead stain was washed out with PBS. Cells were either unstained or stained with 300 ng antibody in 50 uL final volume anti-ROR1-A647 in FACS Stain Buffer (BD Biosciences) for 30 minutes at 4° C. All staining steps were performed in the dark. Unstained sample was used as negative control; fluorescence minus one (FMO). Cells were washed twice with PBS and reconstituted in Stain Buffer for acquisition on the BD FACS Canto cytometer.

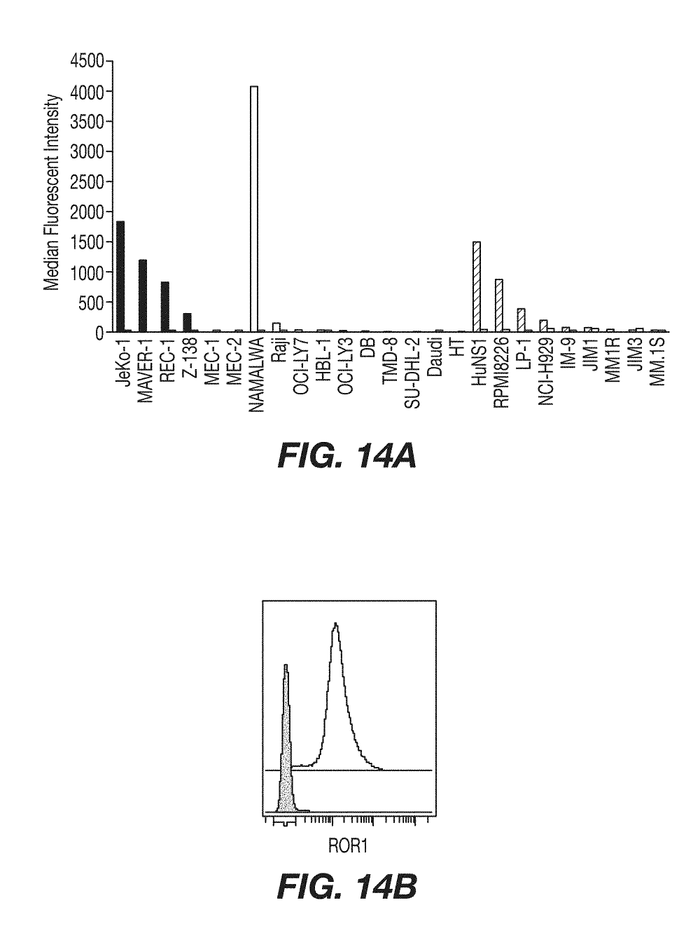

ROR1 expression was assessed in 25 cell lines from selected hematological malignancies (FIG. 14). Specific ROR1 expression was detected in all MCL lines tested and about half of MM lines. However, only two B lymphoma lines showed ROR1 expression.

### ROR1 Receptor Density in Heme Cancer Cell Lines

Confluent Mantle Cell Lymphoma (MCL) cell lines were stained for ROR1 expression using anti-ROR1 MAb conjugated to Phycoerythrin (PE) (clone 2A2, Biolegend). Cells were washed twice in phosphate-buffered saline (PBS) and stained for Live/Dead (Aqua; ThermoFisher) for 10 minutes at room temperature. Live/Dead stain was washed out with PBS. Cells were either unstained or stained with 1 uL/sample in 50 uL final volume anti-ROR1-PE in FACS Stain Buffer (BD Biosciences) for 30 minutes at 4° C. All staining steps were performed in the dark. Unstained sample was used as negative control; fluorescence minus one (FMO). Receptor density was measured using PE Quantibrite Beads according to manufacture's instructions (BD). Cells were washed twice with PBS and reconstituted in Stain Buffer for acquisition on the BD FACS Canto cytometer.

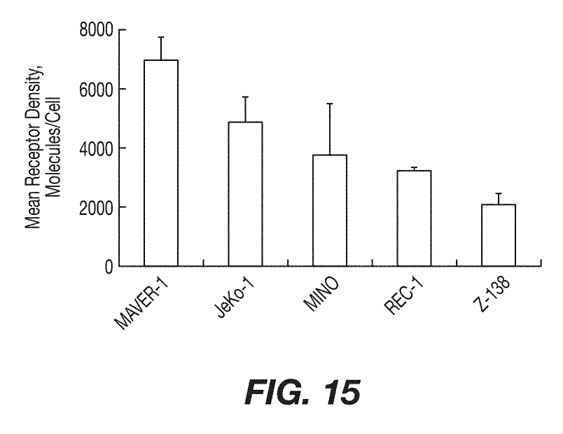

ROR1 expression was assessed in 5 MCL cell lines and receptor density (number of ROR1 molecules per cell) was quantified using the ABC method (FIG. 15). Data are presented as averages of two independent experiments (mean±SD) per cell line.

### ROR1 Expression in Normal Human Tissues

A tumor microarray (TMA) comprising an extensive array of normal tissues was also assessed for ROR1 positivity using the 4102s polyclonal antibody (Cell Signaling Technology). The results suggested that ROR1 expression was present on some normal tissues, including breast, colon, kidney, prostate and uterus (Table 2). Notably, out of 12 lung specimens analyzed, two showed some degree of positivity, with one demonstrating some level of membrane staining. In addition, fallopian tube sections appeared to be the only tissue with consistent evidence of membranous expression, in which 5 of 7 samples exhibited this staining profile. Overall, a substantial number of normal tissues exhibited some frequency of positivity, which has not been described in the literature. Due to this and the appreciable level of non-specific staining that was noted on control tumor cell lines, it was postulated that much of the positivity seen with this antibody was caused by non-specific staining.

TABLE 2
ROR1 Positive IHC Staining in a Normal Human TMA

| Tissue | Results | Tissue | Results |
|---|---|---|---|
| Adrenal gland | 2/15— likely glomerulosa cells | Skeletal Muscle: | 0/3 |
| | (40% intensity) | Quadriceps | |
| Breast | 4/6—no staining in stroma | Ovary | 3/12 (70-80% intensity) |
| Brain | 0/29 | Pancreas | 3/12 (20-40% intensity) |
| Blood-vessel: | 0/10 | Pituitary | 0/9 |
| Aorta | | | |
| Blood-vessel: | 0/3 | Prostate | 8/13 (40-80% intensity) |
| Renal artery | | | |
| Esophagus | 0/3 | Skin | 0/7 |
| Fallopian tube | 5/77^{†} (70-90% intensity) | | |
| Heart | 0/12 | Small | 3/6 (60-80% intensity) |
| | | Intestine | |
| Kidney | 12/15 (90% intensity) | Nerve | 0/3 |
| Large colon | 5/5—(80% intensity) | Spleen | 0/11 |
| Liver | 3/8—kupfer cells, | Stomach | 6/14—oxyntic cells (20- |
| | hepatocytes negative. | | 70% intensity) |
| Lung | 2/12^{§}—bronchial epithelium | Testis | 3/15 |
| Lymph node | 3/13 | Ureter | 2/9 (70% intensity) |
| Skeletal Muscle | 0/4 | Uterus: Corpus | 6/8 (80% intensity) |
| Skeletal Muscle: | 0/2 | Uterus: Cervix | 0/3 |
| Diaphragm | | | |

^{†}Membranous staining on all positive samples
^{§}Membranous staining on 1 of 2 positive samples
Expression of ROR1 on primary normal tissue sections by IHC. A rabbit anti-ROR1 polyclonal antibody was used for detection of ROR1 on formalin-fixed primary tumor samples. Staining was scored positive or negative and determined to be cytoplasmic or membranous by a pathologist.

ROR1 Expression on Circulating Tumor Cells (CTCs) from Small Cell Lung Cancer (SCLC) Patients

Since a significant number of small cell lung cancer (SCLC) cell lines had demonstrated ROR1 expression, analyses were performed on primary tumor cells from patients with this disease. SCLC is characterized by a high propensity for metastases, and therefore patients have a much higher incidence of circulating tumor cells (CTC) in the peripheral blood, which enabled the analysis of the incidence of ROR1 in this type of lung malignancy. For this analysis, fresh blood was fixed using a slow release formulation of formaldehyde in CellSave tubes. Two detection antibodies, 2A2 and the anti-ROR1 antibody RR1B78 (see infra), which binds the frizzled domain of ROR1, were tested. Spiking of control ROR1⁺ cell lines into whole blood demonstrated that RR1B78 was markedly more sensitive than 2A2. Therefore, analysis of CTCs in primary SCLC samples was performed using RR1B78. Analysis of peripheral blood samples from 7 samples showed that 5 patients had detectable ROR1 CTCs. Of these specimens, 3 samples had 10 or more CTCs that could be analyzed for ROR1 expression. Tumor cells from all three patients demonstrated detectable ROR1 on between 7-45% of cells, suggesting that ROR1 expression is a feature of some SCLC tumors (Table 3).

TABLE 3
ROR1 Expression on CTCs from SCLC patients

| | # of total CTCs | # ROR1⁺ CTCs |
|---|---|---|
| Patient ID# | detected^{§} | (%) |
| B11-SCLC | 296 | 21 (7%) |
| MT0820-SCLC | 1 | 0 |
| CNV265-SCLC | 1 | 1 (100%) |
| B12-SCLC | 0 | 0 |
| CNV505-SCLC | 0 | 0 |
| CNV678-SCLC | 10 | 1 (10%) |
| B13-SCLC | 234 | 106 (45%) |

§CTCs were identified as having a cytokeratin⁺CD45⁻ phenotype.
Detection of ROR1 on circulating tumor cells from SCLC patients. Patient blood samples were fixed in CellSave collection tubes from primary breast, colon, lung and protate tumors by IHC. RR1B78 was used for detection of ROR1 on formalin-fixed primary tumor samples. Staining was scored positive or negative by a pathologist. All expression was scored as being cytoplasmic.

ROR1 Expression on Tumor Cells from Chronic Lymphocytic Leukemia and Mantle Cell Lymphoma Patients

Frozen peripheral blood mononuclear cells (PBMC) or bone marrow mononuclear cells (BMMC) from patients with Chronic lymphocytic leukemia (CLL, all BMMC) or Mantle cell lymphoma (MCL, 2 donors matched PBMC and BMMC) were purchased from Conversant Bio. Samples were thawed quickly at 37 C and transferred to warm 12 mL of RPMI medium (containing 10% fetal bovine serum) (Invitrogen). After wash in ice cold PBS (Invitrogen) and filtration, cells were counted and seeded at 1×10⁶ live cells/well in 96-well round bottom plate. First, cells were stained with 50 uL of NearIR L/D IR (ThermoFisher) according to manufacture's protocol at room temperature in the dark for 10-15 min. After wash with ice cold PBS and FACs Stain Buffer (BD) cells were stained with antibody cocktail, anti-CD45-PerCP-Cy5.5 (eBioscience), anti-CD5-FITC and anti-CD19-PE-Cy7 (BD Bioscience), anti-CD38-PE, anti-CD40-BV605 and anti-CD137-BV421 (BioLegend) in 50 uL final volume Brilliant Stain buffer (BD Bioscience) for 30 min at 4° C. in the dark. Following a wash with FACs buffer, cells were reconstituted with 200 uL FACS buffer and analyzed by Fortessa II cytometer. Data analysis was performed using FlowJo and Prism software. Tumor cells were identified as CD19+CD5+ of live lymphocytes.

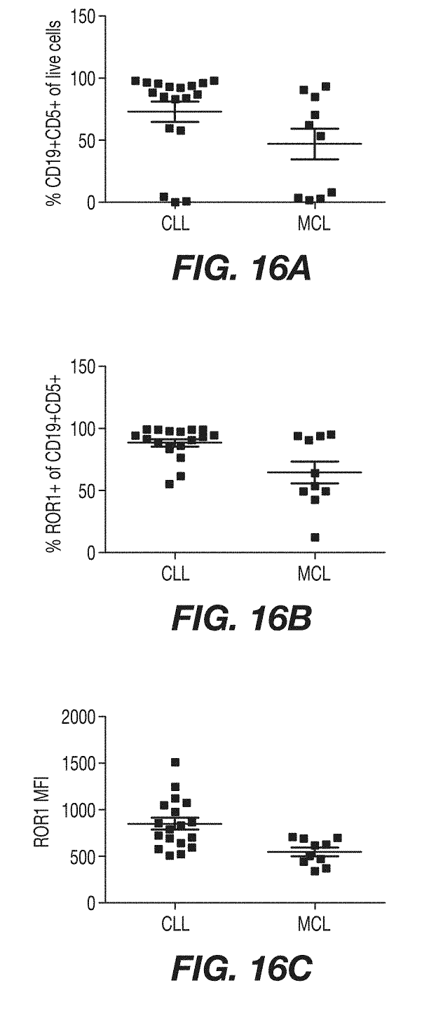

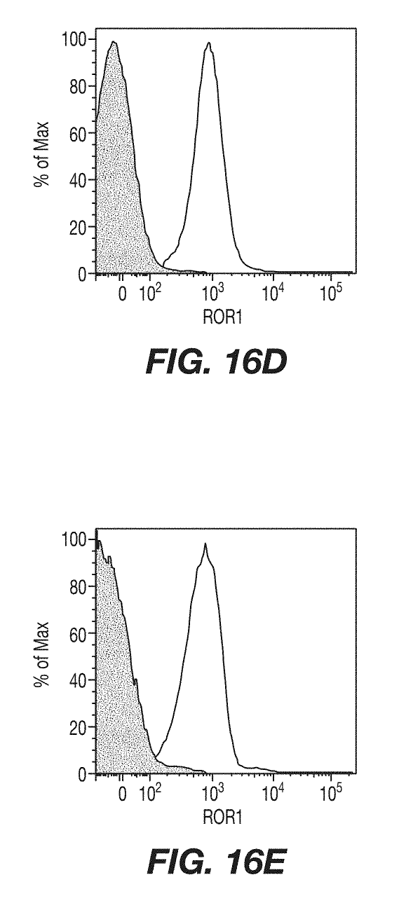

ROR1 was highly expressed in all of the CLL samples on the majority of tumor cells (MFI_{avg}=853) and independent of % tumor cells in the sample (FIG. 16). ROR1 was expressed at ˜1.5 fold lower level in MCL, as compared to CLL, (MFI_{avg}=575) on >50% tumor cells (FIG. 16).

Generation of Anti-ROR1 mAbs from Phage Panels

### Phage Panning and Panel Selection

ROR1-binding Fabs were selected from de novo pIX phage display libraries as described in Shi, L., et al. (2010) De novo selection of high-affinity antibodies from synthetic fab libraries displayed on phage as pIX fusion proteins. J Mol Biol 397, 385-396. Briefly, the libraries were generated by diversifying human scaffolds where germline V_{H} genes IGHV1-69*01, IGHV3-23*01, and IGHV5-51*01 were recombined with the human IGHJ-4 minigene via the H-CDR3 loop, and human germline V_{L} kappa genes O12 (IGKV1-39*01), L6 (IGKV3-11*01), A27 (IGKV3-20*01), and B3 (IGKV4-1*01) were recombined with the IGKJ-1 minigene to assemble complete V_{H} and V_{L} domains. Library design is detailed in Shi et al., J Mol Biol 397:385-96, 2010. The three heavy chain libraries were combined with the four germline light chains, known as Version 2, or combined with the diversified light chain libraries, known as Version 3, to generate 12 unique V_{H}:V_{L} combinations. These libraries were later combined further based on heavy chain gene to generate six libraries for panning experiments against ROR1.

Additional de novo pIX libraries based on the same heavy chains (1-69, 3-23, 5-51) and germline light chains (A27, B3, L6, 012), were generated by Sloning Biotech mutagenesis technologies. These were made as phage libraries, and were combined based on heavy chain gene to generate three additional libraries for panning experiments against ROR1.

The libraries were panned against biotinylated human ROR1-Fc (Sino Biological Inc Cat #13968-H02H1). Biotinylated antigen was captured on streptavidin magnetic beads (Dynal) and exposed to the de novo pIX Fab libraries at a final concentration of 100 nM or 10 nM. Non-specific phage were washed away in PBS-Tween and bound phage were recovered by infection of MC1061F′ E. coli cells. Phage were amplified from these cells overnight and panning was repeated for a total of four rounds. Following four rounds of biopanning, monoclonal Fabs were screened for binding to human ROR1 Fc in two formats: 1) in an ELISA where Fabs were captured on an ELISA plate by sheep anti-human FD, biotinylated ROR1-Fc was added to the captured Fabs, followed by detection of btROR-Fc with Streptavidin HRP; and 2) in an ELISA where btROR1-Fc was captured on an ELISA plate by Streptavidin, Fab supernatant was added to the captured antigen, followed by detection of the Fabs with goat AntiFab′2:HRP. Clones that demonstrated 10-fold over background binding to btROR1-Fc in either format were sequenced in the heavy and light chain variable regions.

A total of 69 clones were selected from the de novo selections based on human ROR1-Fc binding. 64 of the 69 Fabs (labeled RR1B1-RR1B64) were cloned into IgG2sigma/κ backbone to generate full length antibodies, expressed, and further characterized in the sections below.

### ROR1 Antibody Affinity Maturation:

To affinity mature the ROR1 antibodies, light chain libraries, generated using Sloning Biotech mutagenesis technologies, were constructed. The heavy chain variable regions from ROR1 Kringle domain binders—RR1B66, RR1B67, RR1B69, RR1B82, RR1B83, and RR1B84 (Table 9)—were cloned into a pIX phagemid vector containing this diversified VLk3-11 library. Once expressed and displayed these phage libraries were then panned stringently against ROR1 to obtain higher affinity binders.

Specifically, the libraries were panned against biotinylated human ROR1-Fc (Sino Biological Inc Cat #13968-H02H1). Biotinylated ROR1 was captured on streptavidin magnetic beads (Dynal) and exposed to the maturation pIX Fab libraries at a final concentration of 10 nM or 1 nM. Non-specific phage were washed away in PBS-Tween and bound phage were recovered by infection of MC1061F′ E. coli cells. Phage were amplified from these cells overnight and panning was repeated for a total of three rounds. Following three rounds of biopanning, monoclonal Fabs were screened for binding to human ROR1 Fc in three formats: 1) in an ELISA where Fabs were captured on an ELISA plate by sheep anti-human FD, biotinylated ROR1-Fc was added to the captured Fabs, followed by detection of btROR-Fc with Streptavidin HRP; 2) in an ELISA where btROR1-Fc was captured on an ELISA plate by Streptavidin, Fab supernatant was added to the captured antigen, followed by detection of the Fabs with goat AntiFab′2:HRP; and 3) in a proximity based luminescence immunoassay where the Fabs were allowed to bind in solution with bt-ROR-1, antiFab′2:HRP, and SA-acridin (BMG LabTech Lumistar Omega). Clones that demonstrated a signal to background ratio greater than 10-fold were sequenced in the heavy and light chain variable regions. Those clones that were unique and not matching the parental VL sequence were then selected for further characterization. These Fabs were tested in a ranking ELISA where Fabs were captured on an ELISA plate by sheep anti-human FD, biotinylated ROR1-Fc was added in a dilution series to the captured Fabs, followed by detection of bt-ROR-1-Fc with Streptavidin HRP. Fabs that demonstrated improved binding curves relative to the parental Fabs were then selected for conversion to mAb, also considering absence of possible PTM risks, and diversity of the LC sequence. A total of 36 clones were cloned into IgG4-PAA backbone to generate full length antibodies, expressed, and further characterized (see Table 22).

SPR experiments were performed using a ProteOn XPR36 system (Bio-Rad) at 25° C. to measure the binding of affinity matured anti-human ROR1 antibodies and the parental RR1B67 to human ROR1. Goat anti-human Fc IgG (Jackson Immunoresearch, cat #109-005-098) was directly immobilized via amine coupling at 30 μg/mL in acetate buffer, pH 5.0 on all 6 ligand channels in horizontal orientation on GLC Sensor Chip (Bio-Rad, catalog no. 176-5011) with a flow rate of 30 μL/min for 300 seconds in PBS containing 0.005% Tween-20. The immobilization densities averaged about 6000 Response Units (RU) with less than 5% variation among different channels. Five different mAbs were captured on the anti-human Fc IgG surface at 0.5 ug/ml (˜400 RU) in vertical ligand orientation, with the 6th ligand channel as no ligand surface control. Recombinant Human ROR1-ECD with a C terminal human serum albumin (HSA) fusion and a histag, RR1W27. in house) at 300 nM concentration in 3-fold dilution series of 5 concentrations flew in as analyte to bind to captured mAbs in the horizontal orientation. A buffer sample was also injected to monitor the dissociation of captured mAb and baseline stability. The dissociation phase for all concentrations of Ag was monitored at a flow rate of 100 μL/min for 30 minutes. The binding surface was regenerated for the next interaction cycle using a 18 second pulse of 0.8% phosphoric acid. The raw data were processed by subtracting two sets of reference data from the response data: 1) the inter-spot signals to correct for the non-specific interactions between the Ag and the empty chip surface; 2) the buffer channel signals to correct for baseline drifting due to the dissociation of captured mAb over time. The processed data at all concentrations for each mAb were globally fit to a 1:1 simple Langmuir binding model to extract the kinetic (kₒₙ, k_{off}) and affinity (K_{D}) constants. An arbitrary criteria using the % Chi2/Rmax<30% was set to measure the quality of fit, quantitative kinetic results for only those with valid fit in the summary Table 3A should be considered quantiatively reliable.

In some experiments a mammalian expression construct encoding the extracellular domain of human ROR1 (Uniprot Accession #Q01973|residues 30-406) fused at the N-terminus of human serum albumin (C34S)-6×His (RR1W27) was used to transiently transfect Expi293F cells. Six days post-transfection the culture supernatant was harvested by centrifugation. The RR1W27 was purified from the Expi293F supernatant by immobilized metal affinity chromatography (IMAC) followed by buffer exchange into 1×PBS by exhaustive dialysis.

TABLE 3A
Summary of binding results of affinity matured anti-ROR1
antibodies measured by ProteOn SPR.

| | | | | Fold of | |
|---|---|---|---|---|---|
| | | | | affinity | |
| | | | | increase | |
| | | | | over | |
| Protein | | | KD | parental | |
| AA | ka(M-1s-1) | kd (s-1) | (nM) | mAb | Comments |
| RR1B177 | 2.89E+05 | 1.90E−04 | 0.7 | 2.4 | |
| RR1B178 | 1.43E+05 | 5.87E−04 | 4.1 | 0.4 | |
| RR1B179 | 8.15E+04 | 4.00E−04 | 4.9 | 0.3 | |
| RR1B181 | 1.71E+05 | 2.30E−04 | 1.4 | 1.2 | |
| RR1B183 | 1.85E+05 | 3.49E−04 | 1.9 | 0.8 | |
| RR1B184 | 1.74E+05 | 1.95E−04 | 1.1 | 1.4 | |
| RR1B185 | 1.91E+05 | 2.91E−04 | 1.5 | 1.1 | |
| RR1B186 | 1.63E+05 | 1.94E−04 | 1.2 | 1.3 | |
| RR1B187 | 2.02E+05 | 1.04E−03 | 5.1 | 0.3 | |
| RR1B188 | 1.89E+05 | 6.32E−04 | 3.4 | 0.5 | |
| RR1B189 | 1.12E+05 | 6.13E−04 | 5.5 | 0.3 | |
| RR1B190 | 1.23E+05 | 2.54E−04 | 2.1 | 0.8 | |
| RR1B191 | 2.79E+05 | 3.40E−04 | 1.2 | 1.3 | |
| RR1B193 | 2.24E+05 | 6.23E−04 | 2.8 | 0.6 | |
| RR1B194 | 2.06E+05 | 4.90E−04 | 2.4 | 0.7 | |
| RR1B196 | 1.87E+05 | 4.45E−04 | 2.4 | 0.7 | |
| RR1B197 | 1.74E+05 | 7.85E−04 | 4.5 | 0.4 | |
| RR1B199 | 3.75E+05 | 7.44E−04 | 2.0 | 0.8 | |
| RR1B200 | 1.58E+05 | 2.91E−04 | 1.8 | 0.9 | |
| RR1B201 | 2.25E+05 | 9.97E−05 | 0.4 | 3.6 | |
| RR1B202 | 1.47E+05 | 1.07E−03 | 7.2 | 0.2 | |
| RR1B203 | 1.25E+05 | 2.75E−04 | 2.2 | 0.7 | |
| RR1B206 | 1.06E+05 | 7.28E−05 | 0.7 | 2.3 | |
| RR1B207 | 9.66E+04 | 1.47E−04 | 1.5 | 1.1 | |
| RR1B208 | 8.66E+04 | 1.37E−03 | 15.9 | 0.1 | |
| RR1B210 | 3.41E+05 | 7.85E−04 | 2.3 | 0.7 | |
| RR1B211 | 8.19E+04 | 7.08E−04 | 8.7 | 0.2 | |
| RR1B213 | 2.48E+05 | 1.30E−04 | 0.5 | 3.1 | |
| RR1B67 | 1.51E+05 | 2.48E−04 | 1.6 | 1.0 | parental molecule |
| RR1B180 | | | | | Binding observed; |
| | | | | | fit not valid |
| RR1B212 | | | | | Binding observed; |
| | | | | | fit not valid |
| RR1B209 | | | | | Binding observed; |
| | | | | | fit not valid |
| RR1B205 | | | | | Binding observed; |
| | | | | | fit not valid |
| RR1B204 | | | | | Binding observed; |
| | | | | | fit not valid |
| RR1B198 | | | | | Binding observed; |
| | | | | | fit not valid |
| RR1B192 | | | | | Binding observed; |
| | | | | | fit not valid |
| RR1B182 | | | | | Binding observed; |
| | | | | | fit not valid |

### Anti-ROR1 Antibody Cell Binding Characterization

The 64 anti-ROR1 antibodies were characterized by binding to CHO-S cell lines that expressed ROR1 or ROR2. Transfected CHO-S cells ROR1 (RR1W1) and ROR2 (RR1W2) and mock control CHO-S were provided for one set of binding experiments. In addition to flow based analysis, Western blot was used to confirm the expression of ROR1 on the CHO-S cells before characterization of the phage-derived hits.

The anti-ROR1 antibodies were screened using transiently transfected CHO-S cells 24 hours following transfection. Four commercial monoclonal anti-ROR1 antibodies (Creative Diagnostics catalog # DMAB8606MH; Biolegend (2A2 Ab) catalog #357803, 357804; AVIVA Systems Biology catalog # OAAD00316; ACRIS Antibodies, Inc. catalog #AM06399SU-N) and the goat polyclonal antibody from R&D systems (catalog # AF2000) were used as positive controls. Parental and mock-transfected CHO cells, as well as RSV isotype controls (B23B31) were used as negative controls. Binding of the test antibodies was determined using a polyclonal anti-human antibody labeled with alexafluor 674. Of the 64 antibodies tested, 63 showed robust binding to the ROR1 transfected CHO-S cells. Binding affinity was measured using standard deviation (SD) from the mean of the test articles with those less than the mean minus 1 SD being called low binders, those within 1 SD of the mean being called intermediate binders, and those with stronger binding than the mean plus 1 SD being called strong binders. Using this approach, 12 low, 44 intermediate and 7 strong ROR1 binders were identified. This set of antibodies was further characterized using endogenously expressing tumor cell lines by flow and western blot.

Transiently transfected ROR2 CHO cells were also analyzed in this experiment. No ROR2 expression was detected by any of the positive control antibodies tested (data not shown). With the anti-ROR1 antibodies, 11/64 were considered to be high ROR2 binders; RR1B3, B11, B14, B15, B17, B32, B43, B46, B51, B55 and B61 were eliminated based on the data.

The binding of anti-ROR1 antibodies to CHO-S cells transiently transfected with the ROR1 extracellular domain (ROR1-ECD) was repeated. An anti-human secondary (Goat Anti-human IgG AlexaFluor 647; Life Technologies catalog #A21445) was used for visualization of binding. A monoclonal anti-ROR1 antibody from Biolegend (A2A; Biolegend) was run in triplicate as a positive control. Parental and mock-transfected CHO cells, as well as isotype controls (B23B31) were run as negative controls. Binding of the antibodies were read out using a polyclonal anti-human antibody labeled with alexafluor 674 (Goat Anti-human IgG AlexaFluor 647; Life Technologies cat#A21445). Of the 64 antibodies tested, 62 showed robust binding to a fraction of the ROR1-ECD transfected cells that correlated with that shown by the positive control antibody (data not shown). This is in comparison with 63 of 64 which were identified in the previous experiment. RR1B31, which appeared to bind well in the previous experiment, did not bind in this experiment. Data was compared to that from the initial experiment using MFI values to rank the antibodies (Table 4). There were differences in the ranking between the two experiments probably due to the fact that the binders mostly bound within a very small range of MFI values. However, given the intrastudy variation, the data suggests that the majority of these antibodies bind ROR1 expressed by CHO cells following transient transfection.

TABLE 4
Activity of anti-ROR1 antibodies against ROR transfected CHO cells

| | MFI Values | | |
|---|---|---|---|
| | Parental | ROR2-Transfected | ROR1-Transfected |
| Antibody | CHO | CHO | CHO |
| Isotype Control | 1630 | 8903 | 1735 |
| RR1B1 | 2930 | 85223 | 1807 |
| RR1B2 | 4317 | 28981 | 5072 |
| RR1B3 | 5386 | 72524 | 11694 |
| RR1B4 | 5006 | 85459 | 4586 |
| RR1B5 | 4749 | 42152 | 5400 |
| RR1B6 | 5036 | 65166 | 4007 |
| RR1B7 | 4813 | 47028 | 4517 |
| RR1B8 | 5591 | 49271 | 5809 |
| RR1B9 | 7438 | 44950 | 6107 |
| RR1B10 | 6386 | 68761 | 8408 |
| RR1B11 | 3756 | 88144 | 15011 |
| RR1B12 | 7904 | 42268 | 6375 |
| RR1B13 | 1769 | 36671 | 1479 |
| RR1B14 | 3534 | 93611 | 11635 |
| RR1B15 | 3240 | 66981 | 12490 |
| RR1B16 | 2618 | 42469 | 4481 |
| RR1B17 | 4407 | 68602 | 12085 |
| RR1B18 | 2386 | 44097 | 3022 |
| RR1B19 | 1716 | 24990 | 1529 |
| RR1B20 | 2554 | 5420 | 1790 |
| RR1B21 | 5158 | 60139 | 4065 |
| RR1B22 | 3757 | 45408 | 3920 |
| RR1B23 | 4824 | 58357 | 4464 |
| RR1B24 | 5478 | 65416 | 5081 |
| RR1B25 | 4301 | 56058 | 4144 |
| RR1B26 | 3558 | 64312 | 5197 |
| RR1B27 | 6307 | 34511 | 7196 |
| RR1B28 | 3020 | 29951 | 2540 |
| RR1B29 | 3199 | 31030 | 4296 |
| RR1B30 | 3010 | 33126 | 2733 |
| RR1B31 | 4237 | 61410 | 3163 |
| RR1B32 | 3914 | 47957 | 13480 |
| RR1B33 | 4489 | 61314 | 4710 |
| RR1B34 | 4434 | 41971 | 4402 |
| RR1B35 | 5435 | 57284 | 4635 |
| RR1B36 | 4559 | 59947 | 4793 |
| RR1B37 | 3154 | 27367 | 3789 |
| RR1B38 | 21015 | 60174 | 12327 |
| RR1B39 | 3087 | 33765 | 3327 |
| RR1B40 | 3487 | 30990 | 2930 |
| RR1B41 | 1701 | 25963 | 1697 |
| RR1B42 | 3852 | 64131 | 10862 |
| RR1B43 | 5697 | 87621 | 5603 |
| RR1B44 | 6929 | 133040 | 3313 |
| RR1B45 | 6561 | 112695 | 5408 |
| RR1B46 | 6775 | 133631 | 12699 |
| RR1B47 | 4289 | 55285 | 3474 |
| RR1B48 | 14354 | 52064 | 9804 |
| RR1B49 | 3158 | 74413 | 5091 |
| RR1B50 | 8189 | 74438 | 7165 |
| RR1B51 | 4946 | 89028 | 11147 |
| RR1B52 | 5954 | 56117 | 2125 |
| RR1B53 | 14248 | 28490 | 9006 |
| RR1B54 | 9508 | 72969 | 8183 |
| RR1B55 | 5283 | 77480 | 7709 |
| RR1B56 | 4329 | 79419 | 3473 |
| RR1B57 | 2624 | 76578 | 2216 |
| RR1B58 | 13984 | 57324 | 8851 |
| RR1B59 | 3497 | 85595 | 2828 |
| RR1B60 | 4588 | 62703 | 3137 |
| RR1B61 | 3893 | 50742 | 10919 |
| RR1B62 | 3850 | 24006 | 3632 |
| RR1B63 | 4647 | 23244 | 3244 |
| RR1B64 | 1532 | 17221 | 1181 |

In addition to these CHO-S transfected cells, various cell lines were evaluated for use in characterization of the panel of the anti-ROR1 antibodies. MCF-7 cells were determined to be negative for ROR1 and ROR2 (data not shown). The cell line U266 was determined to be negative for ROR1 and positive for ROR2 (data not shown). MDA-MB231 cells were strongly positive for ROR1 (Table 5 and Table 6). HEK293 appeared to be positive for ROR2 (data not shown). CHO-S showed a relatively small shift compared to HEK293 and 3T3 cells, which correlated with the absence of a specific band by western blotting (data not shown). Interestingly, a smaller band was present in the blots indicating that the antibody (R&D Systems catalog #AF2000; Goat polyclonal) could bind to a protein expressed by these cells. Whether this is a truncated form of ROR1 or a different protein is not clear and needs further investigation. However, the shift seen in all cell lines could be caused by binding to this smaller species. It was also shown that HEK293 cells could be transiently transfected with ROR1 and cell surface expression detected (data not shown).

The anti-ROR1 antibodies were assessed for binding to two breast tumor lines: MDA-MB231 breast tumor cells expressing endogenous ROR1 (Table 5 and Table 6) and MCF-7 which are ROR1 negative (data not shown). An anti-human secondary was used for visualization of binding. Four monoclonal anti-ROR1 commercial antibodies (Creative Diagnostics catalog # DMAB8606MH; Biolegend (2A2 Ab) catalog #357803, 357804; AVIVA Systems Biology catalog # OAAD00316; ACRIS Antibodies, Inc. catalog #AM06399SU-N) and the goat polyclonal antibody from R&D systems (catalog # AF2000) were used as positive controls. A panel of six RSV controls (B23B31) were run to gauge background binding. Binding of the test antibodies was read out using a polyclonal anti-human antibody labeled with alexafluor 674. Mouse and goat positive control antibodies were detected using the appropriate polyclonal secondary antibodies conjugated to the same fluorochrome. Of the 64 antibodies tested, 61 showed binding above the mean of the isotype controls. Thirty-nine (39) antibodies demonstrated binding better than the best positive control antibody. Relative affinity was measured using standard deviation (SD) from the mean of the test articles, with those greater than the mean being called intermediate and those greater than the mean plus 1 SD being called high binders. Using this approach, 24 intermediate and 7 strong ROR1 binders were identified.

Next, the anti-ROR1 antibodies were assessed for binding to the ROR1 positive cell line, SKMES-1 and CHO-S ROR1 by Western Blot (data not shown). No bands were detected in the SKMES-1 lysates using the Phage hit supernatants. The ROR1 polyclonal antibody served as the control. Multiple bands were observed in the ROR1-CHO-S transfected lysate possibly due to glycosylation. A band of approximately 70 kDa was observed in the Western with phage RR1B31, RR1B20, RR1B51, RR1B61 and RR1B28, respectively. This observed band is believed to be a glycosylated form of ROR1. A faint band was observed using a control antibody in the SKMES-1 lysate at ˜130 kDa. This band is believed to be full length ROR1.

The anti-ROR1 antibodies were assessed for binding to two lung tumor cell lines: H358 and SKMES-1, as well as a mantle cell carcinoma line, JEKO-1 using flow cytometry. All of these lines have been shown to express endogenous ROR1 protein. The JEKO-1 (a mantle cell line) showed significant background (data not shown). SK-SH5Y cells, a neuroblastoma line shown in the literature to express ROR2, was also run, although the ROR1 status of these cells is not known (data not shown). Four monoclonal anti-ROR1 commercial antibodies (Creative Diagnostics catalog # DMAB8606MH; Biolegend (2A2 Ab) catalog #357803, 357804; AVIVA Systems Biology catalog # OAAD00316; ACRIS Antibodies, Inc. catalog #AM06399SU-N) and the goat polyclonal antibody from R&D systems (catalog # AF2000) were used as positive controls. A panel of six RSV isotype controls (B23B31) was used to gauge background binding. Binding of the test antibodies was read out using a polyclonal anti-human antibody labeled with alexafluor 674. Mouse and goat anti-ROR1 positive control antibodies were detected using the appropriate polyclonal secondary antibodies conjugated to the same fluorochrome.

Of the 64 anti-ROR1 antibodies tested, over half showed binding above the mean of the isotype controls. A handful of antibodies showed binding above that seen in the positive controls. RR1B48 showed consistently good binding across the cell lines tested, as did RR1B46, RR1B11, RR1B55 and RR1B58, which were all ranked in the top 10 for SKMES-1, H358 and MDA-MB231 (Table 6). RR1B48 and RR1B11 did not appear to be affected by formaldehyde-based fixation as much as RR1B46, RR1B55 and RR1B58. This may suggest that these two groups bind to non-fixation and fixation-sensitive epitopes, respectively. RR1B48 and RR1B11 may be useful for IHC which requires fixation prior to labeling. Overall, this data suggests that over half of the panel binds to endogenous ROR1 and allows the selection of the best binders for affinity testing and epitope binning. The fixation data also allows the selection of several antibodies for further testing as tool reagents for IHC.

TABLE 5
ROR1 expression in SKMES-1 and MDA-MB231 cell lines

| | MFI Values | |
|---|---|---|
| Antibody | SKMES-1 | MDA-MB231 |
| Isotype Control | 1372 | 1608 |
| RR1B1 | 6174 | 8105 |
| RR1B2 | 7735 | 6669 |
| RR1B3 | 6727 | 7158 |
| RR1B4 | 12921 | 10954 |
| RR1B5 | 9977 | 13225 |
| RR1B6 | 10234 | 17264 |
| RR1B7 | 5185 | 10501 |
| RR1B8 | 10711 | 15299 |
| RR1B9 | 7628 | 12996 |
| RR1B10 | 9225 | 16057 |
| RR1B11 | 26803 | 29792 |
| RR1B12 | 10346 | 17252 |
| RR1B13 | 9615 | 14810 |
| RR1B14 | 9176 | 14233 |
| RR1B15 | 10626 | 15113 |
| RR1B16 | 5453 | 8586 |
| RR1B17 | 4216 | 7933 |
| RR1B18 | 2528 | 4704 |
| RR1B19 | 1658 | 3097 |
| RR1B20 | 646 | 736 |
| RR1B21 | 5372 | 8057 |
| RR1B22 | 3375 | 5596 |
| RR1B23 | 7643 | 10673 |
| RR1B24 | 10483 | 13518 |
| RR1B25 | 8762 | 11918 |
| RR1B26 | 12992 | 15803 |
| RR1B27 | 4281 | 5641 |
| RR1B28 | 1558 | 1461 |
| RR1B29 | 2155 | 5743 |
| RR1B30 | 2253 | 3968 |
| RR1B31 | 2710 | 3855 |
| RR1B32 | 7218 | 12210 |
| RR1B33 | 6183 | 9347 |
| RR1B34 | 11124 | 12108 |
| RR1B35 | 5368 | 11495 |
| RR1B36 | 4912 | 9761 |
| RR1B37 | 2752 | 5543 |
| RR1B38 | 9102 | 10194 |
| RR1B39 | 2178 | 3382 |
| RR1B40 | 1695 | 2842 |
| RR1B41 | 1910 | 3014 |
| RR1B42 | 11407 | 7704 |
| RR1B43 | 8884 | 18478 |
| RR1B44 | 13163 | 18825 |
| RR1B45 | 18460 | 20915 |
| RR1B46 | 29141 | 31312 |
| RR1B47 | 12139 | 15842 |
| RR1B48 | 30169 | 40620 |
| RR1B49 | 21938 | 23400 |
| RR1B50 | 6932 | 7006 |
| RR1B51 | 4790 | 7504 |
| RR1B52 | 6039 | 8420 |
| RR1B53 | 18675 | 8125 |
| RR1B54 | 9302 | 14947 |
| RR1B55 | 15690 | 18968 |
| RR1B56 | 7453 | 8512 |
| RR1B57 | 16212 | 17335 |
| RR1B58 | 20628 | 22610 |
| RR1B59 | 18977 | 15606 |
| RR1B60 | 13838 | 12539 |
| RR1B61 | 3179 | 3434 |
| RR1B62 | 8360 | 7631 |
| RR1B63 | 6096 | 6574 |
| RR1B64 | 2910 | 1863 |

TABLE 6
ROR1 expression in SKMES-1, H358, and MDA-MB231 cell lines

| | | Weighted 2X for Lung (SKMES and H358) | | | | | | |
|---|---|---|---|---|---|---|---|---|
| | | SKMES | | H358 | | MDA-MB231 | | |
| | Binding Domain | Expt 1 | Expt2 | Expt 1 | Expt2 | Expt 1 | Expt2 | Score |
| RR1B01 | Not Determined | 50 | 22 | 32 | 62 | 14 | 25 | 205 |
| RR1B02 | Ig Domain | 66 | 24 | 20 | 102 | 11 | 17 | 240 |
| RR1B03 | Frz Domain | 54 | 20 | 26 | 86 | 12 | 19 | 217 |
| RR1B04 | Frz Domain | 102 | 72 | 72 | 108 | 49 | 35 | 438 |
| RR1B05 | Frz Domain | 84 | 44 | 36 | 84 | 22 | 42 | 312 |
| RR1B06 | Frz Domain | 86 | 64 | 84 | 74 | 59 | 54 | 421 |
| RR1B07 | Frz Domain | 38 | 26 | 30 | 48 | 28 | 33 | 203 |
| RR1B08 | Frz Domain | 94 | 48 | 58 | 50 | 50 | 48 | 348 |
| RR1B09 | Frz Domain | 62 | 40 | 38 | 30 | 51 | 41 | 262 |
| RR1B10 | Frz Domain | 78 | 108 | 98 | 116 | 40 | 52 | 492 |
| RR1B11 | Frz Domain | 124 | 116 | 120 | 122 | 60 | 62 | 604 |
| RR1B12 | Ig Domain | 88 | 62 | 62 | 94 | 18 | 53 | 377 |
| RR1B13 | Ig Domain | 82 | 82 | 70 | 68 | 15 | 45 | 362 |
| RR1B14 | Frz Domain | 76 | 80 | 102 | 64 | 24 | 44 | 390 |
| RR1B15 | Frz Domain | 92 | 94 | 108 | 52 | 42 | 47 | 435 |
| RR1B16 | Frz Domain | 44 | 78 | 86 | 38 | 34 | 29 | 309 |
| RR1B17 | Frz Domain | 30 | 68 | 68 | 24 | 32 | 23 | 245 |
| RR1B18 | Frz Domain | 18 | 34 | 40 | 10 | 16 | 11 | 129 |
| RR1B19 | Frz Domain | 6 | 18 | 22 | 6 | 9 | 6 | 67 |
| RR1B20 | Not Determined | 2 | 2 | 2 | 2 | 2 | 1 | 11 |
| RR1B21 | Krg Domain | 42 | 58 | 48 | 54 | 45 | 24 | 271 |
| RR1B22 | Krg Domain | 28 | 74 | 94 | 82 | 37 | 13 | 328 |
| RR1B23 | Krg Domain | 64 | 92 | 92 | 112 | 47 | 34 | 441 |
| RR1B24 | Krg Domain | 90 | 100 | 90 | 110 | 41 | 43 | 474 |
| RR1B25 | Krg Domain | 70 | 66 | 66 | 78 | 55 | 37 | 372 |
| RR1B26 | Ig Domain | 104 | 86 | 76 | 96 | 29 | 50 | 441 |
| RR1B27 | Krg Domain | 32 | 42 | 28 | 42 | 25 | 14 | 183 |
| RR1B28 | Frz Domain | 4 | 28 | 24 | 14 | 13 | 2 | 85 |
| RR1B29 | Krg Domain | 12 | 10 | 14 | 20 | 17 | 15 | 88 |
| RR1B30 | Krg Domain | 16 | 14 | 6 | 18 | 10 | 10 | 74 |
| RR1B31 | Ig Domain | 20 | 4 | 4 | 16 | 1 | 9 | 54 |
| RR1B32 | Ig Domain | 58 | 88 | 82 | 92 | 31 | 39 | 390 |
| RR1B33 | Krg Domain | 52 | 52 | 50 | 76 | 19 | 30 | 279 |
| RR1B34 | Krg Domain | 96 | 98 | 46 | 88 | 46 | 38 | 412 |
| RR1B35 | Krg Domain | 40 | 56 | 44 | 98 | 23 | 36 | 297 |
| RR1B36 | Krg Domain | 36 | 90 | 74 | 80 | 57 | 31 | 368 |
| RR1B37 | Krg Domain | 22 | 30 | 34 | 44 | 4 | 12 | 146 |
| RR1B38 | Frz Domain | 74 | 118 | 96 | 66 | 56 | 32 | 442 |
| RR1B39 | Krg Domain | 14 | 16 | 16 | 34 | 8 | 7 | 95 |
| RR1B40 | Krg Domain | 8 | 12 | 12 | 12 | 5 | 4 | 53 |
| RR1B41 | Krg Domain | 10 | 8 | 10 | 8 | 6 | 5 | 47 |
| RR1B42 | Frz Domain | 98 | 32 | 18 | 28 | 26 | 22 | 224 |
| RR1B43 | Frz Domain | 72 | 112 | 110 | 120 | 38 | 56 | 508 |
| RR1B44 | Ig Domain | 106 | 114 | 118 | 128 | 39 | 57 | 562 |
| RR1B45 | Frz Domain | 114 | 124 | 104 | 118 | 52 | 59 | 571 |
| RR1B46 | Ig Domain | 126 | 126 | 126 | 124 | 63 | 51 | 616 |
| RR1B47 | Ig Domain | 100 | 38 | 60 | 90 | 20 | 63 | 371 |
| RR1B48 | Frz Domain | 128 | 128 | 128 | 106 | 64 | 64 | 618 |
| RR1B49 | Ig Domain | 122 | 102 | 88 | 58 | 43 | 61 | 474 |
| RR1B50 | Ig Domain | 56 | 110 | 112 | 46 | 62 | 18 | 404 |
| RR1B51 | Frz Domain | 34 | 54 | 78 | 40 | 53 | 20 | 279 |
| RR1B52 | Ig Domain | 46 | 50 | 52 | 36 | 27 | 27 | 238 |
| RR1B53 | Ig Domain | 116 | 36 | 80 | 22 | 44 | 26 | 324 |
| RR1B54 | Ig Domain | 80 | 104 | 54 | 104 | 33 | 46 | 421 |
| RR1B55 | Ig Domain | 110 | 122 | 116 | 126 | 61 | 58 | 593 |
| RR1B56 | Frz Domain | 60 | 60 | 42 | 70 | 7 | 28 | 267 |
| RR1B57 | Ig Domain | 112 | 96 | 56 | 114 | 36 | 55 | 469 |
| RR1B58 | Ig Domain | 120 | 120 | 114 | 100 | 58 | 60 | 572 |
| RR1B59 | Ig Domain | 118 | 106 | 122 | 72 | 35 | 49 | 502 |
| RR1B60 | Ig Domain | 108 | 70 | 64 | 56 | 21 | 40 | 359 |
| RR1B61 | Frz Domain | 26 | 76 | 106 | 26 | 48 | 8 | 290 |
| RR1B62 | Ig Domain | 68 | 46 | 124 | 60 | 30 | 21 | 349 |
| RR1B63 | Ig Domain | 48 | 84 | 100 | 32 | 54 | 16 | 334 |
| RR1B64 | Ig Domain | 24 | 6 | 8 | 4 | 3 | 3 | 48 |

Most of the phage hit antibodies bound to both ROR1+ cell lines, SKMES-1, and MDA-MB-231. The phage hits were sorted by mean fluorescence index (MFI), first on SKMES, and followed by sorting on MDA-MB-231. Overall, the intensity correlated well in each case. The phage hits could be loosely grouped as high binders, medium binders, and low or non-binders, regardless of whether they were sorted on SKMES-1 or MDA-MB-231. The commercial antibody controls for this assay did not work as well as expected. A similar experiment was carried out with H358 and SH-SY-5Y cells. Generally, the phage hits which were good binders to H358 were also good binders to SH-SY-5Y, and those that were poor binders were poor for both cell lines as well (data not shown). The phage hits were generally in the same “category” whether they were sorted by rank on H358 or on SH-SY-5Y. That is, the high binders were generally the same for both cell lines, the medium binders were generally the same for both, and the low or non-binders were also the same for both cell lines. In addition, the binding rankings generally followed the trend seen in the other ROR1+ cell lines tested, SKMES-1 and MDA-MB-231.

### Binning of the Panel by ROR1 ECD Domain

In order to characterize the initial panel of phage-derived anti-ROR1 antibodies, a set of Fc fusion proteins were created (RR1W4 IgC2domain, RR1W5 frizzled domain, and RR1W6 kringle domain) as key reagents. In this experiment the binding of each mAb to these Fc-fusion proteins was carried out by MSD. Briefly, 5 μl of 10 μg/ml of ROR1 ECD domain constructs (RR1W4 IgC2 domain, RR1W5 frizzled domain, and RR1W6 kringle domain) were absorbed on Meso Scale Discovery (MSD) HighBind plates (Gaithersburg, Md.) for 2 hours then washed 3× with 150 μl 0.1M HEPES. The plate was blocked with 5% BSA buffer overnight at 4° C. The next day, the plate was washed 3× in preparation for addition of anti-ROR mAb supernatants. 25 μl of 10 μg/ml mAb supernatant was added for each variant, and samples were incubated for 2 hrs at room temperature with gentle shaking. The plate was washed 3×, then 25 μl of 20 nM Ru-labeled anti-human Kappa chain (Clone# SB81a, Southernbiotech, Birmingham, Ala.) was added to each well to detect binding of the mAbs to the various ROR1 domain constructs. Incubation was for 1 hour at room temperature with gentle shaking. Domain variants were mixed with 20 nM Ru-labeled anti-human Kappa chain and commercial anti-ROR1 (MAB2000; Clone #291608, R&D Systems) at 200 nM as controls. The plate was then washed 3 times with HEPES wash buffer. MSD read buffer (150 μl) was added to each well, and the plate was then analyzed using an MSD Sector Imager 6000 (MSD, Gaithersburg, Md.).

The results of the binning assessment provided a consistent binning of the mAbs, which was used in subsequent analyses. Frizzled and Kringle Domain selective binding appeared to be strong; IgC2 binding was overall very weak when it was noted. RR1B46 and RR1B55 seemed clearly better than average IgC2 binders. The molecules RR1B01 and RR1B20 clearly did not bind to any of the ROR1 or ROR2 proteins. In Table 7, Tier 1, 2, and 3 refer to sets of mAbs that were grouped together for evaluation. Table 7 shows that the panel of anti-ROR1 antibodies were fairly evenly divided across the 3 domains of the ECD of ROR1.

TABLE 7
Results of binning assessment

| Domain | IgC2-Fc | Fr-Fc | Kr-Fc | ND* |
|---|---|---|---|---|
| 1 | RR1B26 | RR1B04 | RR1B01 | |
| 2 | RR1B44 | RR1B06 | | |
| 3 | RR1B46 | RR1B10 | | |
| 4 | RR1B47 | RR1B11 | | |
| 5 | RR1B49 | RR1B14 | | |
| 6 | RR1B50 | RR1B15 | | |
| 7 | RR1B55 | RR1B19 | | |
| 8 | RR1B57 | RR1B43 | | |
| 9 | RR1B58 | RR1B45 | | |
| 10 | RR1B59 | RR1B48 | | |
| 11 | RR1B60 | | | |
| 1 | RR1B02 | RR1B08 | RR1B22 | |
| 2 | RR1B12 | RR1B09 | RR1B23 | |
| 3 | RR1B13 | RR1B17 | RR1B24 | |
| 4 | RR1B32 | RR1B18 | RR1B34 | |
| 5 | RR1B52 | RR1B38 | RR1B36 | |
| 6 | RR1B53 | RR1B42 | | |
| 7 | RR1B54 | RR1B51 | | |
| 8 | RR1B62 | RR1B61 | | |
| 9 | RR1B63 | | | |
| 1 | RR1B31 | RR1B03 | RR1B21 | RR1B20 |
| 2 | RR1B64 | RR1B05 | RR1B25 | |
| 3 | | RR1B07 | RR1B27 | |
| 4 | | RR1B16 | RR1B29 | |
| 5 | | RR1B28 | RR1B30 | |
| 6 | | RR1B56 | RR1B33 | |
| 7 | | | RR1B35 | |
| 8 | | | RR1B37 | |
| 9 | | | RR1B39 | |
| 10 | | | RR1B40 | |
| 11 | | | RR1B41 | |
| Total No. | 22 | 24 | 16 | 2 |

ND*: Not determined
Tabulation of the binning data for the set of 64 anti-ROR1 antibodies shows that antibodies to each of the three extracellular domains were identified. Molecules that bind to the Ig-Like domain (IgC2-Fc), Frizzled domain (Fz-FC), and Kringle (Kz-Fc) were identified. Two molecules did not appear to bind to ROR1 in this analysis.

Experiments were also carried out to evaluate the binding of the phage-derived panel to ROR1-Fc (Sino Biologics) and ROR2-His (Origene) protein by Meso scale discovery (MSD). The molecules RR1B43, RR1B45 and RR1B55 displayed low levels of ROR2 binding and RR1B11, RR1B44, RR1B46 had measurable ROR2 binding (data not shown). Molecule RR1B36 was identified as a potential ROR2 binder and RR1B25 had very low level of ROR2 binding. Comparison with binding data to CHO-ROR2 cells suggests some overlap (bold): RR1B3, B11, B14, B15, B17, B32, B43, B46, B51, B55 and B61 (data not shown).

A series of antibody supernatants were tested for their ability to bind ROR1 and ROR2 wt, single domain Fc fusion proteins, and ROR1/ROR2 domain-swapped variants in order to identify the binding domain. In the experiment, ROR1-ECD-Fc and ROR2-ECD-Fc binding data were compared to a version of the ROR2 ECD in which the IgC2 domain was replaced with the ROR1 IgC2 domain (IgC2(1)-Fz-(2)-Kg(2)-Fc) and a similar domain reconstructed chimeric ECD in which the ROR2 IgC2 domain would be presented (IgC2(2)-Fz(1)-Kr(2)-Fc). In these experiments, one would expect that ROR1-ECD-Fc binding would correlate with the IgC2(1) chimera. Likewise, the ROR2-ECD-Fc binding would be expected to correlate with IgC2(2) chimera. An expected pairing was observed in some experiments: RR1B26, RR1B49, RR1B50, RR1B58, RR1B59 and RR1B60. However, the binding to the chimera was generally very low and in no case was the chimera above 50% of the binding value for the WT ECD (data not shown).

The binning data for the anti-ROR1 antibodies was used to determine the epitope for the commercial antibody 2A2. A competition experiment was performed using parental anti-ROR1 antibodies. Three cell lines were tested: H520 cells as a negative control, and H358 and SK-MES-1 as ROR1+ test cells. As expected, H520 cells showed an absence of binding as shown in the similarity in the MFI values between the no mAb control (without pretreatment with blocking mAb) and the no 2A2 control (well D12) (data not shown). In contrast the, data from the H358 and SKMES definitively demonstrated that the 2A2 antibody binds to the Ig-like domain. Pre-incubation with antibodies RR1B76 (RR1B58), RR1B85 (RR1B44) and RR1B86 (RR1B47) inhibited 2A2 binding. Interestingly, RR1B65 (RR1B12) only inhibited 2A2 binding only slightly, while RR1B88 did not inhibit 2A2 binding at all, indicating that there are at least two epitopes in the Ig-like region.

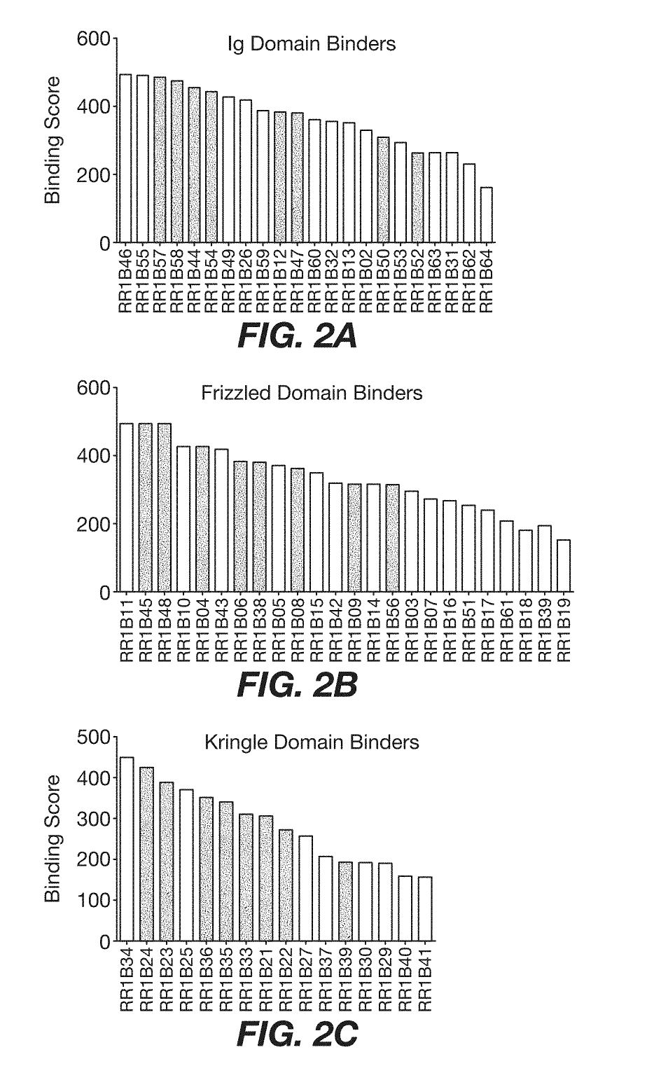

A weight of evidence approach was adopted to enable the selection of a group of anti-ROR1 antibodies that were to be advanced to expression and purification as IgG4 PAA molecules. The data in Table 6 was combined with ROR2 selectivity data. After elimination of non-binders and cross reactive ROR2 binders, a few very weak binders were deprioritized. Then the molecules were binned by epitope and a high binder, medium binder and a weak binder were selected for each of the three epitopes in the ROR1 ECD. Backup molecules were then selected for each of these molecules. This selection exercise resulted in a table of 24 molecules that was divided into two panels of 12 each (Table 8 and FIG. 2). This set of molecules was advanced to expression and purification.

TABLE 8
Anti-ROR1 antibodies from phage display
advanced to expression and purification

| Panel (A) | Panel (B) |
|---|---|
| RR1B06 | RR1B23 |
| RR1B24 | RR1B36 |
| RR1B48 | RR1B33 |
| RR1B35 | RR1B21 |
| RR1B22 | RR1B39 |
| RR1B45 | RR1B04 |
| RR1B38 | RR1B08 |
| RR1B56 | RR1B09 |
| RR1B57 | RR1B44 |
| RR1B58 | RR1B54 |
| RR1B12 | RR1B47 |
| RR1B52 | RR1B50 |

Generation of Anti-ROR1 mAbs on IgG4 PAA Platform

The 24 anti-ROR1 antibodies were prepared on the IgG4 PAA platform, which naturally includes R409 (Table 9).

Some of the selected mAbs did not express well—the mAbs RR1B68, RR1B73, RR1B75, RR1B79, RR1B80, RR1B81 and RR1B87 were not advanced (shaded gray in the above table). This resulted in 17 anti-ROR1 mAbs for evaluation.

### Surface Plasmon Resonance to Test Binding of CD3 Arm to ROR1

Binding of the anti-CD3 antibody CD3B219. Experiments were carried out on H358 cells, primary T-cells, SK-MES-1 cells, and the ROR1 expressed in HEK293F cells. The binding results showed concentration dependent binding of commercial mouse anti-ROR1 antibodies to the ROR1 expressing H358 cells and no specific binding of the anti-CD3 antibodies to these cells. The binding results showed concentration dependent binding of anti-CD3 antibodies to the T cells and no specific binding of anti-ROR1 antibody to the T cells. These results confirm that the anti-CD3 antibodies bind specifically to T cells as expected. The binding results showed concentration dependent binding of anti-ROR1 antibodies to the ROR1 expressing SK-MES-1 cells and no specific binding to the anti-CD3 antibodies to the ROR1 expressing SK-MES-1 cells. The binding results showed concentration dependent binding of anti-ROR1 antibodies to the ROR1 transiently transfected HEK293F cells and parental HEK293F cells and no specific binding to the anti-CD3 antibodies to the ROR1 transiently transfected HEK293F cells and parental HEK293F cells. These data are summarized in Table 10 below.

TABLE 10
Summary of experiments testing anti-CD3 antibody binding to
ROR1 expressing cells

| | ROR1 | Anti-ROR1 | CD3 | Anti-CD3 |
|---|---|---|---|---|
| Cell Line | expression? | binding? | expression? | binding? |
| H358 | Yes, | Yes | No | No |
| | endogenous | | | |
| Primary T cells | No | No | Yes, | Yes |
| | | | endogenous | |
| SK-MES-1 | Yes, | Yes | No | No |
| | endogenous | | | |
| ROR1 transfected | Yes, | Yes | No | No |
| 293F | overexpressed | | | |
| Parental 293F | Yes, | Yes | No | No |
| | endogenous | | | |

### Cross Competition of Anti-ROR1 mAb Panel

The panel of IgG4 PAA K409R anti-ROR1 antibodies were evaluated in a cross-competition experiment. Five μL of recombinant human ROR1-Fc Chimera (10 μg/mL; Sino Biologics, Cat#13968-H02H1) was directly coated on MSD HighBind plates for 2 hours at room temperature then blocked with 5% MSD Blocker A buffer for an additional 2 hours at room temperature. The plates were washed 3× with 0.1 M HEPES buffer, pH 7.4, followed by the addition of a mixture of Ruthenium (Ru)-labeled anti-ROR1 mAb, which was pre-incubated at room temperature for 30 minutes with different concentrations, from 1 μM to 1 nM, of other anti-ROR1 mAbs. After incubation with gentle shaking at room temperature 2 hours, the plates were washed 3× with 0.1M HEPES buffer (pH 7.4). MSD Read Buffer T was diluted with distilled water (4-fold) and dispensed into each well. The plates were analyzed with a SECTOR Imager 6000 (Meso Scale Discovery, Gaithersburg, Md.) and the data were processed with GraphPad.

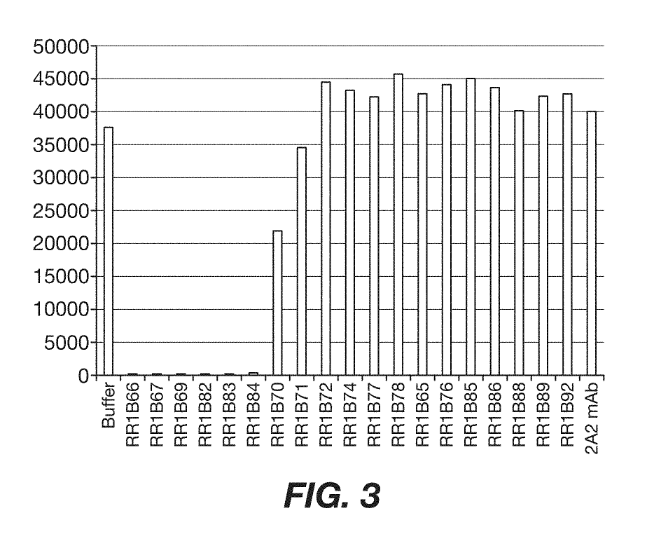

The IgG4 PAA K409R anti-ROR1 antibodies that had been assigned to the Kringle Domain binding family, including RR1B67, gave very clear cross competition readout. RR1B66, RR1B67, RR1B69, RR1B82, RR1B83 and RR1B84 competed for binding of the Ru-labeled RR1B69 molecule (FIG. 3 and Table 11). From this data it was concluded that all of the Kringle binding molecules bound to the same epitope. In addition, the commercial antibody 2A2 was shown to bind to the Ig-like domain (Table 11).

TABLE 11
Summary of cross competition data

| | Ru-labeled anti-ROR1 mAb | | | | | | |
|---|---|---|---|---|---|---|---|
| Competitor | RR1B69 | RR1B71 | RR1B72 | RR1B78 | RR1B85 | RR1B86 | RR1B88 |
| RR1B66 | + | — | — | — | — | — | — |
| RR1B67 | + | — | — | — | — | — | — |
| RR1B69 | + | — | — | — | — | — | — |
| RR1B82 | + | — | — | — | — | — | — |
| RR1B83 | + | — | — | — | — | — | — |
| RR1B84 | + | — | — | — | — | — | — |
| RR1B71 | — | + | — | — | — | — | — |
| RR1B70 | — | — | + | + | — | — | — |
| RR1B72 | — | — | + | + | — | — | — |
| RR1B74 | — | ± | + | + | — | — | — |
| RR1B77 | — | ± | + | + | — | — | — |
| RR1B78 | — | ± | + | + | — | — | — |
| RR1B65 | — | — | — | — | — | — | + |
| RR1B76 | — | — | — | — | — | ± | + |
| RR1B85 | — | — | — | — | + | + | — |
| RR1B86 | — | — | — | — | + | + | — |
| RR1B88 | — | — | — | — | — | — | + |

| | | |
|---|---|---|
| Kringle | Frizzle | Ig-Like |

The cross competition experiment identified 6 binding groups on the ECD of ROR1 (Table 12). One competition group was found for the Kringle binding molecules (Group 1). All of the Frizzled domain binding molecules with the exception of RR1B71 bound to the same epitope (Group 2). RR1B71 formed Group 3. Three epitope groups were identified for the Ig-like domain. The commercial molecule 2A2 formed Group 5.

TABLE 12
Binding Groups on the ECD of ROR1

| Kringle | Frizzled | | Ig-Like | | |
|---|---|---|---|---|---|
| Group | Group | Group | Group | Group | Group |
| I | II | III | IV | V | VI |
| RR1B66 | RR1B70 | RR1B71 | RR1B85 | 2A2 | RR1B65 |
| RR1B67 | RR1B72 | | RR1B86 | | RR1B76 |
| RR1B69 | RR1B74 | | | | RR1B88 |
| RR1B82 | RR1B77 | | | | |
| RR1B83 | RR1B78 | | | | |
| RR1B84 | | | | | |

### Generation of ROR1×CD3 Bispecific Antibodies

ROR1×CD3 bispecific antibodies were prepared using the DuoBody® platform. See, for example, U.S. Patent App. Pub. No. US2014/0170148. Briefly, a controlled Fab arm exchange (cFAE) between 2 intentionally designed monoclonal antibodies, one harboring F405L mutation and another having K409R. The cFAE was initiated by mixing equal molar ratio of the 2 parental antibodies—IgG4 PAA K409R anti-ROR1 antibodies (ROR1 arm) and CD3B219 (CD3 arm)—or in some cases 6% extra of one parental to deplete another, with 75 mM (final concentration) of cysteamine HC1 (2-MEA). After incubation for 5 hrs at 31° C., the antibody mixture was dialyzed against 1×D-PBS, during which period 2-MEA was removed to allow the reduced disulfide bonds to reconnect. The formation of DuoBody® heterodimer was analyzed by either cation exchange (CEX) HPLC or hydrophobic interaction chromatography (HIC) HPLC. The bispecific antibodies were polished by preparative CEX or HIC to remove the residual parental(s). The 17 ROR1×CD3 bispecific antibodies created using this approach are listed in Table 13.

TABLE 13
ROR1 × CD3 bispecific antibodies

| ROR1 × CD3 Bispecific | Parental IgG4 PAA | |
|---|---|---|
| Antibody | anti-ROR1 antibody | Domain |
| RCDB3 | RR1B65 | Ig-Like |
| RCDB11 | RR1B76 | Ig-Like |
| RCDB17 | RR1B85 | Ig-Like |
| RCDB18 | RR1B86 | Ig-Like |
| RCDB19 | RR1B88 | Ig-Like |
| RCDB7 | RR1B70 | Frizzled |
| RCDB8 | RR1B71 | Frizzled |
| RCDB9 | RR1B72 | Frizzled |
| RCDB10 | RR1B74 | Frizzled |
| RCDB12 | RR1B77 | Frizzled |
| RCDB13 | RR1B78 | Frizzled |
| RCDB4 | RR1B66 | Kringle |
| RCDB5 | RR1B67 | Kringle |
| RCDB6 | RR1B69 | Kringle |
| RCDB14 | RR1B82 | Kringle |
| RCDB15 | RR1B83 | Kringle |
| RCDB16 | RR1B84 | Kringle |

The IDs that correspond between phage-hits (column 1) and the IgG4 PAA ROR1 antibodies (column 2), and the ROR1 × CD3 bispecific antibody (DuoBody ®).

ROR1×CD3 DuoBody® molecules used in the remainder of the studies were those having a CD3B219 arm. Thus, as used in the remainder of the Examples section, ROR1×CD3 bispecific antibody refers to those having a CD3B219 arm, unless otherwise stated.

### Relative Cell Binding for the ROR1×CD3 Bispecific Antibodies

The set of 17 ROR1×CD3 bispecific antibodies were tested for binding to ROR1 expressing HCC827 cells. Briefly, cells were harvested using cell dissociation buffer (Gibco, USA) and washed in PBS. Labeling with bispecific antibody was performed for 45 minutes at 4° C. in FACS staining buffer (BD Biosciences, USA). Cells were washed in staining buffer prior to incubation with an alexa-fluor 647-labeled anti-human secondary antibody (Life Technologies, USA). Samples were collected on an IntelliCyt High Throughput Flow Cytometry HTFC system and analyzed using ForeCyt software. EC₅₀ values were calculated in GraphPad Prism V6. Acceptance criteria for non-linear regression curve fitting was a confidence interval (CI) range of less than 1.4.

Control antibodies used were a CD3×B21M isotype and a commercial anti-ROR1 antibody from BioLegend (2A2) and the corresponding isotype. The antibodies were titrated starting from 15 μg/mL or about 100 nM. Each antibody dilution was run in duplicate and the CD3×B21M isotype was included on all plates. All ROR1 CD3 bispecific antibodies bound to the cells in a concentration dependent manner, while the CD3×B21M and BioLegend isotype did not bind to the cells in a concentration dependent manner. The isotype subtracted geo mean fluorescence index (geoMFI) data for all samples was used for analysis. The isotype subtracted values were calculated by subtracting the respective plate CD3×B21M isotype GeoMFI from the antibody GeoMFI at each concentration. The EC50 values were calculated using the isotype subtracted geoMFI values. The data was graphed in Prism, the nM concentration values were LOG transformed and the EC50 values were calculated using non linear regression with the log(agonist) vs. response—Variable slope (four parameters) analysis with bottom constrained to 0 since the data was isotype background subtracted. Some curves did not appear to fully plateau at the highest concentration of antibody tested and some curves also have different maximal binding signals.

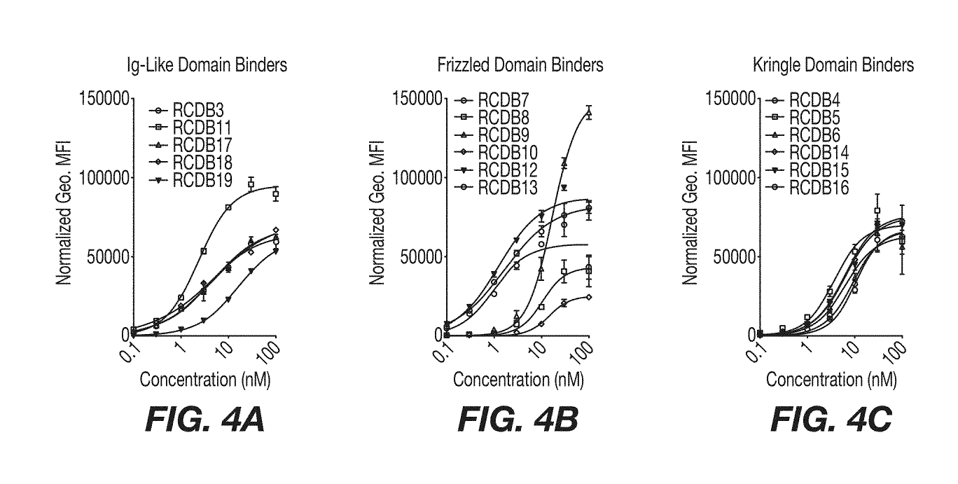

These data were replotted by grouping according to Domain Binding for graphical representation (FIG. 4). The isotype subtracted data was graphed in Prism, the nM concentration values were LOG transformed and the curve was fitted using non linear regression with the log(agonist) vs. response—Variable slope (four parameters) with the bottom constrained to 0 since the data was isotype background subtracted. The EC50 binding values were calculated and are shown in Table 14.

TABLE 14
EC50 of ROR1 × CD3 Bispecific Antibodies

| ROR1 × CD3 | | Binding EC50 |
|---|---|---|
| Bispecific Ab | Domain | (nM) |
| RCDB3 | Ig-Like | 3.77 |
| RCDB11 | Ig-Like | 2.36 |
| RCDB17 | Ig-Like | 4.72 |
| RCDB18 | Ig-Like | 5.07 |
| RCDB19 | Ig-Like | 16.2 |
| RCDB7 | Frizzled | 1 |
| RCDB8 | Frizzled | 10.63 |
| RCDB9 | Frizzled | 16.94 |
| RCDB10 | Frizzled | 14.8 |
| RCDB12 | Frizzled | 1.25 |
| RCDB13 | Frizzled | 1.64 |
| RCDB4 | Kringle | 9.14 |
| RCDB5 | Kringle | 3.74 |
| RCDB6 | Kringle | 6.25 |
| RCDB14 | Kringle | 6.21 |
| RCDB15 | Kringle | 6.34 |
| RCDB16 | Kringle | 10.43 |

The binding data indicated that the relative affinity of the Frizzled and Ig-Like domain molecules were more potent, while the Kringle domain binders were on average less potent. These data were in the context of the CD3 arm and were only determined on one ROR1 expressing cell line. The EC50 values for the Fizzled domain binders were slightly tighter than either the best Ig-like or the best Kringle domain binding ROR1×CD3 bispecific antibodies.

### Functional Evaluation of the ROR1×CD3 Bispecific Antibodies

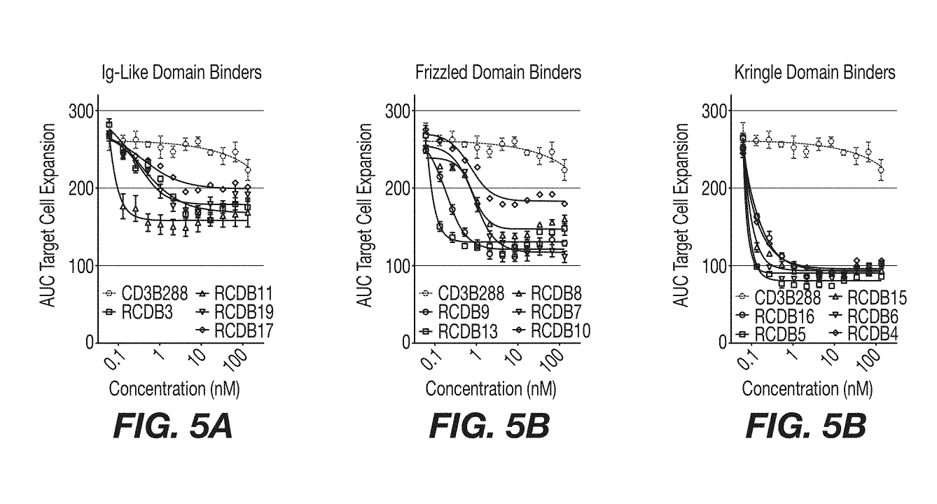

A variety of approaches were utilized to evaluate the functional activity of the set of 17 ROR1×CD3 bispecific antibodies. In one experiment, a subset of these molecules were tested in T cell re-direction killing assay. The IncuCyte based assay was used because it is more amenable to studies measuring killing of adherent cell lines and, in contrast to the flow-based assays, allows the calculation of absolute target cell numbers per well and the kinetics of their expansion. This is possible due to the use of RFP-labeled target cells which were generated using a lentiviral construct from Essen BioSciences. In this experiment, two independent readouts were used to measure cytotoxicity: target cell growth inhibition (FIG. 5) and Caspase 3,7 activity (data not shown). This allowed the loss of target cells and apoptotic target cell death, which is a feature of T cell mediated killing, to be correlated. An analysis protocol was developed which enabled the calculation of growth inhibition based on the expansion index generated from the number of target cells/well at each timepoint of the experiment. These kinetics curves were used to generate area under the curve values that were plotted to generate titration curves for each ROR1×CD3 bispecific antibody. Interpolation was then used to generate single values that could be used to directly compare the potency of the test molecules. Interpolation was used rather than EC50's because the maximum values were very different when comparing the different sets of molecules binned based on binding domain. Rankings from the growth inhibition and Caspase readouts were compared and, although not identical, showed a strong correlation (data not shown).

The growth inhibition data clearly showed that there was a difference between the groups of molecules binding to distinct domains in their ability to kill (FIG. 5 and Table 15). The curves generated by the Kringle domain binders had higher maximum value compared to the Frizzled domain binders. The Ig-like domain binders demonstrated the lowest maximum values for target cell growth inhibition, suggesting that there was a relationship between the position of the epitope and killing potential, with those binding the more membrane proximal region being more efficacious than those binding more distal parts of the ECD molecule. Within each of the groups of molecules, those with the highest relative affinities demonstrated the best killing, suggesting that affinity is important, but is secondary to the position of the epitope.

TABLE 15
Killing EC50 for ROR1 × CD3 bispecific antibodies

| ROR1 × CD3 | | Killing EC50 |
|---|---|---|
| Bispecific Ab | Domain | (nM) |
| RCDB3 | Ig-Like | 2.485 |
| RCDB11 | Ig-Like | 0.2473 |
| RCDB17 | Ig-Like | 5.166 |
| RCDB7 | Frizzled | 16.92 |
| RCDB9 | Frizzled | 3.213 |
| RCDB13 | Frizzled | 0.5392 |
| RCDB4 | Kringle | 0.6769 |
| RCDB5 | Kringle | 0.4543 |
| RCDB6 | Kringle | 0.4371 |
| RCDB15 | Kringle | 1.278 |
| RCDB16 | Kringle | 2.913 |

The caspase readout also showed that the Kringle binders generally had greater killing potential compared to those binding the Frizzled and Ig-like domains. There were, however, some differences in the data. Curve fits were not generated for RCDB11 and RCDB5 although the individual data points demonstrated good killing similar to that seen with the target cell growth inhibition readout. RCDB13 and RCDB9 were the best killers in the Frizzled group, while RCDB5 and RCDB15 appeared to be slightly better than RCDB4 and RCDB16 within the Kringle group. These observations are similar to that seen for the other readout.

Overall, this data suggests strongly that membrane proximity is a key determinant of killing potential, with affinity having a secondary impact. Of the panel of ROR1×CD3 bispecific antibodies tested, the Kringle domain binders showed the best killing efficacy. The higher affinity molecules in the Frizzled and Ig-like domain binding groups also showed good activity, suggesting that several distinct molecules are available for lead selection.

The ROR1×CD3 bispecific antibodies were also assessed in a novel T cell mediated cytotoxicity assay. In this assay, the target cells were engineered with a cytosolic fusion to half-BetaGal by DiscoveRx, which generated target cell lines that were transfected with a fusion protein composed of a non-secreted housekeeping gene fused with part of the b-gal molecule. This would be released into the media following lysis of the cell and could be used to readout (chemiluminescence). The lysis released b-gal would be fully reconstituted following addition of the complementary part of b-gal and a substrate. Three cell lines were transfected: H520 (negative control), H358 and SKMES-1.

Two different housekeeping proteins were used in the cell lines. Based on preliminary data, the background signal in the non-lysed ADO-fusion transfected cells was higher than desired. Hence, the FKBP1A cells were selected for a pilot T cell killing experiment. The H520 cell line was examined by Flow Cytometry analysis and shown to be negative for ROR1 (as expected). The H358 and SK-MES-1 cell lines were positive as expected.

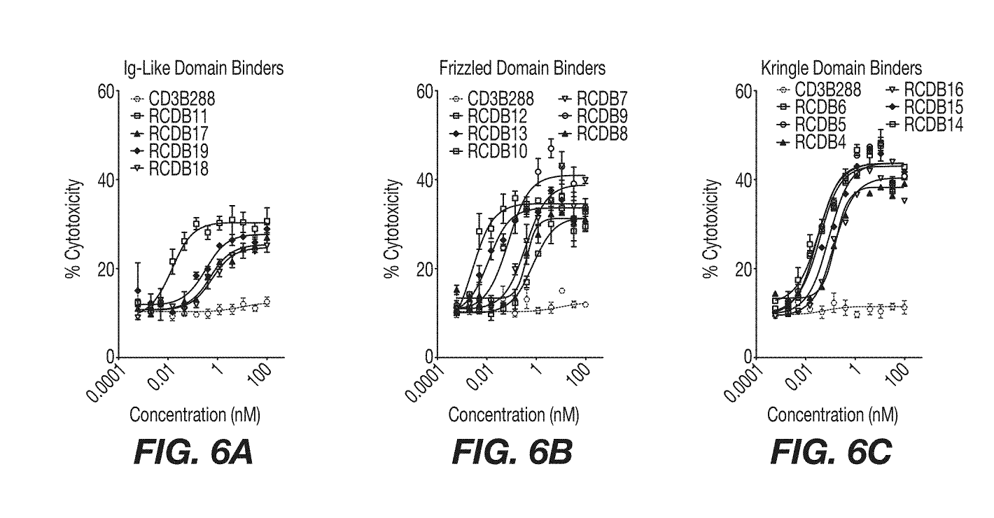

The DiscoveRx killing assay provided comparable data to other T-cell mediated cytotoxicity assays. In this example (FIG. 6), the Kringle domain binding ROR1×CD3 bispecific antibodies were among the best killing molecules and constituted a single group of molecules. More diverse responses were seen with Frizzled and Ig-like domain binders. However, two Frizzled binders and one of the Ig-like binders were comparable to the set of Kringle binding ROR1×CD3 bispecific antibodies.

The ROR1×CD3 bispecific antibodies RR1B69, B67, B78, B76, B72, B77, and B89 were further analyzed.

Cytotoxicity in Comparison with Published ROR1×CD3 Bispecific Antibodies.

The redirected T cell killing of lung tumor cells activated by ROR1×CD3 bispecific antibodies was compared between RCDB5 and a prior art antibody, published in WO2014167022A1, Example 3C, Bispecific (Fab)₂×(Fab) antibody bivalent for ROR1 and monovalent for CD3, with Fc, for brevity termed “Engmab” antibody. Flow cytometry-based assays were used to measure cytotoxicity. Triplicate samples were set up per condition. Briefly, 10,000 GFP-transfected NCI-H1975 lung tumor cells were plated per well in 96 well flat-bottomed plates and left to adhere for 4-6 hours. Cryopreserved, purified T cells were then thawed and added to each well at 50,000 cells/well (5:1 E:T) concomitant with bi-specific antibodies, which were plated at a final titration range of 0.0064-6667 pM. Plates were incubated at 37° C. for 72 hours.

At harvest, cells were released with trypsin, labelled with fixable viability dye (Thermo Fisher Scientific, Bridgewater) and anti-human CD25 (clone M-A251; BioLegend, San Diego), and collected using the IntelliCyt iQue high throughput flow cytometer. Data analysis was performed in ForeCyt software (IntelliCyt, Albuquerque) and exported to Microsoft Excel. Log-transformed values were then used for non-linear regression using the “sigmoidal dose-response (variable slope)” function in Prism v6.02 (GraphPad, La Jolla). A Log EC50 95% confidence interval range of less than 1.4. was set as acceptance criteria for curve fitting. Charts show mean values ±SEM. Statistical analysis was performed using an unpaired two tail t-test.

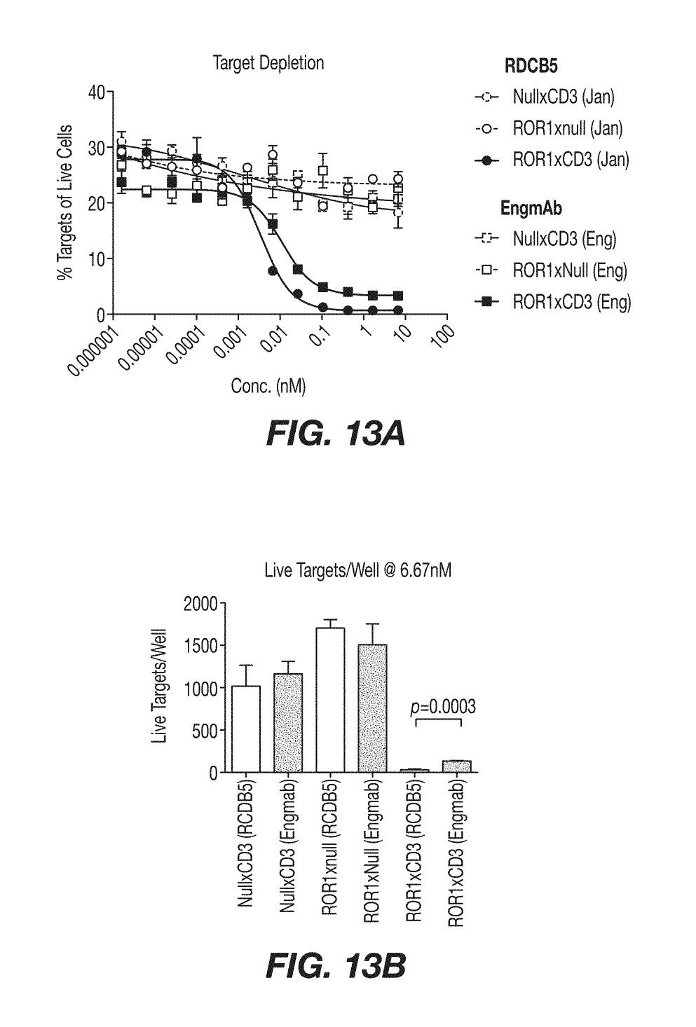

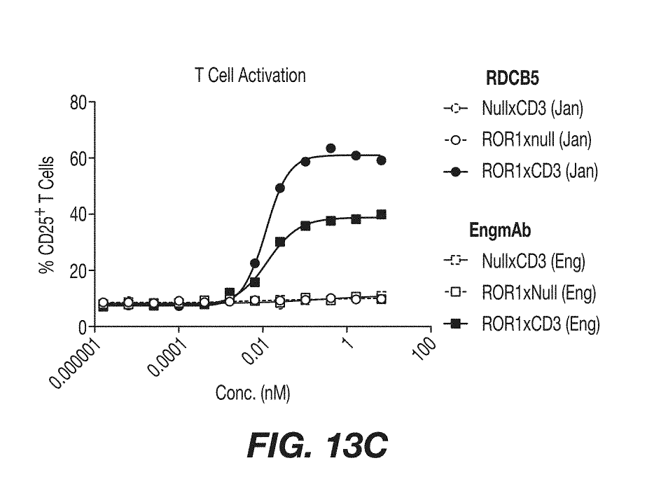

The data demonstrates that the RCDB5-activated cytotoxicity was greater than Engmab-activated cytotoxicity at various antibody concentrations (FIG. 13A). The number of live lung tumor cells per well was 4.6-fold lower in RCDB5-treated wells than in Engmab-treated wells at the highest concentration tested (6.67 nM, FIG. 13B). Because the induction of CD25 expression on T cells is associated with their activation, our data suggested that loss of target cells was mediated in a T cell-dependent manner (FIG. 13C).

### ROR1×CD3 Mediates Potent Killing of MCL Lines in Whole Blood Cytotoxicity Assay In Vitro

Confluent MCL cells, MAVER-1, JeKo-1, Z-138 and NAMALWA, Burkitt's lymphoma line (data not shown) were washed in PBS and labeled with 500 nM carboxyfluorescein succinimidyl ester (CFSE) dye (Invitrogen) for 5 min at room temperature. Reaction was quenched with heat-inactivated fetal bovine serum for 1 min. CFSE-labeled tumor cells were washed with culture media and reconstituted at 1×10⁶ cells/mL. 20,000 tumor cells were co-cultured with 40 uL whole blood from 4 healthy donors in the presence of either ROR1×CD3 RCDB13 or null arm control MAbs (null×CD3 or ROR1×null) reconstituted in RPMI medium (containing 10% FBS). Cells were cultured in 96-well plates at a final volume of 200 uL/well at 37° C. 5% CO₂ for 60 hours. Red blood cells were lysed using Multispecies Red Cell Lysis Buffer (eBioscience) diluted 1:10 with water on ice for 3 minutes. Cells were washed with PBS and stained with Live/Dead (Near-IR; ThermoFisher) according to manufacture's protocol. Cell pellets were stained with anti-CD4-PerCP/Cy5.5, anti-CD8-PE-Cy7, anti-CD25-PE (BioLegend) in 50 uL volumes of FACs Stain Buffer (BD) for 30 min at 4° C. in the dark. Cells were washed twice with Stain Buffer and reconstituted with Stain Buffer. % Cytotoxicity (100%−% Live CFSE+) and T cell activation (% CD25 expression on CD4+ and CD8+ lymphoid fractions) were measured by flow cytometry. Data were acquired on the BD FACs Canto cytometer and analyzed by CytoBank software and GraphPad Prism 6. Data are representative of two independent experiments.

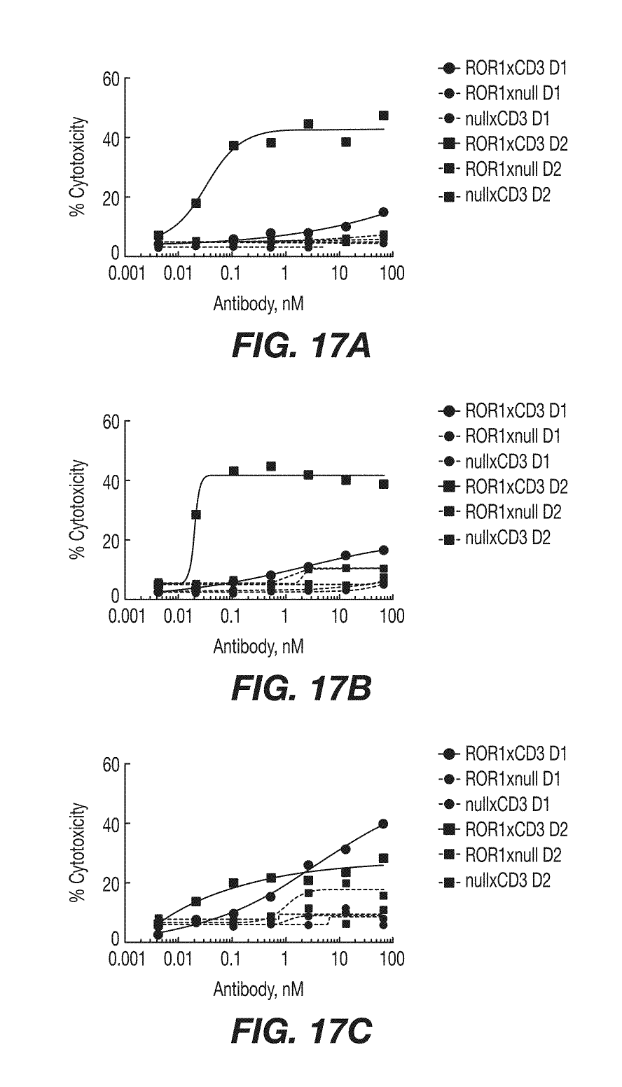

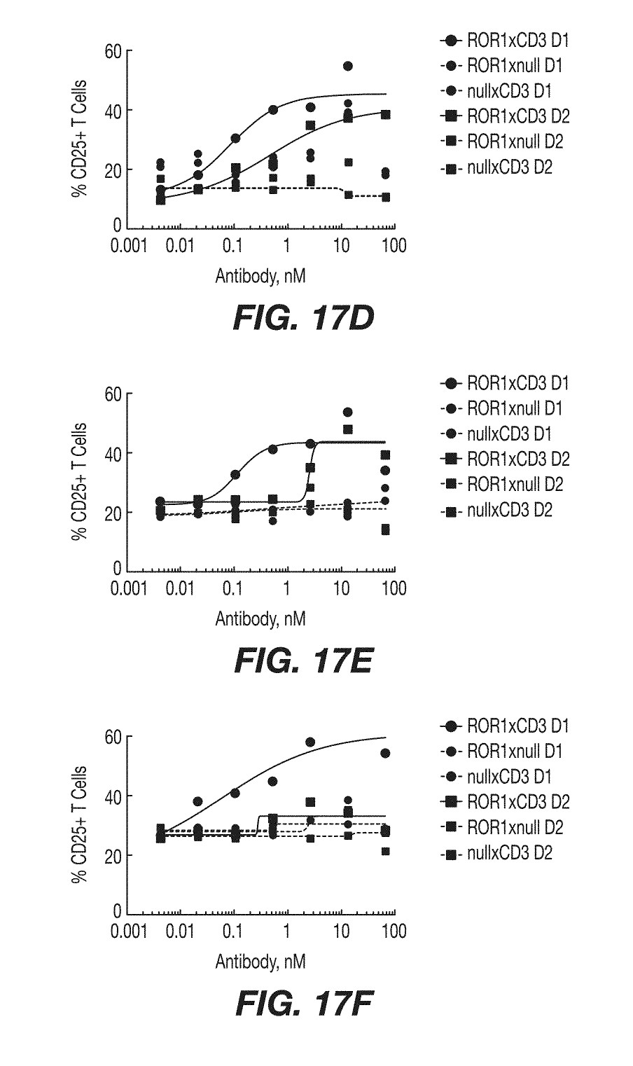

ROR1×CD3 mediated potent killing of MCL lines in whole blood cytotoxicity assay in vitro (FIG. 17A-F). Specific engagement of T cells with tumor cells mediated by ROR1×CD3 was required to induce dose-dependent T cell activation (activation marker, CD25 upregulation, FIG. 17D-F) and cytotoxicity (FIG. 17A-C). Specificity of T cell—tumor engagement was also confirmed by the lack of cytotoxicity below 1 nM when null controls were used. Although ΔEₘₐₓ (% of maximum cytotoxicity−% spontaneous cytotoxicity in the absence of antibody) varied between donors, EC50 were comparable between all cell lines tested and ranged between 0.2-1 nM.

### ROR1×CD3 Mediates Potent Killing of MCL Lines in Cytotoxicity Assay In Vitro Using PBMC as Effector Cells

Confluent MCL cells, MAVER-1, Z-138, JeKo-1, REC-1, Mino (data not shown for JeKo-1, REC-1, Mino) were washed in PBS and labeled with 500 nM carboxyfluorescein succinimidyl ester (CFSE) dye (Invitrogen) for 5 min at room temperature. Reaction was quenched with heat-inactivated fetal bovine serum for 1 min. CFSE-labeled tumor cells were washed with RPMI culture media (containing 10% fetal bovine serum) and reconstituted at 1×10⁶ cells/mL. PBMCs were washed in RPMI culture medium and adjusted to 2.5×10⁶ cells/mL. 20,000 tumor cells were co-cultured with 100,000 PBMC from 2 healthy donors (E:T=1:5) in the presence of either ROR1×CD3 RCDB13 or null arm control MAbs (null×CD3 or ROR1×null) reconstituted in culture medium. Cells were cultured in in 96-well plates, final volume 200 ul/well at 37° C. 5% CO₂ for 60 hours. Staining and data analysis were performed as in FIG. 4. Data are representative of 2 independent experiments.

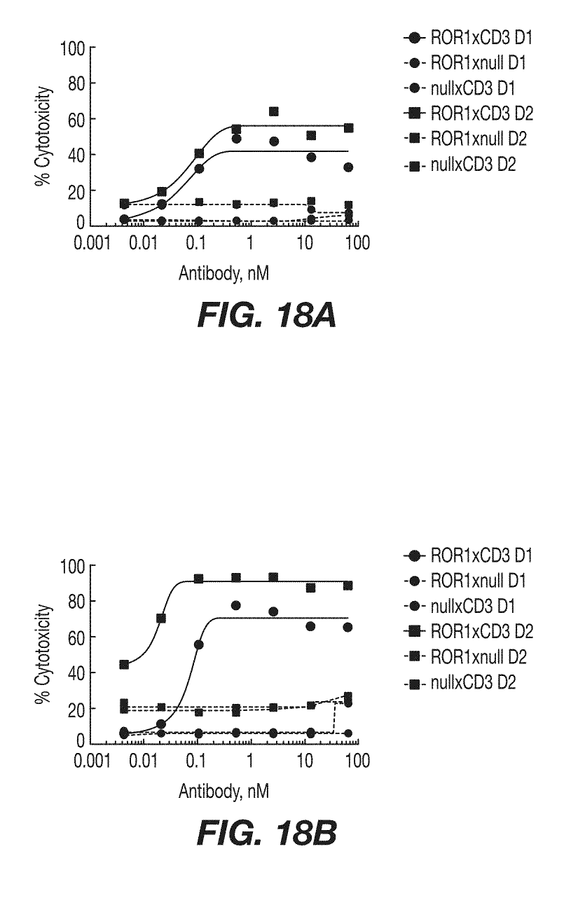

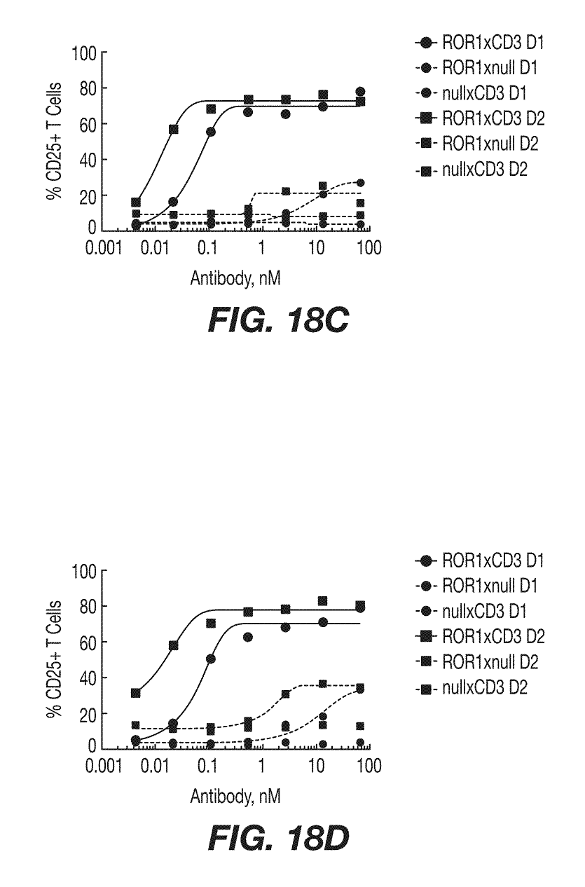

ROR1×CD3 mediated potent killing of MCL lines when PBMCs were used as effector cells in cytotoxicity assay in vitro (FIG. 18A, C show data for MAVER-1 cells, and FIG. 18B, D show data for Z-138 cells). Specific engagement of T cells with tumor cells mediated by ROR1×CD3 DuoBody was required to induce dose-dependent T cell activation (activation marker, CD25 upregulation, FIG. 18C-D) and cytotoxicity (FIG. 18A-B). Specificity of T cell—tumor engagement was also confirmed by the lack of cytotoxicity below 1 nM when null controls were used. EC50 were comparable between all cell lines tested and ranged between 0.2-1 nM similar to EC50 derived from whole blood assays using the same MCL lines as target cells. These data also suggested lack of association between ROR1 receptor density (FIG. 15) and EC50.

### Deamidation of the CD3 Arm

In the analysis of ROR1×CD3 bispecific antibodies, CD3 deamidation was observed on the CD3 Heavy Chain CDR3 in all release samples, which increases upon stress at high pH. Analytical evaluation of the CD3 deamidation was carried out twice. RCDB5 and RCDB11 release samples exhibited similar amount of deamidation levels (17±2% and 19±3%) compared to previous study (17±2% and 20±4%). A slightly higher level was observed in RCDB13 (24±2% vs. 19±5%), although within the error range (Table 16).

TABLE 16
Anti-CD3 antibody CD3B219 deamidation levels

| | | | | | | % PTM | % PTM |
|---|---|---|---|---|---|---|---|
| mAb | parental | PTM | CDR | Motif | Residue | Release | HightPH |
| RCDB5 | CD3B219 | Deamid. | H3 | NS | N106 + N103 | 17 ± 2 | 72 ± 6 |
| RCDB11 | CD3B219 | Deamid. | H3 | NS | N106 + N103 | 19 ± 3 | 75 ± 5 |
| RCDB13 | CD3B219 | Deamid. | H3 | NS | N106 + N103 | 24 ± 2 | 78 ± 5 |

### Biophysical and Cell Binding of ROR1×CD3 Bispecific Antibodies

Based upon early results for cell binding (FIG. 5) it was expected that Ig-like and Frizzled Domain binders might bind to ROR1 expression cells tighter than Kringle Domain binders. Since Kringle Domain binders were apparently better mediators of T cell directed cytotoxicity, a solid understanding of the cell binding was desired.

The affinity of select ROR1×CD3 bispecific antibodies to ROR1 was evaluated with the cell affinity technique using MesoScale Discovery technology (MSD-CAT). The MSD-CAT experiments were performed either using H358 cells endogenously expressing ROR1 or HEK293 transfected human ROR1. The receptor density of HEK293 transfected human ROR1 was measured by flow cytometry, which was then translated to molar concentration of receptor in the reaction mixture and was used in these studies. DuoBody® CD3B288 with single null arm of anti-RSV was used as negative control. Parental (mock) HEK293 cells were used to evaluate nonspecific binding to the cell surface. In order to measure the affinity of an interaction using the MSD-CAT method, a series of mixtures with a fixed concentration of the soluble reactant (A) and varying concentrations of cells (B) were prepared and allowed to reach equilibrium. After equilibration, the concentration of free A was determined and the data was analyzed for affinity. For these studies, the reaction mixture was prepared by adding antibody at four different but fixed concentrations and the cells were serially diluted starting at 6 e⁷ cells/mL. After incubation and equilibration of the reaction mixture, free anti-ROR1 was captured via biotin-ROR1-DDK captured on SA plate and followed by detection using ruthenium-labeled anti-human IgG. The binding profiles of the ROR1×CD3 bispecific antibody interactions with ROR1 were performed by nonlinear least-square fitting of the binding curves using 1:1 binding model.

In order to perform the MSD-CAT experiments, an assay for detection of free mAb in the reaction mixture was developed. Two detection methods were tested. The first method in which ROR1 flag-tagged (aka ROR1-DDK) was captured using an anti-flag tag antibody, followed by binding of free anti-ROR1 to the captured ROR1-DDK and detection with ruthenium-labeled anti-human IgG failed due to poor assay performance: low assay signal and narrow assay window. Therefore, a second detection assay was developed and finally used for the MSD-CAT experiment. In this detection assay, free anti-ROR1 was captured via biotin-ROR1-DDK on SA plate and the captured mAb detected using ruthenium-labeled anti-human IgG.

MSD-CAT for ROR1 DuoBody binding to HEK cells expressing hu ROR1 (Lentiviral plasmid pDR25226) were generated and used to transfect HEK293F cells. The ROR1×CD3 bispecific antibodies (RCDB5, RCDB6, RCDB9, RCDB11, RCDB12, RCDB13) were analyzed using this cell line (Table 17). These data were fit using the receptor density of 1.8 million human ROR1 per HEK293 as measured by flow cytometry (data not shown). Example data are shown in FIGS. 7A and 7B. From these measurements the DuoBody RCDB5 had a K_{D} of 331+/−152 pM and the RR1B67 parental (bivalent) antibody had an apparent K_{D} of 55+/−13 pM. RR1B67 Mab displayed a ˜6-fold tighter binding than RCDB5 (the DuoBody for RR1B67×CD3B219). This higher affinity of the parental to the HEK293 ROR1 expressing cells was attributed to avidity effects. The binding profiles of RCDB13, RCDB11, RCDB12, RCDB5, RCDB6 were similar with affinity in the sub-nM range; however, weaker affinity was observed for RCDB9 in the nM range. For RCDB9, there was a cell concentration dependent titration on the control (mock) cells that showed non-specific binding for RCDB9. The negative DuoBody®, CD3B288, showed no binding to ROR1 expressing cells nor to HEK293 mock cell.

TABLE 17
Compiled cell affinity of ROR1 × CD3 DuoBody ® Abs to
HEK293 cells expressing human ROR1

| | Human ROR1 Cell (Ave ± SD) | |
|---|---|---|
| Antibody | K_{D} [pm] | n |
| RCDB5 | 331 ± 152 | 20 |
| RCDB6 | 648 ± 325 | 12 |
| RCDB9 | 2268 ± 1748 | 8 |
| RCDB11 | 382 ± 142 | 12 |
| RCDB12 | 236 ± 170 | 12 |
| RCDB13 | 254 ± 122 | 12 |
| *RR1B67 | *55 ± 13 | 6 |

Summary of cell affinity (MSD-CAT) data for the binding of six ROR1 × CD3 bispecific antibodies and the anti-ROR1 antibody RR1B67 to HEK293 cells engineered to overexpress ROR1 (# human ROR1 receptor/cell = 1.8 × 10⁶). The data corresponds to the average and standard deviation of all acceptable (total indicated by n) affinity values obtained in 3 independent experiments. All of the molecules had sub-nanomolar affinity, with the exception of RCDB9. The binding of the parental molecule RR1B67 suggest avidity effect in binding due to the bivalent nature of the antibody which could potentially cross-link two neighboring ROR1 in the cell surface.
*Referred to as the “apparent K_{D}” because it could be affected by avidity due to bivalent binding

Biacore (surface plasmon resonance; SPR) was also used to determine the affinity of the ROR1×CD3 bispecific antibodies (RCDB5, RCDB6, RCDB9, RCDB11, RCDB12, RCDB13) to a recombinant human ROR1 (Table 18). Example SPR sensograms are shown in FIGS. 7A and 7B.

TABLE 18
Biacore analysis of ROR1 × CD3 bispecific antibodies

| | kₒₙ Ave | kₒₙ STDEV | k_{off} ave | k_{off} STDEV | K_{D} ave | K_{D} Stdev | |
|---|---|---|---|---|---|---|---|
| Ab ID | (M⁻¹s⁻¹) | (M⁻¹s⁻¹) | (s⁻¹) | (s⁻¹) | (nM) | (nM) | n |
| RCDB5 | 1.54E+04 | 1.21E+03 | 1.17E-04 | 9.33E-06 | 8 | 0.9 | 3 |
| RCDB13 | 1.84E+04 | 2.29E+03 | 1.95E-04 | 3.49E-05 | 11 | 2.3 | 3 |
| RCDB6 | 1.84E+04 | 2.05E+03 | 2.22E-04 | 5.80E-06 | 12 | 1.4 | 3 |
| RCDB11 | 1.94E+04 | 2.52E+03 | 3.01E-04 | 5.36E-05 | 15 | 3.4 | 3 |
| RCDB12 | 2.33E+04 | 5.82E+02 | 6.45E-04 | 1.61E-05 | 28 | 1.0 | 3 |
| RCDB9 | 2.79E+04 | 1.65E+03 | 2.20E-03 | 2.70E-04 | 79 | 10.7 | 3 |
| RRIB67 | 2.09E+04 | (1.8-2.3)E+04 | 1.33E-04 | (1.2-1.4)E-04 | 6 | 7.8-5.3 | 2 |
| RRIB69 | 1.91E+04 | (1.9-1.9)E+04 | 2.13E-04 | (1.0-2.2)E-04 | 11 | 11.8-10.4 | 2 |

Summary of Biacore data for the binding of six ROR1 × CD3 bispecific antibodies and the anti-ROR1 antibodies RR1B67 and RRIB69 to recombinant human ROR1. The data corresponds to the average and standard deviation of all acceptable (total indicated by n) affinity values obtained in 3 independent experiments. All of the molecules had sub-nanomolar affinity, with the exception of RCDB9. The binding of the parental molecule RR1B67 suggest avidity effect in binding due to the bivalent nature of the antibody which could potentially cross-link two neighboring ROR1 in the cell surface.

Both Biacore (SPR) and MSD-CAT demonstrated that the weakest binder to ROR1 is RCDB9 regardless of the form of antigen used (recombinant or cell-surface expressed). In addition, both methods show that the other 5 ROR1×CD3 bispecific antibodies (RCDB5, RCDB13, RCDB6, RCDB11, RCDB12) bind with affinities that are within 3.6-fold or less of each other. MSD-CAT affinities of ROR1×CD3 bispecific antibodies for cell-surface ROR1 are >20-fold tighter than SPR data for recombinant ROR1. The difference in SPR versus MSD-CAT affinities for cell-surface ROR1 is most likely due to the presentation of the antigen on the cell surface in comparison to the recombinant antigen. The binding observed by SPR is representative of monovalent affinity which is not influenced by avidity effects regardless of the type of molecule analyzed (monospecific or bispecific antibody), but in the context of the cellular assay, the binding of the monospecific antibody, in contrast to the ROR1×CD3 bispecific antibodies, is affected by avidity due to the bivalent nature of the antibody. An example of this avidity effect is shown by the binding of RCDB5 and its parental anti-ROR1 mAb (RR1B67) (FIG. 7). The parental mAb showed twice the response of RCDB5 in Biacore (left panels) indicating occupation of the two arms in the parental mAb, but the binding profiles are similar. However, in the case of MSD-CAT (right panels) the curves are shifted to the left and the slope is steeper for the parental mAb indicating tighter binding.

In summary, the SPR data for RCDB5 and RR1B67 suggested a low nanomolar K_{D}. This was consistent with the expectation of monovalency in this experiment. ROR1-ECD was bound to the sensor-surface captured antibodies in a monomeric format. Binding of RCDB5 to cells was about 25 fold more potent (˜300 pM). It could be possible that the membrane anchored format of ROR1 as expressed on the cell surface presents the antigen in a more favorable conformation for binding. The large shift in potency for the RR1B67 molecule to cells was most likely due to an avidity effect. The high density of exogenous ROR1 surface expression on HEK293 cells would suggest that receptor crosslinking could occur and increase the apparent binding constant.

### In Vivo Activity of ROR1×CD3 Bispecific Antibodies

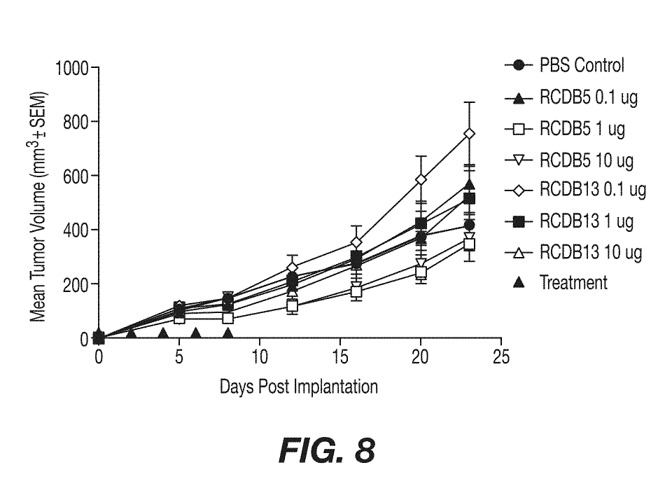

An admixture mouse model was used to evaluate the in vivo efficacy of RCDB13 and RCDB5. In the model, 5 million H1975 cells were mixed with 1 million human T cells (E:T ratio of 5:1) in 50% Cultrex. The mixture was implanted into the right flank of female athymic nude mice (0.1 ml/mouse). Treatment with ROR1×CD3 bispecific antibodies began on day zero and continued for 5 doses (IV). A PBS control group, and 0.1, 1, and 10 ug/mouse were administered for each ROR1×CD3 bispecific antibody (FIG. 8). The ROR1×CD3 bispecific antibody RCDB5 at 1 and 10 ug/mouse surpressed the growth of the H1975 tumor when compared to the PBS control. An overall weaker effect was observed with RCDB13.

### Fc Receptor Binding of RCDB5

The purpose of this study was to characterize by competition AlphaScreen the interaction of the ROR1×CD3 bispecific antibody RCDB5 with human FcγR1, FcγRIIa, FcγRIIb, FcγRIIIa, and FcRn relative to wild type hIgG1 and a collection of related IgG4 PAA control parental (bivalent) and null-arm (monovalent) molecules. The antibodies used in the AlphaScreen are provided in Table 19. The AlphaScreen is a bead-based assay system used to study biomolecular interactions in microplate format. AlphaScreen assays used two types of bead: Donor beads and Acceptor beads. Both bead types were coated with a hydrogel which minimized non-specific binding and self-aggregation while providing reactive groups for conjugating molecules to the bead surface. Donor beads contained a photosensitizer, which converted ambient oxygen to singlet oxygen upon illumination at 680 nm. Singlet oxygen is not a free-radical and, like other excited molecules, has a limited lifetime before it reverts to ground state. Within its 4 μsec half-life, singlet oxygen can diffuse approximately 200 nm in solution. If an Acceptor bead is within that distance, chemical energy can be transferred from singlet oxygen to thioxene derivatives within the Acceptor bead which results in light production at 520-620 nm. Proximity-dependent chemical energy transfer is the basis for the homogeneous nature of AlphaScreen. In the disclosed experiments, competition was scored as a reduction in AlphaScreen signal. Briefly, control biotinylated IgG bound Streptavidin Donor beads bring His-tagged FcγR/FcRn bound Ni2+ Acceptor beads into proximity producing, upon illumination, a chemiluminescent signal. Unlabeled competitor test Abs were serially diluted and applied. Signal was reduced when a test Ab competed with control biotinylated IgG for binding to FcγR/FcRn.

TABLE 19
Roster of Competitor Abs Used in AlphaScreen

| FcγR | Supplier | Cat. No. | Comments |
|---|---|---|---|
| FcγRI | | | Formulated w/o |
| (CD64) | R&D Systems | 1257-FC | BSA |
| FcγRIIa | | | Formulated w/o |
| (CD32a) | R&D Systems | 1330-CD/CF | BSA |
| FcγRIIb/c | | | Formulated w/o |
| (CD32b/c) | R&D Systems | 1875-CD | BSA |
| FcγRIIIa | | | Formulated w/o |
| (CD16a) | R&D Systems | 4325-FC | BSA |
| FcRn | Supplier | Cat. No. | Comments |
| FCGRT/B2M | Sino Biological | CT009-H08H | Formulated w/o |
| Heterodimer | | | BSA |

Sample
B21M IgG1, CNTO3930, ICAB7.005, 11.7 mg/mL
B21M IgG4PAA, I3CB15.I3CB15.001, 7.26 mg/mL
CD3B219 × B23B39 huIgG4 PAA null arm DuoBody, 11.4 mg/mL
Control DuoBody for ROR1, RR1B78xRSV B21M huIgG4_PAA_F405L,
CBIS ID: RR1B120.001, DPR lot: 8661-B120, 3.99 mg/mL
RR1B67 ELM, anti-ROR1, huIgG4_PAA, DPR lot: 9057, CBIS ID:
RR1B67.006, 6.13 mg/mL
RR1B67 × CD3B219, huIgG4_PAA Duobody, DPR lot: 9051, CBIS
ID: RCDB5.005, 6.9 mg/mL

Ranking in competition binding assays is often carried out on the basis of the EC50 values derived from dose-response curves. In AlphaScreen assays, however, competitor Abs do not always generate sigmoidal dose-response curves. Hence EC50 values cannot always be derived. Instead, degree of silence was established by comparing the % maximum signal at any given concentration across the entire cohort of Abs tested. Test Abs were assayed over two replicate experimental rounds carried out on different days. Only one (representative) set of results is shown.

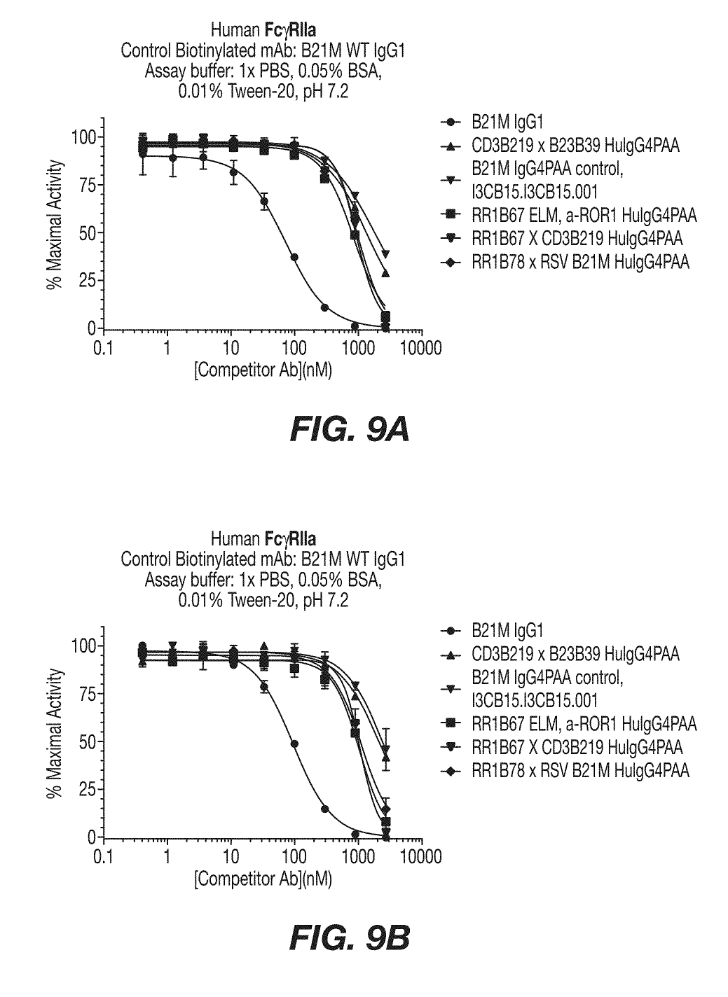

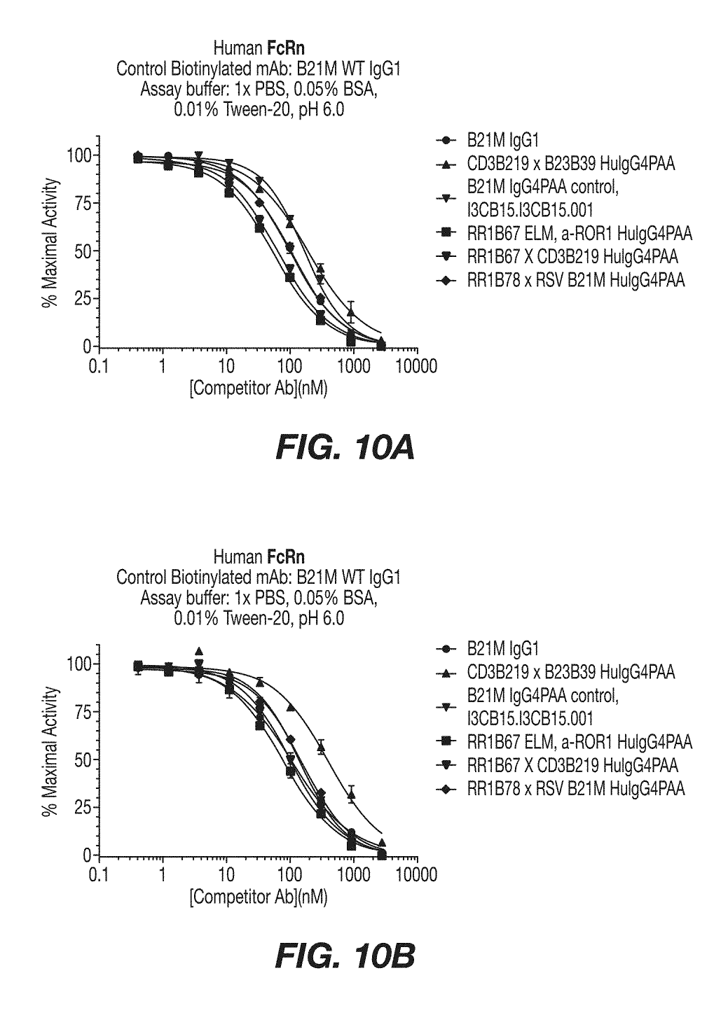

On FcγR1, RCDB5 bound to the same extent as the hIgG4 PAA isotype controls (not shown). RCDB5 bound to FcγRIIa (FIG. 9) and FcγRIIb (not shown) in a manner that is nearly identical to that of IgG4 PAA isotype control. On FcγRIIIa, RCDB5 was no more competitive than the hIgG4 PAA isotype Abs (FIG. 10). RCDB5 binds FcRn as efficiently as hIgG1 WT (see FIG. 10A; B21M IgG1 represents RSV IgG1 WT control). In summary, the ROR1×CD3 bispecific antibody RCDB5 bound all Fc receptors tested to essentially the same extent as matched IgG4 PAA isotype.

### Crystallization of the Kringle Domain of Human ROR1 and RR1B67

The human ROR1 Kringle Domain/RR1B67 Fab complex was prepared by a four-step procedure. First, the Fab and Kringle Domain were mixed together (1.2 molar excess of Fab) and incubated over the weekend at 4° C. while dialyzing into 20 mM Tris pH 8.0. Second, the complex was bound to a monoS 5/50 column (GE Healthcare) in 20 mM Tris pH 8.0 and eluted with a NaCl gradient using an ÄKTA purifier system (GE Healthcare). Due to poor separation, the peak fractions were pooled together, diluted 8 times in 20 mM Hepes pH 7.5, and the complex was purified using the mono S 5/50 column in 20 mM Hepes pH 7.5 and a NaCl gradient. Finally, the complex was concentrated to 7.8 mg/mL by ultrafiltration (Amicon Ultra-4 3 kDa).

Crystallization trials for the free Fab and Kringle/Fab complex were carried out using the sitting drop vapor-diffusion method at 20° C., Corning 3550 96-well plates (Hampton, cat no. HR8-146), and the crystallization screens IH1M, IH2M, JCSG+, CS, and. The screen solutions were dispensed into the plate wells with a Liquidator 96, model 200 uL (Mettler Toledo) and the nanodrops were prepared with a Mosquito LCP robot (TTP Labtech) at room temperature. The crystallization drops were monitored for at least 21 days using a Formulatrix imager that automatically recorded the drop image. Diffraction quality crystals of the ROR1 Kringle/RR1B67 Fab complex were grown from 18% PEG 3k, 0.2M (NH₄)₂SO₄, 0.1M acetate pH 4.5 with the complex initially at 7.8 mg/mL. Crystals of RR1B67 Fab were obtained from 2M (NH₄)₂SO₄, 5% MPD, 0.1M MES pH 6.5 with the Fab initially at 13 mg/mL. For data collection, the crystals were soaked for few seconds in a cryo-protectant solution containing the corresponding mother liquor supplemented with 20% glycerol and then, flash frozen in liquid nitrogen.

X-ray diffraction datasets were collected with a Pilatus 6M detector at beamline 22-ID of the Advanced Photon Source (APS) at Argonne National Laboratory. The X-ray diffraction datasets were processed with the program HKL2000. The structures were solved by molecular replacement with the program Phaser, human germline antibody 1-69/B3 (PDB code 3QOT) as search model for the free RR1B67 Fab, and human plasminogen kringle 3 domain (PDB code: 2LOS, NMR model 1) and free RR1B67 Fab as search models for the ROR1 kringle/RR1B67 Fab complex. The structures were refined with the program PHENIX. Model adjustments and structural overlays were carried out using the program COOT. Crystallographic calculations, contact distances for definition of epitope and paratope residues, and epitope area calculations were performed with the CCP4 suite of programs. All molecular graphics were generated with PyMol (PyMOL Molecular Graphics System, Version 1.4.1, Schröclinger, LLC) and the CDRs were determined using the Kabat definition.

Crystal structures for the Fab of RR1B67 bound to the isolated human ROR1 Kringle domain (with Fc removed by TEV digestion) was determined to 3.2 Å resolution. The structure of the antibody/antigen complex allowed the characterization of the interactions in atomic detail, increased the understanding of the antibody mechanism of action, and enhanced the knowledge about the ROR1 extracellular region. There is no available structure of any part of the ROR1 extracellular domain in public literature. The ROR1 structure includes residues 310-391, corresponding to the whole Kringle domain, and one N-linked glycan in Asn-315. The structure reveals all important interactions with the Fab. Some protein residues were present in the crystalline environment but are not ordered: six N-terminal and 15 C-terminal ROR1 residues, as well as 14 amino acid residues leftover from the Fc fusion. These disordered residues were not resolved in the structure and were excluded from the final model. The RR1B67 Fab structures included light chain residues 1-213 and heavy chain residues 1-220 (except for heavy chain residues 134-140, which are disordered and were not included). There was one RR1B67/Kringle complex in the asymmetric unit with the Fab/antigen combining site well defined by the electron density map, which allowed reliable positioning of the binding residues. The Fab was numbered sequentially in all Figures and ROR1 numbering starts at the signal peptide.

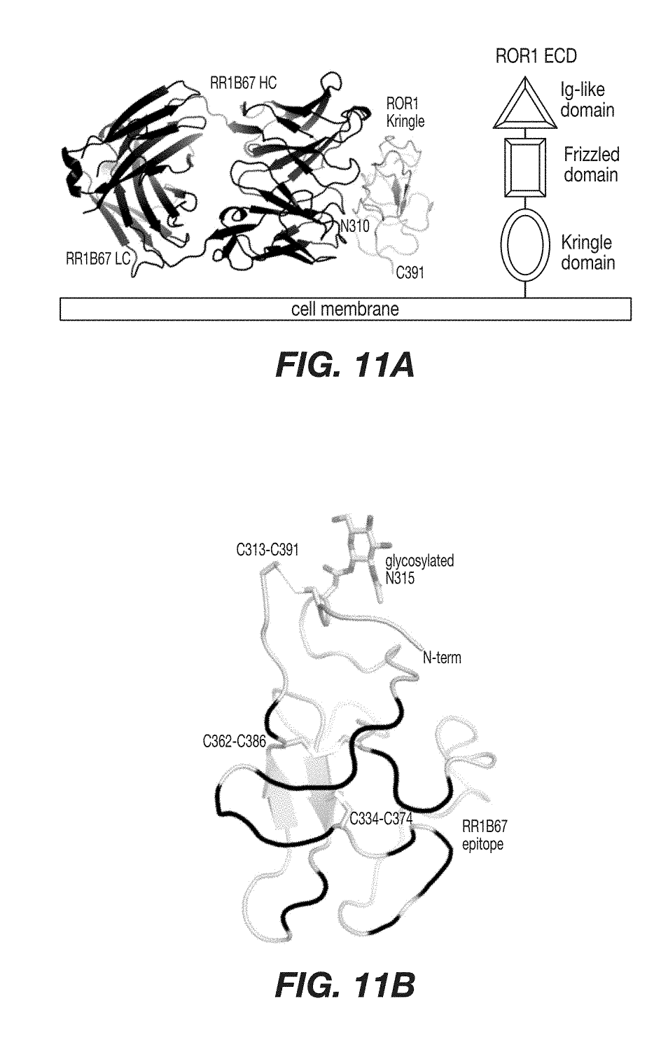

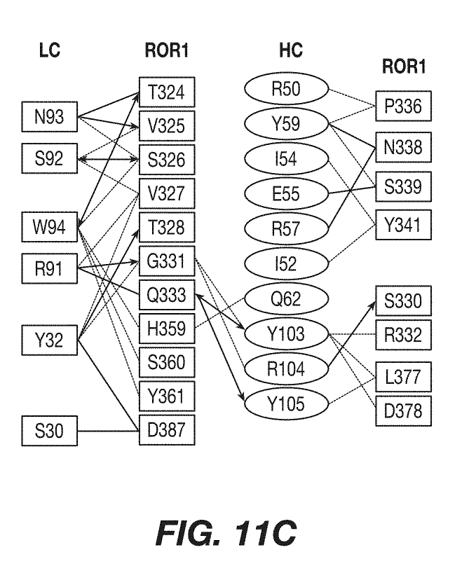

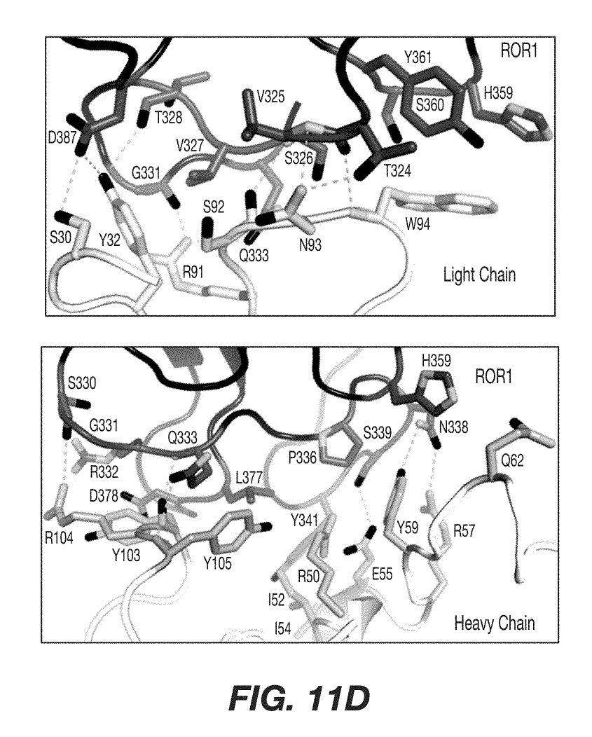

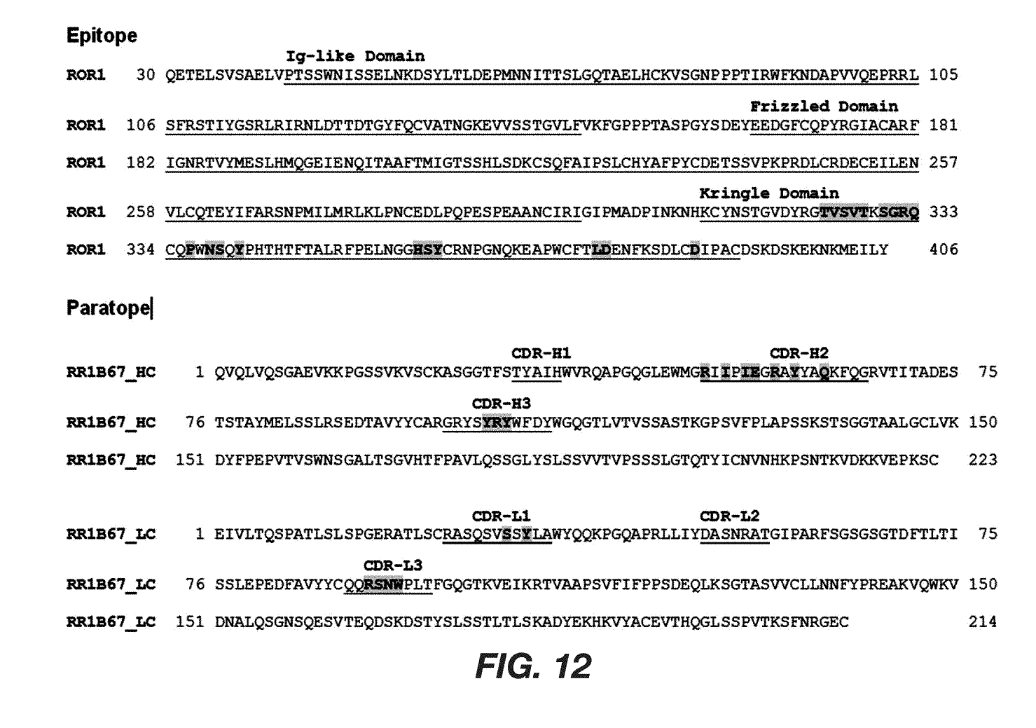

The structure of the complex between RR1B67 Fab and the ROR1 Kringle domain demonstrated that the epitope is discontinuous and involves residues distributed throughout loop regions of the membrane-proximal Kringle domain (residues T324-T328, S330-Q333, P336, N338, S339, Y341, H359-Y361, L377, D378, and D387) (FIG. 11 and FIG. 12). The Kringle surface area buried by the Fab was around 882 Å² and there were extensive contacts between the Kringle and Fab (FIG. 11C-FIG. 11D), which was consistent with the antibody/antigen subnanomolar affinity determined by SPR. The RR1B67 paratope was composed of residues in CDR-L1, -L3, -H2, and -H3 (FIG. 12). Gly331, Gln333, and His359 contact both the heavy and light chains of RR1B67, while all the other epitope residues contact either heavy or light chain. (FIG. 11C-FIG. 11D). The only residue that differ between human/cyno and mouse ROR1 Kringle is T396S. This residue is not in the antibody/antigen interface.

Those skilled in the art will appreciate that numerous changes and modifications can be made to the preferred embodiments of the disclosed isolated anti-ROR1 antibodies, ROR1×CD3 bispecific antibodies, and methods of using the same, and that such changes and modifications can be made without departing from the spirit of the invention. It is, therefore, intended that the appended claims cover all such equivalent variations as fall within the true spirit and scope of the invention.

The disclosures of each patent, patent application, and publication cited or described in this document are hereby incorporated herein by reference, in its entirety.

TABLE 20
Anti-ROR1 Ab Sequences (CDRs defined by Kabat)

| Anti- | Chain | |
|---|---|---|
| ROR1 | Peptide Id | |
| mAb | Isotype | Sequence |
| RR1B65 | Heavy Chain | EVQLLESGGGLVQPGGSLRLSCAASGFTFSNYWMSWVRQAPGKGLEWVSAISGSGGSTY |
| | RR1H31 | YADSVKGRFTISRDNSKNTLYLQMNSLRAEDTAVYYCAKDLIGSWGMYFDYWGQGTLV |
| | huIgG4_nG4m(a)_PAA | TVSSASTKGPSVFPLAPCSRSTSESTAALGCLVKDYFPEPVTVSWNSGALTSGVHTFPAVL |
| | | QSSGLYSLSSVVTVPSSSLGTKTYTCNVDHKPSNTKVDKRVESKYGPPCPPCPAPEAAGGP |
| | | SVFLFPPKPKDTLMISRTPEVTCVVVDVSQEDPEVQFNWYVDGVEVHNAKTKPREEQFNS |
| | | TYRVVSVLTVLHQDWLNGKEYKCKVSNKGLPSSIEKTISKAKGQPREPQVYTLPPSQEEM |
| | | TKNQVSLTCLVKGFYPSDIAVEWESNGQPENNYKTTPPVLDSDGSFFLYSRLTVDKSRWQ |
| | | EGNVFSCSVMHEALHNHYTQKSLSLSLGK |
| | | SEQ ID NO: 1 |
| | HC CDR1 | NYWMS |
| | | SEQ ID NO: 2 |
| | HC CDR2 | AISGSGGSTYYADSVKG |
| | | SEQ ID NO: 3 |
| | HC CDR3 | DLIGSWGMYFDY |
| | | SEQ ID NO: 4 |
| | Light chain | EIVLTQSPATLSLSPGERATLSCRASQSVSSYLAWYQQKPGQAPRLLIYDASNRATGIPARF |
| | PH9L3 | SGSGSGTDFTLTISSLEPEDFAVYYCQQRSNWPLTFGQGTKVEIKRTVAAPSVFIFPPSDEQ |
| | huKappa_Km3 | LKSGTASVVCLLNNFYPREAKVQWKVDNALQSGNSQESVTEQDSKDSTYSLSSTLTLSKA |
| | | DYEKHKVYACEVTHQGLSSPVTKSFNRGEC |
| | | SEQ ID NO: 5 |
| | LC CDR1 | RASQSVSSYLA |
| | | SEQ ID NO: 6 |
| | LC CDR2 | DASNRAT |
| | | SEQ ID NO: 7 |
| | LC CDR3 | QQRSNWPLT |
| | | SEQ ID NO: 8 |
| RR1B66 | Heavy Chain | QVQLVQSGAEVKKPGSSVKVSCKASGGTFSTDAIHWVRQAPGQGLEWMGRIIPISGTANY |
| | RR1H51 | AQKFQGRVTITADESTSTAYMELSSLRSEDTAVYYCARGRYSYYYYFDYWGQGTLVTVS |
| | huIgG4_nG4m(a)_PAA | SASTKGPSVFPLAPCSRSTSESTAALGCLVKDYFPEPVTVSWNSGALTSGVHTFPAVLQSS |
| | | GLYSLSSVVTVPSSSLGTKTYTCNVDHKPSNTKVDKRVESKYGPPCPPCPAPEAAGGPSVF |
| | | LFPPKPKDTLMISRTPEVTCVVVDVSQEDPEVQFNWYVDGVEVHNAKTKPREEQFNSTYR |
| | | VVSVLTVLHQDWLNGKEYKCKVSNKGLPSSIEKTISKAKGQPREPQVYTLPPSQEEMTKN |
| | | QVSLTCLVKGFYPSDIAVEWESNGQPENNYKTTPPVLDSDGSFFLYSRLTVDKSRWQEGN |
| | | VFSCSVMHEALHNHYTQKSLSLSLGK |
| | | SEQ ID NO: 9 |
| | HC CDR1 | TDAIH |
| | | SEQ ID NO: 10 |
| | HC CDR2 | RIIPISGTANYAQKFQG |
| | | SEQ ID NO: 11 |
| | HC CDR3 | GRYSYYYYFDY |
| | | SEQ ID NO: 12 |
| | Light Chain | EIVLTQSPATLSLSPGERATLSCRASQSVSSYLAWYQQKPGQAPRLLIYDASNRATGIPARF |
| | PH9L3 | SGSGSGTDFTLTISSLEPEDFAVYYCQQRSNWPLTFGQGTKVEIKRTVAAPSVFIFPPSDEQ |
| | huKappa_Km3 | LKSGTASVVCLLNNFYPREAKVQWKVDNALQSGNSQESVTEQDSKDSTYSLSSTLTLSKA |
| | | DYEKHKVYACEVTHQGLSSPVTKSFNRGEC |
| | | SEQ ID NO: 5 |
| | LC CDR1 | RASQSVSSYLA |
| | | SEQ ID NO: 6 |
| | LC CDR2 | DASNRAT |
| | | SEQ ID NO: 7 |
| | LC CDR3 | QQRSNWPLT |
| | | SEQ ID NO: 8 |
| RR1B67 | Heavy Chain | QVQLVQSGAEVKKPGSSVKVSCKASGGTFSTYAIHWVRQAPGQGLEWMGRIIPIEGRAY |
| | RR1H52 | YAQKFQGRVTITADESTSTAYMELSSLRSEDTAVYYCARGRYSYRYWFDYWGQGTLVT |
| | huIgG4_nG4m(a)_PAA | VSSASTKGPSVFPLAPCSRSTSESTAALGCLVKDYFPEPVTVSWNSGALTSGVHTFPAVLQ |
| | | SSGLYSLSSVVTVPSSSLGTKTYTCNVDHKPSNTKVDKRVESKYGPPCPPCPAPEAAGGPS |
| | | VFLFPPKPKDTLMISRTPEVTCVVVDVSQEDPEVQFNWYVDGVEVHNAKTKPREEQFNST |
| | | YRVVSVLTVLHQDWLNGKEYKCKVSNKGLPSSIEKTISKAKGQPREPQVYTLPPSQEEMT |
| | | KNQVSLTCLVKGFYPSDIAVEWESNGQPENNYKTTPPVLDSDGSFFLYSRLTVDKSRWQE |
| | | GNVFSCSVMHEALHNHYTQKSLSLSLGK |
| | | SEQ ID NO: 13 |
| | HC CDR1 | TYAIH |
| | | SEQ ID NO: 14 |
| | HC CDR2 | RIIPIEGRAYYAQKFQG |
| | | SEQ ID NO: 15 |
| | HC CDR3 | GRYSYRYWFDY |
| | | SEQ ID NO: 16 |
| | Light Chain | EIVLTQSPATLSLSPGERATLSCRASQSVSSYLAWYQQKPGQAPRLLIYDASNRATGIPARF |
| | PH9L3 | SGSGSGTDFTLTISSLEPEDFAVYYCQQRSNWPLTFGQGTKVEIKRTVAAPSVFIFPPSDEQ |
| | huKappa_Km3 | LKSGTASVVCLLNNFYPREAKVQWKVDNALQSGNSQESVTEQDSKDSTYSLSSTLTLSKA |
| | | DYEKHKVYACEVTHQGLSSPVTKSFNRGEC |
| | | SEQ ID NO: 5 |
| | LC CDR1 | RASQSVSSYLA |
| | | SEQ ID NO: 6 |
| | LC CDR2 | DASNRAT |
| | | SEQ ID NO: 7 |
| | LC CDR3 | QQRSNWPLT |
| | | SEQ ID NO: 8 |
| RR1B69 | Heavy Chain | QVQLVQSGAEVKKPGSSVKVSCKASGGTFSDAAIHWVRQAPGQGLEWMGYIIPISGTAN |
| | RR1H65 | YAQKFQGRVTITADESTSTAYMELSSLRSEDTAVYYCARGRARYRWAFDYWGQGTLVT |
| | huIgG4_nG4m(a)_PAA | VSSASTKGPSVFPLAPCSRSTSESTAALGCLVKDYFPEPVTVSWNSGALTSGVHTFPAVLQ |
| | | SSGLYSLSSVVTVPSSSLGTKTYTCNVDHKPSNTKVDKRVESKYGPPCPPCPAPEAAGGPS |
| | | VFLFPPKPKDTLMISRTPEVTCVVVDVSQEDPEVQFNWYVDGVEVHNAKTKPREEQFNST |
| | | YRVVSVLTVLHQDWLNGKEYKCKVSNKGLPSSIEKTISKAKGQPREPQVYTLPPSQEEMT |
| | | KNQVSLTCLVKGFYPSDIAVEWESNGQPENNYKTTPPVLDSDGSFFLYSRLTVDKSRWQE |
| | | GNVFSCSVMHEALHNHYTQKSLSLSLGK |
| | | SEQ ID NO: 17 |
| | HC CDR1 | DAAIH |
| | | SEQ ID NO: 18 |
| | HC CDR2 | YIIPISGTANYAQKFQG |
| | | SEQ ID NO: 19 |
| | HC CDR3 | GRARYRWAFDY |
| | | SEQ ID NO: 20 |
| | Light Chain | EIVLTQSPATLSLSPGERATLSCRASQSVSSYLAWYQQKPGQAPRLLIYDASNRATGIPARF |
| | PH9L3 | SGSGSGTDFTLTISSLEPEDFAVYYCQQRSNWPLTFGQGTKVEIKRTVAAPSVFIFPPSDEQ |
| | huKappa_Km3 | LKSGTASVVCLLNNFYPREAKVQWKVDNALQSGNSQESVTEQDSKDSTYSLSSTLTLSKA |
| | | DYEKHKVYACEVTHQGLSSPVTKSFNRGEC |
| | | SEQ ID NO: 5 |
| | LC CDR1 | RASQSVSSYLA |
| | | SEQ ID NO: 6 |
| | LC CDR2 | DASNRAT |
| | | SEQ ID NO: 7 |
| | LC CDR3 | QQRSNWPLT |
| | | SEQ ID NO: 8 |
| RR1B70 | Heavy Chain | QVQLVQSGAEVKKPGSSVKVSCKASGGTFSSYAISWVRQAPGQGLEWMGGIIPIFGTANY |
| | RR1H68 | AQKFQGRVTITADESTSTAYMELSSLRSEDTAVYYCARDGYYSRYRGYFLYYGMDVWG |
| | huIgG4_nG4m(a)_PAA | QGTLVTVSSASTKGPSVFPLAPCSRSTSESTAALGCLVKDYFPEPVTVSWNSGALTSGVHT |
| | | FPAVLQSSGLYSLSSVVTVPSSSLGTKTYTCNVDHKPSNTKVDKRVESKYGPPCPPCPAPE |
| | | AAGGPSVFLFPPKPKDTLMISRTPEVTCVVVDVSQEDPEVQFNWYVDGVEVHNAKTKPR |
| | | EEQFNSTYRVVSVLTVLHQDWLNGKEYKCKVSNKGLPSSIEKTISKAKGQPREPQVYTLP |
| | | PSQEEMTKNQVSLTCLVKGFYPSDIAVEWESNGQPENNYKTTPPVLDSDGSFFLYSRLTV |
| | | DKSRWQEGNVFSCSVMHEALHNHYTQKSLSLSLGK |
| | | SEQ ID NO: 21 |
| | HC CDR1 | SYAIS |
| | | SEQ ID NO: 22 |
| | HC CDR2 | GIIPIFGTANYAQKFQG |
| | | SEQ ID NO: 23 |
| | HC CDR3 | DGYYSRYRGYFLYYGMDV |
| | | SEQ ID NO: 24 |
| | Light Chain | EIVLTQSPATLSLSPGERATLSCRASQSVSSYLAWYQQKPGQAPRLLIYDASNRATGIPARF |
| | PH9L3 | SGSGSGTDFTLTISSLEPEDFAVYYCQQRSNWPLTFGQGTKVEIKRTVAAPSVFIFPPSDEQ |
| | huKappa_Km3 | LKSGTASVVCLLNNFYPREAKVQWKVDNALQSGNSQESVTEQDSKDSTYSLSSTLTLSKA |
| | | DYEKHKVYACEVTHQGLSSPVTKSFNRGEC |
| | | SEQ ID NO: 5 |
| | LC CDR1 | RASQSVSSYLA |
| | | SEQ ID NO: 6 |
| | LC CDR2 | DASNRAT |
| | | SEQ ID NO: 7 |
| | LC CDR3 | QQRSNWPLT |
| | | SEQ ID NO: 8 |
| RR1B71 | Heavy Chain | EVQLLESGGGLVQPGGSLRLSCAASGFTFSSYAMSWVRQAPGKGLEWVSAIRYDGGSTY |
| | RR1H11 | YADSVKGRFTISRDNSKNTLYLQMNSLRAEDTAVYYCAKMFGGQLDYWGQGTLVTVSS |
| | huIgG4_nG4m(a)_PAA | ASTKGPSVFPLAPCSRSTSESTAALGCLVKDYFPEPVTVSWNSGALTSGVHTFPAVLQSSG |
| | | LYSLSSVVTVPSSSLGTKTYTCNVDHKPSNTKVDKRVESKYGPPCPPCPAPEAAGGPSVFL |
| | | FPPKPKDTLMISRTPEVTCVVVDVSQEDPEVQFNWYVDGVEVHNAKTKPREEQFNSTYR |
| | | VVSVLTVLHQDWLNGKEYKCKVSNKGLPSSIEKTISKAKGQPREPQVYTLPPSQEEMTKN |
| | | QVSLTCLVKGFYPSDIAVEWESNGQPENNYKTTPPVLDSDGSFFLYSRLTVDKSRWQEGN |
| | | VFSCSVMHEALHNHYTQKSLSLSLGK |
| | | SEQ ID NO: 25 |
| | HC CDR1 | SYAMS |
| | | SEQ ID NO: 26 |
| | HC CDR2 | AIRYDGGSTYYADSVKG |
| | | SEQ ID NO: 27 |
| | HC CDR3 | MFGGQLDY |
| | | SEQ ID NO: 28 |
| | Light Chain | DIQMTQSPSSLSASVGDRVTITCRASQSIDRHLNWYQQKPGKAPKLLIYKASSLQSGVPSR |
| | RR1L10 | FSGSGSGTDFTLTISSLQPEDFATYYCQQPHTFPLTFGQGTKVEIKRTVAAPSVFIFPPSDEQ |
| | huKappa_Km3 | LKSGTASVVCLLNNFYPREAKVQWKVDNALQSGNSQESVTEQDSKDSTYSLSSTLTLSKA |
| | | DYEKHKVYACEVTHQGLSSPVTKSFNRGEC |
| | | SEQ ID NO: 29 |
| | LC CDR1 | RASQSIDRHLN |
| | | SEQ ID NO: 30 |
| | LC CDR2 | KASSLQS |
| | | SEQ ID NO: 31 |
| | LC CDR3 | QQPHTFPLT |
| | | SEQ ID NO: 32 |
| RR1B72 | Heavy Chain | EVQLLESGGGLVQPGGSLRLSCAASGFTFSNYWMSWVRQAPGKGLEWVSGISYNGGSTY |
| | RR1H14 | YADSVKGRFTISRDNSKNTLYLQMNSLRAEDTAVYYCAKPYFGFDYWGQGTLVTVSSAS |
| | huIgG4_nG4m(a)_PAA | TKGPSVFPLAPCSRSTSESTAALGCLVKDYFPEPVTVSWNSGALTSGVHTFPAVLQSSGLY |
| | | SLSSVVTVPSSSLGTKTYTCNVDHKPSNTKVDKRVESKYGPPCPPCPAPEAAGGPSVFLFP |
| | | PKPKDTLMISRTPEVTCVVVDVSQEDPEVQFNWYVDGVEVHNAKTKPREEQFNSTYRVV |
| | | SVLTVLHQDWLNGKEYKCKVSNKGLPSSIEKTISKAKGQPREPQVYTLPPSQEEMTKNQV |
| | | SLTCLVKGFYPSDIAVEWESNGQPENNYKTTPPVLDSDGSFFLYSRLTVDKSRWQEGNVF |
| | | SCSVMHEALHNHYTQKSLSLSLGK |
| | | SEQ ID NO: 33 |
| | HC CDR1 | NYWMS |
| | | SEQ ID NO: 2 |
| | HC CDR2 | GISYNGGSTYYADSVKG |
| | | SEQ ID NO: 34 |
| | HC CDR3 | PYFGFDY |
| | | SEQ ID NO: 35 |
| | Light Chain | EIVLTQSPATLSLSPGERATLSCRASQSVADFLAWYQQKPGQAPRLLIYDASNRATGIPAR |
| | RR1L13 | FSGSGSGTDFTLTISSLEPEDFAVYYCQQGNRWPITFGQGTKVEIKRTVAAPSVFIFPPSDEQ |
| | huKappa_Km3 | LKSGTASVVCLLNNFYPREAKVQWKVDNALQSGNSQESVTEQDSKDSTYSLSSTLTLSKA |
| | | DYEKHKVYACEVTHQGLSSPVTKSFNRGEC |
| | | SEQ ID NO: 36 |
| | LC CDR1 | RASQSVADFLA |
| | | SEQ ID NO: 37 |
| | LC CDR2 | DASNRAT |
| | | SEQ ID NO: 7 |
| | LC CDR3 | QQGNRWPIT |
| | | SEQ ID NO: 38 |
| RR1B74 | Heavy Chain | QVQLVQSGAEVKKPGSSVKVSCKASGGTFSSYAISWVRQAPGQGLEWMGGIIPIFGTANY |
| | RR1H24 | AQKFQGRVTITADESTSTAYMELSSLRSEDTAVYYCARTGNKLDYWGQGTLVTVSSAST |
| | huIgG4_nG4m(a)_PAA | KGPSVFPLAPCSRSTSESTAALGCLVKDYFPEPVTVSWNSGALTSGVHTFPAVLQSSGLYS |
| | | LSSVVTVPSSSLGTKTYTCNVDHKPSNTKVDKRVESKYGPPCPPCPAPEAAGGPSVFLFPP |
| | | KPKDTLMISRTPEVTCVVVDVSQEDPEVQFNWYVDGVEVHNAKTKPREEQFNSTYRVVS |
| | | VLTVLHQDWLNGKEYKCKVSNKGLPSSIEKTISKAKGQPREPQVYTLPPSQEEMTKNQVS |
| | | LTCLVKGFYPSDIAVEWESNGQPENNYKTTPPVLDSDGSFFLYSRLTVDKSRWQEGNVFS |
| | | CSVMHEALHNHYTQKSLSLSLGK |
| | | SEQ ID NO: 39 |
| | HC CDR1 | SYAIS |
| | | SEQ ID NO: 22 |
| | HC CDR2 | GIIPIFGTANYAQKFQG |
| | | SEQ ID NO: 23 |
| | HC CDR3 | TGNKLDY |
| | | SEQ ID NO: 40 |
| | Light Chain | EIVLTQSPATLSLSPGERATLSCRASQSVAKFLAWYQQKPGQAPRLLIYFASNRATDIPARF |
| | RR1L22 | SGSGSGTDFTLTISSLEPEDFAVYYCQQRDHWPLTFGQGTKVEIKRTVAAPSVFIFPPSDEQ |
| | huKappa_Km3 | LKSGTASVVCLLNNFYPREAKVQWKVDNALQSGNSQESVTEQDSKDSTYSLSSTLTLSKA |
| | | DYEKHKVYACEVTHQGLSSPVTKSFNRGEC |
| | | SEQ ID NO: 41 |
| | LC CDR1 | RASQSVAKFLA |
| | | SEQ ID NO: 42 |
| | LC CDR2 | FASNRAT |
| | | SEQ ID NO: 43 |
| | LC CDR3 | QQRDHWPLT |
| | | SEQ ID NO: 44 |
| RR1B76 | Heavy Chain | QVQLVQSGAEVKKPGSSVKVSCKASGGTFSSYGISWVRQAPGQGLEWMGWISAIFGTAN |
| | RR1H23 | YAQKFQGRVTITADESTSTAYMELSSLRSEDTAVYYCARGVASWYLDYWGQGTLVTVSS |
| | huIgG4_nG4m(a)_PAA | ASTKGPSVFPLAPCSRSTSESTAALGCLVKDYFPEPVTVSWNSGALTSGVHTFPAVLQSSG |
| | | LYSLSSVVTVPSSSLGTKTYTCNVDHKPSNTKVDKRVESKYGPPCPPCPAPEAAGGPSVFL |
| | | FPPKPKDTLMISRTPEVTCVVVDVSQEDPEVQFNWYVDGVEVHNAKTKPREEQFNSTYR |
| | | VVSVLTVLHQDWLNGKEYKCKVSNKGLPSSIEKTISKAKGQPREPQVYTLPPSQEEMTKN |
| | | QVSLTCLVKGFYPSDIAVEWESNGQPENNYKTTPPVLDSDGSFFLYSRLTVDKSRWQEGN |
| | | VFSCSVMHEALHNHYTQKSLSLSLGK |
| | | SEQ ID NO: 45 |
| | HC CDR1 | SYGIS |
| | | SEQ ID NO: 46 |
| | HC CDR2 | WISAIFGTANYAQKFQG |
| | | SEQ ID NO: 47 |
| | HC CDR3 | GVASWYLDY |
| | | SEQ ID NO: 48 |
| | Light Chain | DIQMTQSPSSLSASVGDRVTITCRASQSINKWLNWYQQKPGKAPKLLIYAASSLQSGVPSR |
| | RR1L24 | FSGSGSGTDFTLTISSLQPEDFATYYCQQDYNFPYTFGQGTKVEIKRTVAAPSVFIFPPSDE |
| | huKappa_Km3 | QLKSGTASVVCLLNNFYPREAKVQWKVDNALQSGNSQESVTEQDSKDSTYSLSSTLTLSK |
| | | ADYEKHKVYACEVTHQGLSSPVTKSFNRGEC |
| | | SEQ ID NO: 49 |
| | LC CDR1 | RASQSINKWLN |
| | | SEQ ID NO: 50 |
| | LC CDR2 | AASSLQS |
| | | SEQ ID NO: 51 |
| | LC CDR3 | QQDYNFPYT |
| | | SEQ ID NO: 52 |
| RR1B77 | Heavy Chain | EVQLVQSGAEVKKPGESLKISCKGSGYSFTSYWIGWVRQMPGKGLEWMGIIYPGDSDTR |
| | RR1H2 | YSPSFQGQVTISADKSISTAYLQWSSLKASDTAMYYCARVFASRSGYGIPFDYWGQGTLV |
| | huIgG4_nG4m(a)_PAA | TVSSASTKGPSVFPLAPCSRSTSESTAALGCLVKDYFPEPVTVSWNSGALTSGVHTFPAVL |
| | | QSSGLYSLSSVVTVPSSSLGTKTYTCNVDHKPSNTKVDKRVESKYGPPCPPCPAPEAAGGP |
| | | SVFLFPPKPKDTLMISRTPEVTCVVVDVSQEDPEVQFNWYVDGVEVHNAKTKPREEQFNS |
| | | TYRVVSVLTVLHQDWLNGKEYKCKVSNKGLPSSIEKTISKAKGQPREPQVYTLPPSQEEM |
| | | TKNQVSLTCLVKGFYPSDIAVEWESNGQPENNYKTTPPVLDSDGSFFLYSRLTVDKSRWQ |
| | | EGNVFSCSVMHEALHNHYTQKSLSLSLGK |
| | | SEQ ID NO: 53 |
| | HC CDR1 | SYWIG |
| | | SEQ ID NO: 54 |
| | HC CDR2 | IIYPGDSDTRYSPSFQG |
| | | SEQ ID NO: 55 |
| | HC CDR3 | VFASRSGYGIPFDY |
| | | SEQ ID NO: 56 |
| | Light Chain | DIVMTQSPDSLAVSLGERATINCKSSQSVLYSSNNKNYLAWYQQKPGQPPKLLIYWASTR |
| | PH9L2 | ESGVPDRFSGSGSGTDFTLTISSLQAEDVAVYYCQQYYSTPLTFGQGTKVEIKRTVAAPSV |
| | hu\LC\kappa\constant | FIFPPSDEQLKSGTASVVCLLNNFYPREAKVQWKVDNALQSGNSQESVTEQDSKDSTYSL |
| | | SSTLTLSKADYEKHKVYACEVTHQGLSSPVTKSFNRGEC |
| | | SEQ ID NO: 57 |
| | LC CDR1 | KSSQSVLYSSNNKNYLA |
| | | SEQ ID NO: 58 |
| | LC CDR2 | WASTRES |
| | | SEQ ID NO: 59 |
| | LC CDR3 | QQYYSTPLT |
| | | SEQ ID NO: 60 |
| RR1B78 | Heavy Chain | EVQLVQSGAEVKKPGESLKISCKGSGYSFTSYWIGWVRQMPGKGLEWMGIIYPGDSDTR |
| | RR1H27 | YSPSFQGQVTISADKSISTAYLQWSSLKASDTAMYYCARDVGRWAPGGFLDYWGQGTLV |
| | huIgG4_nG4m(a)_PAA | TVSSASTKGPSVFPLAPCSRSTSESTAALGCLVKDYFPEPVTVSWNSGALTSGVHTFPAVL |
| | | QSSGLYSLSSVVTVPSSSLGTKTYTCNVDHKPSNTKVDKRVESKYGPPCPPCPAPEAAGGP |
| | | SVFLFPPKPKDTLMISRTPEVTCVVVDVSQEDPEVQFNWYVDGVEVHNAKTKPREEQFNS |
| | | TYRVVSVLTVLHQDWLNGKEYKCKVSNKGLPSSIEKTISKAKGQPREPQVYTLPPSQEEM |
| | | TKNQVSLTCLVKGFYPSDIAVEWESNGQPENNYKTTPPVLDSDGSFFLYSRLTVDKSRWQ |
| | | EGNVFSCSVMHEALHNHYTQKSLSLSLGK |
| | | SEQ ID NO: 61 |
| | HC CDR1 | SYWIG |
| | | SEQ ID NO: 54 |
| | HC CDR2 | IIYPGDSDTRYSPSFQG |
| | | SEQ ID NO: 55 |
| | HC CDR3 | DVGRWAPGGFLDY |
| | | SEQ ID NO: 62 |
| | Light Chain | DIVMTQSPDSLAVSLGERATINCKSSQSVLYSSNNKNYLAWYQQKPGQPPKLLIYWASTR |
| | PH9L2 | ESGVPDRFSGSGSGTDFTLTISSLQAEDVAVYYCQQYYSTPLTFGQGTKVEIKRTVAAPSV |
| | hu\LC\kappa\constant | FIFPPSDEQLKSGTASVVCLLNNFYPREAKVQWKVDNALQSGNSQESVTEQDSKDSTYSL |
| | | SSTLTLSKADYEKHKVYACEVTHQGLSSPVTKSFNRGEC |
| | | SEQ ID NO: 57 |
| | LC CDR1 | KSSQSVLYSSNNKNYLA |
| | | SEQ ID NO: 58 |
| | LC CDR2 | WASTRES |
| | | SEQ ID NO: 59 |
| | LC CDR3 | QQYYSTPLT |
| | | SEQ ID NO: 60 |
| RR1B82 | Heavy Chain | QVQLVQSGAEVKKPGSSVKVSCKASGGTFDSDAIHWVRQAPGQGLEWMGYIIPISGTAN |
| | RR1H62 | YAQKFQGRVTITADESTSTAYMELSSLRSEDTAVYYCARGRYSYRWTFDYWGQGTLVTV |
| | huIgG4_nG4m(a)_PAA | SSASTKGPSVFPLAPCSRSTSESTAALGCLVKDYFPEPVTVSWNSGALTSGVHTFPAVLQS |
| | | SGLYSLSSVVTVPSSSLGTKTYTCNVDHKPSNTKVDKRVESKYGPPCPPCPAPEAAGGPSV |
| | | FLFPPKPKDTLMISRTPEVTCVVVDVSQEDPEVQFNWYVDGVEVHNAKTKPREEQFNSTY |
| | | RVVSVLTVLHQDWLNGKEYKCKVSNKGLPSSIEKTISKAKGQPREPQVYTLPPSQEEMTK |
| | | NQVSLTCLVKGFYPSDIAVEWESNGQPENNYKTTPPVLDSDGSFFLYSRLTVDKSRWQEG |
| | | NVFSCSVMHEALHNHYTQKSLSLSLGK |
| | | SEQ ID NO: 63 |
| | HC CDR1 | SDAIH |
| | | SEQ ID NO: 64 |
| | HC CDR2 | YIIPISGTANYAQKFQG |
| | | SEQ ID NO: 19 |
| | HC CDR3 | GRYSYRWTFDY |
| | | SEQ ID NO: 65 |
| | Light Chain | EIVLTQSPATLSLSPGERATLSCRASQSVSSYLAWYQQKPGQAPRLLIYDASNRATGIPARF |
| | PH9L3 | SGSGSGTDFTLTISSLEPEDFAVYYCQQRSNWPLTFGQGTKVEIKRTVAAPSVFIFPPSDEQ |
| | huKappa_Km3 | LKSGTASVVCLLNNFYPREAKVQWKVDNALQSGNSQESVTEQDSKDSTYSLSSTLTLSKA |
| | | DYEKHKVYACEVTHQGLSSPVTKSFNRGEC |
| | | SEQ ID NO: 5 |
| | LC CDR1 | RASQSVSSYLA |
| | | SEQ ID NO: 6 |
| | LC CDR2 | DASNRAT |
| | | SEQ ID NO: 7 |
| | LC CDR3 | QQRSNWPLT |
| | | SEQ ID NO: 8 |
| RR1B83 | Heavy Chain | QVQLVQSGAEVKKPGSSVKVSCKASGGTFKSAYIHWVRQAPGQGLEWMGYISPYYGSA |
| | RR1H66 | YYAQKFQGRVTITADESTSTAYMELSSLRSEDTAVYYCARGRIAYYYVFDYWGQGTLVT |
| | huIgG4_nG4m(a)_PAA | VSSASTKGPSVFPLAPCSRSTSESTAALGCLVKDYFPEPVTVSWNSGALTSGVHTFPAVLQ |
| | | SSGLYSLSSVVTVPSSSLGTKTYTCNVDHKPSNTKVDKRVESKYGPPCPPCPAPEAAGGPS |
| | | VFLFPPKPKDTLMISRTPEVTCVVVDVSQEDPEVQFNWYVDGVEVHNAKTKPREEQFNST |
| | | YRVVSVLTVLHQDWLNGKEYKCKVSNKGLPSSIEKTISKAKGQPREPQVYTLPPSQEEMT |
| | | KNQVSLTCLVKGFYPSDIAVEWESNGQPENNYKTTPPVLDSDGSFFLYSRLTVDKSRWQE |
| | | GNVFSCSVMHEALHNHYTQKSLSLSLGK |
| | | SEQ ID NO: 66 |
| | HC CDR1 | SAYIH |
| | | SEQ ID NO: 67 |
| | HC CDR2 | YISPYYGSAYYAQKFQG |
| | | SEQ ID NO: 68 |
| | HC CDR3 | GRIAYYYVFDY |
| | | SEQ ID NO: 69 |
| | Light Chain | EIVLTQSPATLSLSPGERATLSCRASQSVSSYLAWYQQKPGQAPRLLIYDASNRATGIPARF |
| | PH9L3 | SGSGSGTDFTLTISSLEPEDFAVYYCQQRSNWPLTFGQGTKVEIKRTVAAPSVFIFPPSDEQ |
| | huKappa_Km3 | LKSGTASVVCLLNNFYPREAKVQWKVDNALQSGNSQESVTEQDSKDSTYSLSSTLTLSKA |
| | | DYEKHKVYACEVTHQGLSSPVTKSFNRGEC |
| | | SEQ ID NO: 5 |
| | LC CDR1 | RASQSVSSYLA |
| | | SEQ ID NO: 6 |
| | LC CDR2 | DASNRAT |
| | | SEQ ID NO: 7 |
| | LC CDR3 | QQRSNWPLT |
| | | SEQ ID NO: 8 |
| RR1B84 | Heavy Chain | QVQLVQSGAEVKKPGSSVKVSCKASGGTFSTDAIHWVRQAPGQGLEWMGYIIPISGNAN |
| | RR1H69 | YAQKFQGRVTITADESTSTAYMELSSLRSEDTAVYYCARGRYAYNWGFDYWGQGTLVT |
| | huIgG4_nG4m(a)_PAA | VSSASTKGPSVFPLAPCSRSTSESTAALGCLVKDYFPEPVTVSWNSGALTSGVHTFPAVLQ |
| | | SSGLYSLSSVVTVPSSSLGTKTYTCNVDHKPSNTKVDKRVESKYGPPCPPCPAPEAAGGPS |
| | | VFLFPPKPKDTLMISRTPEVTCVVVDVSQEDPEVQFNWYVDGVEVHNAKTKPREEQFNST |
| | | YRVVSVLTVLHQDWLNGKEYKCKVSNKGLPSSIEKTISKAKGQPREPQVYTLPPSQEEMT |
| | | KNQVSLTCLVKGFYPSDIAVEWESNGQPENNYKTTPPVLDSDGSFFLYSRLTVDKSRWQE |
| | | GNVFSCSVMHEALHNHYTQKSLSLSLGK |
| | | SEQ ID NO: 70 |
| | HC CDR1 | TDAIH |
| | | SEQ ID NO: 10 |
| | HC CDR2 | YIIPISGNANYAQKFQG |
| | | SEQ ID NO: 71 |
| | HC CDR3 | GRYAYNWGFDY |
| | | SEQ ID NO: 72 |
| | Light Chain | EIVLTQSPATLSLSPGERATLSCRASQSVSSYLAWYQQKPGQAPRLLIYDASNRATGIPARF |
| | PH9L3 | SGSGSGTDFTLTISSLEPEDFAVYYCQQRSNWPLTFGQGTKVEIKRTVAAPSVFIFPPSDEQ |
| | huKappa_Km3 | LKSGTASVVCLLNNFYPREAKVQWKVDNALQSGNSQESVTEQDSKDSTYSLSSTLTLSKA |
| | | DYEKHKVYACEVTHQGLSSPVTKSFNRGEC |
| | | SEQ ID NO: 5 |
| | LC CDR1 | RASQSVSSYLA |
| | | SEQ ID NO: 6 |
| | LC CDR2 | DASNRAT |
| | | SEQ ID NO: 7 |
| | LC CDR3 | QQRSNWPLT |
| | | SEQ ID NO: 8 |
| RR1B85 | Heavy Chain | EVQLVQSGAEVKKPGESLKISCKGSGYSFTSYWIGWVRQMPGKGLEWMGIIDPGDSDTR |
| | RR1H30 | YSPSFQGQVTISADKSISTAYLQWSSLKASDTAMYYCARGNDPWPDFDYWGQGTLVTVS |
| | huIgG4_nG4m(a)_PAA | SASTKGPSVFPLAPCSRSTSESTAALGCLVKDYFPEPVTVSWNSGALTSGVHTFPAVLQSS |
| | | GLYSLSSVVTVPSSSLGTKTYTCNVDHKPSNTKVDKRVESKYGPPCPPCPAPEAAGGPSVF |
| | | LFPPKPKDTLMISRTPEVTCVVVDVSQEDPEVQFNWYVDGVEVHNAKTKPREEQFNSTYR |
| | | VVSVLTVLHQDWLNGKEYKCKVSNKGLPSSIEKTISKAKGQPREPQVYTLPPSQEEMTKN |
| | | QVSLTCLVKGFYPSDIAVEWESNGQPENNYKTTPPVLDSDGSFFLYSRLTVDKSRWQEGN |
| | | VFSCSVMHEALHNHYTQKSLSLSLGK |
| | | SEQ ID NO: 73 |
| | HC CDR1 | SYWIG |
| | | SEQ ID NO: 54 |
| | HC CDR2 | IIDPGDSDTRYSPSFQG |
| | | SEQ ID NO: 74 |
| | HC CDR3 | GNDPWPDFDY |
| | | SEQ ID NO: 75 |
| | Light Chain | DIQMTQSPSSLSASVGDRVTITCRASQSISSYLNWYQQKPGKAPKLLIYAASSLQSGVPSRF |
| | PH9L4 | SGSGSGTDFTLTISSLQPEDFATYYCQQSYSTPLTFGQGTKVEIKRTVAAPSVFIFPPSDEQL |
| | huKappa_Km3 | KSGTASVVCLLNNFYPREAKVQWKVDNALQSGNSQESVTEQDSKDSTYSLSSTLTLSKA |
| | | DYEKHKVYACEVTHQGLSSPVTKSFNRGEC |
| | | SEQ ID NO: 76 |
| | LC CDR1 | RASQSISSYLN |
| | | SEQ ID NO: 77 |
| | LC CDR2 | AASSLQS |
| | | SEQ ID NO: 51 |
| | LC CDR3 | QQSYSTPLT |
| | | SEQ ID NO: 78 |
| RR1B86 | Heavy Chain | QVQLVQSGAEVKKPGSSVKVSCKASGGTFSSYAISWVRQAPGQGLEWMGGIIPIFGTANY |
| | RR1H13 | AQKFQGRVTITADESTSTAYMELSSLRSEDTAVYYCARGQDESGLDYWGQGTLVTVSSA |
| | huIgG4_nG4m(a)_PAA | STKGPSVFPLAPCSRSTSESTAALGCLVKDYFPEPVTVSWNSGALTSGVHTFPAVLQSSGL |
| | | YSLSSVVTVPSSSLGTKTYTCNVDHKPSNTKVDKRVESKYGPPCPPCPAPEAAGGPSVFLF |
| | | PPKPKDTLMISRTPEVTCVVVDVSQEDPEVQFNWYVDGVEVHNAKTKPREEQFNSTYRV |
| | | VSVLTVLHQDWLNGKEYKCKVSNKGLPSSIEKTISKAKGQPREPQVYTLPPSQEEMTKNQ |
| | | VSLTCLVKGFYPSDIAVEWESNGQPENNYKTTPPVLDSDGSFFLYSRLTVDKSRWQEGNV |
| | | FSCSVMHEALHNHYTQKSLSLSLGK |
| | | SEQ ID NO: 79 |
| | HC CDR1 | SYAIS |
| | | SEQ ID NO: 22 |
| | HC CDR2 | GIIPIFGTANYAQKFQG |
| | | SEQ ID NO: 23 |
| | HC CDR3 | GQDESGLDY |
| | | SEQ ID NO: 80 |
| | Light Chain | EIVLTQSPATLSLSPGERATLSCRASQSVSDWLAWYQQKPGQAPRLLIYDASNRATGIPAR |
| | RR1L12 | FSGSGSGTDFTLTISSLEPEDFAVYYCQQSGDWPYTFGQGTKVEIKRTVAAPSVFIFPPSDE |
| | huKappa_Km3 | QLKSGTASVVCLLNNFYPREAKVQWKVDNALQSGNSQESVTEQDSKDSTYSLSSTLTLSK |
| | | ADYEKHKVYACEVTHQGLSSPVTKSFNRGEC |
| | | SEQ ID NO: 81 |
| | LC CDR1 | RASQSVSDWLA |
| | | SEQ ID NO: 82 |
| | LC CDR2 | DASNRAT |
| | | SEQ ID NO: 7 |
| | LC CDR3 | QQSGDWPYT |
| | | SEQ ID NO: 83 |
| RR1B88 | Heavy Chain | QVQLVQSGAEVKKPGSSVKVSCKASGGTFSSYAISWVRQAPGQGLEWMGGISAIFGNAN |
| | RR1H20 | YAQKFQGRVTITADESTSTAYMELSSLRSEDTAVYYCARLRLGNLDYWGQGTLVTVSSA |
| | huIgG4_nG4m(a)_PAA | STKGPSVFPLAPCSRSTSESTAALGCLVKDYFPEPVTVSWNSGALTSGVHTFPAVLQSSGL |
| | | YSLSSVVTVPSSSLGTKTYTCNVDHKPSNTKVDKRVESKYGPPCPPCPAPEAAGGPSVFLF |
| | | PPKPKDTLMISRTPEVTCVVVDVSQEDPEVQFNWYVDGVEVHNAKTKPREEQFNSTYRV |
| | | VSVLTVLHQDWLNGKEYKCKVSNKGLPSSIEKTISKAKGQPREPQVYTLPPSQEEMTKNQ |
| | | VSLTCLVKGFYPSDIAVEWESNGQPENNYKTTPPVLDSDGSFFLYSRLTVDKSRWQEGNV |
| | | FSCSVMHEALHNHYTQKSLSLSLGK |
| | | SEQ ID NO: 84 |
| | HC CDR1 | SYAIS |
| | | SEQ ID NO: 22 |
| | HC CDR2 | GISAIFGNANYAQKFQG |
| | | SEQ ID NO: 85 |
| | HC CDR3 | LRLGNLDY |
| | | SEQ ID NO: 86 |
| | Light Chain | EIVLTQSPATLSLSPGERATLSCRASQSVANFLAWYQQKPGQAPRLLIYFASNRATGIPARF |
| | RR1L20 | SGSGSGTDFTLTISSLEPEDFAVYYCQQYYGAPITFGQGTKVEIKRTVAAPSVFIFPPSDEQL |
| | huKappa_Km3 | KSGTASVVCLLNNFYPREAKVQWKVDNALQSGNSQESVTEQDSKDSTYSLSSTLTLSKA |
| | | DYEKHKVYACEVTHQGLSSPVTKSFNRGEC |
| | | SEQ ID NO: 87 |
| | LC CDR1 | RASQSVANFLA |
| | | SEQ ID NO: 88 |
| | LC CDR2 | FASNRAT |
| | | SEQ ID NO: 43 |
| | LC CDR3 | QQYYGAPIT |
| | | SEQ ID NO: 89 |

TABLE 21
Sequence of CD3B219

| CD3 Chain | Sequence |
|---|---|
| Heavy Chain | EVQLVESGGGLVQPGGSLRLSCAASGFTFNTYAMNWVRQAPGKGLEWVARIRSKYNNYATYYAASVKGRF |
| | TISRDDSKNSLYLQMNSLKTEDTAVYYCARHGNFGNSYVSWFAYWGQGTLVTVSSASTKGPSVFPLAPCSR |
| | STSESTAALGCLVKDYFPEPVTVSWNSGALTSGVHTFPAVLQSSGLYSLSSVVTVPSSSLGTKTYTCNVDHKP |
| | SNTKVDKRVESKYGPPCPPCPAPEAAGGPSVFLFPPKPKDTLMISRTPEVTCVVVDVSQEDPEVQFNWYVDG |
| | VEVHNAKTKPREEQFNSTYRVVSVLTVLHQDWLNGKEYKCKVSNKGLPSSIEKTISKAKGQPREPQVYTLPP |
| | SQEEMTKNQVSLTCLVKGFYPSDIAVEWESNGQPENNYKTTPPVLDSDGSFLLYSKLTVDKSRWQEGNVFS |
| | CSVMHEALHNHYTQKSLSLSLGK |
| | SEQ ID NO: 90 |
| HC CDR1 | GFTFNTYAMN |
| | SEQ ID NO: 92 |
| HC CDR2 | RIRSKYNNYATY |
| | SEQ ID NO: 93 |
| HC CDR3 | HGNFGNSYVSWFAY |
| | SEQ ID NO: 94 |
| Light Chain | QTVVTQEPSLTVSPGGTVTLTCRSSTGAVTTSNYANWVQQKPGQAPRGLIGGTNKRAPGTPARFSGSLLGGK |
| | AALTLSGVQPEDEAEYYCALWYSNLWVFGGGTKLTVLGQPKAAPSVTLFPPSSEELQANKATLVCLISDFYP |
| | GAVTVAWKADSSPVKAGVETTTPSKQSNNKYAASSYLSLTPEQWKSHRSYSCQVTHEGSTVEKTVAPTECS |
| | SEQ ID NO: 91 |
| LC CDR1 | RSSTGAVTTSNYAN |
| | SEQ ID NO: 95 |
| LC CDR2 | GTNKRAP |
| | SEQ ID NO: 96 |
| LC CDR3 | ALWYSNLWV |
| | SEQ ID NO: 97 |

TABLE 22
Sequences for affinity matured anti-ROR1 antibodies (CDRs defined by Kabat)

| Anti- | Chain | |
|---|---|---|
| ROR1 | Peptide Id | |
| mAb | Isotype | Sequence |
| RR1B177 | Heavy Chain | QVQLVQSGAEVKKPGSSVKVSCKASGGTFSTDAIHWVRQAPGQGLEWMGRIIPISGTANY |
| | RR1H51 | AQKFQGRVTITADESTSTAYMELSSLRSEDTAVYYCARGRYSYYYYFDYWGQGTLVTVS |
| | huIgG4_nG4m(a)_PAA | SASTKGPSVFPLAPCSRSTSESTAALGCLVKDYFPEPVTVSWNSGALTSGVHTFPAVLQSS |
| | | GLYSLSSVVTVPSSSLGTKTYTCNVDHKPSNTKVDKRVESKYGPPCPPCPAPEAAGGPSVF |
| | | LFPPKPKDTLMISRTPEVTCVVVDVSQEDPEVQFNWYVDGVEVHNAKTKPREEQFNSTYR |
| | | VVSVLTVLHQDWLNGKEYKCKVSNKGLPSSIEKTISKAKGQPREPQVYTLPPSQEEMTKN |
| | | QVSLTCLVKGFYPSDIAVEWESNGQPENNYKTTPPVLDSDGSFFLYSRLTVDKSRWQEGN |
| | | VFSCSVMHEALHNHYTQKSLSLSLGK |
| | | SEQ ID NO: 101 |
| | HC CDR1 | TDAIH |
| | | SEQ ID NO: 103 |
| | HC CDR2 | RIIPISGTANYAQKFQG |
| | | SEQ ID NO: 104 |
| | HC CDR3 | GRYSYYYYFDY |
| | | SEQ ID NO: 105 |
| | Light chain | EIVLTQSPATLSLSPGERATLSCRASQSVSSYLEWYQQKPGQAPRLLIYDASNRATGIPARF |
| | RR1L231 | SGSGSGTDFTLTISSLEPEDFAVYYCQQRSNWPLTFGQGTKVEIKRTVAAPSVFIFPPSDEQ |
| | huKappa_Km3 | LKSGTASVVCLLNNFYPREAKVQWKVDNALQSGNSQESVTEQDSKDSTYSLSSTLTLSKA |
| | | DYEKHKVYACEVTHQGLSSPVTKSFNRGEC |
| | | SEQ ID NO: 102 |
| | LC CDR1 | RASQSVSSYLE |
| | | SEQ ID NO: 106 |
| | LC CDR2 | DASNRAT |
| | | SEQ ID NO: 107 |
| | LC CDR3 | QQRSNWPLT |
| | | SEQ ID NO: 108 |
| RR1B178 | Heavy Chain | QVQLVQSGAEVKKPGSSVKVSCKASGGTFSDAAIHWVRQAPGQGLEWMGYIIPISGTAN |
| | RR1H65 | YAQKFQGRVTITADESTSTAYMELSSLRSEDTAVYYCARGRARYRWAFDYWGQGTLVT |
| | huIgG4_nG4m(a)_PAA | VSSASTKGPSVFPLAPCSRSTSESTAALGCLVKDYFPEPVTVSWNSGALTSGVHTFPAVLQ |
| | | SSGLYSLSSVVTVPSSSLGTKTYTCNVDHKPSNTKVDKRVESKYGPPCPPCPAPEAAGGPS |
| | | VFLFPPKPKDTLMISRTPEVTCVVVDVSQEDPEVQFNWYVDGVEVHNAKTKPREEQFNST |
| | | YRVVSVLTVLHQDWLNGKEYKCKVSNKGLPSSIEKTISKAKGQPREPQVYTLPPSQEEMT |
| | | KNQVSLTCLVKGFYPSDIAVEWESNGQPENNYKTTPPVLDSDGSFFLYSRLTVDKSRWQE |
| | | GNVFSCSVMHEALHNHYTQKSLSLSLGK |
| | | SEQ ID NO: 109 |
| | HC CDR1 | DAAIH |
| | | SEQ ID NO: 111 |
| | HC CDR2 | YIIPISGTANYAQKFQG |
| | | SEQ ID NO: 112 |
| | HC CDR3 | GRARYRWAFDY |
| | | SEQ ID NO: 113 |
| | Light chain | EIVLTQSPATLSLSPGERATLSCRASQSVRNYLAWYQQKPGQAPRLLIYDASNRATGIPAR |
| | TRGL199 | FSGSGSGTDFTLTISSLEPEDFAVYYCQQRSNWPLTFGQGTKVEIKRTVAAPSVFIFPPSDE |
| | huKappa_Km3 | QLKSGTASVVCLLNNFYPREAKVQWKVDNALQSGNSQESVTEQDSKDSTYSLSSTLTLSK |
| | | ADYEKHKVYACEVTHQGLSSPVTKSFNRGEC |
| | | SEQ ID NO: 110 |
| | LC CDR1 | RASQSVRNYLA |
| | | SEQ ID NO: 114 |
| | LC CDR2 | DASNRAT |
| | | SEQ ID NO: 115 |
| | LC CDR3 | QQRSNWPLT |
| | | SEQ ID NO: 116 |
| RR1B179 | Heavy Chain | QVQLVQSGAEVKKPGSSVKVSCKASGGTFSDAAIHWVRQAPGQGLEWMGYIIPISGTAN |
| | RR1H65 | YAQKFQGRVTITADESTSTAYMELSSLRSEDTAVYYCARGRARYRWAFDYWGQGTLVT |
| | huIgG4_nG4m(a)_PAA | VSSASTKGPSVFPLAPCSRSTSESTAALGCLVKDYFPEPVTVSWNSGALTSGVHTFPAVLQ |
| | | SSGLYSLSSVVTVPSSSLGTKTYTCNVDHKPSNTKVDKRVESKYGPPCPPCPAPEAAGGPS |
| | | VFLFPPKPKDTLMISRTPEVTCVVVDVSQEDPEVQFNWYVDGVEVHNAKTKPREEQFNST |
| | | YRVVSVLTVLHQDWLNGKEYKCKVSNKGLPSSIEKTISKAKGQPREPQVYTLPPSQEEMT |
| | | KNQVSLTCLVKGFYPSDIAVEWESNGQPENNYKTTPPVLDSDGSFFLYSRLTVDKSRWQE |
| | | GNVFSCSVMHEALHNHYTQKSLSLSLGK |
| | | SEQ ID NO: 117 |
| | HC CDR1 | DAAIH |
| | | SEQ ID NO: 119 |
| | HC CDR2 | YIIPISGTANYAQKFQG |
| | | SEQ ID NO: 120 |
| | HC CDR3 | GRARYRWAFDY |
| | | SEQ ID NO: 121 |
| | Light chain | EIVLTQSPATLSLSPGERATLSCRASQSVSTYLAWYQQKPGQAPRLLIYDASNRATGIPARF |
| | PD1L85 | SGSGSGTDFTLTISSLEPEDFAVYYCQQRSNWPLTFGQGTKVEIKRTVAAPSVFIFPPSDEQ |
| | huKappa_Km3 | LKSGTASVVCLLNNFYPREAKVQWKVDNALQSGNSQESVTEQDSKDSTYSLSSTLTLSKA |
| | | DYEKHKVYACEVTHQGLSSPVTKSFNRGEC |
| | | SEQ ID NO: 118 |
| | LC CDR1 | RASQSVSTYLA |
| | | SEQ ID NO: 122 |
| | LC CDR2 | DASNRAT |
| | | SEQ ID NO: 123 |
| | LC CDR3 | QQRSNWPLT |
| | | SEQ ID NO: 124 |
| RR1B180 | Heavy Chain | QVQLVQSGAEVKKPGSSVKVSCKASGGTFSDAAIHWVRQAPGQGLEWMGYIIPISGTAN |
| | RR1H65 | YAQKFQGRVTITADESTSTAYMELSSLRSEDTAVYYCARGRARYRWAFDYWGQGTLVT |
| | huIgG4_nG4m(a)_PAA | VSSASTKGPSVFPLAPCSRSTSESTAALGCLVKDYFPEPVTVSWNSGALTSGVHTFPAVLQ |
| | | SSGLYSLSSVVTVPSSSLGTKTYTCNVDHKPSNTKVDKRVESKYGPPCPPCPAPEAAGGPS |
| | | VFLFPPKPKDTLMISRTPEVTCVVVDVSQEDPEVQFNWYVDGVEVHNAKTKPREEQFNST |
| | | YRVVSVLTVLHQDWLNGKEYKCKVSNKGLPSSIEKTISKAKGQPREPQVYTLPPSQEEMT |
| | | KNQVSLTCLVKGFYPSDIAVEWESNGQPENNYKTTPPVLDSDGSFFLYSRLTVDKSRWQE |
| | | GNVFSCSVMHEALHNHYTQKSLSLSLGK |
| | | SEQ ID NO: 125 |
| | HC CDR1 | DAAIH |
| | | SEQ ID NO: 127 |
| | HC CDR2 | YIIPISGTANYAQKFQG |
| | | SEQ ID NO: 128 |
| | HC CDR3 | GRARYRWAFDY |
| | | SEQ ID NO: 129 |
| | Light chain | EIVLTQSPATLSLSPGERATLSCRASQSVRNYLAWYQQKPGQAPRLLIEDASNRATGIPAR |
| | RR1L185 | FSGSGSGTDFTLTISSLEPEDFAVYYCQQRSNWPLTFGQGTKVEIKRTVAAPSVFIFPPSDE |
| | huKappa_Km3 | QLKSGTASVVCLLNNFYPREAKVQWKVDNALQSGNSQESVTEQDSKDSTYSLSSTLTLSK |
| | | ADYEKHKVYACEVTHQGLSSPVTKSFNRGEC |
| | | SEQ ID NO: 126 |
| | LC CDR1 | RASQSVRNYLA |
| | | SEQ ID NO: 130 |
| | LC CDR2 | DASNRAT |
| | | SEQ ID NO: 131 |
| | LC CDR3 | QQRSNWPLT |
| | | SEQ ID NO: 132 |
| RR1B181 | Heavy Chain | QVQLVQSGAEVKKPGSSVKVSCKASGGTFSTDAIHWVRQAPGQGLEWMGYIIPISGNAN |
| | RR1H69 | YAQKFQGRVTITADESTSTAYMELSSLRSEDTAVYYCARGRYAYNWGFDYWGQGTLVT |
| | huIgG4_nG4m(a)_PAA | VSSASTKGPSVFPLAPCSRSTSESTAALGCLVKDYFPEPVTVSWNSGALTSGVHTFPAVLQ |
| | | SSGLYSLSSVVTVPSSSLGTKTYTCNVDHKPSNTKVDKRVESKYGPPCPPCPAPEAAGGPS |
| | | VFLFPPKPKDTLMISRTPEVTCVVVDVSQEDPEVQFNWYVDGVEVHNAKTKPREEQFNST |
| | | YRVVSVLTVLHQDWLNGKEYKCKVSNKGLPSSIEKTISKAKGQPREPQVYTLPPSQEEMT |
| | | KNQVSLTCLVKGFYPSDIAVEWESNGQPENNYKTTPPVLDSDGSFFLYSRLTVDKSRWQE |
| | | GNVFSCSVMHEALHNHYTQKSLSLSLGK |
| | | SEQ ID NO: 133 |
| | HC CDR1 | TDAIH |
| | | SEQ ID NO: 135 |
| | HC CDR2 | YIIPISGNANYAQKFQG |
| | | SEQ ID NO: 136 |
| | HC CDR3 | GRYAYNWGFDY |
| | | SEQ ID NO: 137 |
| | Light chain | EIVLTQSPATLSLSPGERATLSCRASQSVSSYLAWYQQKPGQAPRLLIKDASNRATGIPARF |
| | RR1L67 | SGSGSGTDFTLTISSLEPEDFAVYYCQQRSNWPLTFGQGTKVEIKRTVAAPSVFIFPPSDEQ |
| | huKappa_Km3 | LKSGTASVVCLLNNFYPREAKVQWKVDNALQSGNSQESVTEQDSKDSTYSLSSTLTLSKA |
| | | DYEKHKVYACEVTHQGLSSPVTKSFNRGEC |
| | | SEQ ID NO: 134 |
| | LC CDR1 | RASQSVSSYLA |
| | | SEQ ID NO: 138 |
| | LC CDR2 | DASNRAT |
| | | SEQ ID NO: 139 |
| | LC CDR3 | QQRSNWPLT |
| | | SEQ ID NO: 140 |
| RR1B182 | Heavy Chain | QVQLVQSGAEVKKPGSSVKVSCKASGGTFSTDAIHWVRQAPGQGLEWMGYIIPISGNAN |
| | RR1H69 | YAQKFQGRVTITADESTSTAYMELSSLRSEDTAVYYCARGRYAYNWGFDYWGQGTLVT |
| | huIgG4_nG4m(a)_PAA | VSSASTKGPSVFPLAPCSRSTSESTAALGCLVKDYFPEPVTVSWNSGALTSGVHTFPAVLQ |
| | | SSGLYSLSSVVTVPSSSLGTKTYTCNVDHKPSNTKVDKRVESKYGPPCPPCPAPEAAGGPS |
| | | VFLFPPKPKDTLMISRTPEVTCVVVDVSQEDPEVQFNWYVDGVEVHNAKTKPREEQFNST |
| | | YRVVSVLTVLHQDWLNGKEYKCKVSNKGLPSSIEKTISKAKGQPREPQVYTLPPSQEEMT |
| | | KNQVSLTCLVKGFYPSDIAVEWESNGQPENNYKTTPPVLDSDGSFFLYSRLTVDKSRWQE |
| | | GNVFSCSVMHEALHNHYTQKSLSLSLGK |
| | | SEQ ID NO: 141 |
| | HC CDR1 | TDAIH |
| | | SEQ ID NO: 143 |
| | HC CDR2 | YIIPISGNANYAQKFQG |
| | | SEQ ID NO: 144 |
| | HC CDR3 | GRYAYNWGFDY |
| | | SEQ ID NO: 145 |
| | Light chain | EIVLTQSPATLSLSPGERATLSCRASQSVRNYLAWYQQKPGQAPRLLIKDASNRATGIPAR |
| | RR1L70 | FSGSGSGTDFTLTISSLEPEDFAVYYCQQRSNWPLTFGQGTKVEIKRTVAAPSVFIFPPSDE |
| | huKappa_Km3 | QLKSGTASVVCLLNNFYPREAKVQWKVDNALQSGNSQESVTEQDSKDSTYSLSSTLTLSK |
| | | ADYEKHKVYACEVTHQGLSSPVTKSFNRGEC |
| | | SEQ ID NO: 142 |
| | LC CDR1 | RASQSVRNYLA |
| | | SEQ ID NO: 146 |
| | LC CDR2 | DASNRAT |
| | | SEQ ID NO: 147 |
| | LC CDR3 | QQRSNWPLT |
| | | SEQ ID NO: 148 |
| RR1B183 | Heavy Chain | QVQLVQSGAEVKKPGSSVKVSCKASGGTFSTDAIHWVRQAPGQGLEWMGYIIPISGNAN |
| | RR1H69 | YAQKFQGRVTITADESTSTAYMELSSLRSEDTAVYYCARGRYAYNWGFDYWGQGTLVT |
| | huIgG4_nG4m(a)_PAA | VSSASTKGPSVFPLAPCSRSTSESTAALGCLVKDYFPEPVTVSWNSGALTSGVHTFPAVLQ |
| | | SSGLYSLSSVVTVPSSSLGTKTYTCNVDHKPSNTKVDKRVESKYGPPCPPCPAPEAAGGPS |
| | | VFLFPPKPKDTLMISRTPEVTCVVVDVSQEDPEVQFNWYVDGVEVHNAKTKPREEQFNST |
| | | YRVVSVLTVLHQDWLNGKEYKCKVSNKGLPSSIEKTISKAKGQPREPQVYTLPPSQEEMT |
| | | KNQVSLTCLVKGFYPSDIAVEWESNGQPENNYKTTPPVLDSDGSFFLYSRLTVDKSRWQE |
| | | GNVFSCSVMHEALHNHYTQKSLSLSLGK |
| | | SEQ ID NO: 149 |
| | HC CDR1 | TDAIH |
| | | SEQ ID NO: 151 |
| | HC CDR2 | YIIPISGNANYAQKFQG |
| | | SEQ ID NO: 152 |
| | HC CDR3 | GRYAYNWGFDY |
| | | SEQ ID NO: 153 |
| | Light chain | EIVLTQSPATLSLSPGERATLSCRASQSVRSYLAWYQQKPGQAPRLLIKDASDRATGIPARF |
| | RR1L65 | SGSGSGTDFTLTISSLEPEDFAVYYCQQRSNWPLTFGQGTKVEIKRTVAAPSVFIFPPSDEQ |
| | huKappa_Km3 | LKSGTASVVCLLNNFYPREAKVQWKVDNALQSGNSQESVTEQDSKDSTYSLSSTLTLSKA |
| | | DYEKHKVYACEVTHQGLSSPVTKSFNRGEC |
| | | SEQ ID NO: 150 |
| | LC CDR1 | RASQSVRSYLA |
| | | SEQ ID NO: 154 |
| | LC CDR2 | DASDRAT |
| | | SEQ ID NO: 155 |
| | LC CDR3 | QQRSNWPLT |
| | | SEQ ID NO: 156 |
| RR1B184 | Heavy Chain | QVQLVQSGAEVKKPGSSVKVSCKASGGTFSTDAIHWVRQAPGQGLEWMGYIIPISGNAN |
| | RR1H69 | YAQKFQGRVTITADESTSTAYMELSSLRSEDTAVYYCARGRYAYNWGFDYWGQGTLVT |
| | huIgG4_nG4m(a)_PAA | VSSASTKGPSVFPLAPCSRSTSESTAALGCLVKDYFPEPVTVSWNSGALTSGVHTFPAVLQ |
| | | SSGLYSLSSVVTVPSSSLGTKTYTCNVDHKPSNTKVDKRVESKYGPPCPPCPAPEAAGGPS |
| | | VFLFPPKPKDTLMISRTPEVTCVVVDVSQEDPEVQFNWYVDGVEVHNAKTKPREEQFNST |
| | | YRVVSVLTVLHQDWLNGKEYKCKVSNKGLPSSIEKTISKAKGQPREPQVYTLPPSQEEMT |
| | | KNQVSLTCLVKGFYPSDIAVEWESNGQPENNYKTTPPVLDSDGSFFLYSRLTVDKSRWQE |
| | | GNVFSCSVMHEALHNHYTQKSLSLSLGK |
| | | SEQ ID NO: 157 |
| | HC CDR1 | TDAIH |
| | | SEQ ID NO: 159 |
| | HC CDR2 | YIIPISGNANYAQKFQG |
| | | SEQ ID NO: 160 |
| | HC CDR3 | GRYAYNWGFDY |
| | | SEQ ID NO: 161 |
| | Light chain | EIVLTQSPATLSLSPGERATLSCRASQSVSSYLAWYQQKPGQAPRLLIKDASTRATGIPARF |
| | RR1L81 | SGSGSGTDFTLTISSLEPEDFAVYYCQQRSNWPLTFGQGTKVEIKRTVAAPSVFIFPPSDEQ |
| | huKappa_Km3 | LKSGTASVVCLLNNFYPREAKVQWKVDNALQSGNSQESVTEQDSKDSTYSLSSTLTLSKA |
| | | DYEKHKVYACEVTHQGLSSPVTKSFNRGEC |
| | | SEQ ID NO: 158 |
| | LC CDR1 | RASQSVSSYLA |
| | | SEQ ID NO: 162 |
| | LC CDR2 | DASTRAT |
| | | SEQ ID NO: 163 |
| | LC CDR3 | QQRSNWPLT |
| | | SEQ ID NO: 164 |
| RR1B185 | Heavy Chain | QVQLVQSGAEVKKPGSSVKVSCKASGGTFSTDAIHWVRQAPGQGLEWMGYIIPISGNAN |
| | RR1H69 | YAQKFQGRVTITADESTSTAYMELSSLRSEDTAVYYCARGRYAYNWGFDYWGQGTLVT |
| | huIgG4_nG4m(a)_PAA | VSSASTKGPSVFPLAPCSRSTSESTAALGCLVKDYFPEPVTVSWNSGALTSGVHTFPAVLQ |
| | | SSGLYSLSSVVTVPSSSLGTKTYTCNVDHKPSNTKVDKRVESKYGPPCPPCPAPEAAGGPS |
| | | VFLFPPKPKDTLMISRTPEVTCVVVDVSQEDPEVQFNWYVDGVEVHNAKTKPREEQFNST |
| | | YRVVSVLTVLHQDWLNGKEYKCKVSNKGLPSSIEKTISKAKGQPREPQVYTLPPSQEEMT |
| | | KNQVSLTCLVKGFYPSDIAVEWESNGQPENNYKTTPPVLDSDGSFFLYSRLTVDKSRWQE |
| | | GNVFSCSVMHEALHNHYTQKSLSLSLGK |
| | | SEQ ID NO: 165 |
| | HC CDR1 | TDAIH |
| | | SEQ ID NO: 167 |
| | HC CDR2 | YIIPISGNANYAQKFQG |
| | | SEQ ID NO: 168 |
| | HC CDR3 | GRYAYNWGFDY |
| | | SEQ ID NO: 169 |
| | Light chain | EIVLTQSPATLSLSPGERATLSCRASQSVNTYLAWYQQKPGQAPRLLIKDASTRATGIPARF |
| | RR1L63 | SGSGSGTDFTLTISSLEPEDFAVYYCQQRSNWPLTFGQGTKVEIKRTVAAPSVFIFPPSDEQ |
| | huKappa_Km3 | LKSGTASVVCLLNNFYPREAKVQWKVDNALQSGNSQESVTEQDSKDSTYSLSSTLTLSKA |
| | | DYEKHKVYACEVTHQGLSSPVTKSFNRGEC |
| | | SEQ ID NO: 166 |
| | LC CDR1 | RASQSVNTYLA |
| | | SEQ ID NO: 170 |
| | LC CDR2 | DASTRAT |
| | | SEQ ID NO: 171 |
| | LC CDR3 | QQRSNWPLT |
| | | SEQ ID NO: 172 |
| RR1B186 | Heavy Chain | QVQLVQSGAEVKKPGSSVKVSCKASGGTFSTDAIHWVRQAPGQGLEWMGYIIPISGNAN |
| | RR1H69 | YAQKFQGRVTITADESTSTAYMELSSLRSEDTAVYYCARGRYAYNWGFDYWGQGTLVT |
| | huIgG4_nG4m(a)_PAA | VSSASTKGPSVFPLAPCSRSTSESTAALGCLVKDYFPEPVTVSWNSGALTSGVHTFPAVLQ |
| | | SSGLYSLSSVVTVPSSSLGTKTYTCNVDHKPSNTKVDKRVESKYGPPCPPCPAPEAAGGPS |
| | | VFLFPPKPKDTLMISRTPEVTCVVVDVSQEDPEVQFNWYVDGVEVHNAKTKPREEQFNST |
| | | YRVVSVLTVLHQDWLNGKEYKCKVSNKGLPSSIEKTISKAKGQPREPQVYTLPPSQEEMT |
| | | KNQVSLTCLVKGFYPSDIAVEWESNGQPENNYKTTPPVLDSDGSFFLYSRLTVDKSRWQE |
| | | GNVFSCSVMHEALHNHYTQKSLSLSLGK |
| | | SEQ ID NO: 173 |
| | HC CDR1 | TDAIH |
| | | SEQ ID NO: 175 |
| | HC CDR2 | YIIPISGNANYAQKFQG |
| | | SEQ ID NO: 176 |
| | HC CDR3 | GRYAYNWGFDY |
| | | SEQ ID NO: 177 |
| | Light chain | EIVLTQSPATLSLSPGERATLSCRASQSVSSYLAWYQQKPGQAPRLLIKDASYRATGIPARF |
| | RR1L73 | SGSGSGTDFTLTISSLEPEDFAVYYCQQRSNWPLTFGQGTKVEIKRTVAAPSVFIFPPSDEQ |
| | | LKSGTASVVCLLNNFYPREAKVQWKVDNALQSGNSQESVTEQDSKDSTYSLSSTLTLSKA |
| | | DYEKHKVYACEVTHQGLSSPVTKSFNRGEC |
| | | SEQ ID NO: 174 |
| | LC CDR1 | RASQSVSSYLA |
| | | SEQ ID NO: 178 |
| | LC CDR2 | DASYRAT |
| | | SEQ ID NO: 179 |
| | LC CDR3 | QQRSNWPLT |
| | | SEQ ID NO: 180 |
| RR1B187 | Heavy Chain | QVQLVQSGAEVKKPGSSVKVSCKASGGTFKSAYIHWVRQAPGQGLEWMGYISPYYGSA |
| | RR1H66 | YYAQKFQGRVTITADESTSTAYMELSSLRSEDTAVYYCARGRIAYYYVFDYWGQGTLVT |
| | huIgG4_nG4m(a)_PAA | VSSASTKGPSVFPLAPCSRSTSESTAALGCLVKDYFPEPVTVSWNSGALTSGVHTFPAVLQ |
| | | SSGLYSLSSVVTVPSSSLGTKTYTCNVDHKPSNTKVDKRVESKYGPPCPPCPAPEAAGGPS |
| | | VFLFPPKPKDTLMISRTPEVTCVVVDVSQEDPEVQFNWYVDGVEVHNAKTKPREEQFNST |
| | | YRVVSVLTVLHQDWLNGKEYKCKVSNKGLPSSIEKTISKAKGQPREPQVYTLPPSQEEMT |
| | | KNQVSLTCLVKGFYPSDIAVEWESNGQPENNYKTTPPVLDSDGSFFLYSRLTVDKSRWQE |
| | | GNVFSCSVMHEALHNHYTQKSLSLSLGK |
| | | SEQ ID NO: 181 |
| | HC CDR1 | SAYIH |
| | | SEQ ID NO: 183 |
| | HC CDR2 | YISPYYGSAYYAQKFQG |
| | | SEQ ID NO: 184 |
| | HC CDR3 | GRIAYYYVFDY |
| | | SEQ ID NO: 185 |
| | Light Chain | EIVLTQSPATLSLSPGERATLSCRASQSVSSYLAWYQQKPGQAPRLLIYDASYRATGIPARF |
| | RR1L102 | SGSGSGTDFTLTISSLEPEDFAVYYCQQRSNWPLTFGQGTKVEIKRTVAAPSVFIFPPSDEQ |
| | huKappa_Km3 | LKSGTASVVCLLNNFYPREAKVQWKVDNALQSGNSQESVTEQDSKDSTYSLSSTLTLSKA |
| | | DYEKHKVYACEVTHQGLSSPVTKSFNRGEC |
| | | SEQ ID NO: 182 |
| | LC CDR1 | RASQSVSSYLA |
| | | SEQ ID NO: 186 |
| | LC CDR2 | DASYRAT |
| | | SEQ ID NO: 187 |
| | LC CDR3 | QQRSNWPLT |
| | | SEQ ID NO: 188 |
| RR1B188 | Heavy Chain | QVQLVQSGAEVKKPGSSVKVSCKASGGTFSTYAIHWVRQAPGQGLEWMGRIIPIEGRAY |
| | RR1H52 | YAQKFQGRVTITADESTSTAYMELSSLRSEDTAVYYCARGRYSYRYWFDYWGQGTLVT |
| | huIgG4_nG4m(a)_PAA | VSSASTKGPSVFPLAPCSRSTSESTAALGCLVKDYFPEPVTVSWNSGALTSGVHTFPAVLQ |
| | | SSGLYSLSSVVTVPSSSLGTKTYTCNVDHKPSNTKVDKRVESKYGPPCPPCPAPEAAGGPS |
| | | VFLFPPKPKDTLMISRTPEVTCVVVDVSQEDPEVQFNWYVDGVEVHNAKTKPREEQFNST |
| | | YRVVSVLTVLHQDWLNGKEYKCKVSNKGLPSSIEKTISKAKGQPREPQVYTLPPSQEEMT |
| | | KNQVSLTCLVKGFYPSDIAVEWESNGQPENNYKTTPPVLDSDGSFFLYSRLTVDKSRWQE |
| | | GNVFSCSVMHEALHNHYTQKSLSLSLGK |
| | | SEQ ID NO: 189 |
| | HC CDR1 | TYAIH |
| | | SEQ ID NO: 191 |
| | HC CDR2 | RIIPIEGRAYYAQKFQG |
| | | SEQ ID NO: 192 |
| | HC CDR3 | GRYSYRYWFDY |
| | | SEQ ID NO: 193 |
| | Light Chain | EIVLTQSPATLSLSPGERATLSCRASQSVSSYLAWYQQKPGQAPRLLIYDASSRATGIPARF |
| | RR1L196 | SGSGSGTDFTLTISSLEPEDFAVYYCQQRSNWPLTFGQGTKVEIKRTVAAPSVFIFPPSDEQ |
| | huKappa_Km3 | LKSGTASVVCLLNNFYPREAKVQWKVDNALQSGNSQESVTEQDSKDSTYSLSSTLTLSKA |
| | | DYEKHKVYACEVTHQGLSSPVTKSFNRGEC |
| | | SEQ ID NO: 190 |
| | LC CDR1 | RASQSVSSYLA |
| | | SEQ ID NO: 194 |
| | LC CDR2 | DASSRAT |
| | | SEQ ID NO: 195 |
| | LC CDR3 | QQRSNWPLT |
| | | SEQ ID NO: 196 |
| RR1B189 | Heavy Chain | QVQLVQSGAEVKKPGSSVKVSCKASGGTFSDAAIHWVRQAPGQGLEWMGYIIPISGTAN |
| | RR1H65 | YAQKFQGRVTITADESTSTAYMELSSLRSEDTAVYYCARGRARYRWAFDYWGQGTLVT |
| | huIgG4_nG4m(a)_PAA | VSSASTKGPSVFPLAPCSRSTSESTAALGCLVKDYFPEPVTVSWNSGALTSGVHTFPAVLQ |
| | | SSGLYSLSSVVTVPSSSLGTKTYTCNVDHKPSNTKVDKRVESKYGPPCPPCPAPEAAGGPS |
| | | VFLFPPKPKDTLMISRTPEVTCVVVDVSQEDPEVQFNWYVDGVEVHNAKTKPREEQFNST |
| | | YRVVSVLTVLHQDWLNGKEYKCKVSNKGLPSSIEKTISKAKGQPREPQVYTLPPSQEEMT |
| | | KNQVSLTCLVKGFYPSDIAVEWESNGQPENNYKTTPPVLDSDGSFFLYSRLTVDKSRWQE |
| | | GNVFSCSVMHEALHNHYTQKSLSLSLGK |
| | | SEQ ID NO: 197 |
| | HC CDR1 | DAAIH |
| | | SEQ ID NO: 199 |
| | HC CDR2 | YIIPISGTANYAQKFQG |
| | | SEQ ID NO: 200 |
| | HC CDR3 | GRARYRWAFDY |
| | | SEQ ID NO: 201 |
| | Light Chain | EIVLTQSPATLSLSPGERATLSCRASQSVNNYLAWYQQKPGQAPRLLIYDASYRATGIPAR |
| | RR1L184 | FSGSGSGTDFTLTISSLEPEDFAVYYCQQRSNWPLTFGQGTKVEIKRTVAAPSVFIFPPSDE |
| | huKappa_Km3 | QLKSGTASVVCLLNNFYPREAKVQWKVDNALQSGNSQESVTEQDSKDSTYSLSSTLTLSK |
| | | ADYEKHKVYACEVTHQGLSSPVTKSFNRGEC |
| | | SEQ ID NO: 198 |
| | LC CDR1 | RASQSVNNYLA |
| | | SEQ ID NO: 202 |
| | LC CDR2 | DASYRAT |
| | | SEQ ID NO: 203 |
| | LC CDR3 | QQRSNWPLT |
| | | SEQ ID NO: 204 |
| RR1B190 | Heavy Chain | QVQLVQSGAEVKKPGSSVKVSCKASGGTFSTYAIHWVRQAPGQGLEWMGRIIPIEGRAY |
| | RR1H52 | YAQKFQGRVTITADESTSTAYMELSSLRSEDTAVYYCARGRYSYRYWFDYWGQGTLVT |
| | huIgG4_nG4m(a)_PAA | VSSASTKGPSVFPLAPCSRSTSESTAALGCLVKDYFPEPVTVSWNSGALTSGVHTFPAVLQ |
| | | SSGLYSLSSVVTVPSSSLGTKTYTCNVDHKPSNTKVDKRVESKYGPPCPPCPAPEAAGGPS |
| | | VFLFPPKPKDTLMISRTPEVTCVVVDVSQEDPEVQFNWYVDGVEVHNAKTKPREEQFNST |
| | | YRVVSVLTVLHQDWLNGKEYKCKVSNKGLPSSIEKTISKAKGQPREPQVYTLPPSQEEMT |
| | | KNQVSLTCLVKGFYPSDIAVEWESNGQPENNYKTTPPVLDSDGSFFLYSRLTVDKSRWQE |
| | | GNVFSCSVMHEALHNHYTQKSLSLSLGK |
| | | SEQ ID NO: 205 |
| | HC CDR1 | TYAIH |
| | | SEQ ID NO: 207 |
| | HC CDR2 | RIIPIEGRAYYAQKFQG |
| | | SEQ ID NO: 208 |
| | HC CDR3 | GRYSYRYWFDY |
| | | SEQ ID NO: 209 |
| | Light Chain | EIVLTQSPATLSLSPGERATLSCRASQSVSTYLAWYQQKPGQAPRLLIHDASYRATGIPARF |
| | RR1L212 | SGSGSGTDFTLTISSLEPEDFAVYYCQQRAHWPITFGQGTKVEIKRTVAAPSVFIFPPSDEQ |
| | huKappa_Km3 | LKSGTASVVCLLNNFYPREAKVQWKVDNALQSGNSQESVTEQDSKDSTYSLSSTLTLSKA |
| | | DYEKHKVYACEVTHQGLSSPVTKSFNRGEC |
| | | SEQ ID NO: 206 |
| | LC CDR1 | RASQSVSTYLA |
| | | SEQ ID NO: 210 |
| | LC CDR2 | DASYRAT |
| | | SEQ ID NO: 211 |
| | LC CDR3 | QQRAHWPIT |
| | | SEQ ID NO: 212 |
| RR1B191 | Heavy Chain | QVQLVQSGAEVKKPGSSVKVSCKASGGTFSTDAIHWVRQAPGQGLEWMGRIIPISGTANY |
| | RR1H51 | AQKFQGRVTITADESTSTAYMELSSLRSEDTAVYYCARGRYSYYYYFDYWGQGTLVTVS |
| | huIgG4_nG4m(a)_PAA | SASTKGPSVFPLAPCSRSTSESTAALGCLVKDYFPEPVTVSWNSGALTSGVHTFPAVLQSS |
| | | GLYSLSSVVTVPSSSLGTKTYTCNVDHKPSNTKVDKRVESKYGPPCPPCPAPEAAGGPSVF |
| | | LFPPKPKDTLMISRTPEVTCVVVDVSQEDPEVQFNWYVDGVEVHNAKTKPREEQFNSTYR |
| | | VVSVLTVLHQDWLNGKEYKCKVSNKGLPSSIEKTISKAKGQPREPQVYTLPPSQEEMTKN |
| | | QVSLTCLVKGFYPSDIAVEWESNGQPENNYKTTPPVLDSDGSFFLYSRLTVDKSRWQEGN |
| | | VFSCSVMHEALHNHYTQKSLSLSLGK |
| | | SEQ ID NO: 213 |
| | HC CDR1 | TDAIH |
| | | SEQ ID NO: 215 |
| | HC CDR2 | RIIPISGTANYAQKFQG |
| | | SEQ ID NO: 216 |
| | HC CDR3 | GRYSYYYYFDY |
| | | SEQ ID NO: 217 |
| | Light Chain | EIVLTQSPATLSLSPGERATLSCRASQSVSTYLAWYQQKPGQAPRLLIYDASNRATGIPARF |
| | RR1L228 | SGSGSGTDFTLTISSLEPEDFAVYYCQQRAHWPNTFGQGTKVEIKRTVAAPSVFIFPPSDEQ |
| | huKappa_Km3 | LKSGTASVVCLLNNFYPREAKVQWKVDNALQSGNSQESVTEQDSKDSTYSLSSTLTLSKA |
| | | DYEKHKVYACEVTHQGLSSPVTKSFNRGEC |
| | | SEQ ID NO: 214 |
| | LC CDR1 | RASQSVSTYLA |
| | | SEQ ID NO: 218 |
| | LC CDR2 | DASNRAT |
| | | SEQ ID NO: 219 |
| | LC CDR3 | QQRAHWPNT |
| | | SEQ ID NO: 220 |
| RR1B192 | Heavy Chain | QVQLVQSGAEVKKPGSSVKVSCKASGGTFSTYAIHWVRQAPGQGLEWMGRIIPIEGRAY |
| | RR1H52 | YAQKFQGRVTITADESTSTAYMELSSLRSEDTAVYYCARGRYSYRYWFDYWGQGTLVT |
| | huIgG4_nG4m(a)_PAA | VSSASTKGPSVFPLAPCSRSTSESTAALGCLVKDYFPEPVTVSWNSGALTSGVHTFPAVLQ |
| | | SSGLYSLSSVVTVPSSSLGTKTYTCNVDHKPSNTKVDKRVESKYGPPCPPCPAPEAAGGPS |
| | | VFLFPPKPKDTLMISRTPEVTCVVVDVSQEDPEVQFNWYVDGVEVHNAKTKPREEQFNST |
| | | YRVVSVLTVLHQDWLNGKEYKCKVSNKGLPSSIEKTISKAKGQPREPQVYTLPPSQEEMT |
| | | KNQVSLTCLVKGFYPSDIAVEWESNGQPENNYKTTPPVLDSDGSFFLYSRLTVDKSRWQE |
| | | GNVFSCSVMHEALHNHYTQKSLSLSLGK |
| | | SEQ ID NO: 221 |
| | HC CDR1 | TYAIH |
| | | SEQ ID NO: 223 |
| | HC CDR2 | RIIPIEGRAYYAQKFQG |
| | | SEQ ID NO: 224 |
| | HC CDR3 | GRYSYRYWFDY |
| | | SEQ ID NO: 225 |
| | Light Chain | EIVLTQSPATLSLSPGERATLSCRASQSVSTYLAWYQQKPGQAPRLLIYDASNRATGIPARF |
| | RR1L202 | SGSGSGTDFTLTISSLEPEDFAVYYCQQRAHSPLTFGQGTKVEIKRTVAAPSVFIFPPSDEQL |
| | huKappa_Km3 | KSGTASVVCLLNNFYPREAKVQWKVDNALQSGNSQESVTEQDSKDSTYSLSSTLTLSKA |
| | | DYEKHKVYACEVTHQGLSSPVTKSFNRGEC |
| | | SEQ ID NO: 222 |
| | LC CDR1 | RASQSVSTYLA |
| | | SEQ ID NO: 226 |
| | LC CDR2 | DASNRAT |
| | | SEQ ID NO: 227 |
| | LC CDR3 | QQRAHSPLT |
| | | SEQ ID NO: 228 |
| RR1B193 | Heavy Chain | QVQLVQSGAEVKKPGSSVKVSCKASGGTFSTYAIHWVRQAPGQGLEWMGRIIPIEGRAY |
| | RR1H52 | YAQKFQGRVTITADESTSTAYMELSSLRSEDTAVYYCARGRYSYRYWFDYWGQGTLVT |
| | huIgG4_nG4m(a)_PAA | VSSASTKGPSVFPLAPCSRSTSESTAALGCLVKDYFPEPVTVSWNSGALTSGVHTFPAVLQ |
| | | SSGLYSLSSVVTVPSSSLGTKTYTCNVDHKPSNTKVDKRVESKYGPPCPPCPAPEAAGGPS |
| | | VFLFPPKPKDTLMISRTPEVTCVVVDVSQEDPEVQFNWYVDGVEVHNAKTKPREEQFNST |
| | | YRVVSVLTVLHQDWLNGKEYKCKVSNKGLPSSIEKTISKAKGQPREPQVYTLPPSQEEMT |
| | | KNQVSLTCLVKGFYPSDIAVEWESNGQPENNYKTTPPVLDSDGSFFLYSRLTVDKSRWQE |
| | | GNVFSCSVMHEALHNHYTQKSLSLSLGK |
| | | SEQ ID NO: 229 |
| | HC CDR1 | TYAIH |
| | | SEQ ID NO: 231 |
| | HC CDR2 | RIIPIEGRAYYAQKFQG |
| | | SEQ ID NO: 232 |
| | HC CDR3 | GRYSYRYWFDY |
| | | SEQ ID NO: 233 |
| | Light Chain | EIVLTQSPATLSLSPGERATLSCRASQSVSNYLAWYQQKPGQAPRLLIHDASNRATGIPAR |
| | RR1L190 | FSGSGSGTDFTLTISSLEPEDFAVYYCQQRAHSPNTFGQGTKVEIKRTVAAPSVFIFPPSDEQ |
| | huKappa_Km3 | LKSGTASVVCLLNNFYPREAKVQWKVDNALQSGNSQESVTEQDSKDSTYSLSSTLTLSKA |
| | | DYEKHKVYACEVTHQGLSSPVTKSFNRGEC |
| | | SEQ ID NO: 230 |
| | LC CDR1 | RASQSVSNYLA |
| | | SEQ ID NO: 234 |
| | LC CDR2 | DASNRAT |
| | | SEQ ID NO: 235 |
| | LC CDR3 | QQRAHSPNT |
| | | SEQ ID NO: 236 |
| RR1B194 | Heavy Chain | QVQLVQSGAEVKKPGSSVKVSCKASGGTFSTYAIHWVRQAPGQGLEWMGRIIPIEGRAY |
| | RR1H52 | YAQKFQGRVTITADESTSTAYMELSSLRSEDTAVYYCARGRYSYRYWFDYWGQGTLVT |
| | huIgG4_nG4m(a)_PAA | VSSASTKGPSVFPLAPCSRSTSESTAALGCLVKDYFPEPVTVSWNSGALTSGVHTFPAVLQ |
| | | SSGLYSLSSVVTVPSSSLGTKTYTCNVDHKPSNTKVDKRVESKYGPPCPPCPAPEAAGGPS |
| | | VFLFPPKPKDTLMISRTPEVTCVVVDVSQEDPEVQFNWYVDGVEVHNAKTKPREEQFNST |
| | | YRVVSVLTVLHQDWLNGKEYKCKVSNKGLPSSIEKTISKAKGQPREPQVYTLPPSQEEMT |
| | | KNQVSLTCLVKGFYPSDIAVEWESNGQPENNYKTTPPVLDSDGSFFLYSRLTVDKSRWQE |
| | | GNVFSCSVMHEALHNHYTQKSLSLSLGK |
| | | SEQ ID NO: 237 |
| | HC CDR1 | TYAIH |
| | | SEQ ID NO: 239 |
| | HC CDR2 | RIIPIEGRAYYAQKFQG |
| | | SEQ ID NO: 240 |
| | HC CDR3 | GRYSYRYWFDY |
| | | SEQ ID NO: 241 |
| | Light Chain | EIVLTQSPATLSLSPGERATLSCRASQSVSSYLAWYQQKPGQAPRLLIEDASDRATGIPARF |
| | RR1L194 | SGSGSGTDFTLTISSLEPEDFAVYYCQQRSHWPLTFGQGTKVEIKRTVAAPSVFIFPPSDEQ |
| | huKappa_Km3 | LKSGTASVVCLLNNFYPREAKVQWKVDNALQSGNSQESVTEQDSKDSTYSLSSTLTLSKA |
| | | DYEKHKVYACEVTHQGLSSPVTKSFNRGEC |
| | | SEQ ID NO: 238 |
| | LC CDR1 | RASQSVSSYLA |
| | | SEQ ID NO: 242 |
| | LC CDR2 | DASDRAT |
| | | SEQ ID NO: 243 |
| | LC CDR3 | QQRSHWPLT |
| | | SEQ ID NO: 244 |
| RR1B196 | Heavy Chain | QVQLVQSGAEVKKPGSSVKVSCKASGGTFSTDAIHWVRQAPGQGLEWMGRIIPISGTANY |
| | RR1H51 | AQKFQGRVTITADESTSTAYMELSSLRSEDTAVYYCARGRYSYYYYFDYWGQGTLVTVS |
| | huIgG4_nG4m(a)_PAA | SASTKGPSVFPLAPCSRSTSESTAALGCLVKDYFPEPVTVSWNSGALTSGVHTFPAVLQSS |
| | | GLYSLSSVVTVPSSSLGTKTYTCNVDHKPSNTKVDKRVESKYGPPCPPCPAPEAAGGPSVF |
| | | LFPPKPKDTLMISRTPEVTCVVVDVSQEDPEVQFNWYVDGVEVHNAKTKPREEQFNSTYR |
| | | VVSVLTVLHQDWLNGKEYKCKVSNKGLPSSIEKTISKAKGQPREPQVYTLPPSQEEMTKN |
| | | QVSLTCLVKGFYPSDIAVEWESNGQPENNYKTTPPVLDSDGSFFLYSRLTVDKSRWQEGN |
| | | VFSCSVMHEALHNHYTQKSLSLSLGK |
| | | SEQ ID NO: 245 |
| | HC CDR1 | TDAIH |
| | | SEQ ID NO: 247 |
| | HC CDR2 | RIIPISGTANYAQKFQG |
| | | SEQ ID NO: 248 |
| | HC CDR3 | GRYSYYYYFDY |
| | | SEQ ID NO: 249 |
| | Light Chain | EIVLTQSPATLSLSPGERATLSCRASQSVSNYLAWYQQKPGQAPRLLIYDASNRATGIPAR |
| | RR1L218 | FSGSGSGTDFTLTISSLEPEDFAVYYCQQRSHWPNTFGQGTKVEIKRTVAAPSVFIFPPSDE |
| | huKappa_Km3 | QLKSGTASVVCLLNNFYPREAKVQWKVDNALQSGNSQESVTEQDSKDSTYSLSSTLTLSK |
| | | ADYEKHKVYACEVTHQGLSSPVTKSFNRGEC |
| | | SEQ ID NO: 246 |
| | LC CDR1 | RASQSVSNYLA |
| | | SEQ ID NO: 250 |
| | LC CDR2 | DASNRAT |
| | | SEQ ID NO: 251 |
| | LC CDR3 | QQRSHWPNT |
| | | SEQ ID NO: 252 |
| RR1B197 | Heavy Chain | QVQLVQSGAEVKKPGSSVKVSCKASGGTFSTDAIHWVRQAPGQGLEWMGRIIPISGTANY |
| | RR1H51 | AQKFQGRVTITADESTSTAYMELSSLRSEDTAVYYCARGRYSYYYYFDYWGQGTLVTVS |
| | huIgG4_nG4m(a)_PAA | SASTKGPSVFPLAPCSRSTSESTAALGCLVKDYFPEPVTVSWNSGALTSGVHTFPAVLQSS |
| | | GLYSLSSVVTVPSSSLGTKTYTCNVDHKPSNTKVDKRVESKYGPPCPPCPAPEAAGGPSVF |
| | | LFPPKPKDTLMISRTPEVTCVVVDVSQEDPEVQFNWYVDGVEVHNAKTKPREEQFNSTYR |
| | | VVSVLTVLHQDWLNGKEYKCKVSNKGLPSSIEKTISKAKGQPREPQVYTLPPSQEEMTKN |
| | | QVSLTCLVKGFYPSDIAVEWESNGQPENNYKTTPPVLDSDGSFFLYSRLTVDKSRWQEGN |
| | | VFSCSVMHEALHNHYTQKSLSLSLGK |
| | | SEQ ID NO: 253 |
| | HC CDR1 | TDAIH |
| | | SEQ ID NO: 255 |
| | HC CDR2 | RIIPISGTANYAQKFQG |
| | | SEQ ID NO: 256 |
| | HC CDR3 | GRYSYYYYFDY |
| | | SEQ ID NO: 257 |
| | Light Chain | EIVLTQSPATLSLSPGERATLSCRASQSVNNYLAWYQQKPGQAPRLLIYDASNRATGIPAR |
| | RR1L233 | FSGSGSGTDFTLTISSLEPEDFAVYYCQQRSHWPNTFGQGTKVEIKRTVAAPSVFIFPPSDE |
| | huKappa_Km3 | QLKSGTASVVCLLNNFYPREAKVQWKVDNALQSGNSQESVTEQDSKDSTYSLSSTLTLSK |
| | | ADYEKHKVYACEVTHQGLSSPVTKSFNRGEC |
| | | SEQ ID NO: 254 |
| | LC CDR1 | RASQSVNNYLA |
| | | SEQ ID NO: 258 |
| | LC CDR2 | DASNRAT |
| | | SEQ ID NO: 259 |
| | LC CDR3 | QQRSHWPNT |
| | | SEQ ID NO: 260 |
| RR1B198 | Heavy Chain | QVQLVQSGAEVKKPGSSVKVSCKASGGTFSTYAIHWVRQAPGQGLEWMGRIIPIEGRAY |
| | RR1H52 | YAQKFQGRVTITADESTSTAYMELSSLRSEDTAVYYCARGRYSYRYWFDYWGQGTLVT |
| | huIgG4_nG4m(a)_PAA | VSSASTKGPSVFPLAPCSRSTSESTAALGCLVKDYFPEPVTVSWNSGALTSGVHTFPAVLQ |
| | | SSGLYSLSSVVTVPSSSLGTKTYTCNVDHKPSNTKVDKRVESKYGPPCPPCPAPEAAGGPS |
| | | VFLFPPKPKDTLMISRTPEVTCVVVDVSQEDPEVQFNWYVDGVEVHNAKTKPREEQFNST |
| | | YRVVSVLTVLHQDWLNGKEYKCKVSNKGLPSSIEKTISKAKGQPREPQVYTLPPSQEEMT |
| | | KNQVSLTCLVKGFYPSDIAVEWESNGQPENNYKTTPPVLDSDGSFFLYSRLTVDKSRWQE |
| | | GNVFSCSVMHEALHNHYTQKSLSLSLGK |
| | | SEQ ID NO: 261 |
| | HC CDR1 | TYAIH |
| | | SEQ ID NO: 263 |
| | HC CDR2 | RIIPIEGRAYYAQKFQG |
| | | SEQ ID NO: 264 |
| | HC CDR3 | GRYSYRYWFDY |
| | | SEQ ID NO: 265 |
| | Light Chain | EIVLTQSPATLSLSPGERATLSCRASQSVSSYLAWYQQKPGQAPRLLIYDASYRATGIPARF |
| | RR1L216 | SGSGSGTDFTLTISSLEPEDFAVYYCQQRSHSPLTFGQGTKVEIKRTVAAPSVFIFPPSDEQL |
| | huKappa_Km3 | KSGTASVVCLLNNFYPREAKVQWKVDNALQSGNSQESVTEQDSKDSTYSLSSTLTLSKA |
| | | DYEKHKVYACEVTHQGLSSPVTKSFNRGEC |
| | | SEQ ID NO: 262 |
| | LC CDR1 | RASQSVSSYLA |
| | | SEQ ID NO: 266 |
| | LC CDR2 | DASYRAT |
| | | SEQ ID NO: 267 |
| | LC CDR3 | QQRSHSPLT |
| | | SEQ ID NO: 268 |
| RR1B199 | Heavy Chain | QVQLVQSGAEVKKPGSSVKVSCKASGGTFDSDAIHWVRQAPGQGLEWMGYIIPISGTAN |
| | RR1H62 | YAQKFQGRVTITADESTSTAYMELSSLRSEDTAVYYCARGRYSYRWTFDYWGQGTLVTV |
| | huIgG4_nG4m(a)_PAA | SSASTKGPSVFPLAPCSRSTSESTAALGCLVKDYFPEPVTVSWNSGALTSGVHTFPAVLQS |
| | | SGLYSLSSVVTVPSSSLGTKTYTCNVDHKPSNTKVDKRVESKYGPPCPPCPAPEAAGGPSV |
| | | FLFPPKPKDTLMISRTPEVTCVVVDVSQEDPEVQFNWYVDGVEVHNAKTKPREEQFNSTY |
| | | RVVSVLTVLHQDWLNGKEYKCKVSNKGLPSSIEKTISKAKGQPREPQVYTLPPSQEEMTK |
| | | NQVSLTCLVKGFYPSDIAVEWESNGQPENNYKTTPPVLDSDGSFFLYSRLTVDKSRWQEG |
| | | NVFSCSVMHEALHNHYTQKSLSLSLGK |
| | | SEQ ID NO: 269 |
| | HC CDR1 | SDAIH |
| | | SEQ ID NO: 271 |
| | HC CDR2 | YIIPISGTANYAQKFQG |
| | | SEQ ID NO: 272 |
| | HC CDR3 | GRYSYRWTFDY |
| | | SEQ ID NO: 273 |
| | Light Chain | EIVLTQSPATLSLSPGERATLSCRASQSVRNYLAWYQQKPGQAPRLLIEDASNRATGIPAR |
| | RR1L167 | FSGSGSGTDFTLTISSLEPEDFAVYYCQQRSHSPITFGQGTKVEIKRTVAAPSVFIFPPSDEQ |
| | huKappa_Km3 | LKSGTASVVCLLNNFYPREAKVQWKVDNALQSGNSQESVTEQDSKDSTYSLSSTLTLSKA |
| | | DYEKHKVYACEVTHQGLSSPVTKSFNRGEC |
| | | SEQ ID NO: 270 |
| | LC CDR1 | RASQSVRNYLA |
| | | SEQ ID NO: 274 |
| | LC CDR2 | DASNRAT |
| | | SEQ ID NO: 275 |
| | LC CDR3 | QQRSHSPIT |
| | | SEQ ID NO: 276 |
| RR1B200 | Heavy Chain | QVQLVQSGAEVKKPGSSVKVSCKASGGTFKSAYIHWVRQAPGQGLEWMGYISPYYGSA |
| | RR1H66 | YYAQKFQGRVTITADESTSTAYMELSSLRSEDTAVYYCARGRIAYYYVFDYWGQGTLVT |
| | huIgG4_nG4m(a)_PAA | VSSASTKGPSVFPLAPCSRSTSESTAALGCLVKDYFPEPVTVSWNSGALTSGVHTFPAVLQ |
| | | SSGLYSLSSVVTVPSSSLGTKTYTCNVDHKPSNTKVDKRVESKYGPPCPPCPAPEAAGGPS |
| | | VFLFPPKPKDTLMISRTPEVTCVVVDVSQEDPEVQFNWYVDGVEVHNAKTKPREEQFNST |
| | | YRVVSVLTVLHQDWLNGKEYKCKVSNKGLPSSIEKTISKAKGQPREPQVYTLPPSQEEMT |
| | | KNQVSLTCLVKGFYPSDIAVEWESNGQPENNYKTTPPVLDSDGSFFLYSRLTVDKSRWQE |
| | | GNVFSCSVMHEALHNHYTQKSLSLSLGK |
| | | SEQ ID NO: 277 |
| | HC CDR1 | SAYIH |
| | | SEQ ID NO: 279 |
| | HC CDR2 | YISPYYGSAYYAQKFQG |
| | | SEQ ID NO: 280 |
| | HC CDR3 | GRIAYYYVFDY |
| | | SEQ ID NO: 281 |
| | Light Chain | EIVLTQSPATLSLSPGERATLSCRASQSVSSYLAWYQQKPGQAPRLLIYDASNRATGIPARF |
| | RR1L106 | SGSGSGTDFTLTISSLEPEDFAVYYCQQRSNWPITFGQGTKVEIKRTVAAPSVFIFPPSDEQL |
| | huKappa_Km3 | KSGTASVVCLLNNFYPREAKVQWKVDNALQSGNSQESVTEQDSKDSTYSLSSTLTLSKA |
| | | DYEKHKVYACEVTHQGLSSPVTKSFNRGEC |
| | | SEQ ID NO: 278 |
| | LC CDR1 | RASQSVSSYLA |
| | | SEQ ID NO: 282 |
| | LC CDR2 | DASNRAT |
| | | SEQ ID NO: 283 |
| | LC CDR3 | QQRSNWPIT |
| | | SEQ ID NO: 284 |
| RR1B201 | Heavy Chain | QVQLVQSGAEVKKPGSSVKVSCKASGGTFDSDAIHWVRQAPGQGLEWMGYIIPISGTAN |
| | RR1H62 | YAQKFQGRVTITADESTSTAYMELSSLRSEDTAVYYCARGRYSYRWTFDYWGQGTLVTV |
| | huIgG4_nG4m(a)_PAA | SSASTKGPSVFPLAPCSRSTSESTAALGCLVKDYFPEPVTVSWNSGALTSGVHTFPAVLQS |
| | | SGLYSLSSVVTVPSSSLGTKTYTCNVDHKPSNTKVDKRVESKYGPPCPPCPAPEAAGGPSV |
| | | FLFPPKPKDTLMISRTPEVTCVVVDVSQEDPEVQFNWYVDGVEVHNAKTKPREEQFNSTY |
| | | RVVSVLTVLHQDWLNGKEYKCKVSNKGLPSSIEKTISKAKGQPREPQVYTLPPSQEEMTK |
| | | NQVSLTCLVKGFYPSDIAVEWESNGQPENNYKTTPPVLDSDGSFFLYSRLTVDKSRWQEG |
| | | NVFSCSVMHEALHNHYTQKSLSLSLGK |
| | | SEQ ID NO: 285 |
| | HC CDR1 | SDAIH |
| | | SEQ ID NO: 287 |
| | HC CDR2 | YIIPISGTANYAQKFQG |
| | | SEQ ID NO: 288 |
| | HC CDR3 | GRYSYRWTFDY |
| | | SEQ ID NO: 289 |
| | Light Chain | EIVLTQSPATLSLSPGERATLSCRASQSVSNYLAWYQQKPGQAPRLLIYDASNRATGIPAR |
| | RR1L143 | FSGSGSGTDFTLTISSLEPEDFAVYYCQQRSNWPITFGQGTKVEIKRTVAAPSVFIFPPSDEQ |
| | huKappa_Km3 | LKSGTASVVCLLNNFYPREAKVQWKVDNALQSGNSQESVTEQDSKDSTYSLSSTLTLSKA |
| | | DYEKHKVYACEVTHQGLSSPVTKSFNRGEC |
| | | SEQ ID NO: 286 |
| | LC CDR1 | RASQSVSNYLA |
| | | SEQ ID NO: 290 |
| | LC CDR2 | DASNRAT |
| | | SEQ ID NO: 291 |
| | LC CDR3 | QQRSNWPIT |
| | | SEQ ID NO: 292 |
| RR1B202 | Heavy Chain | QVQLVQSGAEVKKPGSSVKVSCKASGGTFSTDAIHWVRQAPGQGLEWMGRIIPISGTANY |
| | RR1H51 | AQKFQGRVTITADESTSTAYMELSSLRSEDTAVYYCARGRYSYYYYFDYWGQGTLVTVS |
| | huIgG4_nG4m(a)_PAA | SASTKGPSVFPLAPCSRSTSESTAALGCLVKDYFPEPVTVSWNSGALTSGVHTFPAVLQSS |
| | | GLYSLSSVVTVPSSSLGTKTYTCNVDHKPSNTKVDKRVESKYGPPCPPCPAPEAAGGPSVF |
| | | LFPPKPKDTLMISRTPEVTCVVVDVSQEDPEVQFNWYVDGVEVHNAKTKPREEQFNSTYR |
| | | VVSVLTVLHQDWLNGKEYKCKVSNKGLPSSIEKTISKAKGQPREPQVYTLPPSQEEMTKN |
| | | QVSLTCLVKGFYPSDIAVEWESNGQPENNYKTTPPVLDSDGSFFLYSRLTVDKSRWQEGN |
| | | VFSCSVMHEALHNHYTQKSLSLSLGK |
| | | SEQ ID NO: 293 |
| | HC CDR1 | TDAIH |
| | | SEQ ID NO: 295 |
| | HC CDR2 | RIIPISGTANYAQKFQG |
| | | SEQ ID NO: 296 |
| | HC CDR3 | GRYSYYYYFDY |
| | | SEQ ID NO: 297 |
| | Light Chain | EIVLTQSPATLSLSPGERATLSCRASQSVRSYLAWYQQKPGQAPRLLIYDASNRATGIPARF |
| | RR1L225 | SGSGSGTDFTLTISSLEPEDFAVYYCQQRSNWPITFGQGTKVEIKRTVAAPSVFIFPPSDEQL |
| | huKappa_Km3 | KSGTASVVCLLNNFYPREAKVQWKVDNALQSGNSQESVTEQDSKDSTYSLSSTLTLSKA |
| | | DYEKHKVYACEVTHQGLSSPVTKSFNRGEC |
| | | SEQ ID NO: 294 |
| | LC CDR1 | RASQSVRSYLA |
| | | SEQ ID NO: 298 |
| | LC CDR2 | DASNRAT |
| | | SEQ ID NO: 299 |
| | LC CDR3 | QQRSNWPIT |
| | | SEQ ID NO: 300 |
| RR1B203 | Heavy Chain | QVQLVQSGAEVKKPGSSVKVSCKASGGTFKSAYIHWVRQAPGQGLEWMGYISPYYGSA |
| | RR1H66 | YYAQKFQGRVTITADESTSTAYMELSSLRSEDTAVYYCARGRIAYYYVFDYWGQGTLVT |
| | huIgG4_nG4m(a)_PAA | VSSASTKGPSVFPLAPCSRSTSESTAALGCLVKDYFPEPVTVSWNSGALTSGVHTFPAVLQ |
| | | SSGLYSLSSVVTVPSSSLGTKTYTCNVDHKPSNTKVDKRVESKYGPPCPPCPAPEAAGGPS |
| | | VFLFPPKPKDTLMISRTPEVTCVVVDVSQEDPEVQFNWYVDGVEVHNAKTKPREEQFNST |
| | | YRVVSVLTVLHQDWLNGKEYKCKVSNKGLPSSIEKTISKAKGQPREPQVYTLPPSQEEMT |
| | | KNQVSLTCLVKGFYPSDIAVEWESNGQPENNYKTTPPVLDSDGSFFLYSRLTVDKSRWQE |
| | | GNVFSCSVMHEALHNHYTQKSLSLSLGK |
| | | SEQ ID NO: 301 |
| | HC CDR1 | SAYIH |
| | | SEQ ID NO: 303 |
| | HC CDR2 | YISPYYGSAYYAQKFQG |
| | | SEQ ID NO: 304 |
| | HC CDR3 | GRIAYYYVFDY |
| | | SEQ ID NO: 305 |
| | Light Chain | EIVLTQSPATLSLSPGERATLSCRASQSVRNYLAWYQQKPGQAPRLLIYDASNRATGIPAR |
| | RR1L117 | FSGSGSGTDFTLTISSLEPEDFAVYYCQQRSNWPITFGQGTKVEIKRTVAAPSVFIFPPSDEQ |
| | huKappa_Km3 | LKSGTASVVCLLNNFYPREAKVQWKVDNALQSGNSQESVTEQDSKDSTYSLSSTLTLSKA |
| | | DYEKHKVYACEVTHQGLSSPVTKSFNRGEC |
| | | SEQ ID NO: 302 |
| | LC CDR1 | RASQSVRNYLA |
| | | SEQ ID NO: 306 |
| | LC CDR2 | DASNRAT |
| | | SEQ ID NO: 307 |
| | LC CDR3 | QQRSNWPIT |
| | | SEQ ID NO: 308 |
| RR1B204 | Heavy Chain | QVQLVQSGAEVKKPGSSVKVSCKASGGTFSTDAIHWVRQAPGQGLEWMGYIIPISGNAN |
| | RR1H69 | YAQKFQGRVTITADESTSTAYMELSSLRSEDTAVYYCARGRYAYNWGFDYWGQGTLVT |
| | huIgG4_nG4m(a)_PAA | VSSASTKGPSVFPLAPCSRSTSESTAALGCLVKDYFPEPVTVSWNSGALTSGVHTFPAVLQ |
| | | SSGLYSLSSVVTVPSSSLGTKTYTCNVDHKPSNTKVDKRVESKYGPPCPPCPAPEAAGGPS |
| | | VFLFPPKPKDTLMISRTPEVTCVVVDVSQEDPEVQFNWYVDGVEVHNAKTKPREEQFNST |
| | | YRVVSVLTVLHQDWLNGKEYKCKVSNKGLPSSIEKTISKAKGQPREPQVYTLPPSQEEMT |
| | | KNQVSLTCLVKGFYPSDIAVEWESNGQPENNYKTTPPVLDSDGSFFLYSRLTVDKSRWQE |
| | | GNVFSCSVMHEALHNHYTQKSLSLSLGK |
| | | SEQ ID NO: 309 |
| | HC CDR1 | TDAIH |
| | | SEQ ID NO: 311 |
| | HC CDR2 | YIIPISGNANYAQKFQG |
| | | SEQ ID NO: 312 |
| | HC CDR3 | GRYAYNWGFDY |
| | | SEQ ID NO: 313 |
| | Light Chain | EIVLTQSPATLSLSPGERATLSCRASQSVSTYLAWYQQKPGQAPRLLIKDASNRATGIPARF |
| | RR1L95 | SGSGSGTDFTLTISSLEPEDFAVYYCQQRSNWPITFGQGTKVEIKRTVAAPSVFIFPPSDEQL |
| | huKappa_Km3 | KSGTASVVCLLNNFYPREAKVQWKVDNALQSGNSQESVTEQDSKDSTYSLSSTLTLSKA |
| | | DYEKHKVYACEVTHQGLSSPVTKSFNRGEC |
| | | SEQ ID NO: 310 |
| | LC CDR1 | RASQSVSTYLA |
| | | SEQ ID NO: 314 |
| | LC CDR2 | DASNRAT |
| | | SEQ ID NO: 315 |
| | LC CDR3 | QQRSNWPIT |
| | | SEQ ID NO: 316 |
| RR1B205 | Heavy Chain | QVQLVQSGAEVKKPGSSVKVSCKASGGTFKSAYIHWVRQAPGQGLEWMGYISPYYGSA |
| | RR1H66 | YYAQKFQGRVTITADESTSTAYMELSSLRSEDTAVYYCARGRIAYYYVFDYWGQGTLVT |
| | huIgG4_nG4m(a)_PAA | VSSASTKGPSVFPLAPCSRSTSESTAALGCLVKDYFPEPVTVSWNSGALTSGVHTFPAVLQ |
| | | SSGLYSLSSVVTVPSSSLGTKTYTCNVDHKPSNTKVDKRVESKYGPPCPPCPAPEAAGGPS |
| | | VFLFPPKPKDTLMISRTPEVTCVVVDVSQEDPEVQFNWYVDGVEVHNAKTKPREEQFNST |
| | | YRVVSVLTVLHQDWLNGKEYKCKVSNKGLPSSIEKTISKAKGQPREPQVYTLPPSQEEMT |
| | | KNQVSLTCLVKGFYPSDIAVEWESNGQPENNYKTTPPVLDSDGSFFLYSRLTVDKSRWQE |
| | | GNVFSCSVMHEALHNHYTQKSLSLSLGK |
| | | SEQ ID NO: 317 |
| | HC CDR1 | SAYIH |
| | | SEQ ID NO: 319 |
| | HC CDR2 | YISPYYGSAYYAQKFQG |
| | | SEQ ID NO: 320 |
| | HC CDR3 | GRIAYYYVFDY |
| | | SEQ ID NO: 321 |
| | Light Chain | EIVLTQSPATLSLSPGERATLSCRASQSVSSYLAWYQQKPGQAPRLLIKDASDRATGIPARF |
| | RR1L107 | SGSGSGTDFTLTISSLEPEDFAVYYCQQRSNWPITFGQGTKVEIKRTVAAPSVFIFPPSDEQL |
| | huKappa_Km3 | KSGTASVVCLLNNFYPREAKVQWKVDNALQSGNSQESVTEQDSKDSTYSLSSTLTLSKA |
| | | DYEKHKVYACEVTHQGLSSPVTKSFNRGEC |
| | | SEQ ID NO: 318 |
| | LC CDR1 | RASQSVSSYLA |
| | | SEQ ID NO: 322 |
| | LC CDR2 | DASDRAT |
| | | SEQ ID NO: 323 |
| | LC CDR3 | QQRSNWPIT |
| | | SEQ ID NO: 324 |
| RR1B206 | Heavy Chain | QVQLVQSGAEVKKPGSSVKVSCKASGGTFSTYAIHWVRQAPGQGLEWMGRIIPIEGRAY |
| | RR1H52 | YAQKFQGRVTITADESTSTAYMELSSLRSEDTAVYYCARGRYSYRYWFDYWGQGTLVT |
| | huIgG4_nG4m(a)_PAA | VSSASTKGPSVFPLAPCSRSTSESTAALGCLVKDYFPEPVTVSWNSGALTSGVHTFPAVLQ |
| | | SSGLYSLSSVVTVPSSSLGTKTYTCNVDHKPSNTKVDKRVESKYGPPCPPCPAPEAAGGPS |
| | | VFLFPPKPKDTLMISRTPEVTCVVVDVSQEDPEVQFNWYVDGVEVHNAKTKPREEQFNST |
| | | YRVVSVLTVLHQDWLNGKEYKCKVSNKGLPSSIEKTISKAKGQPREPQVYTLPPSQEEMT |
| | | KNQVSLTCLVKGFYPSDIAVEWESNGQPENNYKTTPPVLDSDGSFFLYSRLTVDKSRWQE |
| | | GNVFSCSVMHEALHNHYTQKSLSLSLGK |
| | | SEQ ID NO: 325 |
| | HC CDR1 | TYAIH |
| | | SEQ ID NO: 327 |
| | HC CDR2 | RIIPIEGRAYYAQKFQG |
| | | SEQ ID NO: 328 |
| | HC CDR3 | GRYSYRYWFDY |
| | | SEQ ID NO: 329 |
| | Light Chain | EIVLTQSPATLSLSPGERATLSCRASQSVSNYLAWYQQKPGQAPRLLIYDASNRATGIPAR |
| | RR1L209 | FSGSGSGTDFTLTISSLEPEDFAVYYCQQRSNWPNTFGQGTKVEIKRTVAAPSVFIFPPSDE |
| | huKappa_Km3 | QLKSGTASVVCLLNNFYPREAKVQWKVDNALQSGNSQESVTEQDSKDSTYSLSSTLTLSK |
| | | ADYEKHKVYACEVTHQGLSSPVTKSFNRGEC |
| | | SEQ ID NO: 326 |
| | LC CDR1 | RASQSVSNYLA |
| | | SEQ ID NO: 330 |
| | LC CDR2 | DASNRAT |
| | | SEQ ID NO: 331 |
| | LC CDR3 | QQRSNWPNT |
| | | SEQ ID NO: 332 |
| RR1B207 | Heavy Chain | QVQLVQSGAEVKKPGSSVKVSCKASGGTFSTYAIHWVRQAPGQGLEWMGRIIPIEGRAY |
| | RR1H52 | YAQKFQGRVTITADESTSTAYMELSSLRSEDTAVYYCARGRYSYRYWFDYWGQGTLVT |
| | huIgG4_nG4m(a)_PAA | VSSASTKGPSVFPLAPCSRSTSESTAALGCLVKDYFPEPVTVSWNSGALTSGVHTFPAVLQ |
| | | SSGLYSLSSVVTVPSSSLGTKTYTCNVDHKPSNTKVDKRVESKYGPPCPPCPAPEAAGGPS |
| | | VFLFPPKPKDTLMISRTPEVTCVVVDVSQEDPEVQFNWYVDGVEVHNAKTKPREEQFNST |
| | | YRVVSVLTVLHQDWLNGKEYKCKVSNKGLPSSIEKTISKAKGQPREPQVYTLPPSQEEMT |
| | | KNQVSLTCLVKGFYPSDIAVEWESNGQPENNYKTTPPVLDSDGSFFLYSRLTVDKSRWQE |
| | | GNVFSCSVMHEALHNHYTQKSLSLSLGK |
| | | SEQ ID NO: 333 |
| | HC CDR1 | TYAIH |
| | | SEQ ID NO: 335 |
| | HC CDR2 | RIIPIEGRAYYAQKFQG |
| | | SEQ ID NO: 336 |
| | HC CDR3 | GRYSYRYWFDY |
| | | SEQ ID NO: 337 |
| | Light Chain | EIVLTQSPATLSLSPGERATLSCRASQSVSNYLAWYQQKPGQAPRLLIHDASNRATGIPAR |
| | RR1L213 | FSGSGSGTDFTLTISSLEPEDFAVYYCQQRSNWPNTFGQGTKVEIKRTVAAPSVFIFPPSDE |
| | huKappa_Km3 | QLKSGTASVVCLLNNFYPREAKVQWKVDNALQSGNSQESVTEQDSKDSTYSLSSTLTLSK |
| | | ADYEKHKVYACEVTHQGLSSPVTKSFNRGEC |
| | | SEQ ID NO: 334 |
| | LC CDR1 | RASQSVSNYLA |
| | | SEQ ID NO: 338 |
| | LC CDR2 | DASNRAT |
| | | SEQ ID NO: 339 |
| | LC CDR3 | QQRSNWPNT |
| | | SEQ ID NO: 340 |
| RR1B208 | Heavy Chain | QVQLVQSGAEVKKPGSSVKVSCKASGGTFSTDAIHWVRQAPGQGLEWMGRIIPISGTANY |
| | RR1H51 | AQKFQGRVTITADESTSTAYMELSSLRSEDTAVYYCARGRYSYYYYFDYWGQGTLVTVS |
| | huIgG4_nG4m(a)_PAA | SASTKGPSVFPLAPCSRSTSESTAALGCLVKDYFPEPVTVSWNSGALTSGVHTFPAVLQSS |
| | | GLYSLSSVVTVPSSSLGTKTYTCNVDHKPSNTKVDKRVESKYGPPCPPCPAPEAAGGPSVF |
| | | LFPPKPKDTLMISRTPEVTCVVVDVSQEDPEVQFNWYVDGVEVHNAKTKPREEQFNSTYR |
| | | VVSVLTVLHQDWLNGKEYKCKVSNKGLPSSIEKTISKAKGQPREPQVYTLPPSQEEMTKN |
| | | QVSLTCLVKGFYPSDIAVEWESNGQPENNYKTTPPVLDSDGSFFLYSRLTVDKSRWQEGN |
| | | VFSCSVMHEALHNHYTQKSLSLSLGK |
| | | SEQ ID NO: 341 |
| | HC CDR1 | TDAIH |
| | | SEQ ID NO: 343 |
| | HC CDR2 | RIIPISGTANYAQKFQG |
| | | SEQ ID NO: 344 |
| | HC CDR3 | GRYSYYYYFDY |
| | | SEQ ID NO: 345 |
| | Light Chain | EIVLTQSPATLSLSPGERATLSCRASQSVSTYLAWYQQKPGQAPRLLIYDASNRATGIPARF |
| | RR1L230 | SGSGSGTDFTLTISSLEPEDFAVYYCQQRSNWPITFGQGTKVEIKRTVAAPSVFIFPPSDEQL |
| | huKappa_Km3 | KSGTASVVCLLNNFYPREAKVQWKVDNALQSGNSQESVTEQDSKDSTYSLSSTLTLSKA |
| | | DYEKHKVYACEVTHQGLSSPVTKSFNRGEC |
| | | SEQ ID NO: 342 |
| | LC CDR1 | RASQSVSTYLA |
| | | SEQ ID NO: 346 |
| | LC CDR2 | DASNRAT |
| | | SEQ ID NO: 347 |
| | LC CDR3 | QQRSNWPIT |
| | | SEQ ID NO: 348 |
| RR1B209 | Heavy Chain | QVQLVQSGAEVKKPGSSVKVSCKASGGTFDSDAIHWVRQAPGQGLEWMGYIIPISGTAN |
| | RR1H62 | YAQKFQGRVTITADESTSTAYMELSSLRSEDTAVYYCARGRYSYRWTFDYWGQGTLVTV |
| | huIgG4_nG4m(a)_PAA | SSASTKGPSVFPLAPCSRSTSESTAALGCLVKDYFPEPVTVSWNSGALTSGVHTFPAVLQS |
| | | SGLYSLSSVVTVPSSSLGTKTYTCNVDHKPSNTKVDKRVESKYGPPCPPCPAPEAAGGPSV |
| | | FLFPPKPKDTLMISRTPEVTCVVVDVSQEDPEVQFNWYVDGVEVHNAKTKPREEQFNSTY |
| | | RVVSVLTVLHQDWLNGKEYKCKVSNKGLPSSIEKTISKAKGQPREPQVYTLPPSQEEMTK |
| | | NQVSLTCLVKGFYPSDIAVEWESNGQPENNYKTTPPVLDSDGSFFLYSRLTVDKSRWQEG |
| | | NVFSCSVMHEALHNHYTQKSLSLSLGK |
| | | SEQ ID NO: 349 |
| | HC CDR1 | SDAIH |
| | | SEQ ID NO: 351 |
| | HC CDR2 | YIIPISGTANYAQKFQG |
| | | SEQ ID NO: 352 |
| | HC CDR3 | GRYSYRWTFDY |
| | | SEQ ID NO: 353 |
| | Light Chain | EIVLTQSPATLSLSPGERATLSCRASQSVSTYLAWYQQKPGQAPRLLIYDASNRATGIPARF |
| | RR1L123 | SGSGSGTDFTLTISSLEPEDFAVYYCQQRSNSPITFGQGTKVEIKRTVAAPSVFIFPPSDEQL |
| | huKappa_Km3 | KSGTASVVCLLNNFYPREAKVQWKVDNALQSGNSQESVTEQDSKDSTYSLSSTLTLSKA |
| | | DYEKHKVYACEVTHQGLSSPVTKSFNRGEC |
| | | SEQ ID NO: 350 |
| | LC CDR1 | RASQSVSTYLA |
| | | SEQ ID NO: 354 |
| | LC CDR2 | DASNRAT |
| | | SEQ ID NO: 355 |
| | LC CDR3 | QQRSNSPIT |
| | | SEQ ID NO: 356 |
| RR1B210 | Heavy Chain | QVQLVQSGAEVKKPGSSVKVSCKASGGTFDSDAIHWVRQAPGQGLEWMGYIIPISGTAN |
| | RR1H62 | YAQKFQGRVTITADESTSTAYMELSSLRSEDTAVYYCARGRYSYRWTFDYWGQGTLVTV |
| | huIgG4_nG4m(a)_PAA | SSASTKGPSVFPLAPCSRSTSESTAALGCLVKDYFPEPVTVSWNSGALTSGVHTFPAVLQS |
| | | SGLYSLSSVVTVPSSSLGTKTYTCNVDHKPSNTKVDKRVESKYGPPCPPCPAPEAAGGPSV |
| | | FLFPPKPKDTLMISRTPEVTCVVVDVSQEDPEVQFNWYVDGVEVHNAKTKPREEQFNSTY |
| | | RVVSVLTVLHQDWLNGKEYKCKVSNKGLPSSIEKTISKAKGQPREPQVYTLPPSQEEMTK |
| | | NQVSLTCLVKGFYPSDIAVEWESNGQPENNYKTTPPVLDSDGSFFLYSRLTVDKSRWQEG |
| | | NVFSCSVMHEALHNHYTQKSLSLSLGK |
| | | SEQ ID NO: 357 |
| | HC CDR1 | SDAIH |
| | | SEQ ID NO: 359 |
| | HC CDR2 | YIIPISGTANYAQKFQG |
| | | SEQ ID NO: 360 |
| | HC CDR3 | GRYSYRWTFDY |
| | | SEQ ID NO: 361 |
| | Light Chain | EIVLTQSPATLSLSPGERATLSCRASQSVSTYLAWYQQKPGQAPRLLIEDASYRATGIPARF |
| | RR1L127 | SGSGSGTDFTLTISSLEPEDFAVYYCQQRSNSPITFGQGTKVEIKRTVAAPSVFIFPPSDEQL |
| | huKappa_Km3 | KSGTASVVCLLNNFYPREAKVQWKVDNALQSGNSQESVTEQDSKDSTYSLSSTLTLSKA |
| | | DYEKHKVYACEVTHQGLSSPVTKSFNRGEC |
| | | SEQ ID NO: 358 |
| | LC CDR1 | RASQSVSTYLA |
| | | SEQ ID NO: 362 |
| | LC CDR2 | DASYRAT |
| | | SEQ ID NO: 363 |
| | LC CDR3 | QQRSNSPIT |
| | | SEQ ID NO: 364 |
| RR1B211 | Heavy Chain | QVQLVQSGAEVKKPGSSVKVSCKASGGTFSDAAIHWVRQAPGQGLEWMGYIIPISGTAN |
| | RR1H65 | YAQKFQGRVTITADESTSTAYMELSSLRSEDTAVYYCARGRARYRWAFDYWGQGTLVT |
| | huIgG4_nG4m(a)_PAA | VSSASTKGPSVFPLAPCSRSTSESTAALGCLVKDYFPEPVTVSWNSGALTSGVHTFPAVLQ |
| | | SSGLYSLSSVVTVPSSSLGTKTYTCNVDHKPSNTKVDKRVESKYGPPCPPCPAPEAAGGPS |
| | | VFLFPPKPKDTLMISRTPEVTCVVVDVSQEDPEVQFNWYVDGVEVHNAKTKPREEQFNST |
| | | YRVVSVLTVLHQDWLNGKEYKCKVSNKGLPSSIEKTISKAKGQPREPQVYTLPPSQEEMT |
| | | KNQVSLTCLVKGFYPSDIAVEWESNGQPENNYKTTPPVLDSDGSFFLYSRLTVDKSRWQE |
| | | GNVFSCSVMHEALHNHYTQKSLSLSLGK |
| | | SEQ ID NO: 365 |
| | HC CDR1 | DAAIH |
| | | SEQ ID NO: 367 |
| | HC CDR2 | YIIPISGTANYAQKFQG |
| | | SEQ ID NO: 368 |
| | HC CDR3 | GRARYRWAFDY |
| | | SEQ ID NO: 369 |
| | Light Chain | EIVLTQSPATLSLSPGERATLSCRASQSVSNYLAWYQQKPGQAPRLLIEDASNRATGIPARF |
| | RR1L182 | SGSGSGTDFTLTISSLEPEDFAVYYCQQRSNYPLTFGQGTKVEIKRTVAAPSVFIFPPSDEQL |
| | huKappa_Km3 | KSGTASVVCLLNNFYPREAKVQWKVDNALQSGNSQESVTEQDSKDSTYSLSSTLTLSKA |
| | | DYEKHKVYACEVTHQGLSSPVTKSFNRGEC |
| | | SEQ ID NO: 366 |
| | LC CDR1 | RASQSVSNYLA |
| | | SEQ ID NO: 370 |
| | LC CDR2 | DASNRAT |
| | | SEQ ID NO: 371 |
| | LC CDR3 | QQRSNYPLT |
| | | SEQ ID NO: 372 |
| RR1B212 | Heavy Chain | QVQLVQSGAEVKKPGSSVKVSCKASGGTFDSDAIHWVRQAPGQGLEWMGYIIPISGTAN |
| | RR1H62 | YAQKFQGRVTITADESTSTAYMELSSLRSEDTAVYYCARGRYSYRWTFDYWGQGTLVTV |
| | huIgG4_nG4m(a)_PAA | SSASTKGPSVFPLAPCSRSTSESTAALGCLVKDYFPEPVTVSWNSGALTSGVHTFPAVLQS |
| | | SGLYSLSSVVTVPSSSLGTKTYTCNVDHKPSNTKVDKRVESKYGPPCPPCPAPEAAGGPSV |
| | | FLFPPKPKDTLMISRTPEVTCVVVDVSQEDPEVQFNWYVDGVEVHNAKTKPREEQFNSTY |
| | | RVVSVLTVLHQDWLNGKEYKCKVSNKGLPSSIEKTISKAKGQPREPQVYTLPPSQEEMTK |
| | | NQVSLTCLVKGFYPSDIAVEWESNGQPENNYKTTPPVLDSDGSFFLYSRLTVDKSRWQEG |
| | | NVFSCSVMHEALHNHYTQKSLSLSLGK |
| | | SEQ ID NO: 373 |
| | HC CDR1 | SDAIH |
| | | SEQ ID NO: 375 |
| | HC CDR2 | YIIPISGTANYAQKFQG |
| | | SEQ ID NO: 376 |
| | HC CDR3 | GRYSYRWTFDY |
| | | SEQ ID NO: 377 |
| | Light Chain | EIVLTQSPATLSLSPGERATLSCRASQSVRNYLAWYQQKPGQAPRLLIYDASSRATGIPARF |
| | RR1L174 | SGSGSGTDFTLTISSLEPEDFAVYYCQQRSNYPITFGQGTKVEIKRTVAAPSVFIFPPSDEQL |
| | huKappa_Km3 | KSGTASVVCLLNNFYPREAKVQWKVDNALQSGNSQESVTEQDSKDSTYSLSSTLTLSKA |
| | | DYEKHKVYACEVTHQGLSSPVTKSFNRGEC |
| | | SEQ ID NO: 374 |
| | LC CDR1 | RASQSVRNYLA |
| | | SEQ ID NO: 378 |
| | LC CDR2 | DASSRAT |
| | | SEQ ID NO: 379 |
| | LC CDR3 | QQRSNYPIT |
| | | SEQ ID NO: 380 |
| RR1B213 | Heavy Chain | QVQLVQSGAEVKKPGSSVKVSCKASGGTFDSDAIHWVRQAPGQGLEWMGYIIPISGTAN |
| | RR1H62 | YAQKFQGRVTITADESTSTAYMELSSLRSEDTAVYYCARGRYSYRWTFDYWGQGTLVTV |
| | huIgG4_nG4m(a)_PAA | SSASTKGPSVFPLAPCSRSTSESTAALGCLVKDYFPEPVTVSWNSGALTSGVHTFPAVLQS |
| | | SGLYSLSSVVTVPSSSLGTKTYTCNVDHKPSNTKVDKRVESKYGPPCPPCPAPEAAGGPSV |
| | | FLFPPKPKDTLMISRTPEVTCVVVDVSQEDPEVQFNWYVDGVEVHNAKTKPREEQFNSTY |
| | | RVVSVLTVLHQDWLNGKEYKCKVSNKGLPSSIEKTISKAKGQPREPQVYTLPPSQEEMTK |
| | | NQVSLTCLVKGFYPSDIAVEWESNGQPENNYKTTPPVLDSDGSFFLYSRLTVDKSRWQEG |
| | | NVFSCSVMHEALHNHYTQKSLSLSLGK |
| | | SEQ ID NO: 381 |
| | HC CDR1 | SDAIH |
| | | SEQ ID NO: 383 |
| | HC CDR2 | YIIPISGTANYAQKFQG |
| | | SEQ ID NO: 384 |
| | HC CDR3 | GRYSYRWTFDY |
| | | SEQ ID NO: 385 |
| | Light Chain | EIVLTQSPATLSLSPGERATLSCRASQSVRNYLAWYQQKPGQAPRLLIYDASNRATGIPAR |
| | RR1L117 | FSGSGSGTDFTLTISSLEPEDFAVYYCQQRSNWPITFGQGTKVEIKRTVAAPSVFIFPPSDEQ |
| | huKappa_Km3 | LKSGTASVVCLLNNFYPREAKVQWKVDNALQSGNSQESVTEQDSKDSTYSLSSTLTLSKA |
| | | DYEKHKVYACEVTHQGLSSPVTKSFNRGEC |
| | | SEQ ID NO: 382 |
| | LC CDR1 | RASQSVRNYLA |
| | | SEQ ID NO: 386 |
| | LC CDR2 | DASNRAT |
| | | SEQ ID NO: 387 |
| | LC CDR3 | QQRSNWPIT |
| | | SEQ ID NO: 388 |

What is claimed:

1. An isolated antibody, or antigen-binding fragment thereof, that immunospecifically binds to ROR1, the antibody or antigen-binding fragment thereof comprising:
a. a heavy chain CDR1 comprising the amino acid sequence of SEQ ID NO:2, a heavy chain CDR2 comprising the amino acid sequence of SEQ ID NO:3, a heavy chain CDR3 comprising the amino acid sequence of SEQ ID NO:4, a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8;
b. a heavy chain CDR1 comprising the amino acid sequence of SEQ ID NO:10, a heavy chain CDR2 comprising the amino acid sequence of SEQ ID NO:11, a heavy chain CDR3 comprising the amino acid sequence of SEQ ID NO:12, a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8;
c. a heavy chain CDR1 comprising the amino acid sequence of SEQ ID NO:14, a heavy chain CDR2 comprising the amino acid sequence of SEQ ID NO:15, a heavy chain CDR3 comprising the amino acid sequence of SEQ ID NO:16, a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8;
d. a heavy chain CDR1 comprising the amino acid sequence of SEQ ID NO:18, a heavy chain CDR2 comprising the amino acid sequence of SEQ ID NO:19, a heavy chain CDR3 comprising the amino acid sequence of SEQ ID NO:20, a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8;
e. a heavy chain CDR1 comprising the amino acid sequence of SEQ ID NO:22, a heavy chain CDR2 comprising the amino acid sequence of SEQ ID NO:23, a heavy chain CDR3 comprising the amino acid sequence of SEQ ID NO:24, a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8;
f. a heavy chain CDR1 comprising the amino acid sequence of SEQ ID NO:26, a heavy chain CDR2 comprising the amino acid sequence of SEQ ID NO:27, a heavy chain CDR3 comprising the amino acid sequence of SEQ ID NO:28, a light chain CDR1 comprising the amino acid sequence of SEQ ID NO:30, a light chain CDR2 comprising the amino acid sequence of SEQ ID NO:31, and a light chain CDR3 comprising the amino acid sequence of SEQ ID NO:32;
g. a heavy chain CDR1 comprising the amino acid sequence of SEQ ID NO:2, a heavy chain CDR2 comprising the amino acid sequence of SEQ ID NO:34, a heavy chain CDR3 comprising the amino acid sequence of SEQ ID NO:35, a light chain CDR1 comprising the amino acid sequence of SEQ ID NO:37, a light chain CDR2 comprising the amino acid sequence of SEQ ID NO:7, and a light chain CDR3 comprising the amino acid sequence of SEQ ID NO:38;
h. a heavy chain CDR1 comprising the amino acid sequence of SEQ ID NO:22, a heavy chain CDR2 comprising the amino acid sequence of SEQ ID NO:23, a heavy chain CDR3 comprising the amino acid sequence of SEQ ID NO:40, a light chain CDR1 comprising the amino acid sequence of SEQ ID NO:42, a light chain CDR2 comprising the amino acid sequence of SEQ ID NO:43, and a light chain CDR3 comprising the amino acid sequence of SEQ ID NO:44;
i. a heavy chain CDR1 comprising the amino acid sequence of SEQ ID NO:46, a heavy chain CDR2 comprising the amino acid sequence of SEQ ID NO:47, a heavy chain CDR3 comprising the amino acid sequence of SEQ ID NO:48, a light chain CDR1 comprising the amino acid sequence of SEQ ID NO:50, a light chain CDR2 comprising the amino acid sequence of SEQ ID NO:51, and a light chain CDR3 comprising the amino acid sequence of SEQ ID NO:52;
j. a heavy chain CDR1 comprising the amino acid sequence of SEQ ID NO:54, a heavy chain CDR2 comprising the amino acid sequence of SEQ ID NO:55, a heavy chain CDR3 comprising the amino acid sequence of SEQ ID NO:56, a light chain CDR1 comprising the amino acid sequence of SEQ ID NO:58, a light chain CDR2 comprising the amino acid sequence of SEQ ID NO:59, and a light chain CDR3 comprising the amino acid sequence of SEQ ID NO:60;
k. a heavy chain CDR1 comprising the amino acid sequence of SEQ ID NO:54, a heavy chain CDR2 comprising the amino acid sequence of SEQ ID NO:55, a heavy chain CDR3 comprising the amino acid sequence of SEQ ID NO:62, a light chain CDR1 comprising the amino acid sequence of SEQ ID NO:58, a light chain CDR2 comprising the amino acid sequence of SEQ ID NO:59, and a light chain CDR3 comprising the amino acid sequence of SEQ ID NO:60;
l. a heavy chain CDR1 comprising the amino acid sequence of SEQ ID NO:64, a heavy chain CDR2 comprising the amino acid sequence of SEQ ID NO:19, a heavy chain CDR3 comprising the amino acid sequence of SEQ ID NO:65, a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8;
m. a heavy chain CDR1 comprising the amino acid sequence of SEQ ID NO:67, a heavy chain CDR2 comprising the amino acid sequence of SEQ ID NO:68, a heavy chain CDR3 comprising the amino acid sequence of SEQ ID NO:69, a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8;
n. a heavy chain CDR1 comprising the amino acid sequence of SEQ ID NO:10, a heavy chain CDR2 comprising the amino acid sequence of SEQ ID NO:71, a heavy chain CDR3 comprising the amino acid sequence of SEQ ID NO:72, a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8;
o. a heavy chain CDR1 comprising the amino acid sequence of SEQ ID NO:54, a heavy chain CDR2 comprising the amino acid sequence of SEQ ID NO:74, a heavy chain CDR3 comprising the amino acid sequence of SEQ ID NO:75, a light chain CDR1 comprising the amino acid sequence of SEQ ID NO:77, a light chain CDR2 comprising the amino acid sequence of SEQ ID NO:51, and a light chain CDR3 comprising the amino acid sequence of SEQ ID NO:78;
p. a heavy chain CDR1 comprising the amino acid sequence of SEQ ID NO:22, a heavy chain CDR2 comprising the amino acid sequence of SEQ ID NO:23, a heavy chain CDR3 comprising the amino acid sequence of SEQ ID NO:80, a light chain CDR1 comprising the amino acid sequence of SEQ ID NO:82, a light chain CDR2 comprising the amino acid sequence of SEQ ID NO:7, and a light chain CDR3 comprising the amino acid sequence of SEQ ID NO:83; or
q. a heavy chain CDR1 comprising the amino acid sequence of SEQ ID NO:22, a heavy chain CDR2 comprising the amino acid sequence of SEQ ID NO:85, a heavy chain CDR3 comprising the amino acid sequence of SEQ ID NO:86, a light chain CDR1 comprising the amino acid sequence of SEQ ID NO:88, a light chain CDR2 comprising the amino acid sequence of SEQ ID NO:43, and a light chain CDR3 comprising the amino acid sequence of SEQ ID NO:89;
wherein the CDRs are defined according to Kabat.

2. The isolated antibody, or antigen-binding fragment thereof, of claim 1 wherein the antibody, or antigen-binding fragment thereof, comprises a heavy chain CDR1 comprising the amino acid sequence of SEQ ID NO:14, a heavy chain CDR2 comprising the amino acid sequence of SEQ ID NO:15, a heavy chain CDR3 comprising the amino acid sequence of SEQ ID NO:16, a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8, wherein the CDRs are defined according to Kabat.

3. The isolated antibody, or antigen-binding fragment thereof, of claim 1 wherein the antibody, or antigen-binding fragment thereof, comprises a heavy chain CDR1 comprising the amino acid sequence of SEQ ID NO:54, a heavy chain CDR2 comprising the amino acid sequence of SEQ ID NO:55, a heavy chain CDR3 comprising the amino acid sequence of SEQ ID NO:62, a light chain CDR1 comprising the amino acid sequence of SEQ ID NO:58, a light chain CDR2 comprising the amino acid sequence of SEQ ID NO:59, and a light chain CDR3 comprising the amino acid sequence of SEQ ID NO:60, wherein the CDRs are defined according to Kabat.

4. The isolated antibody, or antigen-binding fragment thereof, of claim 1 wherein:
a. the antibody, or antigen-binding fragment thereof, of (a) has a heavy chain comprising an amino acid sequence that is at least 90% identical to the amino acid sequence of SEQ ID NO:1 and a light chain comprising an amino acid sequence that is at least 90% identical to the amino acid sequence of SEQ ID NO:5;
b. the antibody, or antigen-binding fragment thereof, of (b) has a heavy chain comprising an amino acid sequence that is at least 90% identical to the amino acid sequence of SEQ ID NO:9 and a light chain comprising an amino acid sequence that is at least 90% identical to the amino acid sequence of SEQ ID NO:5;
c. the antibody, or antigen-binding fragment thereof, of (c) has a heavy chain comprising an amino acid sequence that is at least 90% identical to the amino acid sequence of SEQ ID NO:13 and a light chain comprising an amino acid sequence that is at least 90% identical to the amino acid sequence of SEQ ID NO:5;
d. the antibody, or antigen-binding fragment thereof, of (d) has a heavy chain comprising an amino acid sequence that is at least 90% identical to the amino acid sequence of SEQ ID NO:17 and a light chain comprising an amino acid sequence that is at least 90% identical to the amino acid sequence of SEQ ID NO:5;
e. the antibody, or antigen-binding fragment thereof, of (e) has a heavy chain comprising an amino acid sequence that is at least 90% identical to the amino acid sequence of SEQ ID NO:21 and a light chain comprising an amino acid sequence that is at least 90% identical to the amino acid sequence of SEQ ID NO:5;
f. the antibody, or antigen-binding fragment thereof, of (0 has a heavy chain comprising an amino acid sequence that is at least 90% identical to the amino acid sequence of SEQ ID NO:25 and a light chain comprising an amino acid sequence that is at least 90% identical to the amino acid sequence of SEQ ID NO:29;
g. the antibody, or antigen-binding fragment thereof, of (g) has a heavy chain comprising an amino acid sequence that is at least 90% identical to the amino acid sequence of SEQ ID NO:33 and a light chain comprising an amino acid sequence that is at least 90% identical to the amino acid sequence of SEQ ID NO:36;
h. the antibody, or antigen-binding fragment thereof, of (h) has a heavy chain comprising an amino acid sequence that is at least 90% identical to the amino acid sequence of SEQ ID NO:39 and a light chain comprising an amino acid sequence that is at least 90% identical to the amino acid sequence of SEQ ID NO:41;
i. the antibody, or antigen-binding fragment thereof, of (i) has a heavy chain comprising an amino acid sequence that is at least 90% identical to the amino acid sequence of SEQ ID NO:45 and a light chain comprising an amino acid sequence that is at least 90% identical to the amino acid sequence of SEQ ID NO:49;
j. the antibody, or antigen-binding fragment thereof, of (j) has a heavy chain comprising an amino acid sequence that is at least 90% identical to the amino acid sequence of SEQ ID NO:53 and a light chain comprising an amino acid sequence that is at least 90% identical to the amino acid sequence of SEQ ID NO:57;
k. the antibody, or antigen-binding fragment thereof, of (k) has a heavy chain comprising an amino acid sequence that is at least 90% identical to the amino acid sequence of SEQ ID NO:61 and a light chain comprising an amino acid sequence that is at least 90% identical to the amino acid sequence of SEQ ID NO:57;
l. the antibody, or antigen-binding fragment thereof, of (l) has a heavy chain comprising an amino acid sequence that is at least 90% identical to the amino acid sequence of SEQ ID NO:63 and a light chain comprising an amino acid sequence that is at least 90% identical to the amino acid sequence of SEQ ID NO:5;
m. the antibody, or antigen-binding fragment thereof, of (m) has a heavy chain comprising an amino acid sequence that is at least 90% identical to the amino acid sequence of SEQ ID NO:66 and a light chain comprising an amino acid sequence that is at least 90% identical to the amino acid sequence of SEQ ID NO:5;
n. the antibody, or antigen-binding fragment thereof, of (n) has a heavy chain comprising an amino acid sequence that is at least 90% identical to the amino acid sequence of SEQ ID NO:70 and a light chain comprising an amino acid sequence that is at least 90% identical to the amino acid sequence of SEQ ID NO:5;
o. the antibody, or antigen-binding fragment thereof, of (o) has a heavy chain comprising an amino acid sequence that is at least 90% identical to the amino acid sequence of SEQ ID NO:73 and a light chain comprising an amino acid sequence that is at least 90% identical to the amino acid sequence of SEQ ID NO:76;
p. the antibody, or antigen-binding fragment thereof, of (p) has a heavy chain comprising an amino acid sequence that is at least 90% identical to the amino acid sequence of SEQ ID NO:79 and a light chain comprising an amino acid sequence that is at least 90% identical to the amino acid sequence of SEQ ID NO:81; or
q. the antibody, or antigen-binding fragment thereof, of (q) has a heavy chain comprising an amino acid sequence that is at least 90% identical to the amino acid sequence of SEQ ID NO:84 and a light chain comprising an amino acid sequence that is at least 90% identical to the amino acid sequence of SEQ ID NO:87.

5. The isolated antibody or antigen-binding portion thereof of claim 4, wherein the heavy chain has the amino acid sequence of SEQ ID NO:13 and a light chain has the amino acid sequence of SEQ ID NO:5.

6. The isolated antibody or antigen-binding portion thereof of claim 4, wherein the heavy chain has the amino acid sequence of SEQ ID NO:61 and a light chain has the amino acid sequence of SEQ ID NO:57.

7. An isolated antibody, or antigen-binding fragment thereof, that immunospecifically binds to ROR1, the antibody or antigen-binding fragment thereof comprising:
a. a heavy chain comprising an amino acid sequence that is at least 90% identical to the amino acid sequence of SEQ ID NO:1 and a light chain comprising an amino acid sequence that is at least 90% identical to the amino acid sequence of SEQ ID NO:5;
b. a heavy chain comprising an amino acid sequence that is at least 90% identical to the amino acid sequence of SEQ ID NO:9 and a light chain comprising an amino acid sequence that is at least 90% identical to the amino acid sequence of SEQ ID NO:5;
c. a heavy chain comprising an amino acid sequence that is at least 90% identical to the amino acid sequence of SEQ ID NO:13 and a light chain comprising an amino acid sequence that is at least 90% identical to the amino acid sequence of SEQ ID NO:5;
d. a heavy chain comprising an amino acid sequence that is at least 90% identical to the amino acid sequence of SEQ ID NO:17 and a light chain comprising an amino acid sequence that is at least 90% identical to the amino acid sequence of SEQ ID NO:5;
e. a heavy chain comprising an amino acid sequence that is at least 90% identical to the amino acid sequence of SEQ ID NO:21 and a light chain comprising an amino acid sequence that is at least 90% identical to the amino acid sequence of SEQ ID NO:5;
f. a heavy chain comprising an amino acid sequence that is at least 90% identical to the amino acid sequence of SEQ ID NO:25 and a light chain comprising an amino acid sequence that is at least 90% identical to the amino acid sequence of SEQ ID NO:29;
g. a heavy chain comprising an amino acid sequence that is at least 90% identical to the amino acid sequence of SEQ ID NO:33 and a light chain comprising an amino acid sequence that is at least 90% identical to the amino acid sequence of SEQ ID NO:36;
h. a heavy chain comprising an amino acid sequence that is at least 90% identical to the amino acid sequence of SEQ ID NO:39 and a light chain comprising an amino acid sequence that is at least 90% identical to the amino acid sequence of SEQ ID NO:41;
i. a heavy chain comprising an amino acid sequence that is at least 90% identical to the amino acid sequence of SEQ ID NO:45 and a light chain comprising an amino acid sequence that is at least 90% identical to the amino acid sequence of SEQ ID NO:49;
j. a heavy chain comprising an amino acid sequence that is at least 90% identical to the amino acid sequence of SEQ ID NO:53 and a light chain comprising an amino acid sequence that is at least 90% identical to the amino acid sequence of SEQ ID NO:57;
k. a heavy chain comprising an amino acid sequence that is at least 90% identical to the amino acid sequence of SEQ ID NO:61 and a light chain comprising an amino acid sequence that is at least 90% identical to the amino acid sequence of SEQ ID NO:57;
l. a heavy chain comprising an amino acid sequence that is at least 90% identical to the amino acid sequence of SEQ ID NO:63 and a light chain comprising an amino acid sequence that is at least 90% identical to the amino acid sequence of SEQ ID NO:5;
m. a heavy chain comprising an amino acid sequence that is at least 90% identical to the amino acid sequence of SEQ ID NO:66 and a light chain comprising an amino acid sequence that is at least 90% identical to the amino acid sequence of SEQ ID NO:5;
n. a heavy chain comprising an amino acid sequence that is at least 90% identical to the amino acid sequence of SEQ ID NO:70 and a light chain comprising an amino acid sequence that is at least 90% identical to the amino acid sequence of SEQ ID NO:5;
o. a heavy chain comprising an amino acid sequence that is at least 90% identical to the amino acid sequence of SEQ ID NO:73 and a light chain comprising an amino acid sequence that is at least 90% identical to the amino acid sequence of SEQ ID NO:76;
p. a heavy chain comprising an amino acid sequence that is at least 90% identical to the amino acid sequence of SEQ ID NO:79 and a light chain comprising an amino acid sequence that is at least 90% identical to the amino acid sequence of SEQ ID NO:81; or
q. a heavy chain comprising an amino acid sequence that is at least 90% identical to the amino acid sequence of SEQ ID NO:84 and a light chain comprising an amino acid sequence that is at least 90% identical to the amino acid sequence of SEQ ID NO:87.

8. An isolated antibody, or antigen-binding fragment thereof, that binds to an epitope on ROR1 comprising T324, V325, S326, V327, T328, S330, G331, R332, Q333, P336, N338, S339, Y341, H359, S360, Y361, L377, D378, and D387.

9. The isolated antibody, or antigen-binding fragment thereof, of any one of claims 1-8 wherein the antibody or antigen-binding fragment is a human antibody or antigen-binding fragment.

10. The isolated antibody, or antigen-binding fragment thereof, of any one of claims 1-9 wherein the antibody or antigen-binding fragment is recombinant.

11. The isolated antibody, or antigen-binding fragment thereof, of any one of claims 1-10 wherein the antigen-binding fragment is a Fab fragment, a Fab2 fragment, or a single chain antibody.

12. A nucleic acid molecule encoding the isolated antibody, or antigen-binding fragment thereof, of any one of claims 1-11.

13. A vector comprising the nucleic acid molecule of claim 12.

14. A cell expressing the isolated antibody, or antigen-binding fragment thereof, of any one of claims 1-11.

15. An isolated antibody, or antigen-binding fragment thereof, that competes for binding to ROR1 with a reference antibody or antigen-binding fragment thereof, the reference antibody or antigen-binding fragment thereof comprising:
a. a heavy chain comprising the amino acid sequence of SEQ ID NO:1 and a light chain comprising the amino acid sequence of SEQ ID NO:5;
b. a heavy chain comprising the amino acid sequence of SEQ ID NO:9 and a light chain comprising the amino acid sequence of SEQ ID NO:5;
c. a heavy chain comprising the amino acid sequence of SEQ ID NO:13 and a light chain comprising the amino acid sequence of SEQ ID NO:5;
d. a heavy chain comprising the amino acid sequence of SEQ ID NO:17 and a light chain comprising the amino acid sequence of SEQ ID NO:5;
e. a heavy chain comprising the amino acid sequence of SEQ ID NO:21 and a light chain comprising the amino acid sequence of SEQ ID NO:5;
f. a heavy chain comprising the amino acid sequence of SEQ ID NO:25 and a light chain comprising the amino acid sequence of SEQ ID NO:29;
g. a heavy chain comprising the amino acid sequence of SEQ ID NO:33 and a light chain comprising the amino acid sequence of SEQ ID NO:36;
h. a heavy chain comprising the amino acid sequence of SEQ ID NO:39 and a light chain comprising the amino acid sequence of SEQ ID NO:41;
i. a heavy chain comprising the amino acid sequence of SEQ ID NO:45 and a light chain comprising the amino acid sequence of SEQ ID NO:49;
j. a heavy chain comprising the amino acid sequence of SEQ ID NO:53 and a light chain comprising the amino acid sequence of SEQ ID NO:57;
k. a heavy chain comprising the amino acid sequence of SEQ ID NO:61 and a light chain comprising the amino acid sequence of SEQ ID NO:57;
l. a heavy chain comprising the amino acid sequence of SEQ ID NO:63 and a light chain comprising the amino acid sequence of SEQ ID NO:5;
m. a heavy chain comprising the amino acid sequence of SEQ ID NO:66 and a light chain comprising the amino acid sequence of SEQ ID NO:5;
n. a heavy chain comprising the amino acid sequence of SEQ ID NO:70 and a light chain comprising the amino acid sequence of SEQ ID NO:5;
o. a heavy chain comprising the amino acid sequence of SEQ ID NO:73 and a light chain comprising the amino acid sequence of SEQ ID NO:76;
p. a heavy chain comprising the amino acid sequence of SEQ ID NO:79 and a light chain comprising the amino acid sequence of SEQ ID NO:81; or
q. a heavy chain comprising the amino acid sequence of SEQ ID NO:84 and a light chain comprising the amino acid sequence of SEQ ID NO:87.

16. An isolated antibody, or antigen-binding fragment thereof, that competes for binding to ROR1 with a reference antibody or antigen-binding fragment thereof, wherein the reference antibody or antigen-binding fragment binds to an epitope on ROR1 comprising T324, V325, S326, V327, T328, S330, G331, R332, Q333, P336, N338, S339, Y341, H359, S360, Y361, L377, D378, and D387.

17. The isolated antibody or antigen-binding fragment thereof of claim 16, wherein the reference antibody comprises a heavy chain comprising the amino acid sequence of SEQ ID NO:13 and a light chain comprising the amino acid sequence of SEQ ID NO:5.

18. An isolated ROR1×CD3 bispecific antibody, or a bispecific antigen-binding fragment thereof, comprising:
a) a first antigen-binding site that immunospecifically binds ROR1, the first antigen-binding site comprising a heavy chain CDR1, CDR2, and CDR3 and a light chain CDR1, CDR2, and CDR3; and
b) a second antigen-binding site that immunospecifically binds CD3, the second antigen-binding site comprising a heavy chain CDR1, CDR2, and CDR3 and a light chain CDR1, CDR2, and CDR3.

19. The isolated ROR1×CD3 bispecific antibody, or bispecific antigen-binding fragment thereof, of claim 18, wherein the first antigen-binding site that immunospecifically binds ROR1 has:
a. a heavy chain CDR1 comprising the amino acid sequence of SEQ ID NO:2, a heavy chain CDR2 comprising the amino acid sequence of SEQ ID NO:3, a heavy chain CDR3 comprising the amino acid sequence of SEQ ID NO:4, a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8;
b. a heavy chain CDR1 comprising the amino acid sequence of SEQ ID NO:10, a heavy chain CDR2 comprising the amino acid sequence of SEQ ID NO:11, a heavy chain CDR3 comprising the amino acid sequence of SEQ ID NO:12, a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8;
c. a heavy chain CDR1 comprising the amino acid sequence of SEQ ID NO:14, a heavy chain CDR2 comprising the amino acid sequence of SEQ ID NO:15, a heavy chain CDR3 comprising the amino acid sequence of SEQ ID NO:16, a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8;
d. a heavy chain CDR1 comprising the amino acid sequence of SEQ ID NO:18, a heavy chain CDR2 comprising the amino acid sequence of SEQ ID NO:19, a heavy chain CDR3 comprising the amino acid sequence of SEQ ID NO:20, a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8;
e. a heavy chain CDR1 comprising the amino acid sequence of SEQ ID NO:22, a heavy chain CDR2 comprising the amino acid sequence of SEQ ID NO:23, a heavy chain CDR3 comprising the amino acid sequence of SEQ ID NO:24, a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8;
f. a heavy chain CDR1 comprising the amino acid sequence of SEQ ID NO:26, a heavy chain CDR2 comprising the amino acid sequence of SEQ ID NO:27, a heavy chain CDR3 comprising the amino acid sequence of SEQ ID NO:28, a light chain CDR1 comprising the amino acid sequence of SEQ ID NO:30, a light chain CDR2 comprising the amino acid sequence of SEQ ID NO:31, and a light chain CDR3 comprising the amino acid sequence of SEQ ID NO:32;
g. a heavy chain CDR1 comprising the amino acid sequence of SEQ ID NO:2, a heavy chain CDR2 comprising the amino acid sequence of SEQ ID NO:34, a heavy chain CDR3 comprising the amino acid sequence of SEQ ID NO:35, a light chain CDR1 comprising the amino acid sequence of SEQ ID NO:37, a light chain CDR2 comprising the amino acid sequence of SEQ ID NO:7, and a light chain CDR3 comprising the amino acid sequence of SEQ ID NO:38;
h. a heavy chain CDR1 comprising the amino acid sequence of SEQ ID NO:22, a heavy chain CDR2 comprising the amino acid sequence of SEQ ID NO:23, a heavy chain CDR3 comprising the amino acid sequence of SEQ ID NO:40, a light chain CDR1 comprising the amino acid sequence of SEQ ID NO:42, a light chain CDR2 comprising the amino acid sequence of SEQ ID NO:43, and a light chain CDR3 comprising the amino acid sequence of SEQ ID NO:44;
i. a heavy chain CDR1 comprising the amino acid sequence of SEQ ID NO:46, a heavy chain CDR2 comprising the amino acid sequence of SEQ ID NO:47, a heavy chain CDR3 comprising the amino acid sequence of SEQ ID NO:48, a light chain CDR1 comprising the amino acid sequence of SEQ ID NO:50, a light chain CDR2 comprising the amino acid sequence of SEQ ID NO:51, and a light chain CDR3 comprising the amino acid sequence of SEQ ID NO:52;
j. a heavy chain CDR1 comprising the amino acid sequence of SEQ ID NO:54, a heavy chain CDR2 comprising the amino acid sequence of SEQ ID NO:55, a heavy chain CDR3 comprising the amino acid sequence of SEQ ID NO:56, a light chain CDR1 comprising the amino acid sequence of SEQ ID NO:58, a light chain CDR2 comprising the amino acid sequence of SEQ ID NO:59, and a light chain CDR3 comprising the amino acid sequence of SEQ ID NO:60;
k. a heavy chain CDR1 comprising the amino acid sequence of SEQ ID NO:54, a heavy chain CDR2 comprising the amino acid sequence of SEQ ID NO:55, a heavy chain CDR3 comprising the amino acid sequence of SEQ ID NO:62, a light chain CDR1 comprising the amino acid sequence of SEQ ID NO:58, a light chain CDR2 comprising the amino acid sequence of SEQ ID NO:59, and a light chain CDR3 comprising the amino acid sequence of SEQ ID NO:60;
l. a heavy chain CDR1 comprising the amino acid sequence of SEQ ID NO:64, a heavy chain CDR2 comprising the amino acid sequence of SEQ ID NO:19, a heavy chain CDR3 comprising the amino acid sequence of SEQ ID NO:65, a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8;
m. a heavy chain CDR1 comprising the amino acid sequence of SEQ ID NO:67, a heavy chain CDR2 comprising the amino acid sequence of SEQ ID NO:68, a heavy chain CDR3 comprising the amino acid sequence of SEQ ID NO:69, a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8;
n. a heavy chain CDR1 comprising the amino acid sequence of SEQ ID NO:10, a heavy chain CDR2 comprising the amino acid sequence of SEQ ID NO:71, a heavy chain CDR3 comprising the amino acid sequence of SEQ ID NO:72, a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8;
o. a heavy chain CDR1 comprising the amino acid sequence of SEQ ID NO:54, a heavy chain CDR2 comprising the amino acid sequence of SEQ ID NO:74, a heavy chain CDR3 comprising the amino acid sequence of SEQ ID NO:75, a light chain CDR1 comprising the amino acid sequence of SEQ ID NO:77, a light chain CDR2 comprising the amino acid sequence of SEQ ID NO:51, and a light chain CDR3 comprising the amino acid sequence of SEQ ID NO:78;
p. a heavy chain CDR1 comprising the amino acid sequence of SEQ ID NO:22, a heavy chain CDR2 comprising the amino acid sequence of SEQ ID NO:23, a heavy chain CDR3 comprising the amino acid sequence of SEQ ID NO:80, a light chain CDR1 comprising the amino acid sequence of SEQ ID NO:82, a light chain CDR2 comprising the amino acid sequence of SEQ ID NO:7, and a light chain CDR3 comprising the amino acid sequence of SEQ ID NO:83; or
q. a heavy chain CDR1 comprising the amino acid sequence of SEQ ID NO:22, a heavy chain CDR2 comprising the amino acid sequence of SEQ ID NO:85, a heavy chain CDR3 comprising the amino acid sequence of SEQ ID NO:86, a light chain CDR1 comprising the amino acid sequence of SEQ ID NO:88, a light chain CDR2 comprising the amino acid sequence of SEQ ID NO:43, and a light chain CDR3 comprising the amino acid sequence of SEQ ID NO:89;
wherein the CDRs are defined according to Kabat.

20. The ROR1×CD3 bispecific antibody, or bispecific antigen-binding fragment thereof, of any one of claims 18-19, wherein the second antigen-binding site that immunospecifically binds CD3 has a heavy chain CDR1 comprising the amino acid sequence of SEQ ID NO:92, a heavy chain CDR2 comprising the amino acid sequence of SEQ ID NO:93, a heavy chain CDR3 comprising the amino acid sequence of SEQ ID NO:94, a light chain CDR1 comprising the amino acid sequence of SEQ ID NO:95, a light chain CDR2 comprising the amino acid sequence of SEQ ID NO:96, and a light chain CDR3 comprising the amino acid sequence of SEQ ID NO:97, wherein the CDRs are defined according to Kabat.

21. The isolated ROR1×CD3 bispecific antibody, or bispecific antigen-binding fragment thereof, of claim 20, wherein:
a. the first antigen-binding site that immunospecifically binds ROR1 has a heavy chain CDR1 comprising the amino acid sequence of SEQ ID NO:14, a heavy chain CDR2 comprising the amino acid sequence of SEQ ID NO:15, a heavy chain CDR3 comprising the amino acid sequence of SEQ ID NO:16, a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8; and
b. the second antigen-binding site that immunospecifically binds CD3 has a heavy chain CDR1 comprising the amino acid sequence of SEQ ID NO:92, a heavy chain CDR2 comprising the amino acid sequence of SEQ ID NO:93, a heavy chain CDR3 comprising the amino acid sequence of SEQ ID NO:94, a light chain CDR1 comprising the amino acid sequence of SEQ ID NO:95, a light chain CDR2 comprising the amino acid sequence of SEQ ID NO:96, and a light chain CDR3 comprising the amino acid sequence of SEQ ID NO:97,
wherein the CDRs are defined according to Kabat.

22. The isolated ROR1×CD3 bispecific antibody, or bispecific antigen-binding fragment thereof, of claim 20, wherein:
a. the first antigen-binding site that immunospecifically binds ROR1 has a heavy chain CDR1 comprising the amino acid sequence of SEQ ID NO:54, a heavy chain CDR2 comprising the amino acid sequence of SEQ ID NO:55, a heavy chain CDR3 comprising the amino acid sequence of SEQ ID NO:62, a light chain CDR1 comprising the amino acid sequence of SEQ ID NO:58, a light chain CDR2 comprising the amino acid sequence of SEQ ID NO:59, and a light chain CDR3 comprising the amino acid sequence of SEQ ID NO:60; and
b. the second antigen-binding site that immunospecifically binds CD3 has a heavy chain CDR1 comprising the amino acid sequence of SEQ ID NO:92, a heavy chain CDR2 comprising the amino acid sequence of SEQ ID NO:93, a heavy chain CDR3 comprising the amino acid sequence of SEQ ID NO:94, a light chain CDR1 comprising the amino acid sequence of SEQ ID NO:95, a light chain CDR2 comprising the amino acid sequence of SEQ ID NO:96, and a light chain CDR3 comprising the amino acid sequence of SEQ ID NO:97,
wherein the CDRs are defined according to Kabat.

23. An isolated ROR1×CD3 bispecific antibody, or a bispecific antigen-binding fragment thereof, comprising:
a) a first heavy chain (HC1);
b) a second heavy chain (HC2);
c) a first light chain (LC1); and
d) a second light chain (LC2),
wherein the HC1 and the LC1 form a first antigen-binding site that immunospecifically binds ROR1, and the HC2 and the LC2 form a second antigen-binding site that immunospecifically binds CD3.

24. The ROR1×CD3 bispecific antibody, or bispecific antigen-binding fragment thereof, of claim 23, wherein
a. the HC1 has a heavy chain CDR1 comprising the amino acid sequence of SEQ ID NO:2, a heavy chain CDR2 comprising the amino acid sequence of SEQ ID NO:3, a heavy chain CDR3 comprising the amino acid sequence of SEQ ID NO:4, and the LC1 has a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8;
b. the HC1 has a heavy chain CDR1 comprising the amino acid sequence of SEQ ID NO:10, a heavy chain CDR2 comprising the amino acid sequence of SEQ ID NO:11, a heavy chain CDR3 comprising the amino acid sequence of SEQ ID NO:12, and the LC1 has a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8;
c. the HC1 has a heavy chain CDR1 comprising the amino acid sequence of SEQ ID NO:14, a heavy chain CDR2 comprising the amino acid sequence of SEQ ID NO:15, a heavy chain CDR3 comprising the amino acid sequence of SEQ ID NO:16, and the LC1 has a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8;
d. the HC1 has a heavy chain CDR1 comprising the amino acid sequence of SEQ ID NO:18, a heavy chain CDR2 comprising the amino acid sequence of SEQ ID NO:19, a heavy chain CDR3 comprising the amino acid sequence of SEQ ID NO:20, and the LC1 has a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8;
e. the HC1 has a heavy chain CDR1 comprising the amino acid sequence of SEQ ID NO:22, a heavy chain CDR2 comprising the amino acid sequence of SEQ ID NO:23, a heavy chain CDR3 comprising the amino acid sequence of SEQ ID NO:24, and the LC1 has a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8;
f. the HC1 has a heavy chain CDR1 comprising the amino acid sequence of SEQ ID NO:26, a heavy chain CDR2 comprising the amino acid sequence of SEQ ID NO:27, a heavy chain CDR3 comprising the amino acid sequence of SEQ ID NO:28, and the LC1 has a light chain CDR1 comprising the amino acid sequence of SEQ ID NO:30, a light chain CDR2 comprising the amino acid sequence of SEQ ID NO:31, and a light chain CDR3 comprising the amino acid sequence of SEQ ID NO:32;
g. the HC1 has a heavy chain CDR1 comprising the amino acid sequence of SEQ ID NO:2, a heavy chain CDR2 comprising the amino acid sequence of SEQ ID NO:34, a heavy chain CDR3 comprising the amino acid sequence of SEQ ID NO:35, and the LC1 has a light chain CDR1 comprising the amino acid sequence of SEQ ID NO:37, a light chain CDR2 comprising the amino acid sequence of SEQ ID NO:7, and a light chain CDR3 comprising the amino acid sequence of SEQ ID NO:38;
h. the HC1 has a heavy chain CDR1 comprising the amino acid sequence of SEQ ID NO:22, a heavy chain CDR2 comprising the amino acid sequence of SEQ ID NO:23, a heavy chain CDR3 comprising the amino acid sequence of SEQ ID NO:40, and the LC1 has a light chain CDR1 comprising the amino acid sequence of SEQ ID NO:42, a light chain CDR2 comprising the amino acid sequence of SEQ ID NO:43, and a light chain CDR3 comprising the amino acid sequence of SEQ ID NO:44;
i. the HC1 has a heavy chain CDR1 comprising the amino acid sequence of SEQ ID NO:46, a heavy chain CDR2 comprising the amino acid sequence of SEQ ID NO:47, a heavy chain CDR3 comprising the amino acid sequence of SEQ ID NO:48, and the LC1 has a light chain CDR1 comprising the amino acid sequence of SEQ ID NO:50, a light chain CDR2 comprising the amino acid sequence of SEQ ID NO:51, and a light chain CDR3 comprising the amino acid sequence of SEQ ID NO:52;
j. the HC1 has a heavy chain CDR1 comprising the amino acid sequence of SEQ ID NO:54, a heavy chain CDR2 comprising the amino acid sequence of SEQ ID NO:55, a heavy chain CDR3 comprising the amino acid sequence of SEQ ID NO:56, and the LC1 has a light chain CDR1 comprising the amino acid sequence of SEQ ID NO:58, a light chain CDR2 comprising the amino acid sequence of SEQ ID NO:59, and a light chain CDR3 comprising the amino acid sequence of SEQ ID NO:60;
k. the HC1 has a heavy chain CDR1 comprising the amino acid sequence of SEQ ID NO:54, a heavy chain CDR2 comprising the amino acid sequence of SEQ ID NO:55, a heavy chain CDR3 comprising the amino acid sequence of SEQ ID NO:62, and the LC1 has a light chain CDR1 comprising the amino acid sequence of SEQ ID NO:58, a light chain CDR2 comprising the amino acid sequence of SEQ ID NO:59, and a light chain CDR3 comprising the amino acid sequence of SEQ ID NO:60;
l. the HC1 has a heavy chain CDR1 comprising the amino acid sequence of SEQ ID NO:64, a heavy chain CDR2 comprising the amino acid sequence of SEQ ID NO:19, a heavy chain CDR3 comprising the amino acid sequence of SEQ ID NO:65, and the LC1 has a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8;
m. the HC1 has a heavy chain CDR1 comprising the amino acid sequence of SEQ ID NO:67, a heavy chain CDR2 comprising the amino acid sequence of SEQ ID NO:68, a heavy chain CDR3 comprising the amino acid sequence of SEQ ID NO:69, and the LC1 has a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8;
n. the HC1 has a heavy chain CDR1 comprising the amino acid sequence of SEQ ID NO:10, a heavy chain CDR2 comprising the amino acid sequence of SEQ ID NO:71, a heavy chain CDR3 comprising the amino acid sequence of SEQ ID NO:72, and the LC1 has a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8;
o. the HC1 has a heavy chain CDR1 comprising the amino acid sequence of SEQ ID NO:54, a heavy chain CDR2 comprising the amino acid sequence of SEQ ID NO:74, a heavy chain CDR3 comprising the amino acid sequence of SEQ ID NO:75, and the LC1 has a light chain CDR1 comprising the amino acid sequence of SEQ ID NO:77, a light chain CDR2 comprising the amino acid sequence of SEQ ID NO:51, and a light chain CDR3 comprising the amino acid sequence of SEQ ID NO:78;
p. the HC1 has a heavy chain CDR1 comprising the amino acid sequence of SEQ ID NO:22, a heavy chain CDR2 comprising the amino acid sequence of SEQ ID NO:23, a heavy chain CDR3 comprising the amino acid sequence of SEQ ID NO:80, and the LC1 has a light chain CDR1 comprising the amino acid sequence of SEQ ID NO:82, a light chain CDR2 comprising the amino acid sequence of SEQ ID NO:7, and a light chain CDR3 comprising the amino acid sequence of SEQ ID NO:83; or
q. the HC1 has a heavy chain CDR1 comprising the amino acid sequence of SEQ ID NO:22, a heavy chain CDR2 comprising the amino acid sequence of SEQ ID NO:85, a heavy chain CDR3 comprising the amino acid sequence of SEQ ID NO:86, and the LC1 has a light chain CDR1 comprising the amino acid sequence of SEQ ID NO:88, a light chain CDR2 comprising the amino acid sequence of SEQ ID NO:43, and a light chain CDR3 comprising the amino acid sequence of SEQ ID NO:89;
wherein the CDRs are defined according to Kabat.

25. The ROR1×CD3 bispecific antibody, or bispecific antigen-binding fragment thereof, of claim 24, wherein
a. the HC1 of (a) comprises an amino acid sequence that is at least 90% identical to the amino acid sequence of SEQ ID NO:1 and the LC1 of (a) comprises an amino acid sequence that is at least 90% identical to the amino acid sequence of SEQ ID NO:5;
b. the HC1 of (b) comprises an amino acid sequence that is at least 90% identical to the amino acid sequence of SEQ ID NO:9 and the LC1 of (b) comprises an amino acid sequence that is at least 90% identical to the amino acid sequence of SEQ ID NO:5;
c. the HC1 of (c) comprises an amino acid sequence that is at least 90% identical to the amino acid sequence of SEQ ID NO:13 and the LC1 of (c) comprises an amino acid sequence that is at least 90% identical to the amino acid sequence of SEQ ID NO:5;
d. the HC1 of (d) comprises an amino acid sequence that is at least 90% identical to the amino acid sequence of SEQ ID NO:17 and the LC1 of (d) comprises an amino acid sequence that is at least 90% identical to the amino acid sequence of SEQ ID NO:5;
e. the HC1 of (e) comprises an amino acid sequence that is at least 90% identical to the amino acid sequence of SEQ ID NO:21 and the LC1 of (e) comprises an amino acid sequence that is at least 90% identical to the amino acid sequence of SEQ ID NO:5;
f. the HC1 of (f) comprises an amino acid sequence that is at least 90% identical to the amino acid sequence of SEQ ID NO:25 and the LC1 of (0 comprises an amino acid sequence that is at least 90% identical to the amino acid sequence of SEQ ID NO:29;
g. the HC1 of (g) comprises an amino acid sequence that is at least 90% identical to the amino acid sequence of SEQ ID NO:33 and the LC1 of (g) comprises an amino acid sequence that is at least 90% identical to the amino acid sequence of SEQ ID NO:36;
h. the HC1 of (h) comprises an amino acid sequence that is at least 90% identical to the amino acid sequence of SEQ ID NO:39 and the LC1 of (h) comprises an amino acid sequence that is at least 90% identical to the amino acid sequence of SEQ ID NO:41;
i. the HC1 of (i) comprises an amino acid sequence that is at least 90% identical to the amino acid sequence of SEQ ID NO:45 and the LC1 of (i) comprises an amino acid sequence that is at least 90% identical to the amino acid sequence of SEQ ID NO:49;
j. the HC1 of (j) comprises an amino acid sequence that is at least 90% identical to the amino acid sequence of SEQ ID NO:53 and the LC1 of (j) comprises an amino acid sequence that is at least 90% identical to the amino acid sequence of SEQ ID NO:57;
k. the HC1 of (k) comprises an amino acid sequence that is at least 90% identical to the amino acid sequence of SEQ ID NO:61 and the LC1 of (k) comprises an amino acid sequence that is at least 90% identical to the amino acid sequence of SEQ ID NO:57;
l. the HC1 of (l) comprises an amino acid sequence that is at least 90% identical to the amino acid sequence of SEQ ID NO:63 and the LC1 of (l) comprises an amino acid sequence that is at least 90% identical to the amino acid sequence of SEQ ID NO:5;
m. the HC1 of (m) comprises an amino acid sequence that is at least 90% identical to the amino acid sequence of SEQ ID NO:66 and the LC1 of (m) comprises an amino acid sequence that is at least 90% identical to the amino acid sequence of SEQ ID NO:5;
n. the HC1 of (n) comprises an amino acid sequence that is at least 90% identical to the amino acid sequence of SEQ ID NO:70 and the LC1 of (n) comprises an amino acid sequence that is at least 90% identical to the amino acid sequence of SEQ ID NO:5;
o. the HC1 of (o) comprises an amino acid sequence that is at least 90% identical to the amino acid sequence of SEQ ID NO:73 and the LC1 of (o) comprises an amino acid sequence that is at least 90% identical to the amino acid sequence of SEQ ID NO:76;
p. the HC1 of (p) comprises an amino acid sequence that is at least 90% identical to the amino acid sequence of SEQ ID NO:79 and the LC1 of (p) comprises an amino acid sequence that is at least 90% identical to the amino acid sequence of SEQ ID NO:81; or
q. the HC1 of (q) comprises an amino acid sequence that is at least 90% identical to the amino acid sequence of SEQ ID NO:84 and the LC1 of (q) comprises an amino acid sequence that is at least 90% identical to the amino acid sequence of SEQ ID NO:87.

26. The ROR1×CD3 bispecific antibody, or bispecific antigen-binding fragment thereof, of claim 23, wherein
a. the HC1 of (a) comprises an amino acid sequence that is at least 90% identical to the amino acid sequence of SEQ ID NO:1 and the LC1 of (a) comprises an amino acid sequence that is at least 90% identical to the amino acid sequence of SEQ ID NO:5;
b. the HC1 of (b) comprises an amino acid sequence that is at least 90% identical to the amino acid sequence of SEQ ID NO:9 and the LC1 of (b) comprises an amino acid sequence that is at least 90% identical to the amino acid sequence of SEQ ID NO:5;
c. the HC1 of (c) comprises an amino acid sequence that is at least 90% identical to the amino acid sequence of SEQ ID NO:13 and the LC1 of (c) comprises an amino acid sequence that is at least 90% identical to the amino acid sequence of SEQ ID NO:5;
d. the HC1 of (d) comprises an amino acid sequence that is at least 90% identical to the amino acid sequence of SEQ ID NO:17 and the LC1 of (d) comprises an amino acid sequence that is at least 90% identical to the amino acid sequence of SEQ ID NO:5;
e. the HC1 of (e) comprises an amino acid sequence that is at least 90% identical to the amino acid sequence of SEQ ID NO:21 and the LC1 of (e) comprises an amino acid sequence that is at least 90% identical to the amino acid sequence of SEQ ID NO:5;
f. the HC1 of (f) comprises an amino acid sequence that is at least 90% identical to the amino acid sequence of SEQ ID NO:25 and the LC1 of (0 comprises an amino acid sequence that is at least 90% identical to the amino acid sequence of SEQ ID NO:29;
g. the HC1 of (g) comprises an amino acid sequence that is at least 90% identical to the amino acid sequence of SEQ ID NO:33 and the LC1 of (g) comprises an amino acid sequence that is at least 90% identical to the amino acid sequence of SEQ ID NO:36;
h. the HC1 of (h) comprises an amino acid sequence that is at least 90% identical to the amino acid sequence of SEQ ID NO:39 and the LC1 of (h) comprises an amino acid sequence that is at least 90% identical to the amino acid sequence of SEQ ID NO:41;
i. the HC1 of (i) comprises an amino acid sequence that is at least 90% identical to the amino acid sequence of SEQ ID NO:45 and the LC1 of (i) comprises an amino acid sequence that is at least 90% identical to the amino acid sequence of SEQ ID NO:49;
j. the HC1 of (j) comprises an amino acid sequence that is at least 90% identical to the amino acid sequence of SEQ ID NO:53 and the LC1 of (j) comprises an amino acid sequence that is at least 90% identical to the amino acid sequence of SEQ ID NO:57;
k. the HC1 of (k) comprises an amino acid sequence that is at least 90% identical to the amino acid sequence of SEQ ID NO:61 and the LC1 of (k) comprises an amino acid sequence that is at least 90% identical to the amino acid sequence of SEQ ID NO:57;
l. the HC1 of (l) comprises an amino acid sequence that is at least 90% identical to the amino acid sequence of SEQ ID NO:63 and the LC1 of (l) comprises an amino acid sequence that is at least 90% identical to the amino acid sequence of SEQ ID NO:5;
m. the HC1 of (m) comprises an amino acid sequence that is at least 90% identical to the amino acid sequence of SEQ ID NO:66 and the LC1 of (m) comprises an amino acid sequence that is at least 90% identical to the amino acid sequence of SEQ ID NO:5;
n. the HC1 of (n) comprises an amino acid sequence that is at least 90% identical to the amino acid sequence of SEQ ID NO:70 and the LC1 of (n) comprises an amino acid sequence that is at least 90% identical to the amino acid sequence of SEQ ID NO:5;
o. the HC1 of (o) comprises an amino acid sequence that is at least 90% identical to the amino acid sequence of SEQ ID NO:73 and the LC1 of (o) comprises an amino acid sequence that is at least 90% identical to the amino acid sequence of SEQ ID NO:76;
p. the HC1 of (p) comprises an amino acid sequence that is at least 90% identical to the amino acid sequence of SEQ ID NO:79 and the LC1 of (p) comprises an amino acid sequence that is at least 90% identical to the amino acid sequence of SEQ ID NO:81; or
q. the HC1 of (q) comprises an amino acid sequence that is at least 90% identical to the amino acid sequence of SEQ ID NO:84 and the LC1 of (q) comprises an amino acid sequence that is at least 90% identical to the amino acid sequence of SEQ ID NO:87.

27. The ROR1×CD3 bispecific antibody, or bispecific antigen-binding fragment thereof, of any one of claims 23-26, wherein the HC2 has a heavy chain CDR1 comprising the amino acid sequence of SEQ ID NO:92, a heavy chain CDR2 comprising the amino acid sequence of SEQ ID NO:93, a heavy chain CDR3 comprising the amino acid sequence of SEQ ID NO:94, and the LC2 has a light chain CDR1 comprising the amino acid sequence of SEQ ID NO:95, a light chain CDR2 comprising the amino acid sequence of SEQ ID NO:96, and a light chain CDR3 comprising the amino acid sequence of SEQ ID NO:97, wherein the CDRs are defined according to Kabat.

28. The ROR1×CD3 bispecific antibody, or bispecific antigen-binding fragment thereof, of any one of claims 23-27, wherein the HC2 comprises an amino acid sequence that is at least 90% identical to SEQ ID NO:90 and the LC2 comprises an amino acid sequence that is at least 90% identical to SEQ ID NO:91.

29. The ROR1×CD3 bispecific antibody, or bispecific antigen-binding fragment thereof, of any one of claims 23-27, wherein:
a. the HC1 has a heavy chain CDR1 comprising the amino acid sequence of SEQ ID NO:14, a heavy chain CDR2 comprising the amino acid sequence of SEQ ID NO:15, a heavy chain CDR3 comprising the amino acid sequence of SEQ ID NO:16, and the LC1 has a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8; and
b. the HC2 has a heavy chain CDR1 comprising the amino acid sequence of SEQ ID NO:92, a heavy chain CDR2 comprising the amino acid sequence of SEQ ID NO:93, a heavy chain CDR3 comprising the amino acid sequence of SEQ ID NO:94, and the LC2 has a light chain CDR1 comprising the amino acid sequence of SEQ ID NO:95, a light chain CDR2 comprising the amino acid sequence of SEQ ID NO:96, and a light chain CDR3 comprising the amino acid sequence of SEQ ID NO:97,
wherein the CDRs are defined according to Kabat.

30. The ROR1×CD3 bispecific antibody, or bispecific antigen-binding fragment thereof, of any one of claims 23-27, wherein:
a. the HC1 has a heavy chain CDR1 comprising the amino acid sequence of SEQ ID NO:54, a heavy chain CDR2 comprising the amino acid sequence of SEQ ID NO:55, a heavy chain CDR3 comprising the amino acid sequence of SEQ ID NO:62, and the LC1 has a light chain CDR1 comprising the amino acid sequence of SEQ ID NO:58, a light chain CDR2 comprising the amino acid sequence of SEQ ID NO:59, and a light chain CDR3 comprising the amino acid sequence of SEQ ID NO:60; and
b. the HC2 has a heavy chain CDR1 comprising the amino acid sequence of SEQ ID NO:92, a heavy chain CDR2 comprising the amino acid sequence of SEQ ID NO:93, a heavy chain CDR3 comprising the amino acid sequence of SEQ ID NO:94, and the LC2 has a light chain CDR1 comprising the amino acid sequence of SEQ ID NO:95, a light chain CDR2 comprising the amino acid sequence of SEQ ID NO:96, and a light chain CDR3 comprising the amino acid sequence of SEQ ID NO:97,
wherein the CDRs are defined according to Kabat.

31. The ROR1×CD3 bispecific antibody, or bispecific antigen-binding fragment thereof, of any one of claims 23-28, wherein:
a. the HC1 has the amino acid sequence of SEQ ID NO:13 and the LC1 has the amino acid sequence of SEQ ID NO:5; and
b. the HC2 has the amino acid sequence of SEQ ID NO:90 and the LC2 has the amino acid sequence of SEQ ID NO:91.

32. The ROR1×CD3 bispecific antibody, or bispecific antigen-binding fragment thereof, of any one of claims 23-28, wherein:
a. the HC1 has the amino acid sequence of SEQ ID NO:61 and the LC1 has the amino acid sequence of SEQ ID NO:57; and
b. the HC2 has the amino acid sequence of SEQ ID NO:90 and the LC2 has the amino acid sequence of SEQ ID NO:91.

33. The ROR1×CD3 bispecific antibody, or bispecific antigen-binding fragment thereof, of any one of claims 18-32, wherein the bispecific antigen-binding fragment is a single chain.

34. An isolated cell expressing the ROR1×CD3 bispecific antibody, or bispecific antigen-binding fragment thereof, of any one of claims 18-33.

35. A method of treating a subject having cancer, the method comprising:
administering to the subject a therapeutically effective amount of the ROR1×CD3 bispecific antibody, or bispecific antigen-binding fragment thereof, of any one of claims 18-33.

36. The method of claim 35, wherein the cancer is lung cancer or a hematological cancer.

37. Use of an effective amount of a ROR1×CD3 bispecific antibody or bispecific antigen-binding fragment thereof of any one of claims 18-33 in the treatment of cancer.

38. Use of a ROR1×CD3 bispecific antibody or bispecific antigen-binding fragment thereof of any one of claims 18-33 in the manufacture of a composition for the treatment of cancer.

39. The use of claim 37 or 38, wherein the cancer is lung cancer, hematological cancer, breast cancer, prostate cancer, pancreatic cancer, colon cancer, ovarian cancer, renal cancer, uterine cancer, or melanoma.
